(19) United States
(12) Patent Application Publication
(10) Pub. No.: US 2026/0132469 A1
(43) Pub. Date: May 14, 2026

(54) MULTI-ANALYTE DIAGNOSTIC AND PROGNOSTIC ASSAYS FOR COLORECTAL CANCER AND USES THEREOF

(71) Applicant: NEW DAY DIAGNOSTICS LLC, Knoxville, TN (US)

(72) Inventors: Andrew A. LUKOWIAK, Berlin (DE); Stephani D. HERNANDEZ, San Diego, CA (US); Denise KOTTWITZ, Berlin (DE); Jörn LEWIN, Berlin (DE); Uwe STAUB, Berlin (DE)

(21) Appl. No.: 19/118,741

(22) PCT Filed: Oct. 3, 2023
(86) PCT No.: PCT/IB2023/059933

§ 371 Date: Apr. 4, 2025

Related U.S. Application Data
(60) Provisional application No. 63/413,043, filed on Oct. 4, 2022.
(60) Provisional application No. 63/381,706, filed on Oct. 31, 2022.

(51) Int. Cl. C12Q 1/6886; G01N 33/575; G01N 33/57535

(52) U.S. Cl. CPC C12Q 1/6886; G01N 33/57535; G01N 33/57565; C12Q 2600/154

(57) ABSTRACT

Provided herein are methods, assays and kits for detecting methylated genomic DNA and one or more protein biomarkers in one or more biological samples. In some aspects, the disclosure provides methods, assays and kits for detecting or prognosing a proliferative disease, such as colorectal cancer, in a subject based on the presence of methylated genomic DNA and one or more protein biomarkers in one or more biological samples.

### CROSS-REFERENCE TO RELATED APPLICATIONS

This application claims priority to, and benefit of, U.S. Provisional Application No. 63/413,043, filed on Oct. 4, 2022 and 63/381,706, filed on Oct. 31, 2022, the contents of each of which are incorporated by reference herein in their entireties.

### REFERENCE TO AN ELECTRONIC SEQUENCE LISTING

The contents of the electronic sequence listing (EPAG_001_001WO_SeqList_ST26.xml; Size: 436,187 bytes; and Date of Creation: Sep. 28, 2023) are herein incorporated by reference in its entirety.

### BACKGROUND

Cancer is the second most frequent cause of premature death in the US with colorectal cancer (CRC) being one of the most commonly diagnosed cancers in the west (Murphy, S. L. et al., 2020. NCHS Data Brief, no 427. Hyattsville, MD: National Center for Health Statistics. 2021. doi: dx.doi.org/10.15620/cdc:112079external icon.).

Cancer stages are classified in terms of extent, progression, and severity and groups cancer patients so that generalizations can be made about prognosis and choice of therapy. Colorectal cancer typically advances from benign adenomas (polyps) to malignant carcinomas. One of the greatest determining factors in a patient's chance of survival is early diagnosis and subsequent treatment of CRC. Malignant tumors of the colorectum arise from benign tumors, i.e., from adenoma. Therefore, the best prognoses are from those patients diagnosed at the adenoma stage. Furthermore, the prognosis in advanced colon cancer stages (e.g. Distant SEER stage) is poor and corresponds to a five year relative survival rate of about 14% (SEER cancer.org/cancer/colon-rectal-cancer/detection-diagnosis-staging/survival-rates.html).

Current treatment is only curing a fraction of the patients and clearly has the best effect on those patients diagnosed in an early stage of disease. Current procedures available for early CRC detection involve endoscopic procedures and/or fecal occult blood tests (FOBT). However, these methods can be invasive and have their limitations. FOBT are unreliable for early detection as tumors must typically reach a significant size before blood is detected in feces. The sensitivity of the guaiac-based FOBT is ˜24%, which means nearly ¾ths of patients with malignant lesions will remain undiagnosed and thus untreated (Parra-Blanco A, et al., J Gastroenterol. 2010 July; 45(7):703-12. doi: 10.1007/s00535-010-0214-8. Epub 2010 Feb. 17. PMID: 20157748). In contrast to FOBT, visualization of precancerous and cancerous lesions via colonoscopy is a highly reliable approach for early detection. However, colonoscopies are invasive and carry significant costs and potential for complications. As such, colonoscopies are only recommended every 5-10 years for most patients.

Accordingly, there is a need for methods for the early detection of CRC and other cancers that are also sensitive, specific, noninvasive, and cost-effective.

### SUMMARY OF THE DISCLOSURE

The present disclosure is based in part on the discovery that detecting a combination of methylated DNA and protein biomarkers improves the sensitivity of a method or assay for diagnosing or prognosing a subject with a proliferative disease (e.g., cancer).

Accordingly, in some aspects, the disclosure provides a method for detecting the presence or amount of methylated genomic DNA and protein in one or more biological samples from a subject, comprising:
(i) detecting synthetic DNA generated from one or more methylated genomic DNA sequences associated with a proliferative disease from the one or more biological samples, wherein the synthetic DNA is generated by converting cytosine unmethylated in the 5-position to uracil or another base that does not hybridize to guanine in the genomic DNA, and detecting unconverted cytosine in the synthetic DNA; and
(ii) detecting the presence or amount of one or more protein biomarkers in the one or more biological samples, wherein the one or more protein biomarkers comprise carcinoembryonic antigen (CEA) and/or amphiregulin.

In some embodiments, the proliferative disease is a cancer. In some embodiments, the proliferative disease is a colorectal cancer or a colorectal cell proliferative disease.

In some or any of the foregoing or related embodiments, the one or more methylated genomic DNA sequences associated with a proliferative disease is methylated Septin-9 (mSEPT9). In some embodiments, mSEPT9 comprises one or more CpG dinucleotides within the sequence set forth in SEQ ID NO: 31. In some embodiments, the synthetic DNA is generated using at least one nucleic acid molecule comprising a contiguous sequence at least 9 nucleotides in length that is complementary to, or hybridizes to, SEQ ID NO: 31. In some embodiments, the nucleic acid molecule is a methylation-specific oligonucleotide.

In some or any of the foregoing or related embodiments, the one or more methylated genomic DNA sequences associated with a proliferative disease is methylated ANKRD13B (mANKRD13B). In some embodiments, mANKRD13B comprises one or more CpG dinucleotides within the sequence set forth in SEQ ID NO: 6 or 11. In some embodiments, the synthetic DNA is generated using at least one nucleic acid molecule comprising a contiguous sequence at least 9 nucleotides in length that is complementary to, or hybridizes to, SEQ ID NO: 6 or 11. In some embodiments, the nucleic acid molecule is a methylation-specific oligonucleotide.

In some or any of the foregoing or related embodiments, synthetic DNA is generated by treating the genomic DNA with bisulfite to produce sulfonated DNA.

In some aspects, the disclosure provides a method for detecting the presence or amount of methylated genomic DNA and protein in one or more biological samples, comprising:
(i) detecting DNA methylation within at least one genomic DNA polynucleotide in the one or more biological samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin.

In other aspects, the disclosure provides a method for detecting the presence or amount of methylated genomic DNA and protein in one or more biological samples, comprising:
(i) detecting DNA methylation within SEQ ID NO: 31 (mSEPT9) and SEQ ID NOs: 6 and 11 (mANKDRD13b) in the one or more biological samples, wherein cytosine unmethylated in the 5-position is converted to uracil or another base that does not hybridize to guanine, and unconverted cytosine is detected; and more biological samples, wherein the one or more protein biomarkers comprise CEA and/or amphiregulin.

In some or any of the foregoing or related embodiments, the biological sample is from a subject at risk or suspected of being at risk for having or developing colorectal cancer or a colorectal cell proliferative disease.

In some or any of the foregoing or related embodiments, the one or more protein biomarkers comprise CEA. In some embodiments, the one or more protein biomarkers comprise amphiregulin. In some embodiments, the one or more protein biomarkers comprise CEA and amphiregulin. In some embodiments, the one or more protein biomarkers further comprise CYFRA21-1. In some embodiments, the one or more protein biomarkers further comprise a galectin-3 ligand. In some embodiments, the galectin-3 ligand is haptoglobin.

In some or any of the foregoing or related embodiments, determining the presence or amount of the one or more protein biomarkers comprises contacting the one or more biological samples with an antibody specific for the one or more protein biomarkers, and detecting the antibody. In some embodiments, determining the presence or amount of the one or more protein biomarkers comprises detecting by western blot, enzyme-linked immunosorbent assay (ELISA), immunobead-based format, proximity extension assay (PEA) or mass-spectrometry.

In some or any of the foregoing or related embodiments, the biological sample comprises genomic DNA from a circulating cancer cell. In some embodiments, the one or more biological samples is a blood sample, a serum sample, a plasma sample, a urine sample, a saliva sample, a stool sample, or a combination thereof. In some embodiments, the one or more biological samples is a blood sample, a serum sample, a plasma sample, or a combination thereof. In some embodiments, the one or more biological samples are the same biological sample. In some embodiments, the one or more biological samples are different biological samples. In some embodiments, the one or more biological samples are the same biological sample, separated into two or more separate samples; one sample for detecting the presence of one or more methylated genomic DNA sequences, and one sample for detecting the presence of one or more protein biomarkers.

In some or any of the foregoing or related embodiments, the method further comprises diagnosing the subject as having a proliferative disease, e.g., cancer (e.g., colorectal cancer) or a precancerous lesion (e.g., an adenoma). In some embodiments, the presence of one or more methylated genomic DNA sequences and one or more protein biomarkers is indicative of the presence of a proliferative disease in a subject, or indicative of the presence of a risk of a subject having a proliferative disease. In some embodiments, the method detects proliferative disease in a subject with a sensitivity of at least 0.65 and/or a specificity of at least 0.8. In some embodiments, the method detects proliferative disease in a subject with a sensitivity of at least 0.74 and/or a specificity of at least 0.9. In some embodiments, the method detects proliferative disease in a subject with a sensitivity of at least 0.7-0.75 and/or a specificity of at least 0.85-0.9.

In some or any of the foregoing or related embodiments, the one or more protein biomarkers are detected by a manual, semi-automated, or fully automated system. In some embodiments, the methylated genomic DNA is detected by a manual, semi-automated, or fully automated system. In some embodiments, the one or more protein biomarkers are detected by a monoplex or multiplex system.

In some aspects, the disclosure provides a method for detecting the presence or amount of methylated genomic DNA and protein in one or more biological samples from a subject, comprising:
(i) detecting one or more methylated genomic DNA sequences associated with a proliferative disease from the one or more biological samples; and
(ii) detecting the presence or amount of one or more protein biomarkers in the one or more biological samples, wherein the one or more protein biomarkers comprise CEA and/or amphiregulin.

In some aspects, the disclosure provides a method for detecting the presence or amount of methylated genomic DNA and protein in one or more biological samples from a subject, comprising:
(i) detecting one or more methylated genomic DNA sequences associated with a proliferative disease from the one or more biological samples; and
(ii) detecting the presence or amount of one or more protein biomarkers in the one or more biological samples, wherein the one or more protein biomarkers comprise CEA and/or amphiregulin, and a galectin ligand. In some embodiments, the galectin-3 ligand is haptoglobin.

In further aspects, the disclosure provides a method for detecting the presence or amount of methylated genomic DNA and protein in one or more biological samples, comprising:
(i) detecting DNA methylation within at least one genomic DNA polynucleotide in the one or more biological samples, wherein the at least one genomic DNA polynucleotide comprises mSEPT9 and/or mANKDRD13b; and
(ii) detecting the presence or amount of one or more protein biomarkers, wherein the one or more protein biomarkers comprise CEA and/or amphiregulin.

In other aspects, the disclosure provides a method for detecting the presence or amount of methylated genomic DNA and protein in one or more biological samples, comprising:
(i) detecting DNA methylation within SEQ ID NO: 31 (mSEPT9) and SEQ ID NOs: 6 and 11 (mANKDRD13b) in the one or more biological samples; and
(ii) detecting the presence or amount of one or more protein biomarkers in the one or more biological samples, wherein the one or more protein biomarkers comprise CEA and/or amphiregulin.

In some aspects, the disclosure provides a method for detecting colorectal cancer or a colorectal cell proliferative disease in a subject, comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject.

In some or any of the foregoing or related aspects, the one or more protein biomarkers comprise a galectin-3 ligand. In some embodiments, the one or more protein biomarkers comprise (i) CEA and the galectin-3 ligand; (ii) amphiregulin and the galectin-3 ligand; or (iii) CEA, amphiregulin and the galectin-3 ligand. In some embodiments, the galectin-3 ligand is haptoglobin.

In further aspects, the disclosure provides, a method for detecting colorectal cancer or a colorectal cell proliferative disease in a subject, comprising:
(i) detecting DNA methylation within at least one genomic DNA polynucleotide in one or more plasma or serum samples from the subject, wherein the at least one genomic DNA polynucleotide comprises mSEPT9 and mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more plasma or serum samples, wherein the one or more protein biomarkers comprise CEA and/or amphiregulin,
wherein the presence of DNA methylation and one or more protein biomarkers in the one or more plasma or serum samples is indicative of the presence of colorectal cancer or a colorectal cell proliferative disease in the subject.

In some aspects, the disclosure provides, a method for detecting colorectal cancer or a colorectal cell proliferative disease in a subject, comprising:
(i) detecting DNA methylation within at least one genomic DNA polynucleotide in one or more plasma or serum samples from the subject, wherein the at least one genomic DNA polynucleotide comprises mSEPT9 and mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more plasma or serum samples, wherein the one or more protein biomarkers comprise CEA and/or amphiregulin, and a galectin-3 ligand,
wherein the presence of DNA methylation and one or more protein biomarkers in the one or more plasma or serum samples is indicative of the presence of colorectal cancer or a colorectal cell proliferative disease in the subject. In some embodiments, the galectin-3 ligand is haptoglobin.

In some or any of the foregoing or related embodiments, colorectal cancer or the colorectal cell proliferative disease is detected in the subject with a sensitivity of at least 0.65 and/or a specificity of at least 0.8. In some embodiments, colorectal cancer or the colorectal cell proliferative disease is detected in the subject with a sensitivity of at least 0.74 and/or a specificity of at least 0.9. In some embodiments, colorectal cancer or the colorectal cell proliferative disease is detected in the subject with a sensitivity of at least 0.7-0.75 and/or a specificity of at least 0.85-0.9. In some embodiments, colorectal cancer or the colorectal cell proliferative disease is detected in the subject with a sensitivity of at least 0.8 and/or a specificity of at least 0.85-0.9.

In some aspects, the disclosure provides a method for prognosing colorectal cancer or a colorectal cell proliferative disease in a subject, comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise CEA and/or amphiregulin; and
(iii) prognosing the subject with colorectal cancer or a colorectal cell proliferative disease based on the presence of DNA methylation and one or more protein biomarkers in the one or more biological samples.

In some aspects, the disclosure provides a method for prognosing colorectal cancer or a colorectal cell proliferative disease in a subject, comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise CEA and/or amphiregulin, and a galectin-3 ligand; and
(iii) prognosing the subject with colorectal cancer or a colorectal cell proliferative disease based on the presence of DNA methylation and one or more protein biomarkers in the one or more biological samples. In some embodiments, the galectin-3 ligand is haptoglobin.

In other aspects, the disclosure provides a method for prognosing colorectal cancer or a colorectal cell proliferative disease in a subject, comprising:
(i) detecting DNA methylation within at least one genomic DNA polynucleotide in one or more plasma or serum samples from the subject, wherein the at least one genomic DNA polynucleotide comprises mSEPT9 and mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more plasma or serum samples, wherein the one or more protein biomarkers comprise CEA and/or amphiregulin; and
(iii) prognosing the subject with colorectal cancer or a colorectal cell proliferative disease based on the presence of DNA methylation and one or more protein biomarkers in the one or more serum or plasma samples.

In other aspects, the disclosure provides a method for prognosing colorectal cancer or a colorectal cell proliferative disease in a subject, comprising:
(i) detecting DNA methylation within at least one genomic DNA polynucleotide in one or more plasma or serum samples from the subject, wherein the at least one genomic DNA polynucleotide comprises mSEPT9 and mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more plasma or serum samples, wherein the one or more protein biomarkers comprise CEA and/or amphiregulin, and a galectin-3 ligand; and
(iii) prognosing the subject with colorectal cancer or a colorectal cell proliferative disease based on the presence of DNA methylation and one or more protein biomarkers in the one or more serum or plasma samples. In some embodiments, the galectin-3 ligand is haptoglobin.

In some or any of the foregoing or related embodiments, the method further comprises performing a colonoscopy on the subject to confirm the presence of colorectal cancer or the colorectal cell proliferative disease. In some embodiments, the method further comprises treating the subject after confirmation of the presence of colorectal cancer or the colorectal cell proliferative disease in the subject. In some embodiments, treating the subject comprises surgery, radiation therapy, chemotherapy, or immunotherapy.

In some aspects, the disclosure provides a kit suitable for performing a method described herein, comprising (i) an agent for converting cytosine unmethylated in the 5-position to uracil or another base that does not hybridize to guanine in the genomic DNA; (ii) at least one set of oligonucleotides complementary to the genomic DNA; and (iii) instructions for detecting methylation of genomic DNA in one or more biological samples in combination with instructions for detecting the one or more protein biomarkers in the one or more biological samples, wherein the one or more protein biomarkers is CEA and/or amphiregulin.

In some aspects, the disclosure provides a kit suitable for performing a method described herein, comprising (i) one or more reagents for detecting the one or more protein biomarkers, wherein the one or more protein biomarkers is CEA and/or amphiregulin; and (ii) instructions for detecting the presence or amount of the one or more protein biomarkers in one or more biological samples in combination with instructions for detecting methylated genomic DNA.

In some aspects, the disclosure provides a kit suitable for performing a method described herein, comprising (i) an agent for converting cytosine unmethylated in the 5-position to uracil or another base that does not hybridize to guanine in the genomic DNA; (ii) at least one set of oligonucleotides complementary to the genomic DNA; (iii) one or more reagents for detecting the one or more protein biomarkers, wherein the one or more protein biomarkers is CEA and/or amphiregulin; and (iv) instructions for detecting methylation of genomic DNA and the presence or amount of the one or more protein biomarkers in one or more biological samples.

In some or any of the foregoing or related embodiments, the instructions comprise steps for diagnosing, prognosing, or classifying colorectal cancer or a colorectal cell proliferative disease based on the detection of methylated genomic DNA and the one or more protein biomarkers. In some embodiments, the one or more biological samples is a blood sample, a serum sample, a plasma sample, a urine sample, a saliva sample, or a stool sample. In some embodiments, the one or more biological samples is a blood sample, a serum sample, or a plasma sample. In some embodiments, the one or more biological samples is obtained from a subject at risk or suspected of being at risk for having or developing colorectal cancer or a colorectal cell proliferative disease.

In some aspects, the disclosure provides a method for detecting a pre-cancerous lesion (e.g., an advanced adenoma) in a subject, comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA and/or amphiregulin, and/or a galectin-3 ligand,
wherein the presence of DNA methylation and one or more protein biomarkers in the one or more biological samples is indicative of the presence of a pre-cancerous lesion in the subject.

In some aspects, the disclosure provides a method for detecting a pre-cancerous lesion (e.g., an advanced adenoma) in a subject, comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA and/or amphiregulin, and a galectin-3 ligand,
wherein the presence of DNA methylation and one or more protein biomarkers in the one or more biological samples is indicative of the presence of a pre-cancerous lesion in the subject.

In some aspects, the disclosure provides a method for detecting a pre-cancerous lesion (e.g., an advanced adenoma) in a subject, comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA, amphiregulin, and/or a galectin-3 ligand,
wherein the presence of DNA methylation and one or more protein biomarkers in the one or more biological samples is indicative of the presence of a pre-cancerous lesion in the subject.

### BRIEF DESCRIPTION OF THE FIGURES

FIG. 1 provides a map of target regions in ANKRD13B. See Table 3 for an explanation of the SEQ ID NOs.

FIG. 2 provides a map of target regions in SEPTIN9. See Table 3 for an explanation of the SEQ ID NOs.

FIGS. 3A-3B show primer pairs (listed as A/B, D/E, and G/H) and probe oligomer (C, F, I) either directly or—if matching the antisense strand—as reverse complement, their SEQ ID NO, their oligomer type, their name, their start and end position within the assay, and information for the strand they match on in relation to the genomic reference sequence of the amplified assay. Bisulfite specific base pairing (T on C for converted sense strand, G on A for converted antisense strand) to the genomic reference is displayed by ‘+’. FIG. 3A shows methylation specific assays for ANKRD13BoB1 and ANKRD13BoB2. FIG. 3B shows methylation specific assays for SEPTIN9oB2.

FIGS. 4-13 provide graphs showing receiver operating characteristic (ROC) curves from blood plasma samples based on assessment of single markers and marker combination in the same order as follows: ANB1, ANB2, S9B2, CEA, AR, CYPHRA, ANB1+ANB2+S9B2, ANB1+ANB2+S9B2+CEA, ANB1+ANB2+S9B2+AR, ANB1+ANB2+S9B2+CEA+AR. The Sensitivity at a Specificity of 0.9 is shown and the values are provided in the plot. Numbers in the right lower corner show area under the curve (AUC) of ROC curves. ANB1=ANKRD13BoB1; ANB2=ANKRD13BoB2; S9B2=SEPTIN9oB2; CEA=carcinoembryonic antigen; AR=amphiregulin; CYPHRA=CYFRA21-1; FPR=false positive rate; TPR=true positive rate.

FIGS. 14-18 provide graphs showing ROC curves from blood plasma samples based on assessment of single markers and marker combination in the same order as followings: HG, ANB1+ANB2+S9B2+HG, ANB1+ANB2+S9B2+CEA+HG, ANB1+ANB2+S9B2+AR+HG, and ANB1+ANB2+S9B2+CEA+AR+HG. The Sensitivity at a Specificity of 0.9 is shown and the values are provided in the plot. Numbers in the right lower corner show area under the curve (AUC) of ROC curves. ANB1=ANKRD13BoB1; ANB2=ANKRD13BoB2; S9B2=SEPTIN9oB2; CEA=carcinoembryonic antigen; AR=amphiregulin; HG=haptoglobin; FPR=false positive rate; TPR=true positive rate.

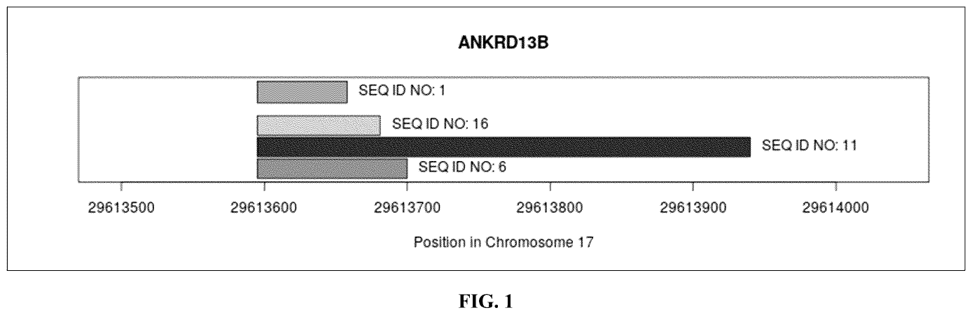

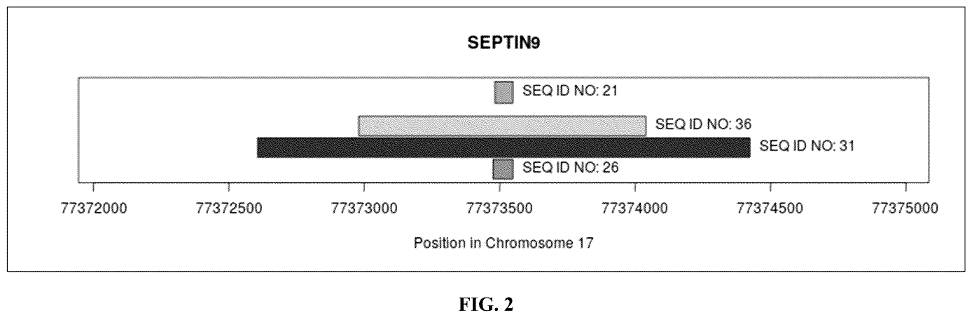

### DETAILED DESCRIPTION

Colonoscopy is the standard for colorectal cancer screening today as it provides the highest likelihood for detecting cancerous and precancerous lesions. However, only ˜70% of the average-risk population in the US is up-to-date with the current screening guidelines, as not all screening eligible patients are willing to participate in an invasive screening methodology (colonoscopy) for a variety of reasons, including inconvenience, discomfort, cost and fear. Accordingly, non-invasive methods and assays for diagnosing or prognosing a subject with a proliferative disorder with sufficient discriminatory ability are needed to improve patient compliance with screening.

At least one blood test is currently being used for colorectal cancer screening, Epi proColon, which detects the methylation status of Septin-9 (SEPT9) in plasma. The present disclosure provides methods, compositions, assays and kits having improved discriminatory ability by detecting methylation status of methylated genomic DNA in combination with detecting the presence or amount of one or more protein biomarkers. As demonstrated herein, detecting methylated SEPT9 (mSEPT9) and methylated Ankrd/3b (mAnkrd13b) provides a sensitivity of 0.671 at a specificity of 0.9 for detecting colorectal cancer in a subject. Incorporating detection of a protein biomarker associated with colorectal cancer combined with the methylated genomic DNA was shown to improve sensitivity. Specifically, detecting carcinoembryonic antigen (CEA) in combination with the methylated genomic DNA improved sensitivity to 0.757. Surprisingly, detecting the presence of a different protein biomarker, CYFRA 21-1, a fragment of cytokeratin 19 that has been shown in some studies to be a marker of colorectal cancer, did not improve sensitivity. As further shown herein, sensitivity was increased to 0.771 when CEA was detected in combination with a protein biomarker amphiregulin and the methylated genomic DNA. In addition, it has been demonstrated sensitivity can be further increased to 0.841 when haptoglobin is detected in combination with protein biomarkers CEA and amphiregulin along with methylated genomic DNA. Accordingly, the methods, assays and kits described herein are useful for detecting colorectal cancer in a subject with a sensitivity of greater than or equal to 0.74 and a specificity of greater than or equal to 0.9.

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The discriminatory ability of biomarkers is evaluated using measures such as sensitivity, specificity and receiver-operating characteristics (ROC) probability curves. Sensitivity is the ability to detect a disease in patients in whom the disease is truly present (i.e., a true positive), and specificity is the ability to rule out the disease in patients whom the disease is truly absent (i.e., a true negative). To plot ROC probability curves, the false positive rate is plotted on the x-axis against the true positive rate on the y-axis. The area under the ROC curve (AUC, or AUROC) ranges from 0.5, indicating no power to separate cases from non-cases, to 1, indicating perfect discrimination. To be clinically meaningful, biomarkers should have an AUC value as close to 1 as possible.

As used herein “sensitivity” refers to the proportion of patients who test positive for the disease among those who actually have the disease; the higher the sensitivity, the lower the proportion of false negative results.

As used herein, “specificity” refers to the proportion of patients who test negative for the disease among those who actually are free of the disease; the higher the specificity, the lower the proportion of false positive results.

The term “AUC” or “AUROC” is an abbreviation for the area under the receive-operating characteristics (ROC) probability curve. The ROC probability curve is generated by plotting the true positive rate and the false positive rate. The true positive rate is the number of true positives divided by the total number of true positives+false negatives. The false positive rate is the number of false positives divided by the total number false positives+true negatives. The AUC provides an aggregate measure of performance across all possible classification models. AUC ranges in value from 0 to 1. A model whose prediction are 100% correct has an AUC of 1.0.

The term “methylated” as used herein refers to a biochemical process involving the addition of a methyl group to cytosine DNA nucleotides. DNA methylation at the 5 position of cytosine, especially in promoter regions, can have the effect of reducing gene expression and has been found in every vertebrate examined. In adult non-gamete cells, DNA methylation typically occurs in a CpG site. The term “CpG site” or “CpG dinucleotide”, as used herein, refers to regions of DNA where a cytosine nucleotide occurs next to a guanine nucleotide in the linear sequence of bases along its length. “CpG” is shorthand for “C-phosphate-G”, that is cytosine and guanine separated by only one phosphate; phosphate links any two nucleosides together in DNA. The “CpG” notation is used to distinguish this linear sequence from the CG base-pairing of cytosine and guanine. Cytosines in CpG dinucleotides can be methylated to form 5-methylcytosine. The term “CpG site” or “CpG site of genomic DNA” is also used with respect to the site of a former (unmethylated) CpG site in DNA in which the unmethylated C of the CpG site was converted to another as described herein (e.g. by bisulfite to uracil). The application provides the genomic sequence of each relevant DNA region as well as the bisulfite converted sequences of each converted strand. CpG sites referred to are always the positions of the CpG sites of the genomic sequence, even if the converted sequence does no longer contain these CpG sites due to the conversion. Specifically, methylation in the context of the present invention means hypermethylation. The term “hypermethylation” refers to an aberrant methylation pattern or status (i.e. the presence or absence of methylation of one or more nucleotides), wherein one or more nucleotides, preferably C(s) of a CpG site(s), are methylated compared to the same genomic DNA of a control, i.e. from a non-cancer cell of the subject or a subject not suffering or having suffered from the cancer the subject is treated for, preferably any cancer (healthy control). The term “control” can also refer to the methylation status, pattern or amount which is the average or median known of or determined from a group of at least 5, preferably at least 10 subjects. In particular, it refers to an increased presence of 5-mCyt at one or a plurality of CpG dinucleotides within a DNA sequence of a test DNA sample, relative to the amount of 5-mCyt found at corresponding CpG dinucleotides within a (healthy) control DNA sample, both samples preferably being of the same type, e.g. both blood plasma, both blood serum, both saliva, or both urine. Hypermethylation as a methylation status/pattern can be determined at one or more CpG site(s). If more than one CpG site is used, hypermethylation can be determined at each site separately or as an average of the CpG sites taken together. Alternatively, all assessed CpG sites must be methylated (comethylation) such that the requirement hypermethylation is fulfilled.

The term “detecting DNA methylation” as used herein refers to at least qualitatively analyzing for the presence or absence of methylated target DNA. “Target DNA” refers to a sequence within the genomic DNA polynucleotide (region) that is generally limited in length, but is preferably a length suitable for PCR amplification, e.g. at least 30 to 1000, more preferably 50 to 300 and even more preferably 75 to 200 or 75 to 150 nucleotides long. This includes primer binding sites if the target region is amplified using primers. Methylation is preferably determined at 1 or more, 2 or more, 3 or more, 4 or more, or 5 or more, most preferably 6 or more (e.g. 10 or more, 15 or more, or 30 or more) CpG sites of the target DNA. Usually, the CpG sites analyzed are comethylated in cancer, such that also CpG sites of neighboring DNA are methylated and can be analyzed in addition or instead. “At least qualitatively” means that also a quantitative determination of methylated target DNA, if present, can be performed. In fact, it is preferred that detecting of the DNA methylation comprises determining the amount of methylated genomic DNA.

The term “biological sample” as used herein refers to material obtained from a subject. A biological sample may be liquid (e.g., blood) or solid (e.g., stool).

The term “circulating DNA” or “free circulating DNA” as used herein refers to cell-free DNA in a body liquid (in particular blood) which circulates in the body.

The term “circulating tumor DNA” or “ctDNA” as used herein refers to circulating DNA that is derived from a tumor (i.e. cell-free DNA derived from tumor cells).

The term “is indicative for” or “indicates” as used herein refers to an act of identifying or specifying the thing to be indicated. As will be understood by persons skilled in the art, such assessment normally may not be correct for 100% of the subjects, although it preferably is correct. The term, however, requires that a correct indication can be made for a statistically significant part of the subjects Whether a part is statistically significant can be determined easily by the person skilled in the art using several well-known statistical evaluation tools, for example, determination of confidence intervals, determination of p values, Student's t-test, Mann-Whitney test, etc. Details are provided in Dowdy and Wearden, Statistics for Research, John Wiley & Sons, New York 1983. The preferred confidence intervals are at least 50%, at least 60%, at least 70%, at least 80%, at least 90%, at least 95%. The p values are preferably 0.05, 0.01, or 0.005.

The term “diagnosis” as used herein refers to a determination whether a subject does or does not have cancer. A diagnosis by methylation analysis of the target DNA as described herein may be supplemented with a further means as described herein to confirm the cancer detected with the methylation analysis. As will be understood by persons skilled in the art, the diagnosis normally may not be correct for 100%/6 of the subjects, although it preferably is correct. The term, however, requires that a correct diagnosis can be made for a statistically significant part of the subjects. For a description of statistic significance and suitable confidence intervals and p values, see above.

The term “prognosis” as used herein refers to an educated prediction of the status of a disease or disorder (e.g., a proliferative disease, e.g., cancer).

The term “treatment” or “treating” with respect to a proliferative disease as used herein refers to a therapeutic treatment, wherein the goal is to reduce progression of a proliferative disease. Beneficial or desired clinical results include, but are not limited to, release of symptoms, reduction of the length of the disease, stabilized pathological state (specifically not deteriorated), slowing down of the disease's progression, improving the pathological state and/or remission (both partial and total), preferably detectable. A successful treatment does not necessarily mean cure, but it can also mean a prolonged survival, compared to the expected survival if the treatment is not applied. In a preferred embodiment, the treatment is a first line treatment, i.e. the cancer was not treated previously. Cancer treatment involves a treatment regimen.

The term “treatment regimen” as used herein refers to how the subject is treated in view of the disease and available procedures and medication. Non-limiting examples of cancer treatment regimens are chemotherapy, surgery and/or irradiation or combinations thereof. The early detection of cancer the present invention enables allows in particular for a surgical treatment, especially for a curative resection. In particular, the term “treatment regimen” refers to administering one or more anti-cancer agents or therapies as defined below. The term “anti cancer agent or therapy” as used herein refers to chemical, physical or biological agents or therapies, or surgery, including combinations thereof, with antiproliferative, antioncogenic and/or carcinostatic properties.

The term “protein biomarker” refers to a polypeptide or fragment thereof that indicates a specific biological state.

The term “colorectal cell proliferative disease” refers to conditions in which unregulated or abnormal growth, or both, of cells can lead to the development of an unwanted condition or disease, which may or may not become cancerous. Exemplary cell proliferative disorders include a variety of conditions wherein cell division is deregulated, including but not limited to, neoplasms, benign tumors, malignant tumors, precancerous conditions, in situ tumors, encapsulated tumors, metastatic tumors, cancers, and carcinomas.

The term “pre-cancerous lesion” is a group of abnormal cells that are neither cancer cells nor normal cells and may develop into cancer. Pre-cancerous lesions are not typically invasive.

The term “adenoma” refers to a tumor that is not cancer and typically originates in gland-like cells of the epithelial tissue (i.e., thin layer of tissue that covers organs, glands, and other structures within the body). An “advanced adenoma” refers to a tumor that bridges the beginning and malignant states and is a neoplastic surrogate marker for present and future cancer risk (e.g., colorectal cancer risk). Tn some embodiments, an advanced adenoma is an adenoma with significant villous features (>25%), size of 1.0 cm or more, high-grade dysplasia or early invasive cancer.

### Multi-Analyte Detection Methods and Assays for Proliferative Diseases

In some aspects, the disclosure provides a method or assay for detecting one or more methylated genomic DNA sequences and one or more protein biomarkers in one or more biological samples obtained from a subject. In some embodiments, the one or more methylated genomic DNA sequences and the one or more protein biomarkers are detected in the same biological sample. In some embodiments, the one or more methylated genomic DNA sequences and the one or more protein biomarkers are detected in different biological samples.

In some embodiments, the one or more methylated genomic DNA sequences and the one or more protein biomarkers are detected simultaneously. In some embodiments, the one or more methylated genomic DNA sequences and the one or more protein biomarkers are detected simultaneously using a semi-automated system. In some embodiments, the one or more methylated genomic DNA sequences and the one or more protein biomarkers are detected simultaneously using a fully automated system. In some embodiments, the one or more methylated genomic DNA sequences and the one or more protein biomarkers are detected sequentially. In some embodiments, the one or more methylated genomic DNA sequences and the one or more protein biomarkers are detected using separate semi-automated systems. In some embodiments, the one or more methylated genomic DNA sequences and the one or more protein biomarkers are detected using separate fully automated systems.

In some embodiments, the method or assay detects colorectal cancer or a colorectal cell proliferative disease in a subject. In some embodiments, the method or assay detects a pre-cancerous lesion (e.g., an advanced adenoma) in a subject.

### Detecting Protein Biomarkers

In some aspects, the disclosure provides methods for detecting one or more protein biomarkers in combination with methods for detecting one or more methylated genomic DNAs. Methods for detecting protein biomarkers are known to those of skill in the art. Exemplary methods are described herein.

In some embodiments, the one or more protein biomarkers are proteins associated with a proliferative disease (e.g., a cancer). In some embodiments, the one or more protein biomarkers are proteins associated with colorectal cancer or a colorectal cell proliferative disease. In some embodiments, the one or more protein biomarkers comprise a protein previously reported as being associated with colorectal cancer. Exemplary protein biomarkers associated with colorectal cancer include, but are not limited to, carcinoembryonic antigen (CEA), autoantibodies (e.g., anti-p53 autoantibodies), C-reactive protein (CRP), macrophage chemoattractant protine-1 (MCP-1), interleukin-6 (IL-6), macrophage inhibitory cytokine 1 (also known as growth differentiation factor 15 (MIC-1/GDF15), amphiregulin (AREG), leucine-rich alpha-2-glycoprotein-1 (LRG1), microtubule-associated protein RP/EP family member 1 (MAPRE1), insulin like growth factor binding protein 2 (IGFBP2), enolase 1, arginine-rich mutated in early-stage tumors (ARMET), protein disulfide isomerase family A member 3 (PDIA3), adiponectin, leptin, circulating vascular cell adhesion molecule-1 (sVCAM-1), soluble intracellular adhesion molecule-1 (sICAM-1), sE-selectin, serum amyloid A (SAA), carbonic anhydrase 12 (CA12), apolipoprotein C-2 (APOC2), clusterin (CLU), complement factor 4 (CO4-B), complement component C9 (CO9), alpha-2-HS-glycoprotein (FETUA), mannan-binding lectin serine protease 2 (MASP2), mannose binding lectin 2 (MBL2), glycine rich protein 2 (GRP2), serum soluble tumor necrosis factor receptor 2 (sTNFR-2), macrophage inflammatory protein-1 (CCL3/MIP1A), chemokine ligand 15 (CCL15), cutaneous T cell-attracting chemokine (CCL27/CTACK), gamma-tubulin complex component 2 (CXCL6/GCP2), BAG cochaperone 4 (BAG4), von Willebrand factor (VWF), epidermal growth factor receptor (EGFR), CD44, IL-6 cytokine family signal transducer (IL6ST), fibroblast growth factor 21 (FGF-21), pancreatic polypeptide (PPY), alpha fetoprotein (AFP), cancer antigen 15-3 (CAI5-3), cancer antigen 125 (CA 125), prostate-specific antigen (PSA), squamous cell carcinoma antigen (SCC), cancer antigen 19-9 (CA 19-9), sCD26, complement C3a, anaphylatoxin, TIMP metallopeptidase inhibitor 1 (TIMP-1), ferritin, seprase, osteopontin (OPN), FasL, Flt-3 ligand (FIt3L), fibroblast growth factor-23 (FGF-23), deltha/notch like EGF-related receptor (DNER), cadherin 5 (CDH5), mannan-binding lectin serine protease 1 (MASP1), paraooxonase 3 (PON3), transferrin receptor protein 1 (TFRC), secreted protein acidic and rich in cysteine (SPARC), proteinase 3 (PRTN3), myeloperoxidase (MPO), matrix metallopeptidase 9 (MMP9), alpha-1 antitrypsin (A1AT), apolipoprotein A-1 (APOA1), haptoglobin (HP), and netrin-1.

In some embodiments, the protein biomarker is carcinoembryonic antigen (CEA). In some embodiments, the protein biomarker is amphiregulin (AREG). In some embodiments, the protein biomarker is cytokeratin 19 fragment (CYFRA 21-1). In some embodiments, the protein biomarker is a galectin-3 ligand. In some embodiments, the galectin-3 ligand is haptoglobin.

In some embodiments, the one or more protein biomarkers is selected from the group of CEA, AREG, CYFRA 21-1, a galectin-3 ligand, and any combination thereof. In some embodiments, the one or more protein biomarkers comprise CEA and/or AREG. In some embodiments, the one or more protein biomarkers comprise CEA and AREG. In some embodiments, the one or more protein biomarkers comprise CEA and CYFRA 21-1. In some embodiments, the one or more protein biomarkers comprise AREG and CYFRA 21-1. In some embodiments, the one or more protein biomarkers comprise CEA, AREG, and CYFRA 21-1. In some embodiments, the one or more protein biomarkers comprise CEA and a galectin-3 ligand. In some embodiments, the one or more protein biomarkers comprise AREG and a galectin-3 ligand. In some embodiments, the one or more protein biomarkers comprise CEA, AREG, and a galectin-3 ligand. In some embodiments, the one or more protein biomarkers comprise CEA and haptoglobin. In some embodiments, the one or more protein biomarkers comprise AREG and haptoglobin. In some embodiments, the one or more protein biomarkers comprise CEA, AREG, and haptoglobin. In some embodiments, the one or more protein biomarkers comprise CEA, CYFRA 21-1 and a galectin-3 ligand. In some embodiments, the one or more protein biomarkers comprise AREG, CYFRA 21-1 and a galectin-3 ligand. In some embodiments, the one or more protein biomarkers comprise CEA, AREG, CYRFA 21-1, and a galectin-3 ligand. In some embodiments, the one or more protein biomarkers comprise CEA, CYFRA 21-1 and haptoglobin. In some embodiments, the one or more protein biomarkers comprise AREG, CYFRA 21-1 and haptoglobin. In some embodiments, the one or more protein biomarkers comprise CEA, AREG, CYRFA 21-1, and haptoglobin.

### CEA

In some embodiments, the disclosure provides a method, assay or kit for detecting CEA. Carcinoembryonic antigen (CEA) is a glycoprotein that inhibits apoptosis and regulates tumor pathogenesis. Primarily produced in fetal gastrointestinal tissue, CEA is typically present at very low levels in the blood or serum of healthy adults. However, the blood and serum levels of CEA are raised in some types of cancer, indicating that elevated CEA levels can serve as a biomarker for certain cancers (Kankanala V L, Mukkamalla S K R. [Updated 2022 Jan. 26]. In: StatPearls [Internet]. Treasure Island (FL): StatPearls Publishing; 2022 January). Currently, CEA levels are monitored during postoperative or postadjuvant chemotherapy surveillance to detect or predict tumor recurrence originating from postsurgery residual tumor cells. (Ozawa T, Matsuda K, Ishihara S, Fukushima Y, Shimada R, Hayama T, Nozawa K, Hashiguchi Y. J Surg Oncol. 2021 July; 124(1):97-105. doi: 10.1002/jso.26497. Epub 2021 Apr. 13. PMID: 33848373.). CEA levels may also be elevated in serum from individuals with medullary thyroid carcinoma, breast carcinoma, gastric carcinoma, pancreatic carcinoma, and lung carcinoma, as well as some non-neoplastic conditions (i.e. COPD, Crohn's disease, ulcerative colitis, cirrhosis, hypothyroidism, etc.) and in smokers (Goldenberg, D. M., et al., J. Natl. Cancer Inst. (Bethesda) 57 (1976) 11-22; Alexander J C, Silverman N A, Chretien P B. JAMA. 1976 May 3; 235(18):1975-9.).

In some embodiments, CEA comprises the amino acid sequence set forth in SEQ ID NO: 50. In some embodiments, the method, assay or kit described herein detects a CEA polypeptide having the sequence set forth in SEQ ID NO: 50. In some embodiments, the method, assay or kit described herein detects a fragment of a CEA polypeptide. In some embodiments, a fragment of a CEA polypeptide is a fragment of SEQ ID NO: 50.

Methods for detecting CEA or fragments thereof in a biological sample are known in the art and described herein.

### Amphiregulin

In some embodiments, the disclosure provides a method, assay or kit for detecting amphiregulin. Amphiregulin (AREG) is an autocrine growth factor, and a mitogen for astrocytes, Schwann cells, and fibroblasts. AREG is encoded by a gene in the epidermal growth factor (EGF) gene family, and is synthesized as a membrane-anchored pre-protein. After cleavage of the pre-protein, AREG exists in several soluble and membrane-bound forms with its predominant glycosylated soluble form being ˜43 kDa in size.

As a ligand for the epidermal receptor growth factor receptor (EGFR), AREG plays a crucial role in immunity and wound repair. Through its interactions with the EGFR, AREG can trigger signaling events that mediate various cellular physiological processes including, but not limited to, metabolism, cell cycle, and proliferation (Simon Melderis et al., Journal of Autoimmunity, 129 (2022) 102829, ISSN 0896-8411, doi.org/10.1016/j.jaut.2022.102829). Under various immunological conditions, AREG is expressed by activated immune cells that control tolerance and resistance mechanisms.

The gene encoding AREG is associated with a psoriasis-like skin phenotype, and is also associated with other pathological disorders, including various types of cancers and inflammatory conditions (Zhang, M. Y., Fang, S., Gao, H. et al. Cell Biosci 11, 40 (2021). doi.org/10.1186/s13578-021-00553-0).

In some embodiments, AREG comprises the amino acid sequence set forth in SEQ ID NO: 51. In some embodiments, the method, assay or kit described herein detects an AREG polypeptide having the sequence set forth in SEQ ID NO: 51. In some embodiments, the method, assay or kit described herein detects a fragment of an AREG polypeptide. In some embodiments, a fragment of an AREG polypeptide is a fragment of SEQ ID NO: 51.

Methods for detecting AREG or fragments thereof in a biological sample are known in the art and described herein.

### CFYRA21-1

In some embodiments, the disclosure provides a method, assay or kit for detecting CYFRA21-1. CYFRA21-1, also known as cytokeratin-19, is a 40 kDa type I keratin. Embryonal hepatocytes contain cytokeratins (CK) 8, 18, and 19. However, healthy adult hepatocytes contain only CK8 and 18, with CYFRA21-1 being negative after the tenth week of gestation. As such, elevated CYFRA21-1 levels may serve as a biomarker for cancers (Rastel D, Ramaioli A, Cornillie F, Thirion B. CYFRA 21-1 Multicentre Study Group. Eur J Cancer. 1994; 30A(5):601-6. doi: 10.1016/0959-8049(94)90528-2. PMID: 7521651).

In some embodiments, CYFRA21-1 comprises the amino acid sequence set forth in SEQ ID NO: 52. In some embodiments, the method, assay or kit described herein detects an CYFRA21-1 polypeptide having the sequence set forth in SEQ ID NO: 52. In some embodiments, the method, assay or kit described herein detects a fragment of an CYFRA21-1 polypeptide. In some embodiments, a fragment of an CYFRA21-1 polypeptide is a fragment of SEQ ID NO: 52.

Methods for detecting CYFRA21-1 or fragments thereof in a biological sample are known in the art and described herein.

### Galecitn-3 Ligands

In some embodiments, the disclosure provides a method, assay or kit for detecting a galectin. In some embodiments, the disclosure provides a method, assay or kit for detecting a galectin ligand. In some embodiments, the disclosure provides a method, assay or kit for detecting a galectin-3 ligand. In some embodiments, the disclosure provides a method, assay or kit for detecting haptoglobin.

Galectins trigger T cell apoptosis, exhaustion, and cytokine synthesis which promotes tumorigenesis and the development of a variety of cancers (Saussez S, Glinoer D, Chantrain G, Pattou F, Carnaille B, Andre S, Gabius H J, Laurent G. Thyroid. 2008; 7:705-712). The galectin Gal-3 serves as a biomarker for poor prognosis of various types of cancer. Additionally, Gal-3 has numerous intracellular ligands which can also serve as biomarkers for various cancer types. Such Gal-3 ligands include, but are not limited to, haptoglobin, Bcl-2, and Ras, which further demonstrate the role of Gal-3 in regulating apoptosis (Yang R Y, Hsu D K, Liu F T. Proc. Natl. Acad. Sci. U.S.A. 1996; 13:6737-6742; Elad-Sfadia G, Haklai R, Balan E, Kloog Y. J. Biol. Chem. 2004; 33:34922-34930).

In some embodiments, haptoglobin comprises the amino acid sequence set forth in SEQ ID NO: 53. In some embodiments, the method, assay or kit described herein detects a haptoglobin polypeptide having the sequence set forth in SEQ ID NO: 53. In some embodiments, the method, assay or kit described herein detects a fragment of a haptoglobin polypeptide. In some embodiments, a fragment of a haptoglobin polypeptide is a fragment of SEQ ID NO: 53.

Methods for detecting a galectin-3 (e.g., haptoglobin) or fragments thereof in a biological sample are known in the art and described herein. For example, methods for measuring haptoglobin are described in U.S. Pat. Nos. 9,810,691 and 10,620,206 and US Patent Publication No. 2021-0148912, each of which is incorporated herein by this reference.

In some embodiments, a specific glycosylated form of a galectin-3 ligand is detected. In some embodiments, a specific glycosylated form of haptoglobin is detected. In some embodiments, the haptoglobin is a 40-kDa glycoprotein that binds to anti-haptoglobin antibodies. Because the 40-kDa protein appears to be present at some level in healthy patients and is immunologically related to haptoglobin, methods described herein allow for quantitative detection only of specific glycosylated forms of the 40-kDa protein. Tn some embodiments, the methods or assay involve obtaining a diluted serum sample from patient and treating the bulk sample to desialylate proteins that are present. The desialylated and diluted serum sample is then subjected to an assay by capturing the 40-kDa protein using an anti-haptoglobin antibody and detecting the captured protein with a detectable lectin that binds to galactose. By using a desialylated serum sample the assay allows for the amount of the 40-kDa glycoform in a sample to be determined quantitatively. In some embodiments, the presence or increased level of the 40-kDa glycoform (e.g., as compared to a control sample or a reference level) is indicative of the presence of colorectal cancer in the patient.

In some embodiments, one or more antibodies are employed that bind to the 40-kDa haptoglobin glycoform. Antibodies include any type of antibody, and specifically refer to antibodies that react immunologically with human haptoglobin or immunologically related proteins such as the 40-kDa haptoglobin glycoform. In particular, these antibodies may be used in various diagnostic applications, such as the ELISA assay methods described herein.

It has long been known that extracts from certain plants could agglutinate red blood cells. Although the term “lectin” was originally a term used to describe agglutinins which could discriminate among types of red blood cells, the term is now generally defined to include sugar-binding proteins from a wide variety of sources. Lectins have been found in plants, viruses, microorganisms and animals. Although lectins share the common property of binding to defined sugar structures, their roles in various organisms are not likely to be the same and remain incompletely understood.

Because of the specificity that each lectin has toward a particular carbohydrate structure, even oligosaccharides with identical sugar compositions can be distinguished or separated. Some lectins will bind only to structures with mannose or glucose residues, while others may recognize only galactose residues. Some lectins require that the particular sugar be in a terminal non-reducing position in the oligosaccharide, while others can bind to sugars within the oligosaccharide chain. Some lectins do not discriminate between alpha and beta anomers, while others require not only the correct anomeric structure but a specific sequence of sugars for binding. The affinity between a lectin and its receptor may vary a great deal due to small changes in the carbohydrate structure of the receptor Thus, lectins can be used in similar detection methods as antibodies, for the detection of specific carbohydrate moieties. In some embodiments, methods or assays for detecting a haptoglobin glycoform (e.g., the 40-kDa glycoform) use lectins as selective binding agents. Generally, the carbohydrate composition of the glycoform is exploited to detect its presence in a sample. For example, in some embodiments, the methods or assays described herein employ galactose-binding lectins to capture or detect the 40-kDa protein comprising such a galactose moiety. In some embodiments, the lectin is mammalian galectin-3. Ricinus communis lectin, Datura stramonium lectin, Erythrina cristagalli lectin, or Lycopersicon esculentum lectin. In some embodiments, the lectin is Erythrina cristagalli lectin. In some embodiments, lectins suitable for the methods and assays described herein are labeled. Methods for labeling antibodies can generally also be applied to lectins.

### Protein Detection Methods

In some aspects, the present disclosure provides methods for detecting protein in a biological sample. In some aspects, the present disclosure provides methods for detecting an amount of a protein in a biological sample. Methods for detecting proteins and fragments thereof are known to those of skill in the art. Exemplary methods include western blot, enzyme-linked immunosorbent assay (ELISA), immunobead-based formats, proximity extension assay (PEA) and mass-spectrometry.

In some embodiments, wherein more than one protein biomarker is detected, the same protein detection method is utilized. In some embodiments, wherein more than one protein biomarker is detected, different protein detection methods are utilized. For example, in some embodiments, a first protein biomarker is detected by a first ELISA and a second protein biomarker is detected by a second ELISA. In some embodiments, wherein more than one protein biomarker is detected, the proteins are detected simultaneously. In some embodiments, wherein more than one protein biomarker is detected, the proteins are detected in a multiplex format. For example, in some embodiments, first and second protein biomarkers are detected in the same assay.

In some embodiments, one or more protein biomarkers are detected manually. In some embodiments, one or more protein biomarkers are detected using a semi-automated system. Semi-automated systems require at least one manual procedure by the operator, such as sample application or reagent dispensation. Exemplary semi-automated systems include programmable thermal cycler or over. In some embodiments, one or more protein biomarkers are detected using a fully automated system. Fully automated systems do not require any operator intervention. Exemplary fully automated systems include TECAN or Hamilton Instrumentation.

### A. Immunodetection Methods

In some embodiments, one or more protein biomarkers are detected with an antibody. Antibodies include any type of antibody, including antibodies that specifically bind unmodified proteins, glycosylated protein variants, or other post-translationally modified proteins. In some embodiments, these antibodies are used in protein detection methods. Methods for generating antibodies suitable for binding a protein of interest are known those of skill in the art and described herein.

In some embodiments, antibodies for detecting one or more protein biomarkers are commercially available. Exemplary commercially available antibodies specific for CEA include, but are not limited to, clone IOE1 (catalog #MBS8504731; MyBioSource), clone 1106 (catalog #MA5-14675; ThermoFisher Scientific), clone II-7 (catalog #M707291; Agilent); clone 1A5C3 and 3C5C10 (GenScript), and REA1158 (catalog 130-120-344; Miltenyi Biotec). Exemplary commercially available antibodies specific for amphiregulin include, but are not limited to, AF262 (R&D Systems); ab89119, ab234750, ab224350, and ab180722 (abcam); and AB_10981232 (Invitrogen). Exemplary commercially available antibodies specific for CYFRA21-1 include, but are not limited to, clone XC42 (QED Bioscience), and catalog #MBS318073 (MyBioSource). Exemplary commercially available antibodies specific for haptoglobin include, but are not limited to, ab256454, ab131236, and ab13429 (abcam); clone GOS11 (LSBio), clone JM10-79 (Novus Biologicals), and AB_2541674 (Invitrogen).

In some embodiments, immunodetection methods for binding, purifying, removing, quantifying and/or otherwise generally detecting biological components, including AREG, CEA, CYFRA21-1, and/or Gal-3 ligands such as a 40-kDa haptoglobin glycoform, are used in the methods, assays and kits described herein. Exemplary immunodetection methods include radioimmunoassay (RIA), enzyme linked immunosorbent assay (ELISA), fluoroimmunoassay, immunoradiometric assay, chemiluminescent assay, immunobead-based formats, and bioluminescent assay.

In general, the immunodetection methods comprise obtaining a sample suspected of containing one or more protein biomarkers (e.g., AREG, CEA, CYFRA21-1, and/or Gal-3) and contacting the sample with one or more antibodies that bind the one or more protein biomarkers under conditions effective to allow the formation of immunocomplexes. In some embodiments, the immunocomplexes are detected to determine the presence or amount of the one or more protein biomarkers of interest.

In some embodiments, a biological sample is contacted with an antibody specific for a protein of interest under effective conditions and for a period of time sufficient to allow the formation of immune complexes. In some embodiments, the sample is combined with the antibody and incubated for a period of time long enough for the antibodies to form immune complexes. After this time, the sample-antibody composition, such as a tissue section, ELISA plate or western blot, will generally be washed to remove any non-specifically bound protein species.

In general, the detection of immunocomplex formation is well known in the art and may be achieved through the application of numerous approaches. These methods are generally based upon the detection of a label or marker, such as any of those radioactive, fluorescent, biological and enzymatic tags.

The antibody employed in the detection may itself be linked to a detectable label, wherein one would then simply detect this label, thereby allowing the amount of the primary immune complexes in the composition to be determined. Alternatively, the first antibody that becomes bound may be detected by means of a second binding ligand that has binding affinity for the antibody. In these cases, the second binding ligand may be linked to a detectable label. The second binding ligand is itself often an antibody, which may thus be termed a “secondary” antibody. In some embodiments, the immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. In some embodiments, a second binding ligand, such as an antibody, that has binding affinity for the antibody is used to form secondary immune complexes, as described above. After washing, the secondary immune complexes are contacted with a third binding ligand or antibody that has binding affinity for the second antibody under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

In some embodiments, a biotinylated monoclonal or polyclonal antibody is used to detect the protein biomarker and a secondary antibody is then used to detect the biotin attached to the complexed biotin. If the protein biomarker is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

In some embodiments, a method suitable for detecting the presence or amount of one or more protein biomarkers is the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

In some embodiments, a method suitable for detecting the presence or amount of one or more protein biomarkers is a proximity extension assay (PEA). In this assay, a pair of antibodies linked to unique oligonucleotides (proximity probes) binds to a protein target. Based on this binding, the probes come in close proximity and hybridize to each other. The method further comprises adding a DNA polymerase to extend the hybridizing oligo and create a DNA amplicon that can subsequently be detected and quantified by quantitative real-time PCR or next generation sequence (NGS).

In some embodiments, a method suitable for detecting the presence or amount of one or more protein biomarkers is an ELISA. In this method, one or more antibodies specific for the one or more protein biomarkers are immobilized onto a selected surface exhibiting protein affinity, such as a well in a polystyrene microtiter plate. Then, a test composition suspected of containing the one or more protein biomarkers, such as a diluted clinical sample, is added to the wells. After binding and/or washing to remove non-specifically bound immune complexes, the bound protein biomarker may be detected. Detection can be achieved by contacting the sample with an agent, such as a secondary antibody, that is linked to a detectable label. This type of ELISA is a “sandwich ELISA”.

Irrespective of the format employed, ELISAs have certain features in common, such as coating, incubating and binding, washing to remove non-specifically bound species, and detecting the bound immune complexes. These are described below.

In some embodiments, the method for detecting one or more protein biomarkers is an immunobead-based assay (e.g., Luminex). In some embodiments, the method for detecting one or more protein biomarkers is multiplexed immunobead-based assay. In some embodiments, an antibody targeting the one or more protein biomarker is conjugated to a bead. In some embodiments, upon binding of the antibody to the one or more protein biomarkers, the bead detected using a method known to those of skill in the art or described herein.

### B. Generating Antibodies for Detection Methods

Provided herein are methods for generating an antibody specific to a protein biomarker described herein. Methods for generating antibodies are known to those of skill in the art and briefly described.

As used herein, the term “antibody” refers broadly to any immunologic binding agent such as IgG, IgM, IgA, IgD and IgE, and includes any antibody-like molecule that has an antigen binding region, and includes antibody fragments such as Fab′, Fab, F(ab′)2, single domain antibodies (DABs), Fv, scFv (single chain Fv), etc. Techniques for preparing, using, and characterizing various antibody-based constructs and fragments are well known in the art (see, e.g., Antibodies: A Laboratory Manual, Cold Spring Harbor Laboratory, 1988; incorporated herein by reference).

In some embodiments, an antibody useful in the methods, assays and kits described herein is a polyclonal antibody. A polyclonal antibody is prepared by immunizing an animal with, for example a human haptoglobin (e.g., a purified haptoglobin) composition and collecting antisera from that immunized animal. A wide range of animal species can be used for the production of antisera. In some embodiments, the animal used for production of antisera is a rabbit, a mouse, a rat, a hamster, a guinea pig or a goat. The choice of animal may be decided upon the ease of manipulation, costs or the desired amount of sera, as would be known to one of skill in the art.

As is also well known in the art, the immunogenicity of a particular immunogen composition can be enhanced by the use of non-specific adjuvants, which stimulate the immune response. Suitable adjuvants include all acceptable immunostimulatory compounds, such as cytokines, chemokines, cofactors, toxins, plasmodia or synthetic compositions. Adjuvants may include, but are not limited to, TL-1, IL-2, IL-4, IL-7, IL-12, interferon, GM-CSF, BCG, aluminum hydroxide, MDP compounds, such as thur-MDP and nor-MDP, CGP (MTP-PE), lipid A, and monophosphoryl lipid A (MPL). Exemplary adjuvants include complete Freund's adjuvant (a non-specific stimulator of the immune response containing killed Mycobacterium tuberculosis), incomplete Freund's adjuvants and aluminum hydroxide adjuvant.

In addition to adjuvants, it may be desirable to coadminister biologic response modifiers (BRM), which have been shown to upregulate T cell immunity or down-regulate suppressor cell activity. Such BRMs include, but are not limited to, Cimetidine (CIM; 1200 mg/d) (Smith/Kline, Pa.); low-dose Cyclophosphamide (CYP; 300 mg/m.sup.2) (Johnson/Mead, NJ), cytokines such as -interferon, IL-2, or IL-12 or genes encoding proteins involved in immune helper functions, such as B-7.

The amount of immunogen composition used in the production of polyclonal antibodies varies upon the nature of the immunogen as well as the animal used for immunization. A variety of routes can be used to administer the immunogen including but not limited to subcutaneous, intramuscular, intradermal, intraepidermal, intravenous and intraperitoneal. The production of polyclonal antibodies may be monitored by sampling blood of the immunized animal at various points following immunization.

A second, booster dose (e.g., provided in an injection), may also be given. The process of boosting and titering is repeated until a suitable titer is achieved. When a desired level of immunogenicity is obtained, the immunized animal can be bled and the serum isolated and stored, and/or the animal can be used to generate MAbs.

In some embodiments, an antibody useful in the methods, assays and kits described herein is a monoclonal antibody (MAb). MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified protein, polypeptide, peptide or domain, be it a wild-type or mutant composition. The immunizing composition is administered in a manner effective to stimulate antibody producing cells. The methods for generating monoclonal antibodies (MAbs) generally begin along the same lines as those for preparing polyclonal antibodies. Rodents such as mice and rats are often used, however, the use of rabbit, sheep or frog cells is also possible. The animals are injected with antigen, generally as described above. The antigen may be mixed with adjuvant, such as Freund's complete or incomplete adjuvant. Booster administrations with the same antigen or DNA encoding the antigen would occur at approximately two-week intervals.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible.

The antibody producing B lymphocytes from the immunized animal are then fused with cells of an immortal myeloma cell, generally one of the same species as the animal that was immunized myeloma cell lines suited for use in hybridoma producing fusion procedures preferably are non-antibody producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984).

Methods for generating hybrids of antibody producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al., (1977). The use of electrically induced fusion methods is also appropriate (Goding pp. 71-74, 1986).

The favored selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity.

The selected hybridomas are then serially diluted and cloned into individual antibody producing cell lines, which clones can then be propagated indefinitely to provide MAbs. The cell lines may be exploited for MAb production in two basic ways. First, a sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration. Second, the individual cell lines could be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the monoclonal antibodies so produced by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It is also contemplated that a molecular cloning approach may be used to generate monoclonals. In some embodiments, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Alternatively, monoclonal antibody fragments encompassed by the present invention can be synthesized using an automated peptide synthesizer, or by expression of full-length gene or of gene fragments in E. coli.

### C. Mass Spectrometry

In some embodiments, the presence or amount of one or more protein biomarkers is determined by mass spectrometry, including SELDI mass spectrometry.

Exemplary mass spectrometry methods include such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

### Detecting DNA Methylation

In some aspects, the disclosure provides methods for detecting one or more methylated genomic DNA in combination with methods for detecting one or more protein biomarkers. Methods for detecting methylated genomic DNA are known to those of skill in the art. Exemplary methods are described herein.

In some embodiments, the one or more methylated genomic DNA are proteins associated with a proliferative disease (e.g., a cancer). In some embodiments, the one or more methylated genomic DNA are associated with colorectal cancer or a colorectal cell proliferative disease. In some embodiments, the one or more methylated genomic DNA comprise a methylated genomic DNA previously reported as being associated with colorectal cancer. Exemplary methylated genomic DNA associated with colorectal cancer include, but are not limited to, mSEPT9, mNKX2, mADCYAP1, mRASFF2, mKHDRBS2, mSND1, mCLEC14A, mTBX18, mANKRD13B, mTFAP2E, mCRMP1, mTMEFF2, mEYA4, mVAX1, mMSC, and mNGFR In these, the first letter “m” means “methylation marker”, and the capital letters refer to the gene the target DNA resides in.

In some embodiments, the methods, assays and kits described herein detect one or more methylated genomic DNA polynucleotides, wherein the methylated genomic DNA polynucleotides are selected from: mSEPT9, mNKX2, mADCYAP1, mRASFF2, mKHDRBS2, mSND1, mCLEC14A, mTBX18, mANKRD13B, mTFAP2E, mCRMP1, mTMEFF2, mEYA4, mVAX1, mMSC, mNGFR, and any combination thereof. In some embodiments, the methods, assays and kits described herein detect one or more methylated genomic DNA polynucleotides, wherein the methylated genomic DNA polynucleotides are selected from: mSEPT9 (SEQ ID NOs: 21, 26, 31 and/or 36), mNKX2 (SEQ ID NOs: 149, 154 and/or 159), mADCYAP1 (SEQ ID NOs: 54, 59 and/or 64), mRASFF2 (SEQ ID NOs: 164, 169 and/or 174), mKHDRBS2 (SEQ ID NOs: 69 and/or 74), mSND1 (SEQ ID NOs: 179, 184 and/or 189), mCLEC14A (SEQ ID NOs: 79, 84 and/or 89), mTBX18 (SEQ ID NOs: 194, 199 and/or 204), mANKRD13B (SEQ ID NOs: 1, 6, 11 and/or 16), mTFAP2E (SEQ ID NOs: 209, 214 and/or 219), mCRMP1 (SEQ ID NOs: 95 and/or 99), mTMEFF2 (SEQ ID NOs: 224, 229 and/or 234), mEYA4 (SEQ ID NOs: 104, 109 and/or 114), mVAX1 (SEQ ID NOs: 239 and/or 244), mMSC (SEQ ID NOs:119, 124 and/or 129), mNGFR (SEQ ID NOs: 134, 139 and/or 144), and any combination thereof.

In some embodiments, methylated genomic DNA comprises one or more CpG dinucleotides within a region of mSEPT9. In some embodiments, a region of mSEPT9 comprising one or more CpG dinucleotides is set forth in SEQ ID NOs: 21, 26, 31, and/or 36. In some embodiments, a region of mSEPT9 comprising one or more CpG dinucleotides is set forth in SEQ ID NO: 31.

In some embodiments, methylated genomic DNA comprises one or more CpG dinucleotides within a region of mANKRD13B. In some embodiments, a region of mANKRD13B comprising one or more CpG dinucleotides is set forth in SEQ ID NOs: 1, 6, 11, and/or 16. In some embodiments, a region of mANKRD13B comprising one or more CpG dinucleotides is set forth in SEQ ID NOs: 6 and 11.

In some embodiments, methylated genomic DNA comprises one or more CpG dinucleotides within a region of mADCYAP1. In some embodiments, a region of mADCYAP1 comprising one or more CpG dinucleotides is set forth in SEQ ID NOs: 54, 59, and/or 64.

In some embodiments, methylated genomic DNA comprises one or more CpG dinucleotides within a region of mKHDRBS2. In some embodiments, a region of mKHDRBS2 comprising one or more CpG dinucleotides is set forth in SEQ ID NOs: 69, and/or 74.

In some embodiments, methylated genomic DNA comprises one or more CpG dinucleotides within a region of mCLEC14A. In some embodiments, a region of mCLEC14A comprising one or more CpG dinucleotides is set forth in SEQ ID NOs: 79, 84, and/or 89.

In some embodiments, methylated genomic DNA comprises one or more CpG dinucleotides within a region of mCRMP1. In some embodiments, a region of mCRMP1 comprising one or more CpG dinucleotides is set forth in SEQ ID NOs: 95 and/or 99.

In some embodiments, methylated genomic DNA comprises one or more CpG dinucleotides within a region of mEYA4. In some embodiments, a region of mEYA4 comprising one or more CpG dinucleotides is set forth in SEQ ID NOs: 104, 109, and/or 114.

In some embodiments, methylated genomic DNA comprises one or more CpG dinucleotides within a region of mMSC. In some embodiments, a region of mMSC comprising one or more CpG dinucleotides is set forth in SEQ ID NOs: 119, 124, and/or 129.

In some embodiments, methylated genomic DNA comprises one or more CpG dinucleotides within a region of mNGFR. In some embodiments, a region of mNGFR comprising one or more CpG dinucleotides is set forth in SEQ ID NOs: 134, 139, and/or 144.

In some embodiments, methylated genomic DNA comprises one or more CpG dinucleotides within a region of mNKX2. In some embodiments, a region of mNKX2 comprising one or more CpG dinucleotides is set forth in SEQ ID NOs: 149, 154, and/or 159.

In some embodiments, methylated genomic DNA comprises one or more CpG dinucleotides within a region of mRASSF2. In some embodiments, a region of mRASSF2 comprising one or more CpG dinucleotides is set forth in SEQ ID NOs: 164, 169, and/or 174.

In some embodiments, methylated genomic DNA comprises one or more CpG dinucleotides within a region of mSND1. In some embodiments, a region of mSND1 comprising one or more CpG dinucleotides is set forth in SEQ ID NOs: 179, 184, and/or 189.

In some embodiments, methylated genomic DNA comprises one or more CpG dinucleotides within a region of mTBX18. In some embodiments, a region of mTBX18 comprising one or more CpG dinucleotides is set forth in SEQ ID NOs: 194, 199, and/or 204.

In some embodiments, methylated genomic DNA comprises one or more CpG dinucleotides within a region of mTFAP2E. In some embodiments, a region of mTFAP2E comprising one or more CpG dinucleotides is set forth in SEQ ID NOs: 209, 214, and/or 219.

In some embodiments, methylated genomic DNA comprises one or more CpG dinucleotides within a region of mTMEFF2. In some embodiments, a region of mTMEFF2 comprising one or more CpG dinucleotides is set forth in SEQ ID NOs: 224, 229, and/or 234.

In some embodiments, methylated genomic DNA comprises one or more CpG dinucleotides within a region of mVAX1. In some embodiments, a region of mVAX1 comprising one or more CpG dinucleotides is set forth in SEQ ID NOs: 239, and/or 244.

In some embodiments, the methods, assays and kits described herein detect mSEPT9 and mANKRD13B. In some embodiments, mSEPT9 and mANKRD13B are detected by detecting one or more CpG dinucleotides within a methylated region of mSEPT9 and mANKRD13B. In some embodiments, one or more CpG dinucleotides are detected within SEQ ID NO: 31 (mSEPT9) and within SEQ ID NOs: 6 and 11 (mANKRD13B).

In some embodiments, the methods, assay and kits described herein detect mSEPT9 using Epi proColon (epiprocoln.com; accessdata.fda.gov/cdrh_docs/pdf13/p13001c.pdf).

Detection Methods Methods for detecting methylated genomic DNA are known to those of skill in the art.

Exemplary methods are disclosed in U.S. Pat. Nos. 7,749,702; 9,965,478; and 11,186,879; US Patent Publication No. 2019-0032148; and PCT Publication No. WO 2021/122799, each of which is hereby incorporated by reference.

In some embodiments, detecting DNA methylation comprises determining the amount of methylated genomic DNA. Any means known in the art can be used to detect DNA methylation or determine its amount. In some embodiments, methylation is detected or the amount of methylated genomic DNA is determined by sequencing, in particular next-generation-sequencing (NGS), by real-time PCR or by digital PCR.

In some embodiments, detecting methylation of a genomic DNA comprises:
a) converting cytosine unmethylated in the 5-position to uracil or another base that does not hybridize to guanine in the genomic DNA of the biological sample; and
(b) detecting DNA methylation within the genomic DNA by detecting unconverted cytosine in the converted DNA of step (a).

In some embodiments, a method for detecting methylation of a genomic DNA comprises:
(a) converting cytosine unmethylated in the 5-position to uracil or another base that does not hybridize to guanine in the genomic DNA;
(b) amplifying methylation-specifically a region of the converted DNA;
(c) detecting the presence or absence of DNA amplified in step (b);
   wherein the presence or absence of amplified DNA indicates the presence or absence, respectively, of methylated genomic DNA.

In some embodiments, amplifying comprises the use of at least one oligonucleotide, preferably as a primer. In some embodiments, the oligonucleotide is bisulfite-specific. In some embodiments, the oligonucleotide is methylation-specific, such as positive methylation-specific. The oligonucleotide may be a primer or a probe oligonucleotide. In some embodiments, a probe has one or more modifications selected from the group consisting of a detectable label and a quencher, and/or a length of 5-40 nucleotides. In some embodiments a primer has a priming region with a length of 10-40 nucleotides.

DNA methylation can be detected or its amount can be determined by various means known in the art, e.g. autoradiography, silver staining or ethidium bromide staining, methylation sensitive single nucleotide extension (MS-SNUPE), methyl-binding proteins, antibodies for methylated DNA, methylation-sensitive restriction enzymes etc., preferably by sequencing, e.g. next-generation-sequencing (NGS), or by real-time PCR, e.g. multiplex real time PCR, or by digital PCR (dPCR). In some embodiments, if 3 or more (e.g. 4 or more or 5 or more) different target DNAs (i.e. markers) are examined in parallel, the presence or absence of methylated DNA is detected by sequencing, such as NGS.

In a real-time PCR, this is done by detecting a methylation-specific oligonucleotide probe during amplifying the converted (e.g. bisulfite converted) target DNA methylation-specifically using methylation-specific primers or a methylation-specific blocker with methylation-specific primers or methylation-unspecific primers.

Digital PCR (dPCR) is a quantitative PCR in which a PCR reaction mixture is partitioned into individual compartments (e.g. wells or water-in-oil emulsion droplets) resulting in either I or 0 targets being present in each compartment. Following PCR amplification, the number of positive vs negative reactions is determined and the quantification is by derived from this result statistically, preferably using Poisson statistics. In some embodiments, dPCR is BEAMing (Beads, Emulsion, Amplification, Magnetics), in which DNA templates (which may be pre amplified) are amplified using primers bound to magnetic beads present compartmentalized in water-in-oil emulsion droplets. Amplification results in the beads being covered with amplified DNA. The beads are then pooled and amplification is analyzed, e.g. using methylation-specific fluorescent probes which can be analyzed by flow cytometry. See for instance Yokoi et al. (Int J Sci. 2017 April; 18(4):735). Applied to methylation analysis, the method is also known as Methyl BEAMing.

In some embodiments, a detection by sequencing is a detection by NGS. Therein, the converted methylated target DNA is amplified, such as methylation-specifically (the target DNA is amplified if it is methylated, in other words if cytosines of the CpG sites are not converted). This can be achieved by bisulfite-specific primers which are methylation-specific. Then, the amplified sequences are sequenced and subsequently counted. The ratio of sequences derived from converted methylated DNA (identified in the sequences by CpG sites) and sequences derived from converted unmethylated DNA is calculated, resulting in a (relative) amount of methylated target DNA.

The term “next-generation-sequencing” (NGS, also known as 2^{nd} or 3^{rd} generation sequencing) refers to a sequencing the bases of a small fragment of DNA are sequentially identified from signals emitted as each fragment is re-synthesized from a DNA template strand. NGS extends this process across millions of reactions in a massively parallel fashion, rather than being limited to a single or a few DNA fragments. This advance enables rapid sequencing of the amplified DNA, with the latest instruments capable of producing hundreds of gigabases of data in a single sequencing run. See, e.g., Shendure and Ji, Nature Biotechnology 26, 1135-1145 (2008) or Mardis, Annu Rev Genomics Hum Genet. 2008; 9.387-402. Suitable NGS platforms are available commercially, e.g. the Roche 454 platform, the Roche 454 Junior platform, the Illumina HiSeq or MiSeq platforms, or the Life Technologies SOLiD 5500 or Ion Torrent platforms.

Generally, a quantification (e.g. determining the amount of methylated target DNA) may be absolute, e.g. in pg per mL or ng per mL sample, copies per mL sample, number of PCR cycles etc., or it may be relative, e.g. 10 fold higher than in a control sample or as percentage of methylation of a reference control (preferably fully methylated DNA). Determining the amount of methylated target DNA in the sample may comprise normalizing for the amount of total DNA in the sample. In some embodiments, normalizing for the amount of total DNA in the test sample comprises calculating the ratio of the amount of methylated target DNA and (i) the amount of DNA of a reference site or (ii) the amount of total DNA of the target (e.g. the amount of methylated target DNA plus the amount of unmethylated target DNA, the latter preferably measured on the reverse strand). A reference site can be any genomic site and does not have to be a gene. In some embodiments, the number of occurrences of the sequence of the reference site is stable or expected to be stable (i.e. constant) over a large population (e.g. is not in a repeat, i.e. in repetitive DNA). The reference site can, for instance be a housekeeping gene such as beta-Actin.

As mentioned above, the amount of methylated target DNA in the sample may be expressed as the proportion of the amount of methylated target DNA relative to the amount of methylated target DNA (reference control) in a reference sample comprising substantially fully methylated genomic DNA. In some embodiments, determining the proportion of methylated target DNA comprises determining the amount of methylated DNA of the same target in a reference sample, inter sample normalization of total methylated DNA, preferably by using the methylation unspecific measurement of a reference site, and dividing the ratio derived from the test sample by the corresponding ratio derived from the reference sample. The proportion can be expressed as a percentage or PMR (Percentage of Methylated Reference) by multiplying the result of the division by 100. The determination of the PMR is described in detail in Ogino et al. (JMD May 2006, Vol. 8, No. 2).

The term “amplifying” or “generating an amplicon” as used herein refers to an increase in the number of copies of the target nucleic acid and its complementary sequence, or particularly a region thereof. The target can be a double-stranded or single-stranded DNA template. The amplification may be performed by using any method known in the art, typically with a polymerase chain reaction (PCR). An “amplicon” is a double-stranded fragment of DNA according to said defined region. In some embodiments, the amplification is performed by methylation-specific PCR (i.e. an amplicon is produced depending on whether one or more CpG sites are converted or not) using (i) methylation-specific primers, or (ii) primers which are methylation-unspecific, but specific to bisulfite-converted DNA (i.e. hybridize only to converted DNA by covering at least one converted C not in a CpG context). Methylation-specificity with (ii) is achieved by using methylation-specific blocker oligonucleotides, which hybridize specifically to converted or non-converted CpG sites and thereby terminate the PCR polymerization. For example, the step of amplifying comprises a real-time PCR, in particular HeavyMethyl™ or HeavyMethyl™-MethyLight™.

The term “genomic DNA” as used herein refers to chromosomal DNA and is used to distinguish from coding DNA. As such, it comprises all coding and non-coding DNA and includes exons, introns as well as regulatory sequences, in particular promoters, belonging to a gene.

The phrase “converting, in DNA, cytosine unmethylated in the 5-position to uracil or another base that does not hybridize to guanine” as used herein refers to a process of chemically treating the DNA in such a way that all or substantially all of the unmethylated cytosine bases are converted to uracil bases, or another base which is dissimilar to cytosine in terms of base pairing behavior, while the 5-methylcytosine bases remain unchanged. The conversion of unmethylated, but not methylated, cytosine bases within the DNA sample is conducted with a converting agent. The term “converting agent” as used herein relates to a reagent capable of converting an unmethylated cytosine to uracil or to another base that is detectably dissimilar to cytosine in terms of hybridization properties. In some embodiments the converting agent is a bisulfite such as disulfite, or hydrogen sulfite. The reaction is performed according to standard procedures (Frommer et al, 1992, Proc Natl Acad Sci USA 89:1827-31, Olek, 1996, Nucleic Acids Res 24:5064-6; EP 1394172). It is also possible to conduct the conversion enzymatically, e.g., by use of methylation specific cytidine deaminases. In some embodiments, the converting agent is sodium bisulfite, ammonium bisulfite or bisulfite.

The term “bisulfite-specific” means specific for bisulfite-converted DNA. Bisulfite-converted DNA is DNA in which at least one C not in a CpG context (e.g., of a CpC, CpA or CpT dinucleotide), has/have been converted into a T or U (chemically converted into U, which by DNA amplification becomes T). With respect to an oligonucleotide, it means that the oligonucleotide covers or hybridizes to at least one nucleotide derived from conversion of a C not in a CpG context (e.g. of a CpC, CpA or CpT dinucleotide) or its complement into a T The term “methylation-specific” as used herein refers generally to the dependency from the presence or absence of CpG methylation.

The term “methylation-specific” as used herein with respect to an oligonucleotide means that the oligonucleotide does or does not anneal to a single-strand of DNA (in which cytosine unmethylated in the 5-position has been converted to uracil or another base that does not hybridize to guanine, and where it comprises at least one CpG site before conversion) without a mismatch regarding the position of the C in the at least one CpG site, depending on whether the C of the at least one CpG sites was unmethylated or methylated prior to the conversion, i.e. on whether the C has been converted or not. The methylation-specificity can be either positive (the oligonucleotide anneals without said mismatch if the C was not converted) or negative (the oligonucleotide anneals without said mismatch if the C was converted). To prevent annealing of the oligonucleotide contrary to its specificity, it preferably covers at least 2, 3, 4, 5 or 6 and preferably 3 to 6 CpG sites before conversion, or if used as a primer, covers at least one CpG site in a position where within a DNA amplification reaction a mismatch would block the oligonucleotide's extension at its 3′ end.

The term “methylation-unspecific” as used herein refers generally to the independency from the presence or absence of CpG methylation.

The term “methylation-unspecific” as used herein with respect to an oligonucleotide means that the oligonucleotide does anneal to a single-strand of DNA (in which cytosine unmethylated in the 5-position has been converted to uracil or another base that does not hybridize to guanine, and where it may or may not comprise at least one CpG site before conversion) irrespective of whether the C of the at least one CpG site was unmethylated or methylated prior to the conversion, i.e. of whether the C has been converted or not. In one case, the region of the single-strand of DNA the oligonucleotide anneals to does not comprise any CpG sites (before and after conversion) and the oligonuclotides is methylation-unspecific solely for this reason. While a methylation-unspecific oligonucleotide may cover one or more CpG dinucleotides, it does so with mismatches and/or spacers. The term “mismatch” as used herein refers to base-pair mismatch in DNA, more specifically a base-pair that is unable to form normal base-pairing interactions (i.e., other than “A” with “T” or “U”, or “G” with “C”).

Methylation is detected within the at least one genomic DNA polynucleotide, i.e. in a particular region of the DNA according to the SEQ ID NO referred to (the “target DNA”). The term “target DNA” as used herein refers to a genomic nucleotide sequence at a specific chromosomal location. In the context of the present invention, it is typically a genetic marker that is known to be methylated in the state of disease (for example in cancer cells vs. non-cancer cells). A genetic marker can be a coding or non-coding region of genomic DNA.

The term “region of the target DNA” or “region of the converted DNA” as used herein refers to a part of the target DNA which is to be analyzed. In some embodiments, the region is at least 40, 50, 60, 70, 80, 90, 100, 150, or 200 or 300 base pairs (bp) long and/or not longer than 500, 600, 700, 800, 900 or 1000 bp (e.g. 25-500, 50-250 or 75-150 bp). In particular, it is a region comprising at least 3, 4, 5, 6, 7, 8, 9, 10, 11, 12, 13, 14, 15 or 16 CpG sites of the genomic DNA.

For an amplification of the target region with at least one methylation-specific primer, the at least one methylation-specific primer covers at least 1, at least 2 or at least 3 CpG sites (e.g., 2-8 or preferably 3-6 CpG sites) of the target region. In some embodiments, at least 1, at least 2 or preferably at least 3 CpG sites of these CpG sites are covered by the 3′ third of the primer (and/or one of these CpG sites is covered by the 3′ end of the primer (last three nucleotides of the primer).

The term “covering a CpG site” as used herein with respect to an oligonucleotide refers to the oligonucleotide annealing to a region of DNA comprising this CpG site, before or after conversion of the C of the CpG site (i.e. the CpG site of the corresponding genomic DNA when it is referred to a bisulfite converted sequence). The annealing may, with respect to the CpG site (or former CpG site if the C was converted), be methylation-specific or methylation-unspecific as described herein.

The term “annealing”, when used with respect to an oligonucleotide, is to be understood as a bond of an oligonucleotide to an at least substantially complementary sequence along the lines of the Watson-Crick base pairings in the sample DNA, forming a duplex structure, under moderate or stringent hybridization conditions. When it is used with respect to a single nucleotide or base, it refers to the binding according to Watson-Crick base pairings, e.g. C-G, A-T and A-U. Stringent hybridization conditions involve hybridizing at 68° C. in 5×SSC/5× Denhardfs solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderate conditions involve washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

“Substantially identical” means that an oligonucleotide does not need to be 100% identical to a reference sequence but can comprise mismatches and/or spacers as defined herein. It is preferred that a substantially identical oligonucleotide, if not 100% identical, comprises 1 to 3, i.e. 1, 2 or 3 mismatches and/or spacers, preferably one mismatch or spacer per oligonucleotide, such that the intended annealing does not fail due to the mismatches and/or spacers. To enable annealing despite mismatches and/or spacers, it is preferred that an oligonucleotide does not comprise more than 1 mismatch per 10 nucleotides (rounded up if the first decimal is 5 or higher, otherwise rounded down) of the oligonucleotide.

The mismatch or a spacer is preferably a mismatch with or a spacer covering an SNP in the genomic DNA of the subject. A mismatch with an SNP is preferably not complementary to any nucleotide at this position in the subject's species. The term “SNP” as used herein refers to the site of an SNP, i.e. a single nucleotide polymorphism, at a particular position in the (preferably human) genome that varies among a population of individuals. SNPs of the genomic DNA the present application refers to are known in the art and can be found in online databases such as dbSNP of NCBI (ncbi.nlm.nih.gov/snp).

The term “spacer” as used herein refers to a non-nucleotide spacer molecule, which increases, when joining two nucleotides, the distance between the two nucleotides to about the distance of one nucleotide (i.e. the distance the two nucleotides would be apart if they were joined by a third nucleotide). Non-limiting examples for spacers are Inosine, d-Uracil, halogenated bases, Amino-dT, C3, Cl 2, Spacer 9, Spacer 18, and dSpacer.

The term “oligonucleotide” as used herein refers to a linear oligomer of 5 to 50 ribonucleotides or preferably deoxyribonucleotides. Preferably, it has the structure of a single-stranded DNA fragment. The “stretch of contiguous nucleotides” referred to herein preferably is as long as the oligonucleotide.

The term “primer oligonucleotide” as used herein refers to a single-stranded oligonucleotide sequence comprising at its 3′ end a priming region which is substantially complementary to a nucleic acid sequence sought to be copied (the template) and serves as a starting point for synthesis of a primer extension product. Preferably, the priming region is 10 to 40 nucleotides, more preferably 15-30 nucleotides and most preferably 19 to 25 nucleotides in length. The “stretch of contiguous nucleotides” referred to herein preferably corresponds to the priming region. The primer oligonucleotide may further comprise, at the 5′ end of the primer oligonucleotide, an overhang region. The overhang region consists of a sequence which is not complementary to the original template, but which is in a subsequent amplification cycle incorporated into the template by extension of the opposite strand. The overhang region has a length that does not prevent priming by the priming region (e.g. annealing of the primer via the priming region to the template). For example, it may be 1-200 nucleotides, preferably 4-100 or 4-50, more preferably 4-25 or most preferably 4-15 nucleotides long. The overhang region usually comprises one or more functional domains, i.e. it has a sequence which encodes (not in the sense of translation into a polypeptide) a function which is or can be used for the method of the first aspect. Examples of functional domains are restriction sites, ligation sites, universal priming sites (e.g. for NGS), annealing sites (not for annealing to the template to be amplified by extension of the priming region, but to other oligonucleotides), and index (barcode) sites. The overhang region does not comprise a “stretch of contiguous nucleotides” as referred to herein with respect to the methylation markers of the invention. It is, as indicated above, understood by the skilled person that the sequence of an overhang region incorporated into a new double-strand generated by amplification. Therefore, the overhang region could be considered part of the priming region for further amplification of the new double-strand.

However, the term “priming region” is used herein to distinguish a region that is the priming region of the initial template, i.e. which has a sequence that substantially corresponds to a methylation marker sequence from an overhang region with respect to the same methylation marker sequence.

It is also understood by the skilled person that the term “template” in the context of amplification of bisulfite converted DNA comprises not only double-stranded DNA, but also a single strand that is the result of bisulfite conversion of genomic DNA (rendering it non complementary to its previous opposite strand) In the first round of amplification, only one of the primers of a primer pair binds to this single-strand and is extended, thereby creating a new complementary opposite strand to which the other primer of the primer pair can bind. Table 3 provides the sequences of the strands that are the result of bisulfite conversion of the genomic DNA of the methylation markers of the invention (bis1 and bis2), as well as corresponding new complementary opposite strands in 5′-3′ orientation (rc).

The term “primer pair” as used herein refers to two oligonucleotides, namely a forward and a reverse primer, that have, with respect to a double-stranded nucleic acid molecule (including a single strand that is the result of bisulfite conversion plus the new complementary opposite strand to be created as explained above), sequences that are (at least substantially) identical to one strand each such that they each anneal to the complementary strand of the strand they are (at least substantially) identical to. The term “forward primer” refers to the primer which is (at least substantially) identical to the forward strand (as defined by the direction of the genomic reference sequence) of the double-stranded nucleic acid molecule, and the term “reverse primer” refers to the primer which is (at least substantially) identical to the reverse complementary strand of the forward strand in the double-stranded nucleic acid molecule. The distance between the sites where forward and reverse primer anneal to their template depends on the length of the amplicon the primers are supposed to allow generating. Typically, with respect to the present invention it is between 40 and 1000 bp. Preferred amplicon sizes are specified herein. In case of single-stranded DNA template that is to be amplified using a pair of primers, only one of the primers anneals to the single strand in the first amplification cycle. The other primer then binds to the newly generated complementary strand such that the result of amplification is a double-stranded DNA fragment.

In some embodiments, the primer pair for detecting mSEPT9 comprises SEQ ID NO: 45 and SEQ ID NO: 46. In some embodiments, the probe for detecting mSEPT9 comprises SEQ ID NO: 49. In some embodiments, the primer pair for detecting mANKRD13B comprises SEQ ID NO: 41 and SEQ ID NO: 42. In some embodiments, the primer pair for detecting mANKRD13B comprises SEQ ID NO: 43 and SEQ ID NO. 44. In some embodiments, the probe for detecting mANRKD13B is selected from SEQ ID NOs. 47 and 48.

In some embodiments, the primer pair for detecting mADCYAP1 comprises SEQ ID NO: 249 and SEQ ID NO. 250. In some embodiments, the primer pair for detecting mKHDRBS2 comprises SEQ ID NO: 251 and SEQ ID NO: 252. In some embodiments, the primer pair for detecting mCLEC14 comprises SEQ ID NO: 253 and SEQ ID NO: 254. In some embodiments, the primer pair for detecting mCRMP1 comprises SEQ ID NO. 255 and SEQ ID NO: 256. In some embodiments, the primer pair for detecting mEYA4 comprises SEQ ID NO: 257 and SEQ ID NO: 258. In some embodiments, the primer pair for detecting mMSC comprises SEQ ID NO: 259 and SEQ ID NO. 260. In some embodiments, the primer pair for detecting mNGFR comprises SEQ ID NO: 261 and SEQ ID NO: 262. In some embodiments, the primer pair for detecting mNKX2 comprises SEQ ID NO: 263 and SEQ ID NO: 264. In some embodiments, the primer pair for detecting mRASSF2 comprises SEQ ID NO: 265 and SEQ ID NO: 266. In some embodiments, the primer pair for detecting mSND1 comprises SEQ ID NO: 267 and SEQ ID NO: 268. In some embodiments, the primer pair for detecting mTBX18 comprises SEQ ID NO: 269 and SEQ ID NO: 270. In some embodiments, the primer pair for detecting mTFAP2E comprises SEQ ID NO: 271 and SEQ ID NO: 272. In some embodiments, the primer pair for detecting mTMEFF2 comprises SEQ ID NO: 273 and SEQ ID NO: 274 In some embodiments, the primer pair for detecting mVAX1 comprises SEQ ID NO: 275 and SEQ ID NO: 276.

The term “blocker” as used herein refers to a molecule which binds in a methylation-specific manner to a single-strand of DNA (i.e. it is specific for either the converted methylated or preferably for the converted unmethylated DNA or the amplified DNA derived from it) and prevents amplification of the DNA by binding to it, for example by preventing a primer to bind or by preventing primer extension where it binds. Non-limiting examples for blockers are sequence and/or methylation specific antibodies (blocking e.g. primer binding or the polymerase) and in particular blocker oligonucleotides.

A “blocker oligonucleotide” may be a blocker that prevents the extension of the primer located upstream of the blocker oligonucleotide. It comprises nucleosides/nucleotides having a backbone resistant to the 5′ nuclease activity of the polymerase. This may be achieved, for example, by comprising peptide nucleic acid (PNA), locked nucleic acid (LNA), Morpholino, glycol nucleic acid (GNA), threose nucleic acid (TNA), bridged nucleic acids (BNA), N3′-P5′ phosphoramidate (NIP) oligomers, minor groove binder-linked-oligonucleotides (MGB-linked oligonucleotides), phosphorothioate (PS) oligomers, CrC₄alkylphosphonate oligomers, phosphoramidates, b-phosphodiester oligonucleotides, a-phosphodiester oligonucleotides or a combination thereof. Alternatively, it may be a non-extendable oligonucleotide with a binding site on the DNA single-strand that overlaps with the binding site of a primer oligonucleotide. When the blocker is bound, the primer cannot bind and therefore the amplicon is not generated. When the blocker is not bound, the primer-binding site is accessible and the amplicon is generated. For such an overlapping blocker, it is preferable that the affinity of the blocker is higher than the affinity of the primer for the DNA. A blocker oligonucleotide is typically 15 to 50, preferably 20 to 40 and more preferably 25 to 35 nucleotides long. “At least one blocker” refers in particular to a number of 1, 2, 3, 4 or 5 blockers, more particularly to 1-2 or 1-3 blockers. Also, a blocker oligonucleotide cannot by itself act as a primer (i.e. cannot be extended by a polymerase) due to a non-extensible 3′ end.

The term “probe oligonucleotide” or “probe” as used herein refers to an oligonucleotide that is used to detect an amplicon by annealing to one strand of the amplicon, usually not where any of the primer oligonucleotides binds (i.e. not to a sequence segment of the one strand which overlaps with a sequence segment a primer oligonucleotide anneals to). Preferably it anneals without a mismatch or spacer, in other words it is preferably complementary to one strand of the amplicon. A probe oligonucleotide is preferably 5-40 nucleotides, more preferably 10 to 25 and most preferably 15 to 20 nucleotides long. The “stretch of contiguous nucleotides” referred to herein preferably is as long as the probe oligonucleotide. Usually, the probe is linked, preferably covalently linked, to at least one detectable label which allows detection of the amplicon and/or at least one quencher which allows quenching the signal of a (preferably the) detectable label. The term “detectable label” as used herein does not exhibit any particular limitation. The detectable label may be selected from the group consisting of radioactive labels, luminescent labels, fluorescent dyes, compounds having an enzymatic activity, magnetic labels, antigens, and compounds having a high binding affinity for a detectable label. For example, fluorescent dyes linked to a probe may serve as a detection label, e.g. in a real-time PCR. Suitable radioactive markers are P-32, S-35, 1-125, and H-3, suitable luminescent markers are chemiluminescent compounds, preferably luminol, and suitable fluorescent markers are preferably dansyl chloride, fluorcein-5-isothiocyanate, and 4-fluor-7-nitrobenz-2-aza-1,3 diazole, in particular 6-Carboxyfluorescein (FAM), 6-Hexachlorofluorescein (HEX), 5(6)-Carboxytetramethylrhodamine (TAMRA), 5(6)-Carboxy-X-Rhodamine (ROX), Cyanin-5-Fluorophor (Cy5) and derivates thereof, suitable enzyme markers are horseradish peroxidase, alkaline phosphatase, a-galactosidase, acetylcholinesterase, or biotin. A probe may also be linked to a quencher. The term “quencher” as used herein refers to a molecule that deactivates or modulates the signal of a corresponding detectable label e.g. by energy transfer, electron transfer, or by a chemical mechanism as defined by IUPAC (see compendium of chemical terminology 2 ed. 1997). In particular, the quencher modulates the light emission of a detectable label that is a fluorescent dye. In some cases, a quencher may itself be a fluorescent molecule that emits fluorescence at a characteristic wavelength distinct from the label whose fluorescence it is quenching. In other cases, the quencher does not itself fluoresce (i.e. the quencher is a “dark acceptor”). Such quenchers include, for example, dabcyl, methyl red, the QSY diarylrhodamine dyes, and the like.

Sequences for detecting a methylated genomic DNA are summarized in the table below:

| SEQ | | | Location | |
|---|---|---|---|---|
| ID NO | Abbr. | Name | GRCh38 | Type |
| 1 | ANB1 | ANKRD13BoB1 | 17: 29613595- | genomic reference |
| 2 | | Assay | 29613658 | converted C to T (bis1) |
| 3 | | | | converted C to T (bis1), reverse complement |
| 4 | | | | converted G to A (bis2 rc) |
| 5 | | | | converted G to A (bis2 rc), reverse complement |
| 6 | | ANKRD13B | 17: 29613595- | genomic reference |
| 7 | | Extended | 29613700 | converted C to T (bis1) |
| 8 | | Assay | | converted C to T (bis1), reverse complement |
| 9 | | Region A | | converted G to A (bis2 rc) |
| 10 | | | | converted G to A (bis2 rc), reverse complement |
| 11 | | ANKRD13B | 17: 29613595- | genomic reference |
| 12 | | Extended | 29613940 | converted C to T (bis1) |
| 13 | | Assay | | converted C to T (bis1), reverse complement |
| 14 | | Region B | | converted G to A (bis2 rc) |
| 15 | | | | converted G to A (bis2 rc), reverse complement |
| 16 | ANB2 | ANKRD13BoB2 | 17: 29613595- | genomic reference |
| 17 | | Assay | 29613681 | converted C to T (bis1) |
| 18 | | | | converted C to T (bis1), reverse complement |
| 19 | | | | converted G to A (bis2 rc) |
| 20 | | | | converted G to A (bis2 rc), reverse complement |
| 21 | S9B2 | SEPTIN9oB2 | 17: 77373482- | genomic reference |
| 22 | | Assay | 77373549 | converted C to T (bis1) |
| 23 | | | | converted C to T (bis1), reverse complement |
| 24 | | | | converted G to A (bis2 rc) |
| 25 | | | | converted G to A (bis2 rc), reverse complement |
| 26 | | SEPTIN9 | 17: 77373475- | genomic reference |
| 27 | | Assay | 77373549 | converted C to T (bis1) |
| 28 | | Region | | converted C to T (bis1), reverse complement |
| 29 | | | | converted G to A (bis2 rc) |
| 30 | | | | converted G to A (bis2 rc), reverse complement |
| 31 | | SEPTIN9 | 17: 77372606- | genomic reference |
| 32 | | Assay + CpG- | 77374424 | converted C to T (bis1) |
| 33 | | island | | converted C to T (bis1), reverse complement |
| 34 | | | | converted G to A (bis2 rc) |
| 35 | | | | converted G to A (bis2 rc), reverse complement |
| 36 | | SEPTIN9 | 17: 77372979- | genomic reference |
| 37 | | Extended | 77374040 | converted C to T (bis1) |
| 38 | | Assay | | converted C to T (bis1), reverse complement |
| 39 | | Region | | converted G to A (bis2 rc) |
| 40 | | | | converted G to A (bis2 rc), reverse complement |
| 54 | | mADCYAP1 | 18: 906256- | genomic reference |
| 55 | | Assay + CpG | 909573 | converted C to T (bis1) |
| 56 | | island | | converted C to T (bis1), reverse complement |
| 57 | | | | converted G to A (bis2 rc) |
| 58 | | | | converted G to A (bis2 rc), reverse complement |
| 59 | | mADCYAP1 | 18: 906345- | genomic reference |
| 60 | | Extended | 907438 | converted C to T (bis1) |
| 61 | | Assay | | converted C to T (bis1), reverse complement |
| 62 | | | | converted G to A (bis2 rc) |
| 63 | | | | converted G to A (bis2 rc), reverse complement |
| 64 | | mADCYAP1 | 18: 906845- | genomic reference |
| 65 | | Assay | 906938 | converted C to T (bis1) |
| 66 | | | | converted C to T (bis1), reverse complement |
| 67 | | | | converted G to A (bis2 rc) |
| 68 | | | | converted G to A (bis2 rc), reverse complement |
| 69 | | mKHDRBS2 | 6: 62285170- | genomic reference |
| 70 | | Extended | 62286248 | converted C to T (bis1) |
| 71 | | Assay | | converted C to T (bis1), reverse complement |
| 72 | | | | converted G to A (bis2 rc) |
| 73 | | | | converted G to A (bis2 rc), reverse complement |
| 74 | | mKHDRBS2 | 6: 62285670- | genomic reference |
| 75 | | Assay | 62285748 | converted C to T (bis1) |
| 76 | | | | converted C to T (bis1), reverse complement |
| 77 | | | | converted G to A (bis2 rc) |
| 78 | | | | converted G to A (bis2 rc), reverse complement |
| 79 | | mCLEC14A | 14: 38255049- | genomic reference |
| 80 | | Assay + CpG | 38256332 | converted C to T (bis1) |
| 81 | | Island | | converted C to T (bis1), reverse complement |
| 82 | | | | converted G to A (bis2 rc) |
| 83 | | | | converted G to A (bis2 rc), reverse complement |
| 84 | | mCLEC14A | 14: 38255401- | genomic reference |
| 85 | | Extended | 38256502 | converted C to T (bis1) |
| 86 | | Assay | | converted C to T (bis1), reverse complement |
| 87 | | | | converted G to A (bis2 rc) |
| 88 | | | | converted G to A (bis2 rc), reverse complement |
| 89 | | mCLEC14A | 14: 38255901- | genomic reference |
| 90 | | | 38256002 | converted C to T (bis1) |
| 91 | | | | converted C to T (bis1), reverse complement |
| 92 | | | | converted G to A (bis2 rc) |
| 93 | | | | converted G to A (bis2 rc), reverse complement |
| 94 | | mCRMP1 | 4: 5890481- | genomic reference |
| 95 | | Extended | 5891551 | converted C to T (bis1) |
| 96 | | Assay | | converted C to T (bis1), reverse complement |
| 97 | | | | converted G to A (bis2 rc) |
| 98 | | | | converted G to A (bis2 rc), reverse complement |
| 99 | | mCRMP1 | 4: 5890981- | genomic reference |
| 100 | | Assay | 5891051 | converted C to T (bis1) |
| 101 | | | | converted C to T (bis1), reverse complement |
| 102 | | | | converted G to A (bis2 rc) |
| 103 | | | | converted G to A (bis2 rc), reverse complement |
| 104 | | mEYA4 | 6: 133240948- | genomic reference |
| 105 | | Assay + CpG | 133242448 | converted C to T (bis1) |
| 106 | | Island | | converted C to T (bis1), reverse complement |
| 107 | | | | converted G to A (bis2 rc) |
| 108 | | | | converted G to A (bis2 rc), reverse complement |
| 109 | | mEYA4 | 6: 133241300- | genomic reference |
| 110 | | Extended | 13242493 | converted C to T (bis1) |
| 111 | | Assay | | converted C to T (bis1), reverse complement |
| 112 | | | | converted G to A (bis2 rc) |
| 113 | | | | converted G to A (bis2 rc), reverse complement |
| 114 | | mEYA4 | 6: 133241800- | genomic reference |
| 115 | | Assay | 133241993 | converted C to T (bis1) |
| 116 | | | | converted C to T (bis1), reverse complement |
| 117 | | | | converted G to A (bis2 rc) |
| 118 | | | | converted G to A (bis2 rc), reverse complement |
| 119 | | mMSC | 8: 71841639- | genomic reference |
| 120 | | Assay + CpG | 71842520 | converted C to T (bis1) |
| 121 | | Island | | converted C to T (bis1), reverse complement |
| 122 | | | | converted G to A (bis2 rc) |
| 123 | | | | converted G to A (bis2 rc), reverse complement |
| 124 | | mMSC | 8: 71841868- | genomic reference |
| 125 | | Extended | 71842937 | converted C to T (bis1) |
| 126 | | Assay | | converted C to T (bis1), reverse complement |
| 127 | | | | converted G to A (bis2 rc) |
| 128 | | | | converted G to A (bis2 rc), reverse complement |
| 129 | | mMSC | 8: 71842368- | genomic reference |
| 130 | | Assay | 71842437 | converted C to T (bis1) |
| 131 | | | | converted C to T (bis1), reverse complement |
| 132 | | | | converted G to A (bis2 rc) |
| 133 | | | | converted G to A (bis2 rc), reverse complement |
| 134 | | mNGFR | 17: 49494983- | genomic reference |
| 135 | | Assay + CpG | 49497954 | converted C to T (bis1) |
| 136 | | Island | | converted C to T (bis1), reverse complement |
| 137 | | | | converted G to A (bis2 rc) |
| 138 | | | | converted G to A (bis2 rc), reverse complement |
| 139 | | mNGFR | 17: 49497163- | genomic reference |
| 140 | | Extended | 49498222 | converted C to T (bis1) |
| 141 | | Assay | | converted C to T (bis1), reverse complement |
| 142 | | | | converted G to A (bis2 rc) |
| 143 | | | | converted G to A (bis2 rc), reverse complement |
| 144 | | mNGFR | 17: 49497663- | genomic reference |
| 145 | | Assay | 49497722 | converted C to T (bis1) |
| 146 | | | | converted C to T (bis1), reverse complement |
| 147 | | | | converted G to A (bis2 rc) |
| 148 | | | | converted G to A (bis2 rc), reverse complement |
| 149 | | mNKX2 | 20: 21510655- | genomic reference |
| 150 | | Assay + CpG | 21513742 | converted C to T (bis1) |
| 151 | | Island | | converted C to T (bis1), reverse complement |
| 152 | | | | converted G to A (bis2 rc) |
| 153 | | | | converted G to A (bis2 rc), reverse complement |
| 154 | | mNKX2 | 20: 21512255- | genomic reference |
| 155 | | Extended | 21513321 | converted C to T (bis1) |
| 156 | | Assay | | converted C to T (bis1), reverse complement |
| 157 | | | | converted G to A (bis2 rc) |
| 158 | | | | converted G to A (bis2 rc), reverse complement |
| 159 | | mNKX2 | 20: 21512755- | genomic reference |
| 160 | | Assay | 21512821 | converted C to T (bis1) |
| 161 | | | | converted C to T (bis1), reverse complement |
| 162 | | | | converted G to A (bis2 rc) |
| 163 | | | | converted G to A (bis2 rc), reverse complement |
| 164 | | mRASSF2 | 20: 1822367- | genomic reference |
| 165 | | Assay + CpG | 4823486 | converted C to T (bis1) |
| 166 | | Island | | converted C to T (bis1), reverse complement |
| 167 | | | | converted G to A (bis2 rc) |
| 168 | | | | converted G to A (bis2 rc), reverse complement |
| 169 | | mRASSF2 | 20: 4822086- | genomic reference |
| 170 | | Extended | 4823162 | converted C to T (bis1) |
| 171 | | Assay | | converted C to T (bis1), reverse complement |
| 172 | | | | converted G to A (bis2 rc) |
| 173 | | | | converted G to A (bis2 rc), reverse complement |
| 174 | | mRASSF2 | 20: 4822586- | genomic reference |
| 175 | | Assay | 4822662 | converted C to T (bis1) |
| 176 | | | | converted C to T (bis1), reverse complement |
| 177 | | | | converted G to A (bis2 rc) |
| 178 | | | | converted G to A (bis2 rc), reverse complement |
| 179 | | mSND1 | 7: 128104142- | genomic reference |
| 180 | | Assay + CpG | 128104502 | converted C to T (bis1) |
| 181 | | Island | | converted C to T (bis1), reverse complement |
| 182 | | | | converted G to A (bis2 rc) |
| 183 | | | | converted G to A (bis2 rc), reverse complement |
| 184 | | mSND1 | 7: 128103804- | genomic reference |
| 185 | | Extended | 128104900 | converted C to T (bis1) |
| 186 | | Assay | | converted C to T (bis1), reverse complement |
| 187 | | | | converted G to A (bis2 rc) |
| 188 | | | | converted G to A (bis2 rc), reverse complement |
| 189 | | mSND1 | 7: 128104304- | genomic reference |
| 190 | | Assay | 128104400 | converted C to T (bis1) |
| 191 | | | | converted C to T (bis1), reverse complement |
| 192 | | | | converted G to A (bis2 rc) |
| 193 | | | | converted G to A (bis2 rc), reverse complement |
| 194 | | mTBX18 | 6: 84762984- | genomic reference |
| 195 | | Assay + CpG | 84764414 | converted C to T (bis1) |
| 196 | | Island | | converted C to T (bis1), reverse complement |
| 197 | | | | converted G to A (bis2 rc) |
| 198 | | | | converted G to A (bis2 rc), reverse complement |
| 199 | | mTBX18 | 6: 84763288- | genomic reference |
| 200 | | Extended | 84764374 | converted C to T (bis1) |
| 201 | | Assay | | converted C to T (bis1), reverse complement |
| 202 | | | | converted G to A (bis2 rc) |
| 203 | | | | converted G to A (bis2 rc), reverse complement |
| 204 | | mTBX18 | 6: 84763788- | genomic reference |
| 205 | | Assay | 84763874 | converted C to T (bis1) |
| 206 | | | | converted C to T (bis1), reverse complement |
| 207 | | | | converted G to A (bis2 rc) |
| 208 | | | | converted G to A (bis2 rc), reverse complement |
| 209 | | mTFAP2E | 1: 35576831- | genomic reference |
| 210 | | Assay + CpG | 35577843 | converted C to T (bis1) |
| 211 | | Island | | converted C to T (bis1), reverse complement |
| 212 | | | | converted G to A (bis2 rc) |
| 213 | | | | converted G to A (bis2 rc), reverse complement |
| 214 | | mTFAP2E | 1: 35577250- | genomic reference |
| 215 | | Extended | 35578318 | converted C to T (bis1) |
| 216 | | Assay | | converted C to T (bis1), reverse complement |
| 217 | | | | converted G to A (bis2 rc) |
| 218 | | | | converted G to A (bis2 rc), reverse complement |
| 219 | | mTFAP2E | 1: 355777750- | genomic reference |
| 220 | | Assay | 35577818 | converted C to T (bis1) |
| 221 | | | | converted C to T (bis1), reverse complement |
| 222 | | | | converted G to A (bis2 rc) |
| 223 | | | | converted G to A (bis2 rc), reverse complement |
| 224 | | mTMEFF2 | 2: 192194269- | genomic reference |
| 225 | | Assay + CpG | 192196086 | converted C to T (bis1) |
| 226 | | Island | | converted C to T (bis1), reverse complement |
| 227 | | | | converted G to A (bis2 rc) |
| 228 | | | | converted G to A (bis2 rc), reverse complement |
| 229 | | mTMEFF2 | 2: 192195336- | genomic reference |
| 230 | | Extended | 192196409 | converted C to T (bis1) |
| 231 | | Assay | | converted C to T (bis1), reverse complement |
| 232 | | | | converted G to A (bis2 rc) |
| 233 | | | | converted G to A (bis2 rc), reverse complement |
| 234 | | mTMEFF2 | 2: 192195836- | genomic reference |
| 235 | | Assay | 192195909 | converted C to T (bis1) |
| 236 | | | | converted C to T (bis1), reverse complement |
| 237 | | | | converted G to A (bis2 rc) |
| 238 | | | | converted G to A (bis2 rc), reverse complement |
| 239 | | mVAX1 | 10: 117131597- | genomic reference |
| 240 | | Extended | 117132727 | converted C to T (bis1) |
| 241 | | Assay | | converted C to T (bis1), reverse complement |
| 242 | | | | converted G to A (bis2 rc) |
| 243 | | | | converted G to A (bis2 rc), reverse complement |
| 244 | | mVAX1 | 10: 117132097- | genomic reference |
| 245 | | Assay | 117132227 | converted C to T (bis1) |
| 246 | | | | converted C to T (bis1), reverse complement |
| 247 | | | | converted G to A (bis2 rc) |
| 248 | | | | converted G to A (bis2 rc), reverse complement |

rc = reverse complement; C to G or G to A means converted by bisulfite conversion of cytosines outside of CpG context into uracil and replaced by thymidine in subsequent amplification. Bis1 refers to the bisulfite converted forward strand (as recited in the SEQ ID of the respective genomic DNA) and bis2 refers to the bisulfite converted reverse complement strand of the forward strand (reverse complement of the SEQ ID of the respective genomic DNA), whereby the direction of the strand is defined by the direction of the genomic reference sequence as e.g., obtained from the genome build (GRCh38).

### Biological Samples

The methods, assays and kits described herein are useful for detecting one or more methylated genomic DNA and one or more protein biomarkers in a biological sample obtained from a subject. Exemplary biological samples include, but are not limited to, plasma, urine, saliva, whole blood, dried blood spot, serum, dried serum spot, stool, and/or hair. In some embodiments, the biological sample is derived from blood. In some embodiments, the biological sample is serum. In some embodiments, the biological is plasma. In some embodiments, the biological sample is whole blood, serum or plasma. In some embodiments, a processed biological sample, e.g., blood plasma or serum, is frozen for transport and/or long-term storage.

In some embodiments, the methods, assays and kits described herein detect one or more methylated genomic DNA and one or more protein biomarkers in one or more biological samples. In some embodiments, one or more methylated genomic DNA and one or more protein biomarkers are detected in different biological samples obtained from the same patient. For example, methylated genomic DNA may be detected in plasma and a protein biomarker is detected in stool, or vice versa. In some embodiments, one or more methylated genomic DNA and one or more protein biomarkers are detected in the same biological obtained from the same patient.

In some embodiments, the biological sample is processed to allow for detecting of the target of interest, i.e., methylated genomic DNA or protein biomarker. In some embodiments, a biological sample is desialylated. Desialylation is a part of sialic acid metabolism, which removes the terminal sialic acid residue on glycans to modulate the structure and function of glycans, glycoproteins or glycolipids. In some embodiments, desialylation of a sample is needed to identify a target of interest, e.g., glycoform of haptoglobin as described herein. In some embodiments, a serum sample is desialylated. In some embodiments, a plasma sample is desialylated. In some embodiments, a blood sample is desialylated. In some embodiments, desialylating a sample comprises treating the sample with a mild acid. In some embodiments, desialylating a sample comprises treating the sample with a neuraminidase.

In some embodiments, a biological sample is processed in a manner consistent with methods for detecting protein or DNA methylation. In some embodiments, a sample is processed to isolate the proteins for detection. Methods for isolating proteins are known to those of skill in the art. In some embodiments, a sample is processed to isolate genomic DNA for methylation detection. Methods for isolating genomic DNA are known to those of skill in the art.

### Subject Screening Eligibility

In some aspects, the methods, assays and kits described herein are useful for screening a subject for a proliferative disease. A subject eligible for screening can be identified by a clinician. In some embodiments, a subject eligible for screening is based on criteria defined by the American Cancer Society for cancer generally or for colorectal cancer. Exemplary criteria for identifying a subject eligible for screening includes, but is not limited to: age, gender, proliferative disease status, treatment of proliferative disease status, family medical history, lifestyle, and weight.

In some embodiments, a subject eligible for screening is suspected of having a proliferative disease. In some embodiments, a subject eligible for screening is suspected of being at risk for having a proliferative disease. In some embodiments, a subject eligible for screening has not been previously diagnosed with a proliferative disease. In some embodiments, a subject eligible for screening has been previously diagnosed with a proliferative disease but is suspected of having or being at risk for having a different proliferative disease. In some embodiments, a subject eligible for screening has not received treatment for a proliferative disease. In some embodiments, a subject eligible for screening has not been subject to surgical treatment for a proliferative disease.

In some embodiments, a subject eligible for screening is suspected of having colorectal cancer or a colorectal cell proliferative disease. In some embodiments, a subject eligible for screening is suspected of having a pre-cancerous lesion (e.g., advanced adenoma). In some embodiments, a subject eligible for screening is suspected of being at risk for having colorectal cancer or a colorectal cell proliferative disease. In some embodiments, a subject eligible for screening is suspected of being at risk for having a pre-cancerous lesion (e.g., advanced adenoma). In some embodiments, a subject eligible for screening has not been previously diagnosed with colorectal cancer or a colorectal cell proliferative disease. In some embodiments, a subject eligible for screening has not been previously diagnosed with a pre-cancerous lesion (e.g., advanced adenoma). In some embodiments, a subject eligible for screening has been previously diagnosed with a pre-cancerous lesion (e.g., advanced adenoma). In some embodiments, a subject eligible for screening has been previously diagnosed with a proliferative disease but is suspected of having or being at risk for having colorectal cancer or a colorectal cell proliferative disease. In some embodiments, a subject eligible for screening has not received treatment for colorectal cancer or a colorectal cell proliferative disease. In some embodiments, a subject eligible for screening is suspected of having a recurrence of colorectal cancer or a colorectal cell proliferative disease. In some embodiments, a subject eligible for screening has not been subject to surgical treatment for colorectal cancer or a colorectal cell proliferative disease. In some embodiments, a subject eligible for screening has not received a colonoscopy. In some embodiments, a subject eligible for screening has not receive a recent colonoscopy for diagnosing colorectal cancer or a colorectal cell proliferative disease.

In some embodiments, a subject eligible for screening for a proliferative disease is at least 45 years of age. In some embodiments, a subject eligible for screening for a proliferative disease is 45-85 years of age. In some embodiments, a subject eligible for screening for a proliferative disease is 45-75 years of age.

In some embodiments, a subject eligible for screening for colorectal cancer or a colorectal cell proliferative disease is at least 45 years of age. In some embodiments, a subject eligible for screening for colorectal cancer or a colorectal cell proliferative disease is 45-85 years of age. In some embodiments, a subject eligible for screening for colorectal cancer or a colorectal cell proliferative disease is 45-75 years of age.

In some embodiments, a subject eligible for screening exhibits at least one risk factor associated with a proliferative disease. In some embodiments, a subject eligible for screening exhibits at least one risk factor associated with colorectal cancer or a colorectal cell proliferative disease. Risk factors can include, but are not limited to, family history of cancer, obesity, alcohol consumption, exposure to chemicals, exposure to radiation, tobacco use, age, lack of physical activity, and poor diet.

### Discriminatory Ability

In some embodiments, the methods, assays and kits described herein have discriminatory ability for detecting a proliferative disease in a subject. In some embodiments, the methods, assays and kits described herein having enhanced discriminatory ability for detecting a proliferative disease in a subject relative to a method, assay or kit that does not include detecting one or more protein biomarkers.

In some embodiments, the methods, assays and kits described herein have discriminatory ability for detecting colorectal cancer or a colorectal cell proliferative disease in a subject. In some embodiments, the methods, assays and kits described herein having enhanced discriminatory ability for detecting colorectal cancer or a colorectal cell proliferative disease in a subject relative to a method, assay or kit that does not include detecting one or more protein biomarkers.

The discriminatory ability of biomarkers, such as the presence of methylated genomic DNA and proteins associated with a proliferative disease, is an evaluation of whether the biomarker can accurately identify subjects with and without the proliferative disease. Failure of a biomarker to have sufficient discriminatory ability can lead to false negatives and false positives, each having detrimental impacts on a subject. Discriminatory ability is determined based on sensitivity, specificity and receiver-operating characteristics (ROC) probability curves. Sensitivity is the ability to detect a disease in patients in whom the disease is truly present (i.e., a true positive), and specificity is the ability to rule out the disease in patients whom the disease is truly absent (i.e., a true negative). To plot ROC probability curves, the false positive rate is plotted on the x-axis against the true positive rate on the y-axis. The area under the ROC curve (AUC, or AUROC) ranges from 0.5, indicating no power to separate cases from non-cases, to 1, indicating perfect discrimination. To be clinically meaningful, biomarkers should have an AUC value as close to 1 as possible.

In some embodiments, the methods, assays and kits described herein have a pre-determined sensitivity and/or specificity for determining the presence or absence of colorectal cancer or a colorectal cell proliferative disease. In some embodiments, the pre-determined sensitivity and/or specificity is determined by a state or country regulatory body.

In some embodiments, the methods, assays and kits described herein have a sensitivity of at least 0.65 for detecting a proliferative disease. In some embodiments, the methods, assays and kits described herein have a sensitivity of at least 0.66 for detecting a proliferative disease. In some embodiments, the methods, assays and kits described herein have a sensitivity of at least 0.67 for detecting a proliferative disease. In some embodiments, the methods, assays and kits described herein have a sensitivity of at least 0.68 for detecting a proliferative disease. In some embodiments, the methods, assays and kits described herein have a sensitivity of at least 0.69 for detecting a proliferative disease. In some embodiments, the methods, assays and kits described herein have a sensitivity of at least 0.70 for detecting a proliferative disease. In some embodiments, the methods, assays and kits described herein have a sensitivity of at least 0.71 for detecting a proliferative disease. In some embodiments, the methods, assays and kits described herein have a sensitivity of at least 0.72 for detecting a proliferative disease. In some embodiments, the methods, assays and kits described herein have a sensitivity of at least 0.73 for detecting a proliferative disease. In some embodiments, the methods, assays and kits described herein have a sensitivity of at least 0.74 for detecting a proliferative disease. In some embodiments, the methods, assays and kits described herein have a sensitivity of at least 0.75 for detecting a proliferative disease. In some embodiments, the methods, assays and kits described herein have a sensitivity of at least 0.76 for detecting a proliferative disease. In some embodiments, the methods, assays and kits described herein have a sensitivity of at least 0.77 for detecting a proliferative disease. In some embodiments, the methods, assays and kits described herein have a sensitivity of at least 0.78 for detecting a proliferative disease. In some embodiments, the methods, assays and kits described herein have a sensitivity of at least 0.79 for detecting a proliferative disease. In some embodiments, the methods, assays and kits described herein have a sensitivity of at least 0.80 for detecting a proliferative disease. In some embodiments, the methods, assays and kits described herein have a sensitivity of at least 0.81 for detecting a proliferative disease. In some embodiments, the methods, assays and kits described herein have a sensitivity of at least 0.82 for detecting a proliferative disease. In some embodiments, the methods, assays and kits described herein have a sensitivity of at least 0.83 for detecting a proliferative disease. In some embodiments, the methods, assays and kits described herein have a sensitivity of at least 0.84 for detecting a proliferative disease. In some embodiments, the methods, assays and kits described herein have a sensitivity of at least 0.85 for detecting a proliferative disease. In some embodiments, the methods, assays and kits described herein have a sensitivity of at least 0.86 for detecting a proliferative disease. In some embodiments, the methods, assays and kits described herein have a sensitivity of at least 0.87 for detecting a proliferative disease. In some embodiments, the methods, assays and kits described herein have a sensitivity of at least 0.88 for detecting a proliferative disease. In some embodiments, the methods, assays and kits described herein have a sensitivity of at least 0.89 for detecting a proliferative disease. In some embodiments, the methods, assays and kits described herein have a sensitivity of at least 0.90 for detecting a proliferative disease. In some embodiments, the methods, assays and kits described herein have a sensitivity of at least 0.91 for detecting a proliferative disease. In some embodiments, the methods, assays and kits described herein have a sensitivity of at least 0.92 for detecting a proliferative disease. In some embodiments, the methods, assays and kits described herein have a sensitivity of at least 0.93 for detecting a proliferative disease. In some embodiments, the methods, assays and kits described herein have a sensitivity of at least 0.94 for detecting a proliferative disease. In some embodiments, the methods, assays and kits described herein have a sensitivity of at least 0.95 for detecting a proliferative disease. In some embodiments, the methods, assays and kits described herein have a sensitivity of at least 0.96 for detecting a proliferative disease. In some embodiments, the methods, assays and kits described herein have a sensitivity of at least 0.97 for detecting a proliferative disease. In some embodiments, the methods, assays and kits described herein have a sensitivity of at least 0.98 for detecting a proliferative disease. In some embodiments, the methods, assays and kits described herein have a sensitivity of at least 0.99 for detecting a proliferative disease. In some embodiments, the methods, assays and kits described herein have a sensitivity of 1.00 for detecting a proliferative disease.

In some embodiments, the methods, assays and kits described herein have a specificity of at least 0.80 for detecting a proliferative disease. In some embodiments, the methods, assays and kits described herein have a specificity of at least 0.81 for detecting a proliferative disease. In some embodiments, the methods, assays and kits described herein have a specificity of at least 0.82 for detecting a proliferative disease. In some embodiments, the methods, assays and kits described herein have a specificity of at least 0.83 for detecting a proliferative disease. In some embodiments, the methods, assays and kits described herein have a specificity of at least 0.84 for detecting a proliferative disease. In some embodiments, the methods, assays and kits described herein have a specificity of at least 0.85 for detecting a proliferative disease. In some embodiments, the methods, assays and kits described herein have a specificity of at least 0.86 for detecting a proliferative disease. In some embodiments, the methods, assays and kits described herein have a specificity of at least 0.87 for detecting a proliferative disease. In some embodiments, the methods, assays and kits described herein have a specificity of at least 0.88 for detecting a proliferative disease. In some embodiments, the methods, assays and kits described herein have a specificity of at least 0.89 for detecting a proliferative disease. In some embodiments, the methods, assays and kits described herein have a specificity of at least 0.90 for detecting a proliferative disease. In some embodiments, the methods, assays and kits described herein have a specificity of at least 0.91 for detecting a proliferative disease. In some embodiments, the methods, assays and kits described herein have a specificity of at least 0.92 for detecting a proliferative disease. In some embodiments, the methods, assays and kits described herein have a specificity of at least 0.93 for detecting a proliferative disease. In some embodiments, the methods, assays and kits described herein have a specificity of at least 0.94 for detecting a proliferative disease. In some embodiments, the methods, assays and kits described herein have a specificity of at least 0.95 for detecting a proliferative disease. In some embodiments, the methods, assays and kits described herein have a specificity of at least 0.96 for detecting a proliferative disease. In some embodiments, the methods, assays and kits described herein have a specificity of at least 0.97 for detecting a proliferative disease. In some embodiments, the methods, assays and kits described herein have a specificity of at least 0.98 for detecting a proliferative disease. In some embodiments, the methods, assays and kits described herein have a specificity of at least 0.99 for detecting a proliferative disease. In some embodiments, the methods, assays and kits described herein have a specificity of 1.00 for detecting a proliferative disease.

In some embodiments, the methods, assays and kits described herein detect a proliferative disease with a sensitivity of at least 0.65 and a specificity of at least 0.8. In some embodiments, the methods, assays and kits described herein detect a proliferative disease with a sensitivity of at least 0.65 and a specificity of at least 0.9. In some embodiments, the methods, assays and kits described herein detect a proliferative disease with a sensitivity of at least 0.70 and a specificity of at least 0.8. In some embodiments, the methods, assays and kits described herein detect a proliferative disease with a sensitivity of at least 0.70 and a specificity of at least 0.9. In some embodiments, the methods, assays and kits described herein detect a proliferative disease with a sensitivity of at least 0.71 and a specificity of at least 0.8. In some embodiments, the methods, assays and kits described herein detect a proliferative disease with a sensitivity of at least 0.71 and a specificity of at least 0.9. In some embodiments, the methods, assays and kits described herein detect a proliferative disease with a sensitivity of at least 0.72 and a specificity of at least 0.8. In some embodiments, the methods, assays and kits described herein detect a proliferative disease with a sensitivity of at least 0.72 and a specificity of at least 0.9. In some embodiments, the methods, assays and kits described herein detect a proliferative disease with a sensitivity of at least 0.73 and a specificity of at least 0.8. In some embodiments, the methods, assays and kits described herein detect a proliferative disease with a sensitivity of at least 0.73 and a specificity of at least 0.9. In some embodiments, the methods, assays and kits described herein detect a proliferative disease with a sensitivity of at least 0.74 and a specificity of at least 0.8. In some embodiments, the methods, assays and kits described herein detect a proliferative disease with a sensitivity of at least 0.74 and a specificity of at least 0.9. In some embodiments, the methods, assays and kits described herein detect a proliferative disease with a sensitivity of at least 0.75 and a specificity of at least 0.8. In some embodiments, the methods, assays and kits described herein detect a proliferative disease with a sensitivity of at least 0.75 and a specificity of at least 0.9.

In some embodiments, the methods, assays and kits described herein have a sensitivity of at least 0.65 for detecting colorectal cancer or a colorectal cell proliferative disease. In some embodiments, the methods, assays and kits described herein have a sensitivity of at least 0.66 for detecting colorectal cancer or a colorectal cell proliferative disease. In some embodiments, the methods, assays and kits described herein have a sensitivity of at least 0.67 for detecting colorectal cancer or a colorectal cell proliferative disease. In some embodiments, the methods, assays and kits described herein have a sensitivity of at least 0.68 for detecting colorectal cancer or a colorectal cell proliferative disease. In some embodiments, the methods, assays and kits described herein have a sensitivity of at least 0.69 for detecting colorectal cancer or a colorectal cell proliferative disease. In some embodiments, the methods, assays and kits described herein have a sensitivity of at least 0.70 for detecting colorectal cancer or a colorectal cell proliferative disease. In some embodiments, the methods, assays and kits described herein have a sensitivity of at least 0.71 for detecting colorectal cancer or a colorectal cell proliferative disease. In some embodiments, the methods, assays and kits described herein have a sensitivity of at least 0.72 for detecting colorectal cancer or a colorectal cell proliferative disease. In some embodiments, the methods, assays and kits described herein have a sensitivity of at least 0.73 for detecting colorectal cancer or a colorectal cell proliferative disease. In some embodiments, the methods, assays and kits described herein have a sensitivity of at least 0.74 for detecting colorectal cancer or a colorectal cell proliferative disease. In some embodiments, the methods, assays and kits described herein have a sensitivity of at least 0.75 for detecting colorectal cancer or a colorectal cell proliferative disease. In some embodiments, the methods, assays and kits described herein have a sensitivity of at least 0.76 for detecting colorectal cancer or a colorectal cell proliferative disease. In some embodiments, the methods, assays and kits described herein have a sensitivity of at least 0.77 for detecting colorectal cancer or a colorectal cell proliferative disease. In some embodiments, the methods, assays and kits described herein have a sensitivity of at least 0.78 for detecting colorectal cancer or a colorectal cell proliferative disease. In some embodiments, the methods, assays and kits described herein have a sensitivity of at least 0.79 for detecting colorectal cancer or a colorectal cell proliferative disease. In some embodiments, the methods, assays and kits described herein have a sensitivity of at least 0.80 for detecting colorectal cancer or a colorectal cell proliferative disease. In some embodiments, the methods, assays and kits described herein have a sensitivity of at least 0.81 for detecting colorectal cancer or a colorectal cell proliferative disease. In some embodiments, the methods, assays and kits described herein have a sensitivity of at least 0.82 for detecting colorectal cancer or a colorectal cell proliferative disease. In some embodiments, the methods, assays and kits described herein have a sensitivity of at least 0.83 for detecting colorectal cancer or a colorectal cell proliferative disease. In some embodiments, the methods, assays and kits described herein have a sensitivity of at least 0.84 for detecting colorectal cancer or a colorectal cell proliferative disease. In some embodiments, the methods, assays and kits described herein have a sensitivity of at least 0.85 for detecting colorectal cancer or a colorectal cell proliferative disease. In some embodiments, the methods, assays and kits described herein have a sensitivity of at least 0.86 for detecting colorectal cancer or a colorectal cell proliferative disease. In some embodiments, the methods, assays and kits described herein have a sensitivity of at least 0.87 for detecting colorectal cancer or a colorectal cell proliferative disease. In some embodiments, the methods, assays and kits described herein have a sensitivity of at least 0.88 for detecting colorectal cancer or a colorectal cell proliferative disease. In some embodiments, the methods, assays and kits described herein have a sensitivity of at least 0.89 for detecting colorectal cancer or a colorectal cell proliferative disease. In some embodiments, the methods, assays and kits described herein have a sensitivity of at least 0.90 for detecting colorectal cancer or a colorectal cell proliferative disease. In some embodiments, the methods, assays and kits described herein have a sensitivity of at least 0.91 for detecting colorectal cancer or a colorectal cell proliferative disease. In some embodiments, the methods, assays and kits described herein have a sensitivity of at least 0.92 for detecting colorectal cancer or a colorectal cell proliferative disease. In some embodiments, the methods, assays and kits described herein have a sensitivity of at least 0.93 for detecting colorectal cancer or a colorectal cell proliferative disease. In some embodiments, the methods, assays and kits described herein have a sensitivity of at least 0.94 for detecting colorectal cancer or a colorectal cell proliferative disease. In some embodiments, the methods, assays and kits described herein have a sensitivity of at least 0.95 for detecting colorectal cancer or a colorectal cell proliferative disease. In some embodiments, the methods, assays and kits described herein have a sensitivity of at least 0.96 for detecting colorectal cancer or a colorectal cell proliferative disease. In some embodiments, the methods, assays and kits described herein have a sensitivity of at least 0.97 for detecting colorectal cancer or a colorectal cell proliferative disease. In some embodiments, the methods, assays and kits described herein have a sensitivity of at least 0.98 for detecting colorectal cancer or a colorectal cell proliferative disease. In some embodiments, the methods, assays and kits described herein have a sensitivity of at least 0.99 for detecting colorectal cancer or a colorectal cell proliferative disease. In some embodiments, the methods, assays and kits described herein have a sensitivity of 1.00 for detecting colorectal cancer or a colorectal cell proliferative disease.

In some embodiments, the methods, assays and kits described herein have a specificity of at least 0.80 for detecting colorectal cancer or a colorectal cell proliferative disease. In some embodiments, the methods, assays and kits described herein have a specificity of at least 0.81 for detecting colorectal cancer or a colorectal cell proliferative disease. In some embodiments, the methods, assays and kits described herein have a specificity of at least 0.82 for detecting colorectal cancer or a colorectal cell proliferative disease. In some embodiments, the methods, assays and kits described herein have a specificity of at least 0.83 for detecting colorectal cancer or a colorectal cell proliferative disease. In some embodiments, the methods, assays and kits described herein have a specificity of at least 0.84 for detecting colorectal cancer or a colorectal cell proliferative disease. In some embodiments, the methods, assays and kits described herein have a specificity of at least 0.85 for detecting colorectal cancer or a colorectal cell proliferative disease. In some embodiments, the methods, assays and kits described herein have a specificity of at least 0.86 for detecting colorectal cancer or a colorectal cell proliferative disease. In some embodiments, the methods, assays and kits described herein have a specificity of at least 0.87 for detecting colorectal cancer or a colorectal cell proliferative disease. In some embodiments, the methods, assays and kits described herein have a specificity of at least 0.88 for detecting colorectal cancer or a colorectal cell proliferative disease. In some embodiments, the methods, assays and kits described herein have a specificity of at least 0.89 for detecting colorectal cancer or a colorectal cell proliferative disease. In some embodiments, the methods, assays and kits described herein have a specificity of at least 0.90 for detecting colorectal cancer or a colorectal cell proliferative disease. In some embodiments, the methods, assays and kits described herein have a specificity of at least 0.91 for detecting colorectal cancer or a colorectal cell proliferative disease. In some embodiments, the methods, assays and kits described herein have a specificity of at least 0.92 for detecting colorectal cancer or a colorectal cell proliferative disease. In some embodiments, the methods, assays and kits described herein have a specificity of at least 0.93 for detecting colorectal cancer or a colorectal cell proliferative disease. In some embodiments, the methods, assays and kits described herein have a specificity of at least 0.94 for detecting colorectal cancer or a colorectal cell proliferative disease. In some embodiments, the methods, assays and kits described herein have a specificity of at least 0.95 for detecting colorectal cancer or a colorectal cell proliferative disease. In some embodiments, the methods, assays and kits described herein have a specificity of at least 0.96 for detecting colorectal cancer or a colorectal cell proliferative disease. In some embodiments, the methods, assays and kits described herein have a specificity of at least 0.97 for detecting colorectal cancer or a colorectal cell proliferative disease. In some embodiments, the methods, assays and kits described herein have a specificity of at least 0.98 for detecting colorectal cancer or a colorectal cell proliferative disease. In some embodiments, the methods, assays and kits described herein have a specificity of at least 0.99 for detecting colorectal cancer or a colorectal cell proliferative disease. In some embodiments, the methods, assays and kits described herein have a specificity of 1.00 for detecting colorectal cancer or a colorectal cell proliferative disease.

In some embodiments, the methods, assays and kits described herein detect colorectal cancer or a colorectal cell proliferative disease with a sensitivity of at least 0.65 and a specificity of at least 0.8. In some embodiments, the methods, assays and kits described herein detect colorectal cancer or a colorectal cell proliferative disease with a sensitivity of at least 0.65 and a specificity of at least 0.9. In some embodiments, the methods, assays and kits described herein detect colorectal cancer or a colorectal cell proliferative disease with a sensitivity of at least 0.70 and a specificity of at least 0.8. In some embodiments, the methods, assays and kits described herein detect colorectal cancer or a colorectal cell proliferative disease with a sensitivity of at least 0.70 and a specificity of at least 0.9. In some embodiments, the methods, assays and kits described herein detect colorectal cancer or a colorectal cell proliferative disease with a sensitivity of at least 0.71 and a specificity of at least 0.8. In some embodiments, the methods, assays and kits described herein detect colorectal cancer or a colorectal cell proliferative disease with a sensitivity of at least 0.71 and a specificity of at least 0.9. In some embodiments, the methods, assays and kits described herein detect colorectal cancer or a colorectal cell proliferative disease with a sensitivity of at least 0.72 and a specificity of at least 0.8. In some embodiments, the methods, assays and kits described herein detect colorectal cancer or a colorectal cell proliferative disease with a sensitivity of at least 0.72 and a specificity of at least 0.9. In some embodiments, the methods, assays and kits described herein detect colorectal cancer or a colorectal cell proliferative disease with a sensitivity of at least 0.73 and a specificity of at least 0.8. In some embodiments, the methods, assays and kits described herein detect colorectal cancer or a colorectal cell proliferative disease with a sensitivity of at least 0.73 and a specificity of at least 0.9. In some embodiments, the methods, assays and kits described herein detect colorectal cancer or a colorectal cell proliferative disease with a sensitivity of at least 0.74 and a specificity of at least 0.8. In some embodiments, the methods, assays and kits described herein detect colorectal cancer or a colorectal cell proliferative disease with a sensitivity of at least 0.74 and a specificity of at least 0.9. In some embodiments, the methods, assays and kits described herein detect colorectal cancer or a colorectal cell proliferative disease with a sensitivity of at least 0.75 and a specificity of at least 0.8. In some embodiments, the methods, assays and kits described herein detect colorectal cancer or a colorectal cell proliferative disease with a sensitivity of at least 0.75 and a specificity of at least 0.9.

In some embodiments, the methods, assays and kits described herein have an AUC of at least 0.70 for detecting a proliferative disease. In some embodiments, the methods, assays and kits described herein have an AUC of at least 0.71 for detecting a proliferative disease. In some embodiments, the methods, assays and kits described herein have an AUC of at least 0.72 for detecting a proliferative disease. In some embodiments, the methods, assays and kits described herein have an AUC of at least 0.73 for detecting a proliferative disease. In some embodiments, the methods, assays and kits described herein have an AUC of at least 0.74 for detecting a proliferative disease. In some embodiments, the methods, assays and kits described herein have an AUC of at least 0.75 for detecting a proliferative disease. In some embodiments, the methods, assays and kits described herein have an AUC of at least 0.76 for detecting a proliferative disease. In some embodiments, the methods, assays and kits described herein have an AUC of at least 0.77 for detecting a proliferative disease. In some embodiments, the methods, assays and kits described herein have an AUC of at least 0.78 for detecting a proliferative disease. In some embodiments, the methods, assays and kits described herein have an AUC of at least 0.79 for detecting a proliferative disease. In some embodiments, the methods, assays and kits described herein have an AUC of at least 0.80 for detecting a proliferative disease. In some embodiments, the methods, assays and kits described herein have an AUC of at least 0.81 for detecting a proliferative disease. In some embodiments, the methods, assays and kits described herein have an AUC of at least 0.82 for detecting a proliferative disease. In some embodiments, the methods, assays and kits described herein have an AUC of at least 0.83 for detecting a proliferative disease. In some embodiments, the methods, assays and kits described herein have an AUC of at least 0.84 for detecting a proliferative disease. In some embodiments, the methods, assays and kits described herein have an AUC of at least 0.85 for detecting a proliferative disease. In some embodiments, the methods, assays and kits described herein have an AUC of at least 0.86 for detecting a proliferative disease. In some embodiments, the methods, assays and kits described herein have an AUC of at least 0.87 for detecting a proliferative disease. In some embodiments, the methods, assays and kits described herein have an AUC of at least 0.88 for detecting a proliferative disease. In some embodiments, the methods, assays and kits described herein have an AUC of at least 0.89 for detecting a proliferative disease. In some embodiments, the methods, assays and kits described herein have an AUC of at least 0.90 for detecting a proliferative disease. In some embodiments, the methods, assays and kits described herein have an AUC of at least 0.91 for detecting a proliferative disease. In some embodiments, the methods, assays and kits described herein have an AUC of at least 0.92 for detecting a proliferative disease. In some embodiments, the methods, assays and kits described herein have an AUC of at least 0.93 for detecting a proliferative disease. In some embodiments, the methods, assays and kits described herein have an AUC of at least 0.94 for detecting a proliferative disease. In some embodiments, the methods, assays and kits described herein have an AUC of at least 0.95 for detecting a proliferative disease. In some embodiments, the methods, assays and kits described herein have an AUC of at least 0.96 for detecting a proliferative disease. In some embodiments, the methods, assays and kits described herein have an AUC of at least 0.97 for detecting a proliferative disease. In some embodiments, the methods, assays and kits described herein have an AUC of at least 0.98 for detecting a proliferative disease. In some embodiments, the methods, assays and kits described herein have an AUC of at least 0.99 for detecting a proliferative disease. In some embodiments, the methods, assays and kits described herein have an AUC of 1.0 for detecting a proliferative disease.

### Exemplary Multi-Analyte Embodiments

In some embodiments, the disclosure provides a method for detecting methylated genomic DNA and protein in one or more biological samples comprising:
(i) detecting one or more methylated genomic DNA sequences associated with a proliferative disease from the one or more biological samples; and
(ii) detecting the presence or amount of one or more protein biomarkers in the one or more biological samples, wherein the one or more protein biomarkers comprise CEA and/or amphiregulin.

In some embodiments, the disclosure provides a method for detecting methylated genomic DNA and protein in one or more biological samples comprising:
(i) detecting DNA methylation within at least one genomic DNA polynucleotide in the one or more biological samples, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA and/or amphiregulin.

In some embodiments, the disclosure provides a method for detecting methylated genomic DNA and protein in one or more biological samples comprising:
(i) detecting DNA methylation within one or more genomic DNA polynucleotide in the one or more biological samples, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise CEA and/or amphiregulin.

In some embodiments, the disclosure provides a method for detecting methylated genomic DNA and protein in one or more biological samples comprising:
(i) detecting one or more methylated genomic DNA sequences associated with a proliferative disease from the one or more biological samples; and
(ii) detecting the presence or amount of one or more protein biomarkers in the one or more biological samples, wherein the one or more protein biomarkers comprise CEA.

In some embodiments, the disclosure provides a method for detecting methylated genomic DNA and protein in one or more biological samples comprising:
(i) detecting DNA methylation within at least one genomic DNA polynucleotide in the one or more biological samples, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA.

In some embodiments, the disclosure provides a method for detecting methylated genomic DNA and protein in one or more biological samples comprising:
(i) detecting DNA methylation within one or more genomic DNA polynucleotide in the one or more biological samples, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise CEA.

In some embodiments, the disclosure provides a method for detecting methylated genomic DNA and protein in one or more biological samples comprising:
(i) detecting one or more methylated genomic DNA sequences associated with a proliferative disease from the one or more biological samples; and
(ii) detecting the presence or amount of one or more protein biomarkers in the one or more biological samples, wherein the one or more protein biomarkers comprise amphiregulin.

In some embodiments, the disclosure provides a method for detecting methylated genomic DNA and protein in one or more biological samples comprising:
(i) detecting DNA methylation within at least one genomic DNA polynucleotide in the one or more biological samples, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise amphiregulin.

In some embodiments, the disclosure provides a method for detecting methylated genomic DNA and protein in one or more biological samples comprising:
(i) detecting DNA methylation within one or more genomic DNA polynucleotide in the one or more biological samples, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise amphiregulin.

In some embodiments, the disclosure provides a method for detecting methylated genomic DNA and protein in one or more biological samples comprising:
(i) detecting one or more methylated genomic DNA sequences associated with a proliferative disease from the one or more biological samples; and
(ii) detecting the presence or amount of one or more protein biomarkers in the one or more biological samples, wherein the one or more protein biomarkers comprise CEA and amphiregulin.

In some embodiments, the disclosure provides a method for detecting methylated genomic DNA and protein in one or more biological samples comprising:
(i) detecting DNA methylation within at least one genomic DNA polynucleotide in the one or more biological samples, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA and amphiregulin.

In some embodiments, the disclosure provides a method for detecting methylated genomic DNA and protein in one or more biological samples comprising:
(i) detecting DNA methylation within one or more genomic DNA polynucleotide in the one or more biological samples, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise CEA and amphiregulin.

In some embodiments, the disclosure provides a method for detecting methylated genomic DNA and protein in one or more serum or plasma samples comprising:
(i) detecting one or more methylated genomic DNA sequences associated with a proliferative disease from the one or more serum or plasma samples; and
(ii) detecting the presence or amount of one or more protein biomarkers in the one or more serum or plasma samples, wherein the one or more protein biomarkers comprise CEA and/or amphiregulin.

In some embodiments, the disclosure provides a method for detecting methylated genomic DNA and protein in one or more serum or plasma samples comprising:
(i) detecting DNA methylation within at least one genomic DNA polynucleotide in the one or more serum or plasma samples, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise CEA and/or amphiregulin.

In some embodiments, the disclosure provides a method for detecting methylated genomic DNA and protein in one or more serum or plasma samples comprising:
(i) detecting DNA methylation within one or more genomic DNA polynucleotide in the one or more serum or plasma samples, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise CEA and/or amphiregulin.

In some embodiments, the disclosure provides a method for detecting methylated genomic DNA and protein in one or more serum or plasma samples comprising:
(i) detecting one or more methylated genomic DNA sequences associated with a proliferative disease from the one or more serum or plasma samples; and
(ii) detecting the presence or amount of one or more protein biomarkers in the one or more serum or plasma samples, wherein the one or more protein biomarkers comprise CEA.

In some embodiments, the disclosure provides a method for detecting methylated genomic DNA and protein in one or more serum or plasma samples comprising:
(i) detecting DNA methylation within at least one genomic DNA polynucleotide in the one or more serum or plasma samples, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise CEA.

In some embodiments, the disclosure provides a method for detecting methylated genomic DNA and protein in one or more serum or plasma samples comprising:
(i) detecting DNA methylation within one or more genomic DNA polynucleotide in the one or more serum or plasma samples, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise CEA.

In some embodiments, the disclosure provides a method for detecting methylated genomic DNA and protein in one or more serum or plasma samples comprising:
(i) detecting one or more methylated genomic DNA sequences associated with a proliferative disease from the one or more serum or plasma samples; and
(ii) detecting the presence or amount of one or more protein biomarkers in the one or more serum or plasma samples, wherein the one or more protein biomarkers comprise amphiregulin.

In some embodiments, the disclosure provides a method for detecting methylated genomic DNA and protein in one or more serum or plasma samples comprising:
(i) detecting DNA methylation within at least one genomic DNA polynucleotide in the one or more serum or plasma samples, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise amphiregulin.

In some embodiments, the disclosure provides a method for detecting methylated genomic DNA and protein in one or more serum or plasma samples comprising:
(i) detecting DNA methylation within one or more genomic DNA polynucleotide in the one or more serum or plasma samples, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise amphiregulin.

In some embodiments, the disclosure provides a method for detecting methylated genomic DNA and protein in one or more serum or plasma samples comprising:
(i) detecting one or more methylated genomic DNA sequences associated with a proliferative disease from the one or more serum or plasma samples; and
(ii) detecting the presence or amount of one or more protein biomarkers in the one or more serum or plasma samples, wherein the one or more protein biomarkers comprise CEA and amphiregulin.

In some embodiments, the disclosure provides a method for detecting methylated genomic DNA and protein in one or more serum or plasma samples comprising:
(i) detecting DNA methylation within at least one genomic DNA polynucleotide in the one or more serum or plasma samples, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise CEA and amphiregulin.

In some embodiments, the disclosure provides a method for detecting methylated genomic DNA and protein in one or more serum or plasma samples comprising:
(i) detecting DNA methylation within one or more genomic DNA polynucleotide in the one or more serum or plasma samples, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise CEA and amphiregulin.

In some embodiments, the disclosure provides a method for detecting methylated genomic DNA and protein in one or more biological samples comprising:
(i) detecting synthetic DNA generated from one or more methylated genomic DNA sequences associated with a proliferative disease from the one or more biological samples,
wherein the synthetic DNA is generated by converting cytosine unmethylated in the 5-position to uracil or another base that does not hybridize to guanine in the genomic DNA, and detecting unconverted cytosine in the synthetic DNA; and
(ii) detecting the presence or amount of one or more protein biomarkers in the one or more biological samples, wherein the one or more protein biomarkers comprise CEA and/or amphiregulin.

In some embodiments, the disclosure provides a method for detecting methylated genomic DNA and protein in one or more biological samples comprising:
(i) detecting DNA methylation within at least one genomic DNA polynucleotide in the one or more biological samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin.

In some embodiments, the disclosure provides a method for detecting methylated genomic DNA and protein in one or more biological samples comprising:
(i) detecting DNA methylation within one or more genomic DNA polynucleotide in the one or more biological samples, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin.

In some embodiments, the disclosure provides a method for detecting methylated genomic DNA and protein in one or more biological samples comprising:
(i) detecting synthetic DNA generated from one or more methylated genomic DNA sequences associated with a proliferative disease from the one or more biological samples,
wherein the synthetic DNA is generated by converting cytosine unmethylated in the 5-position to uracil or another base that does not hybridize to guanine in the genomic DNA, and detecting unconverted cytosine in the synthetic DNA; and
(ii) detecting the presence or amount of one or more protein biomarkers in the one or more biological samples, wherein the one or more protein biomarkers comprise CEA.

In some embodiments, the disclosure provides a method for detecting methylated genomic DNA and protein in one or more biological samples comprising:
(i) detecting DNA methylation within at least one genomic DNA polynucleotide in the one or more biological samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA.

In some embodiments, the disclosure provides a method for detecting methylated genomic DNA and protein in one or more biological samples comprising:
(i) detecting DNA methylation within one or more genomic DNA polynucleotide in the one or more biological samples, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA.

In some embodiments, the disclosure provides a method for detecting methylated genomic DNA and protein in one or more biological samples comprising:
(i) detecting synthetic DNA generated from one or more methylated genomic DNA sequences associated with a proliferative disease from the one or more biological samples,
wherein the synthetic DNA is generated by converting cytosine unmethylated in the 5-position to uracil or another base that does not hybridize to guanine in the genomic DNA, and detecting unconverted cytosine in the synthetic DNA; and
(ii) detecting the presence or amount of one or more protein biomarkers in the one or more biological samples, wherein the one or more protein biomarkers comprise amphiregulin.

In some embodiments, the disclosure provides a method for detecting methylated genomic DNA and protein in one or more biological samples comprising:
(i) detecting DNA methylation within at least one genomic DNA polynucleotide in the one or more biological samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise amphiregulin.

In some embodiments, the disclosure provides a method for detecting methylated genomic DNA and protein in one or more biological samples comprising:
(i) detecting DNA methylation within one or more genomic DNA polynucleotide in the one or more biological samples, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise amphiregulin.

In some embodiments, the disclosure provides a method for detecting methylated genomic DNA and protein in one or more biological samples comprising:
(i) detecting synthetic DNA generated from one or more methylated genomic DNA sequences associated with a proliferative disease from the one or more biological samples,
wherein the synthetic DNA is generated by converting cytosine unmethylated in the 5-position to uracil or another base that does not hybridize to guanine in the genomic DNA, and detecting unconverted cytosine in the synthetic DNA; and
(ii) detecting the presence or amount of one or more protein biomarkers in the one or more biological samples, wherein the one or more protein biomarkers comprise CEA and amphiregulin.

In some embodiments, the disclosure provides a method for detecting methylated genomic DNA and protein in one or more biological samples comprising:
(i) detecting DNA methylation within at least one genomic DNA polynucleotide in the one or more biological samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and amphiregulin.

In some embodiments, the disclosure provides a method for detecting methylated genomic DNA and protein in one or more biological samples comprising:
(i) detecting DNA methylation within one or more genomic DNA polynucleotide in the one or more biological samples, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and amphiregulin.

In some embodiments, the disclosure provides a method for detecting methylated genomic DNA and protein in one or more serum or plasma samples comprising:
(i) detecting synthetic DNA generated from one or more methylated genomic DNA sequences associated with a proliferative disease from the one or more serum or plasma samples, wherein the synthetic DNA is generated by converting cytosine unmethylated in the 5-position to uracil or another base that does not hybridize to guanine in the genomic DNA, and detecting unconverted cytosine in the synthetic DNA; and
(ii) detecting the presence or amount of one or more protein biomarkers in the one or more serum or plasma samples, wherein the one or more protein biomarkers comprise CEA and/or amphiregulin.

In some embodiments, the disclosure provides a method for detecting methylated genomic DNA and protein in one or more serum or plasma samples comprising:
(i) detecting DNA methylation within at least one genomic DNA polynucleotide in the one or more serum or plasma samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and/or amphiregulin.

In some embodiments, the disclosure provides a method for detecting methylated genomic DNA and protein in one or more serum or plasma samples comprising:
(i) detecting DNA methylation within one or more genomic DNA polynucleotide in the one or more serum or plasma samples, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and/or amphiregulin.

In some embodiments, the disclosure provides a method for detecting methylated genomic DNA and protein in one or more serum or plasma samples comprising:
(i) detecting synthetic DNA generated from one or more methylated genomic DNA sequences associated with a proliferative disease from the one or more serum or plasma samples, wherein the synthetic DNA is generated by converting cytosine unmethylated in the 5-position to uracil or another base that does not hybridize to guanine in the genomic DNA, and detecting unconverted cytosine in the synthetic DNA; and
(ii) detecting the presence or amount of one or more protein biomarkers in the one or more serum or plasma samples, wherein the one or more protein biomarkers comprise CEA.

In some embodiments, the disclosure provides a method for detecting methylated genomic DNA and protein in one or more serum or plasma samples comprising:
(i) detecting DNA methylation within at least one genomic DNA polynucleotide in the one or more serum or plasma samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA.

In some embodiments, the disclosure provides a method for detecting methylated genomic DNA and protein in one or more serum or plasma samples comprising:
(i) detecting DNA methylation within one or more genomic DNA polynucleotide in the one or more serum or plasma samples, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA.

In some embodiments, the disclosure provides a method for detecting methylated genomic DNA and protein in one or more serum or plasma samples comprising:
(i) detecting synthetic DNA generated from one or more methylated genomic DNA sequences associated with a proliferative disease from the one or more serum or plasma samples, wherein the synthetic DNA is generated by converting cytosine unmethylated in the 5-position to uracil or another base that does not hybridize to guanine in the genomic DNA, and detecting unconverted cytosine in the synthetic DNA; and
(ii) detecting the presence or amount of one or more protein biomarkers in the one or more serum or plasma samples, wherein the one or more protein biomarkers comprise amphiregulin.

In some embodiments, the disclosure provides a method for detecting methylated genomic DNA and protein in one or more serum or plasma samples comprising:
(i) detecting DNA methylation within at least one genomic DNA polynucleotide in the one or more serum or plasma samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise amphiregulin.

In some embodiments, the disclosure provides a method for detecting methylated genomic DNA and protein in one or more serum or plasma samples comprising:
(i) detecting DNA methylation within one or more genomic DNA polynucleotide in the one or more serum or plasma samples, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise amphiregulin.

In some embodiments, the disclosure provides a method for detecting methylated genomic DNA and protein in one or more serum or plasma samples comprising:
(i) detecting synthetic DNA generated from one or more methylated genomic DNA sequences associated with a proliferative disease from the one or more serum or plasma samples, wherein the synthetic DNA is generated by converting cytosine unmethylated in the 5-position to uracil or another base that does not hybridize to guanine in the genomic DNA, and detecting unconverted cytosine in the synthetic DNA; and more serum or plasma samples, wherein the one or more protein biomarkers comprise CEA and amphiregulin.

In some embodiments, the disclosure provides a method for detecting methylated genomic DNA and protein in one or more serum or plasma samples comprising:
(i) detecting DNA methylation within at least one genomic DNA polynucleotide in the one or more serum or plasma samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and amphiregulin.

In some embodiments, the disclosure provides a method for detecting methylated genomic DNA and protein in one or more serum or plasma samples comprising:
(i) detecting DNA methylation within one or more genomic DNA polynucleotide in the one or more serum or plasma samples, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and amphiregulin.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise CEA,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA and amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise CEA and amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise CEA and/or amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise CEA and/or amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise CEA,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise CEA,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise CEA and amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise CEA and amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and amphiregulin,
wherein the presence of DNA methylation and one or more protein biomarkers in the one or more biological samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and/or amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and/or amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and amphiregulin,
wherein the presence of DNA methylation and one or more protein biomarkers in the one or more serum or plasma samples is indicative of the presence of colorectal cancer or a colorectal cell proliferative disease in the subject.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise CEA,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b; and
(ii) detecting the presence or amount of one or more protein biomarkers in the one or more biological samples, wherein the one or more protein biomarkers comprise CEA and amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more biological samples, wherein the one or more protein biomarkers comprise CEA and amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise CEA and/or amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise CEA and/or amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise CEA,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise CEA,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b; and
(ii) detecting the presence or amount of one or more protein biomarkers in the one or more serum or plasma samples, wherein the one or more protein biomarkers comprise CEA and amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b); and
(ii) detecting the presence or amount of one or more protein biomarkers in the one or more serum or plasma samples, wherein the one or more protein biomarkers comprise CEA and amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and amphiregulin,
wherein the presence of DNA methylation and one or more protein biomarkers in the one or more biological samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and/or amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and/or amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for detecting colorectal cancer or a colorectal cell proliferative disease in a subject comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more serum or plasma samples, wherein the one or more protein biomarkers comprise CEA and amphiregulin,
wherein the presence of DNA methylation and one or more protein biomarkers in the one or more serum or plasma samples is indicative of the presence of colorectal cancer or a colorectal cell proliferative disease in the subject with a sensitivity of at least 0.70-0.75 and/or a specificity of at least 0.85-0.9.

In some embodiments, the disclosure provides a method for prognosing a subject with colorectal cancer or a colorectal cell proliferative disease comprising:
(i) detecting DNA methylation within at least one genomic DNA polynucleotide in one or more biological samples from the subject, wherein the at least one genomic DNA polynucleotide comprises mSEPT9 and/or mANKDRD13b;
(ii) detecting the presence or amount of one or more protein biomarkers in the one or more biological samples, wherein the one or more protein biomarkers comprise CEA and/or amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b);
(ii) detecting the presence or amount of one or more protein biomarkers in the one or more biological samples, wherein the one or more protein biomarkers comprise CEA and/or amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within at least one genomic DNA polynucleotide in one or more biological samples from the subject, wherein the at least one genomic DNA polynucleotide comprises mSEPT9 and/or mANKDRD13b;
(ii) detecting the presence or amount of one or more protein biomarkers in the one or more biological samples, wherein the one or more protein biomarkers comprise CEA; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b);
(ii) detecting the presence or amount of one or more protein biomarkers in the one or more biological samples, wherein the one or more protein biomarkers comprise CEA; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within at least one genomic DNA polynucleotide in one or more biological samples from the subject, wherein the at least one genomic DNA polynucleotide comprises mSEPT9 and/or mANKDRD13b;
(ii) detecting the presence or amount of one or more protein biomarkers in the one or more biological samples, wherein the one or more protein biomarkers comprise amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b);
(ii) detecting the presence or amount of one or more protein biomarkers in the one or more biological samples, wherein the one or more protein biomarkers comprise amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within at least one genomic DNA polynucleotide in one or more biological samples from the subject, wherein the at least one genomic DNA polynucleotide comprises mSEPT9 and/or mANKDRD13b;
(ii) detecting the presence or amount of one or more protein biomarkers in the one or more biological samples, wherein the one or more protein biomarkers comprise CEA and amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b);
(ii) detecting the presence or amount of one or more protein biomarkers in the one or more biological samples, wherein the one or more protein biomarkers comprise CEA and amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b;
(ii) detecting the presence or amount of one or more protein biomarkers in the one or more serum or plasma samples, wherein the one or more protein biomarkers comprise CEA and/or amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b);
(ii) detecting the presence or amount of one or more protein biomarkers in the one or more serum or plasma samples, wherein the one or more protein biomarkers comprise CEA and/or amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b;
(ii) detecting the presence or amount of one or more protein biomarkers in the one or more serum or plasma samples, wherein the one or more protein biomarkers comprise CEA; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b);
(ii) detecting the presence or amount of one or more protein biomarkers in the one or more serum or plasma samples, wherein the one or more protein biomarkers comprise CEA; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b;
(ii) detecting the presence or amount of one or more protein biomarkers in the one or more serum or plasma samples, wherein the one or more protein biomarkers comprise amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b);
(ii) detecting the presence or amount of one or more protein biomarkers in the one or more serum or plasma samples, wherein the one or more protein biomarkers comprise amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b;
(ii) detecting the presence or amount of one or more protein biomarkers in the one or more serum or plasma samples, wherein the one or more protein biomarkers comprise CEA and amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b);
(ii) detecting the presence or amount of one or more protein biomarkers in the one or more serum or plasma samples, wherein the one or more protein biomarkers comprise CEA and amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise CEA and/or amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise CEA and/or amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise CEA; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise CEA; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise CEA and amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within one or more genomic DNA polynucleotide in one or more biological samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise CEA and amphiregulin; and,
(iii) prognosing the subject with colorectal cancer or a colorectal cell proliferative disease based on the presence of DNA methylation and one or more protein biomarkers in the one or more biological samples.

In some embodiments, the disclosure provides a method for prognosing a subject with colorectal cancer or a colorectal cell proliferative disease comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more serum or plasma samples, wherein the one or more protein biomarkers comprise CEA and/or amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more serum or plasma samples, wherein the one or more protein biomarkers comprise CEA and/or amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method prognosing a subject with colorectal cancer or a colorectal cell proliferative disease comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more serum or plasma samples, wherein the one or more protein biomarkers comprise CEA; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more serum or plasma samples, wherein the one or more protein biomarkers comprise CEA; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more serum or plasma samples, wherein the one or more protein biomarkers comprise amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more serum or plasma samples, wherein the one or more protein biomarkers comprise amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within at least one genomic DNA polynucleotide in one or more serum or plasma samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more serum or plasma samples, wherein the one or more protein biomarkers comprise CEA and amphiregulin; and,
(iii) prognosing the subject with colorectal cancer or a colorectal cell proliferative disease based on the presence of DNA methylation and one or more protein biomarkers in the one or more serum or plasma samples.

In some embodiments, the disclosure provides a method for prognosing a subject with colorectal cancer or a colorectal cell proliferative disease comprising:
(i) detecting DNA methylation within one or more genomic DNA polynucleotide in one or more serum or plasma samples from the subject, wherein the one or more genomic DNA polynucleotides comprise SEQ ID NO: 31 (mSEPT9) and SEQ ID NOs: 6 and 11 (mANKDRD13b), wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more serum or plasma samples, wherein the one or more protein biomarkers comprise CEA and amphiregulin; and,
(iii) prognosing the subject with colorectal cancer or a colorectal cell proliferative disease based on the presence of DNA methylation and one or more protein biomarkers in the one or more serum or plasma samples.

In some or embodiments, detection of mSEPT9 is carried out using the Epi proColon assay kit (epiprocoln.com; accessdata.fda.gov/cdrh_docs/pdf13/p13001c.pdf). In some embodiments,

In some embodiments, a method or assay described herein comprises:
(i) detecting DNA methylation of mSEPT9 in one or more biological samples comprising analyzing the one or more biological samples using an Epi proColon assay kit;
(ii) detecting DNA methylation of mANKRD13B in the one or more biological samples; and
(iii) detecting the presence or amount of one or more protein biomarkers in the biological samples, wherein the one or more protein biomarkers comprise CEA and/or amphiregulin.

In some or any of the foregoing embodiments, the one or more protein biomarkers comprises haptoglobin. In some embodiments, the one or more protein biomarkers comprises CEA and haptoglobin. In some embodiments, the one or more protein biomarkers comprises amphiregulin and haptoglobin. In some embodiments, the one or more protein biomarkers comprises CEA, amphiregulin and haptoglobin. In some embodiments, including haptoglobin increases sensitivity for detecting colorectal cancer, advanced adenoma, or a colorectal cell proliferative disease compared to a method or assay that does not detect haptoglobin.

### Methods of Treating Proliferative Disease

In some aspects, the disclosure provides methods of treating a proliferative disease detected by any one of the methods, assays or kits described herein. In some aspects, the disclosure provides methods of treating colorectal cancer or a colorectal cell proliferative disease detected by any one of the methods, assays or kits described herein.

In some embodiments, the methods, assays or kits described here predict whether a subject has a proliferative disease such as colorectal cancer. Accordingly, in some embodiments, prior to treatment, confirmation of the proliferative disease is needed. For example, in some embodiments, wherein a method, assay or kit described herein detects colorectal cancer in a subject, a colonoscopy is administered to confirm the subject has colorectal cancer. Upon confirmation of the presence of a proliferative disorder, treatment may be administered as determined by a clinician.

The proliferative disease can be classified into subtypes (as defined by the corresponding TNM classification(s) in brackets) of the cancer and each of its subtypes: stage 0 (TO, NO, MO), stage 1 (T1, NO, MO), stage II (T2, NO, MO), stage 111 (T3, NO, MO; or T1 to T3, N1, MO), stage IVA (T4a, NO or N1, MO; or T1 to T4a, N2, MO), stage IVB (T4b, any N, MO or any T, N3. MO), and stage IVC (any T, any N, MI). The TNM classification is a staging system for malignant cancer. As used herein the term “TNM classification” refers to the 6^{th} edition of the TNM stage grouping as defined in Sobin et al. (International Union Against Cancer (UICC), TNM Classification of Malignant tumors, 6^{th} ed. New York; Springer, 2002, pp. 191-203).

The goal of cancer treatment is to completely remove tumors, cancers, and adenomas from the body or kill all the cancer cells in the body. Cancer treatments may involve the use of surgery, radiation, medications, and other therapies to cure a cancer, reduce the size of a tumor or cancer, or stop the progression or growth of a tumor or cancer.

Cancer treatments can typically be broken down into three stages. In a first stage, a primary treatment is applied to remove the cancer or kill all the cancer cells. In a second stage, an adjuvant treatment may be applied to kill any cancer cells that remain after the primary treatment. In some cases, the adjuvant treatment may be applied prior to or concurrently with the primary treatment to reduce the size of the cancer or tumor and to make the primary treatment easier or more effective. In a third stage, palliative treatments may be applied to relieve the side effects of the primary and palliative treatments as well as the symptoms caused by the cancer itself. In some cases, the palliative treatment may occur prior to or concurrently with the primary and/or adjuvant treatments.

In some embodiments, a method for treating cancer comprises surgery. Several types of surgery are employed in the identification, characterization, and removal of tumors, cancers, and adenomas from the body. These surgeries may be carried out using a variety of surgical techniques including, but not limited to, microscopically controlled surgery, laser surgery, cryosurgery, and electrosurgery.

In some embodiments, a diagnostic surgery is used to identify the presence of a cancer within a mass of cells. A tissue sample is collected from the subject wherein said tissue sample is then evaluated to determine the presence or absence of cancer, to identify the type of cancer if cancer is present, and to determine the stage of said cancer.

In some embodiments, a staging surgery is employed after a positive diagnosis of cancer. Staging surgery may be prescribed after a positive confirmation of cancer following a diagnostic surgery. Staging surgery serves to uncover the extent of the cancer or size of the tumor in the body. During a laparoscopy, a surgical staging procedure, a camera is inserted through a small incision to examine the tumors or cancers and, in some cases, to also remove tissue samples. In some cases, staging surgery may be combined with a diagnostic surgery.

Upon a positive diagnosis of cancer, a patient may be treated with curative, debulking, palliative, preventive, and/or supportive surgery to aid in the treatment or removal of said cancer, to ease discomfort due to cancer symptoms and/or treatments, and/or to aid in the efficacy of cancer treatment. When the cancer is localized to a specific area of the body, curative surgery can be used to remove the cancerous growth from the body. In cases where removal of the entire tumor mass is deemed too dangerous for the patient, debulking surgery may be employed to partially, but not completely, remove the tumor or cancer.

In some embodiments, a method for treating cancer comprises chemotherapy and/or radiation therapy. Chemotherapy is a drug treatment in which one or more anti-cancer drugs, chemicals, or poisons are delivered to the patient, often intravenously, to kill fast-growing cells in the body. Chemotherapy is often employed during cancer treatments because cancer cells grow and multiply much more quickly than most other cells in the body. Chemotherapy may be employed as a primary, adjuvant, and/or palliative treatment.

Many different chemotherapy drugs are available. Chemotherapy drugs can be used alone or in combination to treat a wide variety of cancers. Chemotherapy drugs can be administered in a variety of formats which included, but are not limited to, intravenous infusions, shots, pills, creams, or via methods of direct application.

Chemotherapy treatment carries a risk of side effects. Some side effects of chemotherapy are mild and treatable, while others can cause serious complications. Common side effects of chemotherapy drugs include nausea, vomiting, diarrhea, hair loss, fatigue, loss of appetite, bruising and bleeding, oral sores, pain, fever, and constipation. Though many of these symptoms recede following the conclusion of treatment, additional palliative treatment may be prescribed to relieve the side effects of chemotherapy. Chemotherapy drugs also carry the risk for chronic complications including, but not limited to, heart and lung problems, infertility, kidney failure, nerve damage, and the risk of a second cancer.

Radiation therapy is a therapy in which ionizing radiation is applied to the body to control or kill tumors or cancerous cells. Radiation therapy damages the DNA within tissues subjected to the radiation and leads to cellular death. As the ionizing radiation can damage normal, healthy cells similarly to tumor or cancer cells, treatment plans seek to optimally target the tumor or cancer site and reduce the exposure of healthy tissues to the ionizing radiation.

In external beam radiation, ionizing radiation beams are aimed to intersect at the cancer or tumor site and provide a much larger dose of radiation within the cancer or tumor site than in the surrounding healthy tissue. Brachytherapy is a form of therapy in which a solid source of radiation is place internally near the cancer or tumor site to locally apply radiation to the cancer or tumor site and reduce exposure of other non-cancerous tissues to the radiation source. Systemic radiation therapy involves the introduction of a liquid radiation source to the subject's circulatory system and/or gastric system.

Radiation therapy is typically combined with other forms of therapy as a primary, adjuvant, and/or palliative treatment.

Some cancer types are promoted by specific hormones, wherein the removal of said hormones or blockage of their effects may reduce or stop growth of the tumor or cancer. Hormone therapy involved the manipulation of the endocrine system through exogenous administration of specific hormones, usually steroid hormones, that either inhibit the production of tumor- or cancer-growth-promoting hormones or blocks the activity of said hormones. In some cases, hormone therapy also involves surgical removal of the endocrine organs. Hormonal therapy is used for several types of cancers derived from hormonally responsive tissues, including the breast, prostate, endometrium, and adrenal cortex.

Cancer immunotherapy involves the treatment of disease by activating or suppressing the immune system to promote anti-tumor and anti-cancer activities within the subject's immune system. Cancer immunotherapy exploits the fact that cancer cells often have tumor-specific antigens present on their surfaces by introducing or promoting the activity of immune cells in the subject. Cell-based immunotherapies mediated by immune effector cells such as natural killer (NK) cells, lymphokine-activated killer cells, cytotoxic T cells and dendritic cells promote anti-tumor and anti-cancer activities within the immune system by targeting abnormal antigens expressed on the surface of tumor cells. Activation immunotherapies are designed to activate or elicit an immune response to target cancer and tumor cells for destruction. Suppression immunotherapies are designed to inhibit or suppress an immune response to prohibit the growth of cancers and tumors.

Targeted therapy is a type of cancer treatment that targets proteins that control the growth, division, and spread of tumor and cancer cells. Targeted therapies can block or turn off signals that control cancer and tumor cell growth, prevents cancer and tumor cells from living longer than normal, and destroys cancer and tumor cells.

Targeted therapy blocks the growth of cancer and tumor cells by interfering with the activity of specific target molecules needed for carcinogenesis and tumor growth, rather than by simply interfering with all rapidly dividing cells (e.g., with traditional chemotherapy). As such, targeted cancer therapies may be more effective and less harmful to normal cells than alternative forms of treatment like radiation therapy and chemotherapy.

The most successful targeted therapies involve entities that specifically or preferentially target a protein or enzyme that carries a mutation or genetic alteration that is associated with cancer cells and not found or less frequently found in normal host tissues. As targeted therapies require the targeting of specific cancer- and tumor-associated factors, most targeted therapies require the use of biomarkers to aid in the selection of patients who will respond to a specific targeted therapy.

There are targeted therapies for lung cancer, colorectal cancer, head and neck cancer, breast cancer, multiple myeloma, lymphoma, prostate cancer, melanoma and other cancers. Targeted therapies may also be combined with other forms of cancer treatment.

Cancer care typically requires a multi-faceted approach in which several of the techniques described above are employed together to create a plan to cure the patient's cancer. Colorectal cancer treatment plans typically involve surgery, radiation therapy, chemotherapy, targeted therapy, and immunotherapy.

Surgical resection is the most common treatment for colorectal cancer in which the tumor and some of the healthy surrounding tissue from the colon or rectum and nearby lymph nodes is removed. In some cases, radiation therapies and/or chemotherapies are employed prior to surgery to reduce tumor or cancer cell size to aid in tumor removal. In some cases, radiation therapy and/or chemotherapy is employed after surgery to destroy any remaining cancer or tumor cells.

Colorectal cancers are also susceptible to targeted therapies. Colorectal cancer often overproduction of epidermal growth factor receptor (EGFR). Drugs that block EGFR may help stop or slow cancer growth. Other options include drugs that inhibit vascular endothelial growth factor (VEGF), tumor-agnostic treatment focusing on an NTRK fusion, and anti-angiogenesis therapy.

In some embodiments, a cancer therapy is selected from the group consisting of antibodies (e.g. antibodies stimulating an immune response destroying cancer cells such as retuximab or alemtuzubab, antibodies stimulating an immune response by binding to receptors of immune cells an inhibiting signals that prevent the immune cell to attack “own” cells, such as ipilimumab, antibodies interfering with the action of proteins necessary for tumor growth such as bevacizumab, cetuximab or panitumumab, or antibodies conjugated to a drug, preferably a cell-killing substance like a toxin, chemotherapeutic or radioactive molecule, such as Y-ibritumomab tiuxetan, I-tositumomab or ado-trastuzumab emtansine), cytokines (e.g. interferons or interleukins such as INF-alpha and IL-2), vaccines (e.g. vaccines comprising cancer-associated antigens, such as sipuleucel-T), oncolytic viruses (e.g. naturally oncolytic viruses such as reovirus, Newcastle disease virus or mumps virus, or viruses genetically engineered viruses such as measles virus, adenovirus, vaccinia virus or herpes virus preferentially targeting cells carrying cancer-associated antigens), gene therapy agents (e.g. DNA or RNA replacing an altered tumor suppressor, blocking the expression of an oncogene, improving a subject's immune system, making cancer cells more sensitive to chemotherapy, radiotherapy or other treatments, inducing cellular suicide or conferring an anti-angiogenic effect) and adoptive T cells (e.g. subject-harvested tumor-invading T-cells selected for antitumor activity, or subject-harvested T-cells genetically modified to recognize a cancer-associated antigen).

In some embodiments, the one or more cancer therapies selected from the group consisting of Abiraterone Acetate, ABVD, ABVE, ABVE-PC, AC, AC-T, ADE, Ado-Trastuzumab Emtansine, Afatinib Dimaleate, Aldesleukin, Alemtuzumab, Aminolevulinic Acid, Anastrozole, Aprepitant, Arsenic Trioxide, Asparaginase Erwinia chrysanthemi, Axitinib, Azacitidine, BEACOPP, Belinostat, Bendamustine Hydrochloride, BEP, Bevacizumab, Bexarotene, Bicalutamide, Bleomycin, Bortezomib, Bosutinib, Brentuximab Vedotin, Busulfan, Cabazitaxel, Cabozantinib-S-Malate, CAFCapecitabine, CAPOX, Carboplatin, CARBOPLATIN-TAXOL, Carfilzomib, Carmustine, Carmustine Implant, Ceritinib, Cetuximab, Chlorambucil, CHLORAMBUCIL-PREDNISONE, CHOP, Cisplatin, Clofarabine, CMF, COPP, COPP-ABV, Crizotimb, CVP, Cyclophosphamide, Cytarabine, Cytarabine, Liposomal, Dabrafenib, Dacarbazine, Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, Doxorubicin Hydrochloride Liposome, Eltrombopag Olamine, Enzalutamide, Epirubicin Hydrochloride, EPOCH, Eribulin Mesylate, Erlotinib Hydrochloride, Etoposide Phosphate, Everolimus, Exemestane, FEC, Filgrastim, Fludarabine Phosphate, Fluorouracil, FU-LV, Fulvestrant, Gefitinib, Gemcitabine Hydrochloride, GEMCITABINE-CISPLATIN, GEMCITABINE-OXALUPLATIN, Gemtuzumab Ozogamicin, Glucarpidase, Goserelin Acetate, HPV Bivalent Vaccine, Recombinant HPV Quadrivalent Vaccine, Hyper-CVAD, Ibritumomab Tiuxetan, Ibrutinib, ICE, Idelalisib, Ifosfamide, Imatinib, Mesylate, Imiquimod, Iodine 131 Tositumomab and Tositumomab, Ipilimumab, Irinotecan Hydrochloride, Ixabepilone, Lapatinib Ditosylate, Lenalidomide, Letrozole, Leucovorin Calcium, Leuprolide Acetate, Liposomal Cytarabine, Lomustine, Mechlorethamine Hydrochloride, Megestrol Acetate, Mercaptopurine, Mesna, Methotrexate, Mitomycin C, Mitoxantrone Hydrochloride, MOPP, Nelarabine, Nilotinib, Obinutuzumab, Ofatumumab, Omacetaxine Mepesuccinate, OEPA, OFF, OPPA, Oxaliplatin, Paclitaxel, Paclitaxel Albumin-stabilized Nanoparticle Formulation, PAD, Palifermin, Pal onosetron Hydrochloride, Pamidronate Disodium, Panitumumab, Pazopanib Hydrochloride, Pegaspargase, Peginterferon Alfa-2b, Pembrolizumab, Pemetrexed Disodium, Pertuzumab, Plerixafor, Pomalidomide, Ponatinib Hydrochloride, Pralatrexate, Prednisone, Procarbazine Hydrochloride, Radium 223 Dichloride, Raloxifene Hydrochloride, Ramucirumab, Rasburicase, R-CHOP, R-CVP, Recombinant HPV Bivalent Vaccine, Recombinant HPV Quadrivalent Vaccine, Recombinant Interferon Alfa-2b, Regorafenib, Rituximab, Romidepsin, Romiplostim, Ruxolitinib Phosphate, Siltuximab, Sipuleucel-T, Sorafenib Tosylate, STANFORD V, Sunitinib Malate, TAC, Talc, Tamoxifen Citrate, Temozolomide, Temsirolimus, Thalidomide, Topotecan Hydrochloride, Toremifene, Tositumomab and I 131 Iodine Tositumomab, TPF, Trametinib, Trastuzumab, Vandetanib, VAMP, VelP, Vemurafenib, Vinblastine Sulfate, Vincristine Sulfate, Vincristine Sulfate Liposome, Vinorelbine Tartrate, Vismodegib, Vorinostat, XELOX, Ziv-Aflibercept, and Zoledronic Acid.

### Kits

In some aspects, the disclosure provides a kit comprising reagents and instructions necessary for carrying out the methods described herein.

In some embodiments, the kit comprises (i) an agent for converting cytosine unmethylated in the 5-position to uracil or another base that does not hybridize to guanine in the genomic DNA; (ii) at least one set of oligonucleotides complementary to the genomic DNA; and (iii) instructions for detecting methylation of genomic DNA in one or more biological samples in combination with instructions for detecting the one or more protein biomarkers in the one or more biological samples.

In some embodiments, the kit comprises (i) one or more reagents for detecting the one or more protein biomarkers; and (ii) instructions for detecting the presence or amount of the one or more protein biomarkers in one or more biological samples in combination with instructions for detecting methylated genomic DNA.

In some embodiments, the kit comprises (i) an agent for converting cytosine unmethylated in the 5-position to uracil or another base that does not hybridize to guanine in the genomic DNA; (ii) at least one set of oligonucleotides complementary to the genomic DNA; (iii) one or more reagents for detecting the one or more protein biomarkers; and (iv) instructions for detecting methylation of genomic DNA and the presence or amount of the one or more protein biomarkers in one or more biological samples.

In some embodiments, the kit comprises (i) an agent for converting cytosine unmethylated in the 5-position to uracil or another base that does not hybridize to guanine in the genomic DNA; (ii) at least one set of oligonucleotides complementary to the genomic DNA; and (iii) instructions for detecting methylation of genomic DNA in one or more plasma or serum samples in combination with instructions for detecting the one or more protein biomarkers in the one or more plasma or serum samples.

In some embodiments, the kit comprises (i) one or more reagents for detecting the one or more protein biomarkers; and (ii) instructions for detecting the presence or amount of the one or more protein biomarkers in one or more plasma or serum samples in combination with instructions for detecting methylated genomic DNA.

In some embodiments, the kit comprises (i) an agent for converting cytosine unmethylated in the 5-position to uracil or another base that does not hybridize to guanine in the genomic DNA; (ii) at least one set of oligonucleotides complementary to the genomic DNA; (iii) one or more reagents for detecting the one or more protein biomarkers; and (iv) instructions for detecting methylation of genomic DNA and the presence or amount of the one or more protein biomarkers in one or more plasma or serum samples.

In some embodiments, the kit comprises (i) an agent for converting cytosine unmethylated in the 5-position to uracil or another base that does not hybridize to guanine in the genomic DNA; (ii) at least one set of oligonucleotides complementary to the genomic DNA; and (iii) instructions for detecting methylation of genomic DNA in one or more biological samples obtained from a subject suspected of having colorectal cancer or a colorectal cell proliferative disease, in combination with instructions for detecting the one or more protein biomarkers in the one or more biological samples.

In some embodiments, the kit comprises (i) one or more reagents for detecting the one or more protein biomarkers; and (ii) instructions for detecting the presence or amount of the one or more protein biomarkers in one or more biological samples obtained from a subject suspected of having colorectal cancer or a colorectal cell proliferative disease, in combination with instructions for detecting methylated genomic DNA.

In some embodiments, the kit comprises (i) an agent for converting cytosine unmethylated in the 5-position to uracil or another base that does not hybridize to guanine in the genomic DNA; (ii) at least one set of oligonucleotides complementary to the genomic DNA; (iii) one or more reagents for detecting the one or more protein biomarkers; and (iv) instructions for detecting methylation of genomic DNA and the presence or amount of the one or more protein biomarkers in one or more biological samples obtained from a subject suspected of having colorectal cancer or a colorectal cell proliferative disease.

In some embodiments, the kit comprises (i) an agent for converting cytosine unmethylated in the 5-position to uracil or another base that does not hybridize to guanine in the genomic DNA; (ii) at least one set of oligonucleotides complementary to the genomic DNA; and (iii) instructions for detecting methylation of genomic DNA in one or more plasma or serum samples obtained from a subject suspected of having colorectal cancer or a colorectal cell proliferative disease, in combination with instructions for detecting the one or more protein biomarkers in the one or more biological samples.

In some embodiments, the kit comprises (i) one or more reagents for detecting the one or more protein biomarkers; and (ii) instructions for detecting the presence or amount of the one or more protein biomarkers in one or more plasma or serum samples obtained from a subject suspected of having colorectal cancer or a colorectal cell proliferative disease in combination with instructions for detecting methylated genomic DNA.

In some embodiments, the kit comprises (i) an agent for converting cytosine unmethylated in the 5-position to uracil or another base that does not hybridize to guanine in the genomic DNA; (ii) at least one set of oligonucleotides complementary to the genomic DNA; (iii) one or more reagents for detecting the one or more protein biomarkers; and (iv) instructions for detecting methylation of genomic DNA and the presence or amount of the one or more protein biomarkers in one or more plasma or serum samples obtained from a subject suspected of having colorectal cancer or a colorectal cell proliferative disease.

In some embodiments, the kit comprises (i) an agent for converting cytosine unmethylated in the 5-position to uracil or another base that does not hybridize to guanine in the genomic DNA; (ii) at least one set of oligonucleotides complementary to methylated genomic DNA selected from mSEPT9, mANKRD13B, and combinations thereof; and (iii) instructions for detecting methylation of genomic DNA in one or more biological samples in combination with instructions for detecting the one or more protein biomarkers comprising CEA and/or amphiregulin in the one or more biological samples.

In some embodiments, the kit comprises (i) one or more reagents for detecting the one or more protein biomarkers comprising CEA and/or amphiregulin; and (ii) instructions for detecting the presence or amount of the one or more protein biomarkers in one or more biological samples in combination with instructions for detecting methylated genomic DNA selected from mSEPT9, mANKRD13B, and combinations thereof.

In some embodiments, the kit comprises (i) an agent for converting cytosine unmethylated in the 5-position to uracil or another base that does not hybridize to guanine in the genomic DNA; (ii) at least one set of oligonucleotides complementary to methylated genomic DNA selected from mSEPT9, mANKRD13B, and combinations thereof; (iii) one or more reagents for detecting the one or more protein biomarkers comprising CEA and/or amphiregulin; and (iv) instructions for detecting methylation of genomic DNA and the presence or amount of the one or more protein biomarkers in one or more biological samples.

In some embodiments, the kit comprises (i) an agent for converting cytosine unmethylated in the 5-position to uracil or another base that does not hybridize to guanine in the genomic DNA; (ii) at least one set of oligonucleotides complementary to methylated genomic DNA selected from mSEPT9, mANKRD13B, and combinations thereof; and (iii) instructions for detecting methylation of genomic DNA in one or more plasma or serum samples in combination with instructions for detecting the one or more protein biomarkers comprising CEA and/or amphiregulin in the one or more plasma or serum samples.

In some embodiments, the kit comprises (i) one or more reagents for detecting the one or more protein biomarkers comprising CEA and/or amphiregulin; and (ii) instructions for detecting the presence or amount of the one or more protein biomarkers in one or more plasma or serum samples in combination with instructions for detecting methylated genomic DNA selected from mSEPT9, mANKRD13B, and combinations thereof.

In some embodiments, the kit comprises (i) an agent for converting cytosine unmethylated in the 5-position to uracil or another base that does not hybridize to guanine in the genomic DNA; (ii) at least one set of oligonucleotides complementary to the genomic DNA; (iii) one or more reagents for detecting the one or more protein biomarkers; and (iv) instructions for detecting methylation of genomic DNA and the presence or amount of the one or more protein biomarkers comprising CEA and/or amphiregulin in one or more plasma or serum samples.

In some embodiments, the kit comprises (i) an agent for converting cytosine unmethylated in the 5-position to uracil or another base that does not hybridize to guanine in the genomic DNA; (ii) at least one set of oligonucleotides complementary to methylated genomic DNA selected from mSEPT9, mANKRD13B, and combinations thereof; and (iii) instructions for detecting methylation of genomic DNA in one or more biological samples obtained from a subject suspected of having colorectal cancer or a colorectal cell proliferative disease, in combination with instructions for detecting the one or more protein biomarkers comprising CEA and/or amphiregulin in the one or more biological samples.

In some embodiments, the kit comprises (i) one or more reagents for detecting the one or more protein biomarkers comprising CEA and/or amphiregulin; and (ii) instructions for detecting the presence or amount of the one or more protein biomarkers in one or more biological samples obtained from a subject suspected of having colorectal cancer or a colorectal cell proliferative disease, in combination with instructions for detecting methylated genomic DNA selected from mSEPT9, mANKRD13B, and combinations thereof.

In some embodiments, the kit comprises (i) an agent for converting cytosine unmethylated in the 5-position to uracil or another base that does not hybridize to guanine in the genomic DNA; (ii) at least one set of oligonucleotides complementary to methylated genomic DNA selected from mSEPT9, mANKRD13B, and combinations thereof; (iii) one or more reagents for detecting the one or more protein biomarkers comprising CEA and/or amphiregulin; and (iv) instructions for detecting methylation of genomic DNA and the presence or amount of the one or more protein biomarkers in one or more biological samples obtained from a subject suspected of having colorectal cancer or a colorectal cell proliferative disease.

In some embodiments, the kit comprises (i) an agent for converting cytosine unmethylated in the 5-position to uracil or another base that does not hybridize to guanine in the genomic DNA; (ii) at least one set of oligonucleotides complementary to methylated genomic DNA selected from mSEPT9, mANKRD13B, and combinations thereof; and (iii) instructions for detecting methylation of genomic DNA in one or more plasma or serum samples obtained from a subject suspected of having colorectal cancer or a colorectal cell proliferative disease, in combination with instructions for detecting the one or more protein biomarkers comprising CEA and/or amphiregulin in the one or more biological samples.

In some embodiments, the kit comprises (i) one or more reagents for detecting the one or more protein biomarkers comprising CEA and/or amphiregulin; and (ii) instructions for detecting the presence or amount of the one or more protein biomarkers in one or more plasma or serum samples obtained from a subject suspected of having colorectal cancer or a colorectal cell proliferative disease in combination with instructions for detecting methylated genomic DNA selected from mSEPT9, mANKRD13B, and combinations thereof.

In some embodiments, the kit comprises (i) an agent for converting cytosine unmethylated in the 5-position to uracil or another base that does not hybridize to guanine in the genomic DNA; (ii) at least one set of oligonucleotides complementary to methylated genomic DNA selected from mSEPT9, mANKRD13B, and combinations thereof; (iii) one or more reagents for detecting the one or more protein biomarkers comprising CEA and/or amphiregulin; and (iv) instructions for detecting methylation of genomic DNA and the presence or amount of the one or more protein biomarkers in one or more plasma or serum samples obtained from a subject suspected of having colorectal cancer or a colorectal cell proliferative disease.

In some embodiments, the one or more protein biomarkers of the kit comprises haptoglobin. In some embodiments, a kit described herein comprises instructions for detecting haptoglobin in a sample (e.g., plasma sample). In some embodiments, a kit described herein comprises reagents for detecting haptoglobin in a sample (e.g., plasma sample).

### EXAMPLES

### Example 1—Multi-Analyte Panel for Detecting Colorectal Carcinoma

To determine whether the specificity and sensitivity of an assay for detecting colorectal carcinoma in a subject could be improved, presence of protein biomarkers were assessed in combination with DNA methylation markers in the blood of subjects diagnosed with colorectal carcinoma relative to expression in control subjects.

### Methods

Colorectal carcinoma (CRC) biomarkers were measured in four aliquots of blood plasma collected from 70 individuals with CRC and 171 individuals without CRC as further described in Tables 1 and 2. Two DNA methylation markers were measured in three assays in one combined multiplex Real-time PCR. Three selected protein biomarkers were individually measured using ELISAs.

TABLE 1
Number of samples by gender for colorectal
cancer (CRC) and for controls.

| | Female | Male | total |
|---|---|---|---|
| control | 97 | 74 | 171 |
| crc | 37 | 33 | 70 |
| | | | 241 |

TABLE 2
Number of samples by gender for different colorectal
cancer (CRC) stages, and for controls from individuals
with no evidence of disease (NED) and with polyps.

| | Female | Male | Total |
|---|---|---|---|
| CRC stage I | 6 | 10 | 16 |
| CRC stage II | 11 | 8 | 19 |
| CRC stage III | 15 | 13 | 28 |
| CRC stage IV | 5 | 2 | 7 |
| control: NED | 78 | 49 | 127 |
| control: polyps | 19 | 25 | 44 |

### DNA Methylation Markers

Blood plasma samples from colorectal carcinoma patients (CRC) and individuals with no cancer (controls) were processed with the Epi BiSKit (Epigenomics AG). Briefly, DNA extraction from 3.5 ml of blood-plasma per individual and subsequent bisulfite conversion of DNA is performed with the Epi BiSKit (Epigenomics AG) according to the workflow as defined in the instructions for use of the Epi BiSKit (Epigenomics AG), including a prior treatment of blood plasma with proteinase K.

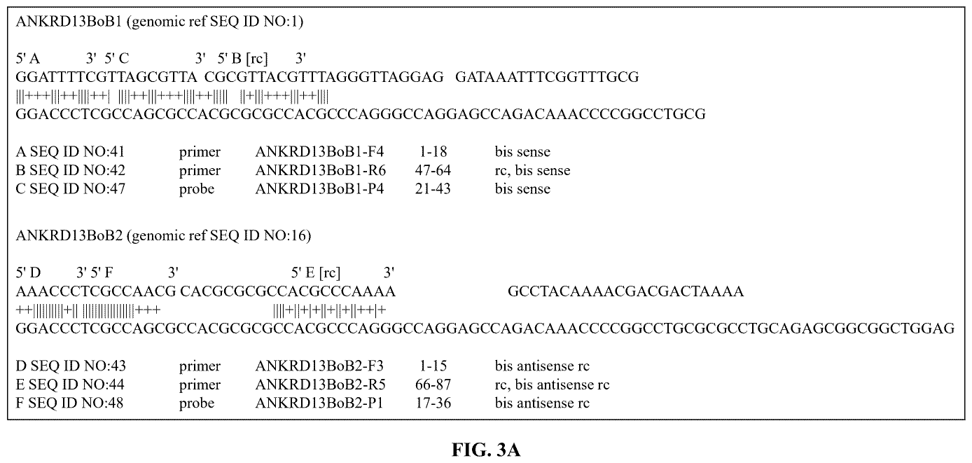

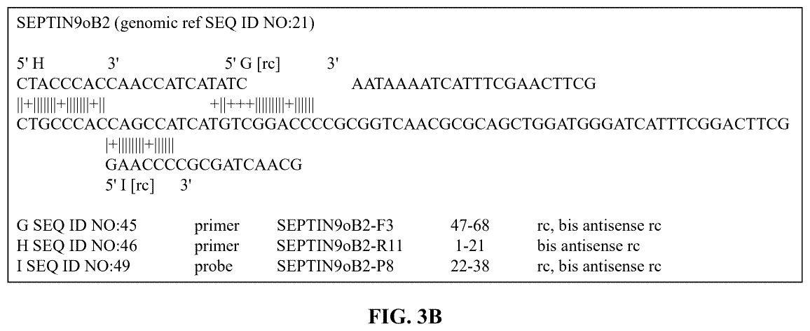

For each sample, 25 μl bisulfite-treated DNA were amplified in duplicate in 50 μl total volume Real-time PCR quadruplex containing three methylation sensitive (MSP) assays for one bisulfite specific and methylation unspecific ACTB control assay and the two DNA-methylation markers mSeptin9 and mANKRD13B, which is measured on two strands (FIGS. 3A-3B). Table 3 lists the genomic reference sequences and derived bisulfite converted sequences. Table 4 lists sequence IDs of oligomers used for Real-time PCR assays. The assays were measured using an Applied Biosystems™ Quant Studio™ 5 Dx Real-Time PCR Instrument, using 45 cycles, and interpreting basic results as cycle thresholds (Cts) of Realtime-PCR amplification curves.

For numerical interpretation, data for un-amplified assays (no curve) were set to the maximum Ct of 45.

TABLE 3
Sequence IDs, abbreviations, names and associated regions in the human genome (GRCh38
build): genomic reference sequences and derived bisulfite converted sequences.

| SEQ | | | Location | |
|---|---|---|---|---|
| ID NO | Abbr. | Name | GRCh38 | Type |
| 1 | ANB1 | ANKRD13BoB1 | 17: 29613595- | genomic reference |
| 2 | | Assay | 29613658 | converted C to T (bis1) |
| 3 | | | | converted C to T (bis1), reverse complement |
| 4 | | | | converted G to A (bis2 rc) |
| 5 | | | | converted G to A (bis2 rc), reverse complement |
| 6 | | ANKRD13B | 17: 29613595- | genomic reference |
| 7 | | Extended | 29613700 | converted C to T (bis1) |
| 8 | | Assay | | converted C to T (bis1), reverse complement |
| 9 | | Region A | | converted G to A (bis2 rc) |
| 10 | | | | converted G to A (bis2 rc), reverse complement |
| 11 | | ANKRD13B | 17: 29613595- | genomic reference |
| 12 | | Extended | 29613940 | converted C to T (bis1) |
| 13 | | Assay | | converted C to T (bis1), reverse complement |
| 14 | | Region B | | converted G to A (bis2 rc) |
| 15 | | | | converted G to A (bis2 rc), reverse complement |
| 16 | ANB2 | ANKRD13BoB2 | 17: 29613595- | genomic reference |
| 17 | | Assay | 29613681 | converted C to T (bis1) |
| 18 | | | | converted C to T (bis1), reverse complement |
| 19 | | | | converted G to A (bis2 rc) |
| 20 | | | | converted G to A (bis2 rc), reverse complement |
| 21 | S9B2 | SEPTIN9oB2 | 17: 77373482- | genomic reference |
| 22 | | Assay | 77373549 | converted C to T (bis1) |
| 23 | | | | converted C to T (bis1), reverse complement |
| 24 | | | | converted G to A (bis2 rc) |
| 25 | | | | converted G to A (bis2 rc), reverse complement |
| 26 | | SEPTIN9 | 17: 77373475- | genomic reference |
| 27 | | Assay | 77373549 | converted C to T (bis1) |
| 28 | | Region | | converted C to T (bis1), reverse complement |
| 29 | | | | converted G to A (bis2 rc) |
| 30 | | | | converted G to A (bis2 rc), reverse complement |
| 31 | | SEPTIN9 | 17: 77372606- | genomic reference |
| 32 | | Assay + CpG- | 77374424 | converted C to T (bis1) |
| 33 | | island | | converted C to T (bis1), reverse complement |
| 34 | | | | converted G to A (bis2 rc) |
| 35 | | | | converted G to A (bis2 rc), reverse complement |
| 36 | | SEPTIN9 | 17: 77372979- | genomic reference |
| 37 | | Extended | 77374040 | converted C to T (bis1) |
| 38 | | Assay | | converted C to T (bis1), reverse complement |
| 39 | | Region | | converted G to A (bis2 rc) |
| 40 | | | | converted G to A (bis2 rc), reverse complement |

TABLE 4
Sequence IDs of oligomers (primers, probes) used for Real-time PCR assays.

| SEQ ID NO | Acronym | Name | Sequence |
|---|---|---|---|
| 41 | ANB1-F4 | ANKRD13BoB1-F4 | ggattttcgttagcgtta |
| 42 | ANB1-R6 | ANKRD13BoB1-R6 | cgcaaaccgaaatttatc |
| 43 | ANB2-F3 | ANKRD13BoB2-F3 | aaaccctcgccaacg |
| 44 | ANB2-R5 | ANKRD13BoB2-R5 | ttttagtcgtcgttttgtaggc |
| 45 | S9B2-F3 | SEPTIN90B2-F3 | cgaagttcgaaatgattttatt |
| 46 | S9B2-R11 | SEPTIN90B2-R11 | ctacccaccaaccatcatatc |
| 47 | ANB1-P4 | ANKRD13BoB1-P4 | cgcgttacgtttagggttaggag |
| 48 | ANB2-P1 | ANKRD13BoB2-P1 | cacgcgcgccacgcccaaaa |
| 49 | S9B2-P8 | SEPTIN90B2-P8 | cgttgatcgggggttc |

### Amphiregulin (AR)

Concentrations [pg/mL] of the protein Amphiregulin in blood plasma samples were assessed using the Human Amphiregulin Quantikine ELISA Kit from R&D Systems according to manufacturer's protocol. In brief, two aliquots of 50 μl of blood plasma were used without previous dilution from each individual. Standard curve is prepared in the range of 0 to 1000 μg/ml. Washing steps were done manually, and final measurement is done with Tecan Infinite F200 PRO microplate reader at absorption of 450 nm. For calculation, the average of the duplicate readings for each standard, control, and sample were subtracted by the average zero standard absorbance (0 pg/ml). For the standard curve the mean absorbance for each standard is plotted on the y-axis against the concentration on the x-axis. Protein concentration is calculated with the slope of best linear fit through the points by crossing the origin (0,0).

### CYFRA

Concentrations [ng/mL] of the protein Human Cytokeratin Fragment Antigen 21-1 (CYFRA21-1) in blood plasma samples were assessed using the Human Cytokeratin Fragment Antigen 21-1 (CYFRA21-1) ELISA kit from Cusabio according to manufacturer's protocol. In brief, two aliquots of 100 μl of blood plasma were used without previous dilution from each individual. Standard curve is prepared in the range of 0 to 20 ng/ml. Washing steps were done using the Biotek 405TS microplate washer, and final measurement is done with Biotek ELx808LBS plate reader at absorption of 450 nm. For calculation, the average of the duplicate readings for each standard, control, and sample were subtracted by the average zero standard absorbance (0 ng/ml). For the standard curve, the mean absorbance for each standard is plotted on the y-axis against the concentration on the x-axis. Protein concentration is calculated using the linear trendline with a log concentration axis.

### CEA

Concentrations [ng/mL] of the protein carcinoembryonic antigen (CEA) in blood plasma samples were assessed using the Human CEA ELISA Kit (PN:EHCEA) from ThermoFisher according to manufacturer's protocol. In brief, 2 aliquots of 50 μl of blood plasma were used without previous dilution from each individual. Standard curve is prepared in the range of 0 to 83.3 ng/ml. Washing steps were done using the Biotek 405TS microplate washer, and final measurement is done with Biotek ELx808LBS plate reader at absorption of 450 nm. For calculation, the average of the duplicate readings for each standard, control, and sample were subtracted by the average zero standard absorbance (0 ng/ml). For the standard curve, the mean absorbance for each standard is plotted on the y-axis against the concentration on the x-axis. Protein concentration is calculated using the linear trendline with a log concentration axis.

The three minimum Cts for all three MSP assays over duplicates were used in logistic regression without or with the protein measurements of the individuals as up to three additional numeric variables. The areas under the curve (AUC) of Receiver operating characteristic (ROC) analysis and the Sensitivity at Specificity of 0.9 using the full data set were used to assess the discrimination of CRC vs. control in the different sets of variables.

### Results

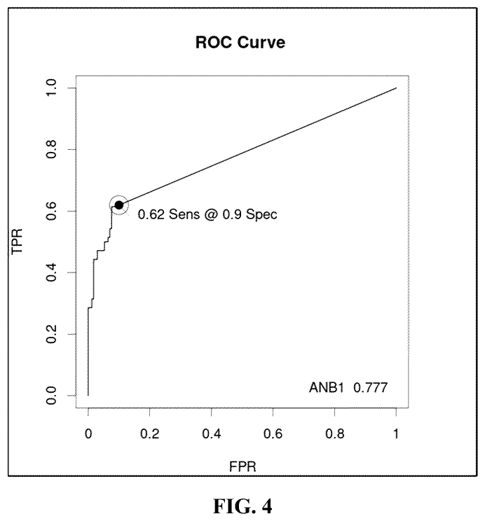

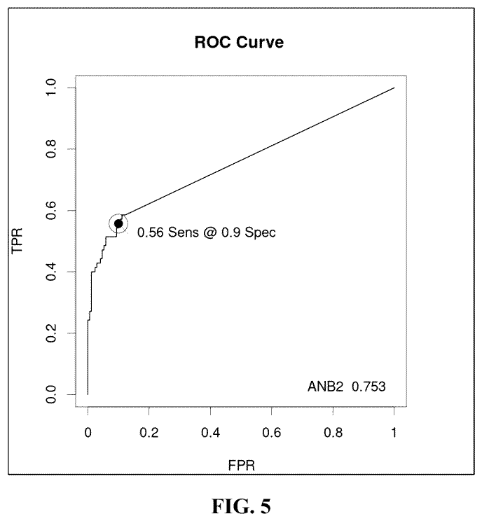

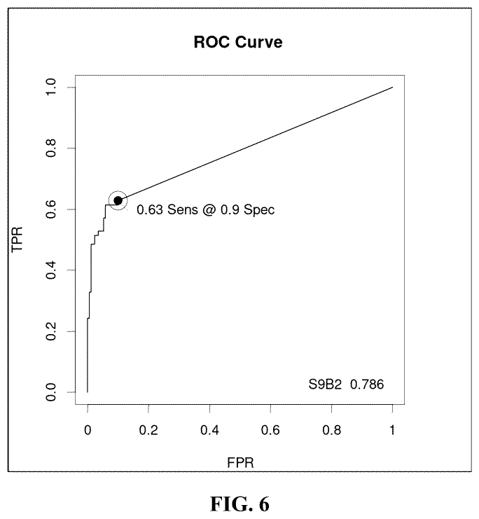

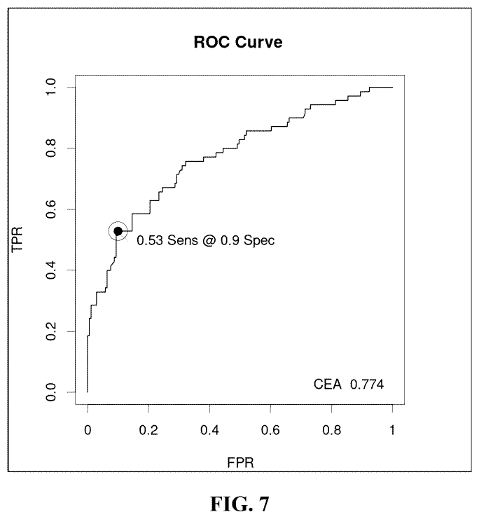

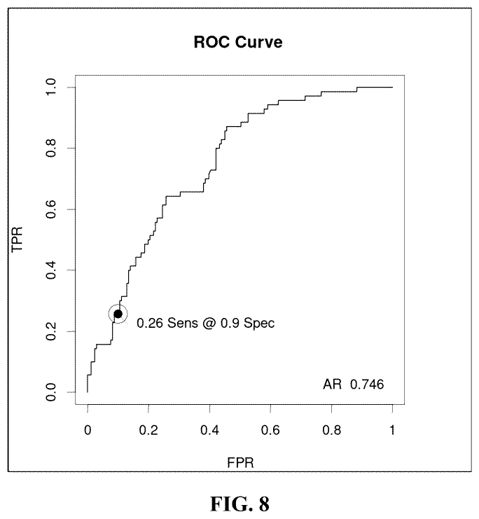

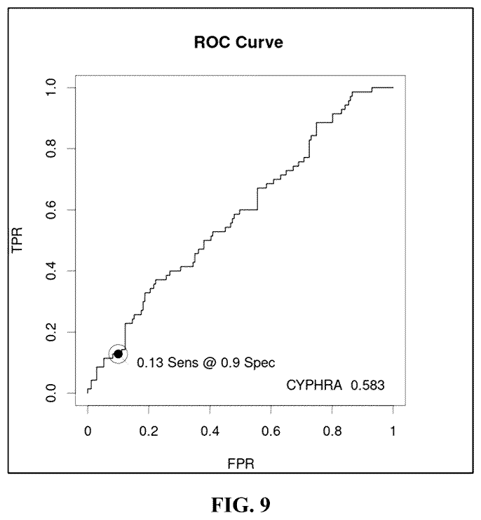

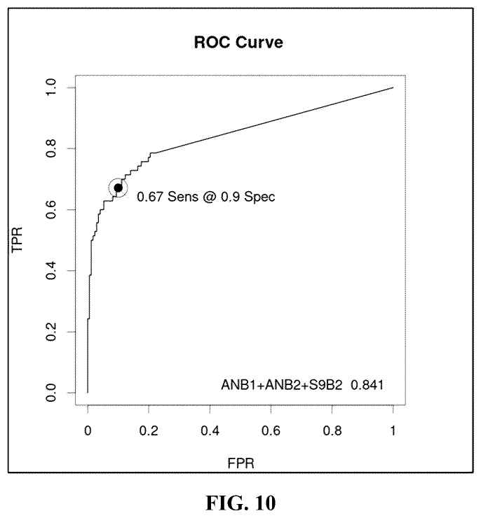

The individual DNA methylation marker measurements by Realtime-PCR as assessed by minimum Cts of duplicates for each of the three MSP marker assays lead to AUCs between 0.75 and 0.79 and to Sensitivities from 0.56 to 0.63 (see FIGS. 4 to 6 and Table 5). The combination of DNA methylation markers lead to an AUC of 0.84 and Sensitivity of 0.67 at Specificity of 0.9 (see FIG. 10). The individual protein measurements lead to AUC of 0.77 for CEA (see FIG. 7), of 0.74 for Amphiregulin (see FIG. 8), and of 0.538 for CYPHRA (see FIG. 9).

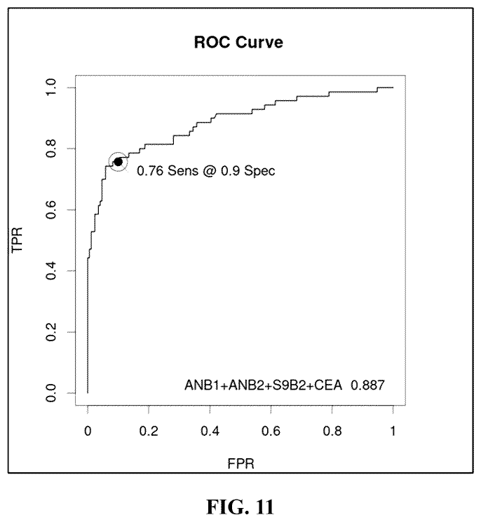

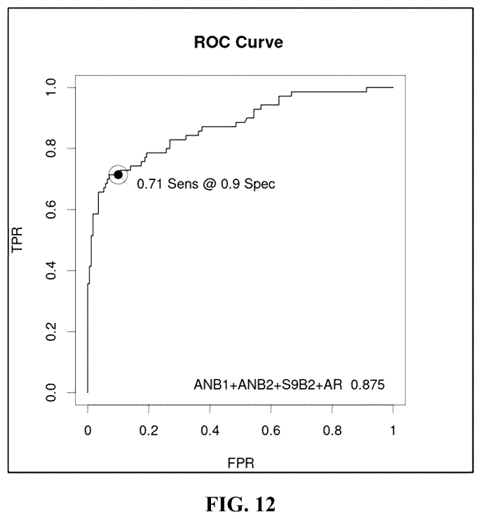

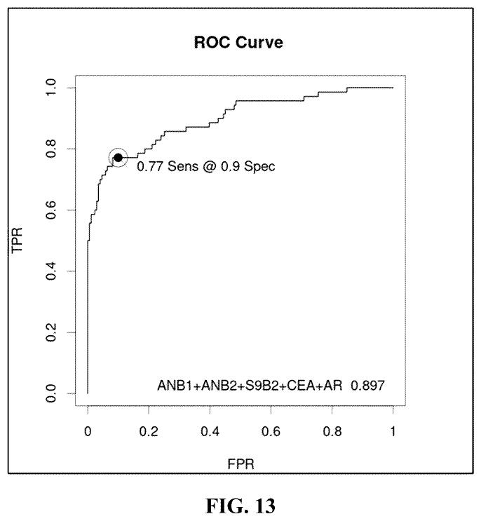

Combinations of data from the three DNA-methylation biomarker assays with protein data from the two proteins with a performance >AUC of 0.7 (CEA and Amphiregulin) lead to higher sensitivities at a Specificity of 0.9 than obtained with the DNA methylation markers alone (see FIGS. 11-13 and Table 6) with a maximum Sensitivity of 0.77 for the combination with CEA and Amphiregulin.

Further, data for the combined markers (SEPT9+ANB1+ANB2+CEA+AREG) was compared with altered data sets to assess the stability of the performance as described as Sensitivity at Specificity of 0.9. The sensitivity remains at levels >0.75 in altered patient data sets (see Table 7) restricted to age subsets (age 45-75) at core screening population without optionally screened older patients, with addition of overrepresented advanced adenomas, or with exclusion of stage IV CRC patients.

Overall, these data indicate including the detection of protein biomarkers in combination with detection of methylation of genomic DNA improves the sensitivity for detecting subjects with CRC.

TABLE 5
Measurements for all biomarkers in all 241 individuals. The
first column contains the diagnostic group. Column 2-4 contain
the three blood plasma derived Ct values of methylation specific
Realtime PCR quadruplex assays: ANKRD13b assays on the bisulfite
converted sense strand (ANB1), ANKRD13b assay on the reverse
complement strand (ANB2), Septin9 assay on the reverse complement
strand (S9B2). The columns 5-7 contain protein measurements
in [pg/mL] for CEA, Amphiregulin (AR) and CYFRA21-1
(CYPHRA) - negative results as obtained by calculations with
the calibration curve were set to 0 (zero).

| Diagnosis | ANB1 | ANB2 | S9B2 | CEA | AR | CYPHRA |
|---|---|---|---|---|---|---|
| crc | 33.28 | 32.26 | 35.92 | 63.45 | 5.45 | 2.38 |
| crc | 34.27 | 33.49 | 33.21 | 16.39 | 4.70 | 7.39 |
| crc | 45.00 | 45.00 | 35.90 | 19.00 | 29.75 | 5.26 |
| crc | 39.95 | 39.74 | 38.89 | 128.34 | 5.79 | 3.98 |
| crc | 45.00 | 38.29 | 45.00 | 1.91 | 10.10 | 1.33 |
| crc | 36.48 | 35.37 | 35.98 | 20.40 | 15.45 | 1.20 |
| crc | 31.58 | 30.70 | 31.07 | 38.83 | 5.00 | 5.31 |
| crc | 40.63 | 45.00 | 38.58 | 5.86 | 6.21 | 2.05 |
| crc | 45.00 | 45.00 | 45.00 | 25.66 | 0.00 | 4.48 |
| crc | 34.61 | 33.93 | 33.42 | 125.26 | 4.35 | 2.33 |
| crc | 45.00 | 40.53 | 45.00 | 19.97 | 17.46 | 4.50 |
| crc | 38.75 | 37.87 | 45.00 | 23.53 | 20.10 | 2.01 |
| crc | 45.00 | 45.00 | 45.00 | 5.58 | 20.37 | 5.45 |
| crc | 45.00 | 45.00 | 45.00 | 2.58 | 14.17 | 2.66 |
| crc | 36.74 | 40.80 | 34.58 | 2.00 | 2.75 | 5.30 |
| crc | 27.16 | 26.17 | 26.87 | 201.18 | 121.95 | 3.48 |
| crc | 39.01 | 36.03 | 36.66 | 0.55 | 27.05 | 9.24 |
| crc | 40.59 | 45.00 | 35.68 | 8.79 | 13.92 | 1.36 |
| crc | 38.16 | 34.45 | 45.00 | 10.49 | 12.40 | 1.33 |
| crc | 45.00 | 45.00 | 45.00 | 1.38 | 5.10 | 2.01 |
| crc | 25.62 | 24.63 | 24.87 | 199.85 | 188.90 | 2.36 |
| crc | 34.35 | 33.54 | 33.53 | 17.17 | 18.05 | 6.62 |
| crc | 38.14 | 45.00 | 37.48 | 30.32 | 1.10 | 5.21 |
| crc | 39.15 | 35.48 | 39.78 | 4.01 | 10.70 | 2.96 |
| crc | 45.00 | 45.00 | 44.34 | 24.80 | 39.40 | 6.56 |
| crc | 36.13 | 38.85 | 34.89 | 42.46 | 8.75 | 1.00 |
| crc | 35.62 | 34.74 | 35.22 | 3.74 | 5.00 | 0.80 |
| crc | 25.69 | 24.85 | 25.46 | 137.15 | 170.75 | 4.86 |
| crc | 39.62 | 45.00 | 45.00 | 15.45 | 11.00 | 1.88 |
| crc | 33.99 | 33.51 | 34.41 | 10.82 | 9.50 | 7.52 |
| crc | 45.00 | 45.00 | 35.96 | 0.00 | 15.75 | 1.61 |
| crc | 45.00 | 45.00 | 45.00 | 18.81 | 9.06 | 9.59 |
| crc | 33.99 | 33.64 | 33.14 | 25.58 | 12.21 | 4.07 |
| crc | 37.20 | 45.00 | 45.00 | 23.53 | 9.12 | 1.73 |
| crc | 33.27 | 32.53 | 33.62 | 15.97 | 19.00 | 4.60 |
| crc | 36.28 | 35.91 | 35.23 | 31.45 | 3.60 | 4.28 |
| crc | 45.00 | 45.00 | 45.00 | 9.12 | 6.25 | 7.99 |
| crc | 27.84 | 26.78 | 27.68 | 32.84 | 110.50 | 4.95 |
| crc | 45.00 | 38.18 | 36.16 | 7.86 | 19.30 | 0.70 |
| crc | 45.00 | 45.00 | 45.00 | 12.72 | 36.60 | 2.03 |
| crc | 45.00 | 45.00 | 45.00 | 9.80 | 3.45 | 2.30 |
| crc | 34.63 | 33.95 | 35.94 | 86.54 | 29.87 | 4.28 |
| crc | 45.00 | 45.00 | 39.07 | 6.69 | 5.08 | 2.71 |
| crc | 36.43 | 36.57 | 38.73 | 19.41 | 8.87 | 1.28 |
| crc | 45.00 | 45.00 | 45.00 | 10.45 | 5.37 | 5.23 |
| crc | 45.00 | 37.72 | 35.60 | 12.44 | 14.25 | 1.49 |
| crc | 37.25 | 38.96 | 45.00 | 11.04 | 11.15 | 5.97 |
| crc | 45.00 | 45.00 | 35.58 | 4.24 | 15.67 | 2.76 |
| crc | 45.00 | 45.00 | 45.00 | 63.37 | 4.79 | 2.59 |
| crc | 34.54 | 33.10 | 32.83 | 1.61 | 13.85 | 1.19 |
| crc | 45.00 | 45.00 | 45.00 | 3.66 | 2.87 | 2.72 |
| crc | 34.69 | 35.72 | 36.30 | 8.93 | 4.67 | 3.39 |
| crc | 29.39 | 28.32 | 29.21 | 15.43 | 7.71 | 0.41 |
| crc | 45.00 | 45.00 | 45.00 | 10.86 | 3.50 | 1.18 |
| crc | 38.48 | 45.00 | 45.00 | 16.03 | 11.62 | 0.92 |
| crc | 38.49 | 45.00 | 45.00 | 17.26 | 0.45 | 3.20 |
| crc | 45.00 | 37.30 | 45.00 | 14.98 | 4.50 | 0.83 |
| crc | 45.00 | 45.00 | 45.00 | 12.79 | 13.75 | 3.89 |
| crc | 33.74 | 32.97 | 33.00 | 15.93 | 2.42 | 1.37 |
| crc | 45.00 | 45.00 | 35.65 | 30.28 | 15.20 | 1.91 |
| crc | 45.00 | 45.00 | 45.00 | 8.45 | 10.79 | 1.69 |
| crc | 26.98 | 25.94 | 28.64 | 173.58 | 13.45 | 2.03 |
| crc | 40.30 | 37.94 | 45.00 | 42.04 | 18.85 | 2.43 |
| crc | 32.56 | 32.13 | 32.32 | 12.59 | 36.00 | 11.43 |
| crc | 45.00 | 45.00 | 36.85 | 1.63 | 9.90 | 0.73 |
| crc | 45.00 | 45.00 | 45.00 | 0.00 | 4.50 | 1.00 |
| crc | 34.72 | 33.40 | 34.84 | 0.00 | 13.55 | 2.92 |
| crc | 36.29 | 36.85 | 34.33 | 86.32 | 23.10 | 2.98 |
| crc | 37.58 | 35.53 | 39.62 | 23.24 | 10.55 | 1.44 |
| crc | 35.77 | 34.56 | 34.91 | 8.09 | 9.00 | 5.27 |
| control | 45.00 | 45.00 | 45.00 | 4.89 | 0.00 | 2.68 |
| control | 45.00 | 45.00 | 45.00 | 0.00 | 0.00 | 1.90 |
| control | 45.00 | 45.00 | 45.00 | 10.81 | 0.00 | 0.84 |
| control | 45.00 | 45.00 | 45.00 | 0.00 | 8.28 | 0.20 |
| control | 45.00 | 37.29 | 45.00 | 3.35 | 0.00 | 5.02 |
| control | 45.00 | 45.00 | 45.00 | 0.00 | 2.71 | 1.17 |
| control | 45.00 | 45.00 | 45.00 | 1.66 | 5.37 | 2.20 |
| control | 45.00 | 45.00 | 45.00 | 0.38 | 10.67 | 0.93 |
| control | 45.00 | 45.00 | 45.00 | 3.80 | 7.45 | 9.79 |
| control | 45.00 | 45.00 | 45.00 | 6.47 | 13.50 | 3.78 |
| control | 45.00 | 45.00 | 45.00 | 2.12 | 30.75 | 5.39 |
| control | 45.00 | 45.00 | 45.00 | 7.01 | 0.00 | 8.33 |
| control | 45.00 | 45.00 | 45.00 | 9.28 | 4.17 | 3.34 |
| control | 45.00 | 45.00 | 45.00 | 0.11 | 3.20 | 0.17 |
| control | 45.00 | 45.00 | 45.00 | 5.67 | 3.00 | 1.89 |
| control | 45.00 | 45.00 | 45.00 | 22.15 | 8.95 | 6.19 |
| control | 45.00 | 45.00 | 45.00 | 1.02 | 0.00 | 1.87 |
| control | 45.00 | 45.00 | 45.00 | 12.29 | 2.40 | 0.30 |
| control | 45.00 | 45.00 | 45.00 | 0.00 | 9.85 | 0.55 |
| control | 45.00 | 45.00 | 45.00 | 3.41 | 0.00 | 2.46 |
| control | 45.00 | 45.00 | 45.00 | 9.40 | 8.45 | 2.31 |
| control | 45.00 | 45.00 | 45.00 | 0.00 | 22.67 | 1.12 |
| control | 45.00 | 45.00 | 45.00 | 9.48 | 6.35 | 2.55 |
| control | 45.00 | 45.00 | 45.00 | 9.85 | 0.00 | 0.99 |
| control | 45.00 | 45.00 | 45.00 | 4.26 | 3.33 | 1.38 |
| control | 45.00 | 45.00 | 45.00 | 1.88 | 0.10 | 1.96 |
| control | 34.97 | 34.00 | 34.53 | 0.95 | 0.00 | 2.41 |
| control | 45.00 | 45.00 | 35.28 | 0.00 | 1.20 | 1.93 |
| control | 45.00 | 45.00 | 45.00 | 0.00 | 7.67 | 2.99 |
| control | 45.00 | 45.00 | 45.00 | 22.92 | 3.63 | 5.52 |
| control | 45.00 | 45.00 | 45.00 | 0.00 | 0.00 | 1.27 |
| control | 45.00 | 45.00 | 45.00 | 1.73 | 4.21 | 3.08 |
| control | 45.00 | 44.08 | 45.00 | 2.03 | 21.54 | 1.78 |
| control | 45.00 | 45.00 | 45.00 | 17.44 | 42.60 | 5.44 |
| control | 45.00 | 45.00 | 45.00 | 10.41 | 4.75 | 2.79 |
| control | 45.00 | 38.33 | 45.00 | 4.45 | 0.00 | 0.13 |
| control | 45.00 | 45.00 | 45.00 | 0.00 | 0.67 | 1.69 |
| control | 45.00 | 45.00 | 45.00 | 24.38 | 8.39 | 2.33 |
| control | 45.00 | 45.00 | 45.00 | 0.97 | 8.35 | 0.54 |
| control | 45.00 | 45.00 | 45.00 | 7.21 | 11.46 | 0.57 |
| control | 45.00 | 45.00 | 45.00 | 4.82 | 3.21 | 0.36 |
| control | 45.00 | 45.00 | 37.21 | 1.74 | 12.75 | 1.90 |
| control | 45.00 | 45.00 | 45.00 | 9.62 | 42.96 | 4.74 |
| control | 45.00 | 45.00 | 45.00 | 1.48 | 20.95 | 1.89 |
| control | 45.00 | 45.00 | 45.00 | 14.63 | 2.22 | 2.54 |
| control | 45.00 | 45.00 | 45.00 | 38.24 | 12.04 | 1.15 |
| control | 45.00 | 45.00 | 45.00 | 6.92 | 0.00 | 1.50 |
| control | 45.00 | 45.00 | 45.00 | 21.48 | 10.83 | 3.18 |
| control | 45.00 | 45.00 | 45.00 | 0.00 | 0.78 | 1.69 |
| control | 45.00 | 45.00 | 45.00 | 1.54 | 0.80 | 1.46 |
| control | 45.00 | 45.00 | 40.30 | 6.35 | 3.60 | 3.92 |
| control | 38.38 | 38.32 | 45.00 | 7.96 | 4.50 | 0.72 |
| control | 45.00 | 45.00 | 45.00 | 3.96 | 22.05 | 0.69 |
| control | 45.00 | 45.00 | 45.00 | 1.63 | 1.00 | 3.49 |
| control | 45.00 | 45.00 | 45.00 | 0.59 | 5.54 | 2.55 |
| control | 45.00 | 45.00 | 45.00 | 0.00 | 5.33 | 4.62 |
| control | 38.24 | 37.07 | 42.09 | 1.30 | 1.83 | 3.16 |
| control | 45.00 | 45.00 | 45.00 | 6.62 | 0.00 | 5.91 |
| control | 45.00 | 45.00 | 37.81 | 20.39 | 0.00 | 8.14 |
| control | 45.00 | 45.00 | 45.00 | 2.24 | 14.75 | 1.16 |
| control | 45.00 | 45.00 | 45.00 | 1.87 | 2.42 | 2.76 |
| control | 35.80 | 36.41 | 36.56 | 5.79 | 0.00 | 0.96 |
| control | 45.00 | 45.00 | 45.00 | 0.27 | 1.00 | 1.94 |
| control | 45.00 | 45.00 | 45.00 | 8.10 | 0.35 | 1.17 |
| control | 45.00 | 45.00 | 45.00 | 14.78 | 1.90 | 1.05 |
| control | 45.00 | 45.00 | 45.00 | 3.35 | 0.00 | 2.53 |
| control | 45.00 | 45.00 | 45.00 | 0.00 | 8.38 | 1.68 |
| control | 45.00 | 45.00 | 45.00 | 1.90 | 0.00 | 2.16 |
| control | 45.00 | 45.00 | 45.00 | 3.68 | 0.00 | 1.41 |
| control | 45.00 | 45.00 | 45.00 | 0.00 | 7.08 | 1.66 |
| control | 40.74 | 45.00 | 45.00 | 1.33 | 0.79 | 3.37 |
| control | 45.00 | 45.00 | 45.00 | 0.00 | 10.62 | 6.17 |
| control | 45.00 | 38.42 | 45.00 | 10.77 | 0.40 | 0.98 |
| control | 45.00 | 45.00 | 45.00 | 5.72 | 7.10 | 2.73 |
| control | 45.00 | 45.00 | 45.00 | 11.45 | 13.29 | 7.27 |
| control | 45.00 | 45.00 | 45.00 | 0.50 | 2.45 | 1.80 |
| control | 45.00 | 45.00 | 36.39 | 0.00 | 7.58 | 2.18 |
| control | 45.00 | 45.00 | 45.00 | 1.09 | 4.63 | 0.42 |
| control | 45.00 | 45.00 | 45.00 | 20.55 | 0.00 | 6.10 |
| control | 45.00 | 45.00 | 45.00 | 6.36 | 12.65 | 2.20 |
| control | 45.00 | 45.00 | 45.00 | 9.19 | 6.06 | 2.47 |
| control | 38.74 | 45.00 | 45.00 | 7.97 | 4.67 | 0.15 |
| control | 45.00 | 45.00 | 45.00 | 14.72 | 0.61 | 0.65 |
| control | 45.00 | 45.00 | 45.00 | 3.28 | 5.20 | 1.49 |
| control | 45.00 | 45.00 | 45.00 | 0.00 | 21.05 | 2.42 |
| control | 45.00 | 45.00 | 38.21 | 2.73 | 3.54 | 6.67 |
| control | 45.00 | 40.55 | 45.00 | 10.53 | 0.46 | 5.67 |
| control | 45.00 | 38.41 | 45.00 | 20.71 | 2.67 | 4.17 |
| control | 45.00 | 45.00 | 44.12 | 16.35 | 0.00 | 1.14 |
| control | 45.00 | 45.00 | 45.00 | 7.14 | 32.75 | 2.68 |
| control | 45.00 | 45.00 | 45.00 | 7.61 | 2.46 | 1.70 |
| control | 45.00 | 45.00 | 37.36 | 12.10 | 21.35 | 1.40 |
| control | 45.00 | 45.00 | 45.00 | 17.17 | 14.75 | 2.96 |
| control | 45.00 | 45.00 | 45.00 | 24.54 | 5.50 | 5.05 |
| control | 39.00 | 39.47 | 45.00 | 0.00 | 0.00 | 0.13 |
| control | 45.00 | 45.00 | 45.00 | 5.31 | 0.70 | 3.68 |
| control | 45.00 | 45.00 | 45.00 | 3.33 | 4.55 | 0.95 |
| control | 45.00 | 45.00 | 45.00 | 11.94 | 2.15 | 1.28 |
| control | 45.00 | 45.00 | 45.00 | 9.64 | 0.00 | 9.77 |
| control | 45.00 | 45.00 | 45.00 | 1.52 | 7.45 | 0.87 |
| control | 45.00 | 45.00 | 45.00 | 0.88 | 6.28 | 0.33 |
| control | 45.00 | 45.00 | 45.00 | 3.53 | 0.00 | 2.39 |
| control | 45.00 | 45.00 | 45.00 | 0.88 | 3.72 | 2.98 |
| control | 45.00 | 45.00 | 45.00 | 0.00 | 17.25 | 1.69 |
| control | 37.88 | 38.40 | 45.00 | 6.34 | 3.25 | 5.06 |
| control | 45.00 | 40.32 | 45.00 | 2.20 | 3.40 | 0.31 |
| control | 45.00 | 45.00 | 45.00 | 7.01 | 4.70 | 4.92 |
| control | 45.00 | 45.00 | 45.00 | 2.57 | 0.00 | 0.53 |
| control | 45.00 | 38.54 | 38.57 | 13.12 | 6.35 | 2.15 |
| control | 45.00 | 45.00 | 45.00 | 6.39 | 9.00 | 0.54 |
| control | 45.00 | 45.00 | 45.00 | 1.75 | 0.00 | 3.98 |
| control | 45.00 | 45.00 | 39.88 | 2.30 | 11.85 | 4.99 |
| control | 45.00 | 45.00 | 45.00 | 0.41 | 7.92 | 3.87 |
| control | 45.00 | 45.00 | 45.00 | 2.25 | 4.29 | 0.31 |
| control | 45.00 | 45.00 | 45.00 | 0.00 | 7.29 | 0.44 |
| control | 45.00 | 45.00 | 45.00 | 8.45 | 18.38 | 8.98 |
| control | 45.00 | 45.00 | 45.00 | 2.22 | 10.25 | 0.78 |
| control | 45.00 | 45.00 | 45.00 | 0.00 | 3.79 | 0.83 |
| control | 45.00 | 45.00 | 45.00 | 13.89 | 1.96 | 5.99 |
| control | 45.00 | 45.00 | 45.00 | 4.06 | 11.87 | 2.35 |
| control | 45.00 | 45.00 | 45.00 | 8.77 | 7.95 | 4.32 |
| control | 45.00 | 45.00 | 45.00 | 11.54 | 2.85 | 2.29 |
| control | 45.00 | 45.00 | 45.00 | 14.23 | 3.65 | 3.09 |
| control | 45.00 | 45.00 | 45.00 | 10.58 | 0.00 | 5.93 |
| control | 45.00 | 45.00 | 45.00 | 15.13 | 1.96 | 2.71 |
| control | 45.00 | 45.00 | 45.00 | 5.89 | 13.90 | 1.61 |
| control | 45.00 | 45.00 | 45.00 | 0.72 | 1.94 | 3.34 |
| control | 45.00 | 45.00 | 45.00 | 18.26 | 2.95 | 1.92 |
| control | 45.00 | 45.00 | 45.00 | 5.54 | 0.00 | 2.38 |
| control | 45.00 | 45.00 | 45.00 | 4.77 | 2.71 | 4.04 |
| control | 45.00 | 45.00 | 45.00 | 2.71 | 11.29 | 5.63 |
| control | 45.00 | 45.00 | 45.00 | 2.98 | 15.25 | 3.29 |
| control | 45.00 | 45.00 | 45.00 | 6.98 | 10.71 | 2.90 |
| control | 45.00 | 45.00 | 45.00 | 11.14 | 9.17 | 3.72 |
| control | 45.00 | 45.00 | 45.00 | 0.38 | 0.00 | 6.24 |
| control | 45.00 | 41.73 | 45.00 | 0.00 | 6.54 | 2.09 |
| control | 45.00 | 45.00 | 45.00 | 0.72 | 0.00 | 1.01 |
| control | 34.89 | 33.82 | 45.00 | 7.72 | 0.00 | 1.22 |
| control | 45.00 | 45.00 | 45.00 | 0.00 | 3.94 | 3.30 |
| control | 45.00 | 45.00 | 45.00 | 3.18 | 3.54 | 1.32 |
| control | 38.52 | 37.58 | 45.00 | 11.32 | 0.11 | 4.66 |
| control | 37.87 | 36.34 | 45.00 | 1.83 | 14.85 | 3.21 |
| control | 45.00 | 45.00 | 45.00 | 11.74 | 0.00 | 3.02 |
| control | 45.00 | 45.00 | 45.00 | 13.86 | 10.95 | 4.55 |
| control | 45.00 | 45.00 | 43.98 | 0.00 | 2.17 | 6.96 |
| control | 38.24 | 45.00 | 45.00 | 10.80 | 0.00 | 1.47 |
| control | 45.00 | 45.00 | 45.00 | 17.17 | 25.95 | 1.14 |
| control | 45.00 | 45.00 | 45.00 | 0.93 | 0.00 | 2.33 |
| control | 45.00 | 45.00 | 45.00 | 1.00 | 0.00 | 1.50 |
| control | 45.00 | 45.00 | 45.00 | 4.90 | 1.65 | 2.63 |
| control | 39.53 | 45.00 | 45.00 | 1.97 | 9.21 | 0.80 |
| control | 45.00 | 45.00 | 45.00 | 13.77 | 22.65 | 3.44 |
| control | 45.00 | 45.00 | 45.00 | 2.54 | 7.05 | 4.17 |
| control | 45.00 | 36.75 | 45.00 | 11.75 | 2.10 | 4.63 |
| control | 45.00 | 45.00 | 41.88 | 0.00 | 7.80 | 1.92 |
| control | 38.27 | 45.00 | 45.00 | 2.79 | 15.90 | 0.58 |
| control | 45.00 | 45.00 | 45.00 | 3.91 | 1.80 | 1.55 |
| control | 45.00 | 45.00 | 45.00 | 2.78 | 7.45 | 2.54 |
| control | 45.00 | 45.00 | 45.00 | 29.43 | 5.20 | 3.66 |
| control | 45.00 | 45.00 | 45.00 | 9.28 | 20.50 | 1.78 |
| control | 45.00 | 45.00 | 45.00 | 24.55 | 16.90 | 3.45 |
| control | 45.00 | 45.00 | 45.00 | 2.73 | 3.35 | 2.12 |
| control | 45.00 | 45.00 | 45.00 | 5.81 | 1.45 | 4.08 |
| control | 45.00 | 45.00 | 45.00 | 11.23 | 1.45 | 6.97 |
| control | 45.00 | 45.00 | 45.00 | 0.12 | 20.25 | 2.67 |
| control | 45.00 | 45.00 | 40.11 | 6.77 | 3.55 | 2.11 |
| control | 45.00 | 45.00 | 45.00 | 9.03 | 2.85 | 2.73 |
| control | 45.00 | 37.89 | 45.00 | 1.12 | 0.00 | 2.08 |
| control | 45.00 | 45.00 | 45.00 | 7.50 | 3.39 | 0.18 |
| control | 45.00 | 45.00 | 45.00 | 2.79 | 9.20 | 0.00 |
| control | 42.42 | 38.08 | 38.89 | 0.00 | 0.00 | 0.63 |

TABLE 6
Performance for single markers (row 1 to 6) and of different marker
combinations by logistic regression analysis (row 7 to 10), characterized
by Sensitivity at Specificity of 0.9 and by area under the curve
(AUC) of receiver operating characteristic (ROC) analysis.

| | Sensitivity | Specificity | AUC |
|---|---|---|---|
| ANB1 | 0.619 | 0.9 | 0.777 |
| ANB2 | 0.557 | 0.9 | 0.753 |
| S9B2 | 0.629 | 0.9 | 0.786 |
| CEA | 0.529 | 0.9 | 0.774 |
| AR | 0.257 | 0.9 | 0.746 |
| CYPHRA | 0.129 | 0.9 | 0.583 |
| ANB1 + ANB2 + S9B2 | 0.671 | 0.9 | 0.841 |
| ANB1 + ANB2 + S9B2 + CEA | 0.757 | 0.9 | 0.887 |
| ANB1 + ANB2 + S9B2 + AR | 0.714 | 0.9 | 0.875 |
| ANB1 + ANB2 + S9B2 + CEA + AR | 0.771 | 0.9 | 0.897 |

TABLE 7
Performance for combined markers ANB1 + ANB2 +
S9B2 + CEA + AR for comparison of performance of the
Example 1 data set from individuals in the age eligible for
screening (row 2) with altered settings with a restriction to
a younger age group (row 2), addition of data from individuals
with advanced adenomas in the control group (row 3) and excluding
stage IV CRC patients (row 4), characterized by Sensitivity
at Specificity of 0.9 and by area under the curve (AUC) of
receiver operating characteristic (ROC) analysis.

| | Sensi- | Speci- | |
|---|---|---|---|
| | tivity | ficity | AUC |
| Example 1 data set (see row 10 in Table 5) | 0.771 | 0.9 | 0.897 |
| Age subgroup: 45-75 (excluding 8 controls | 0.831 | 0.9 | 0.914 |
| and 11 CRC) | | | |
| Including 65 advanced adenomas | 0.757 | 0.9 | 0.892 |
| Excluding seven stage IV CRC | 0.762 | 0.9 | 0.897 |

### Example 2—Expanded Multi-Analyte Panel for Detecting Colorectal Cancer

To determine whether the specificity and sensitivity of an assay for detecting colorectal carcinoma and/or pre-cancerous lesions (e.g., advanced adenoma) in a subject could be improved, presence of protein biomarkers are assessed in combination with DNA methylation markers in the blood of subjects diagnosed with colorectal carcinoma or pre-cancerous lesions relative to expression in control subjects.

### Methods

Colorectal carcinoma (CRC) biomarkers are measured in aliquots of blood plasma collected from individuals with CRC or without CRC as controls. Two DNA methylation markers are measured in three assays in one combined multiplex Real-time PCR Four selected protein biomarkers are individually measured using ELISAs.

### DNA Methylation Markers

DNA methylation markers are assessed as described in Example 1.

For each sample, bisulfite-treated DNA are amplified in duplicate in 50 μl total volume Real-time PCR quadruplex containing three methylation sensitive (MSP) assays for one bisulfite specific and methylation unspecific control (e.g., ACTB) assay and the two DNA-methylation markers mSeptin9 and mANKRD13B, which is measured on two strands. The assays are measured using an Applied Biosystems™ Quant Studio™ 5 Dx Real-Time PCR Instrument, using 45 cycles, and interpreting basic results as Cts of Realtime-PCR amplification curves.

For numerical interpretation, data for un-amplified assays (no curve) are set to the maximum Ct of 45.

### Hantoglobin

Serum or plasma specimens are thawed and samples are collected and diluted with water. NH₂SO₄ is added and mixed with the diluted samples. Samples are heated at 80° C. and then cooled on ice. PBS, NaOH, and water are added and mixed with the samples and the pH of the samples are checked and recorded. The resulting diluted desialylated serum samples are frozen and stored or used for ELISA.

Microtiter plates are marked for serum specimens, PBST blanks, and normal asialohaptoglobin standards. An anti-haptoglobin antibody is diluted into PBS and added to each well of the microtiter plates. The plate is left overnight at 4° C. Anti-haptoglobin is decanted and discarded and each well is washed with PBS. 1% BSA in PBS (prepared fresh daily) is added to each well and incubated at room temp before the BSA/PBS is decanted and discarded. Appropriate dilutions, standards and controls are prepared. Desialyated serum is diluted in PBST. The plate is incubated at room temp. Each well is washed once with PBST.

Biotinyl Erythrina cristagalli lectin in PBST is added to each well. The plate is incubated at room temp and each well is washed with PBST. A VECTASTAIN ABC Reagent Kit (Vector Elite PK-6100) is used for detection as described by the manufacturer's protocol. Avidin-Biotin Complex is added and the plate is incubated at room temp. Each well is washed with PBST. ABTS reagent (0.03% H₂O₂, 1 mM 2,2-azino-di(3-ethylbenzthiazoline) sulfonate in 0.1 M citrate, pH 4.0) is added. The plate is incubated at room temp and read at A405 using an ELISA reader.

### Amphiregulin (AR)

Concentrations [pg/mL] of the protein Amphiregulin in blood plasma samples are assessed using the Human Amphiregulin Quantikine ELISA Kit from R&D Systems according to manufacturer's protocol and as described in Example 1.

### CYFRA

Concentrations [ng/mL] of the protein Human Cytokeratin Fragment Antigen 21-1 (CYFRA21-1) in blood plasma samples are assessed using the Human Cytokeratin Fragment Antigen 21-1 (CYFRA21-1) ELISA kit from Cusabio according to manufacturer's protocol and as described in Example 1.

### CEA

Concentrations [ng/mL] of the protein carcinoembryonic antigen (CEA) in blood plasma samples are assessed using the Human CEA ELISA Kit (PN:EHCEA) from ThermoFisher according to manufacturer's protocol and as described in Example 1.

### Analysis

Protein biomarkers and DNA methylation markers are assessed alone and in combination. Sensitivity and specificity are determined as described in Example 1.

### Example 3—Detection of Haptoglobin Improves Sensitivity of Multi-Analyte Panel for Detecting Colorectal Cancer

The presence of protein biomarkers was assessed in combination with DNA methylation markers in the blood of subjects diagnosed with colorectal carcinoma relative to expression in control subjects.

### Methods

For each sample from Example 1, additional blood plasma aliquots were measured for detection of haptoglobin using an ELISA. The data was analyzed according to methods of Example 1 but included the measurement of haptoglobin. DNA methylation markers, CEA and amphiregulin were assessed as described in Example 1. Haptoglobin was detected as described below.

For each individual two 400 μL of 1:80 dilutions of blood plasma in water were each mixed with 100 μL of 0.5 N NH₂SO₄. Samples were heated at 80° C. and then cooled to and stored at 4° C. for 5 minutes. Each sample was mixed with 100 μL PBS, 100 μL 0.5 N NaOH, and 300 μL of water, leading to 1 mL aliquots containing the plasma in a 1:200 dilution. The pH of the samples was checked to confirm a valid pH between 6.5 and 7.5, dropout samples were replaced by new preparations.

Microtiter plates were marked for plasma specimens, PBST blanks, and normal asialohaptoglobin standards. Microtiter plates were coated by addition of 50 μl of 40 μg/mL Rabbit anti-Haptoglobin antibody in 1×PBS and overnight storage at 4° C. The antibody solution was discarded and each well was washed with 300 μl PBS. 200 μL of 1% BSA in 1×PBS was put to each well and incubated at room temp for one hour before the BSA/PBS was decanted and discarded.

Quantification standards (Asialohaptoglobin in a 0.5 dilution series with 6 steps from 1 mg/mL down to 15.6 ng/mL) were prepared. 20 μL of desialyated plasma per sample was diluted 1:100 in PBST using two 1:10 dilution steps, by first mixing with 180 μL PBST and then using 50 μL of that mix with 450 μL PBST, leading to a final 20,000 dilution of the blood plasma.

Blocking solution was removed from the microtiter plates by discarding but without washing. The wells were filled with 50 μl/well by standards, diluted samples, and blanks in duplicate and the plate was sealed and incubated at 25° C. for one hour. The plate was incubated at room temp. Each well was washed once with 300 μl PBST.

50 μl biotinyl Erythrina cristagalli lectin in 1×PBST was added to each well and the plate was sealed and incubated at 25° C. for one hour. The solution was removed and each well was washed with 300 μl PBST three times.

A VECTASTAIN ABC Reagent Kit (Vector Elite PK-6100) was used for detection as described by the manufacturer's protocol. 50 μl of Avidin-Biotin-HRT Complex was added per well and the plate was sealed and incubated 25° C. for one hour. Each well was washed with 300 μL PBST three times. 100 μL of ABTS reagent (0.03% H₂O₂, 1 mM 2,2-azino-di(3-ethylbenzthiazoline) sulfonate in 0.1 M citrate, pH 4.0) was added. The plate was incubated at 25° C. for exactly 30 minutes, shaken on an ELISA reader for 5 seconds and then read at A405.

Median blank values were subtracted from A405 values for all samples, controls, and standards. Based on the dilution series, a linear function for calibration and quantification was calculated by linear regression for each plate to obtain ng/mL. To obtain final haptoglobin concentrations, results were multiplied by 20,000 and values were expressed as mg/mL.

### Results

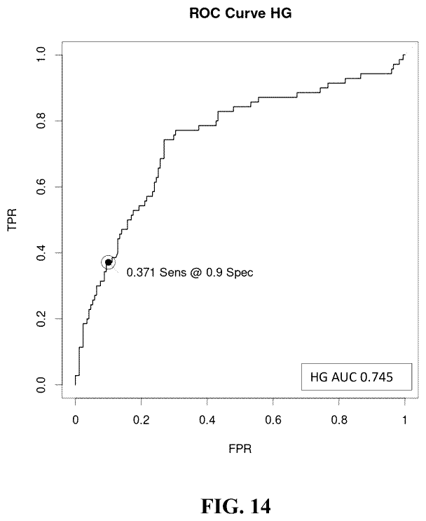

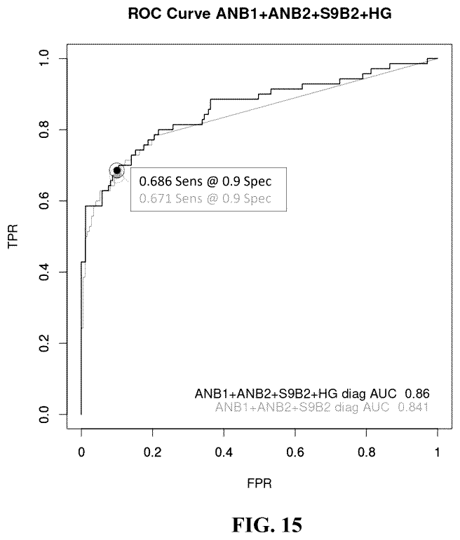

The individual protein measurement of Haptoglobin lead to an AUC of 0.37 (see FIG. 14). Protein measurements are provided in Table 8. The combination of DNA methylation markers with Haptoglobin lead to an AUC of 0.86 and Sensitivity of 0.69 at Specificity of 0.9, increasing both in comparison to methylation marker combinations without Haptoglobin (see FIG. 15).

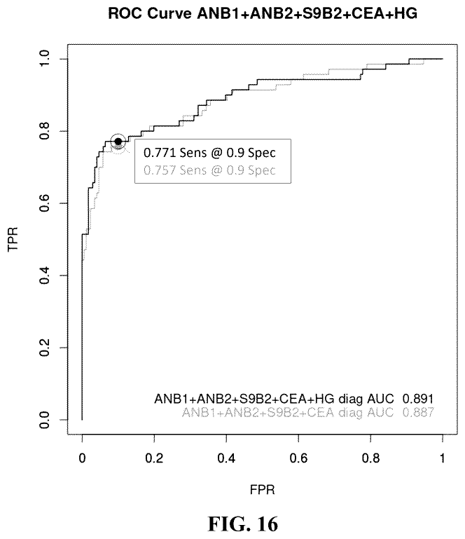

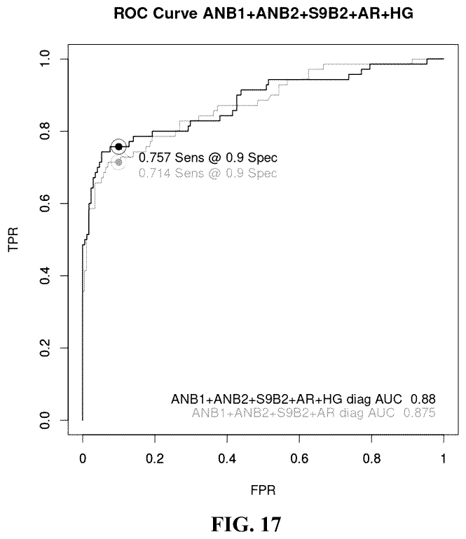

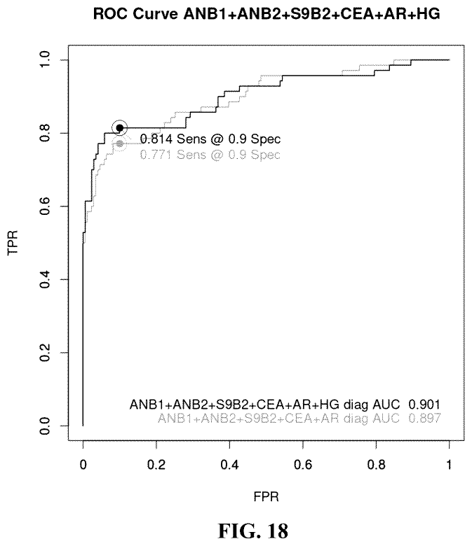

Combinations of data from the three DNA-methylation biomarker assays with protein data from either or both of the two proteins CEA and Amphiregulin, as already described in Example 1, with data from the additional protein haptoglobin lead to higher Sensitivities at a Specificity of 0.9 and higher AUC than obtained with the corresponding marker combinations without haptoglobin (see FIGS. 15-18 and Table 9) with a maximum Sensitivity of 0.81 for the combination of DNA methylation markers with CEA, Amphiregulin and Haptoglobin.

Overall, these data indicate that including Haptoglobin into the combined detection of protein biomarkers and methylation of genomic DNA further improves the Sensitivity for detecting subjects with CRC.

TABLE 8
Measurements for Haptoglobin in all 241
individuals as obtained in Example 3.

| | HG | | HG | | HG |
|---|---|---|---|---|---|
| Diagnosis | (pg/mL) | Diagnosis | (pg/mL) | Diagnosis | (pg/mL) |
| crc | 5.62 | control | 4.99 | control | 3.55 |
| crc | 5.64 | control | 6.00 | control | 4.44 |
| crc | 1.08 | control | 5.25 | control | 5.17 |
| crc | 5.30 | control | 3.71 | control | 4.81 |
| crc | 6.84 | control | 7.35 | control | 4.28 |
| crc | 7.48 | control | 6.89 | control | 6.15 |
| crc | 8.67 | control | 8.68 | control | 4.65 |
| crc | 6.22 | control | 3.27 | control | 6.00 |
| crc | 8.22 | control | 1.60 | control | 8.02 |
| crc | 5.50 | control | 5.57 | control | 3.71 |
| crc | 8.17 | control | 3.74 | control | 3.90 |
| crc | 5.53 | control | 5.15 | control | 2.44 |
| crc | 9.83 | control | 5.25 | control | 2.88 |
| crc | 3.99 | control | 5.24 | control | 6.86 |
| crc | 4.91 | control | 6.75 | control | 3.17 |
| crc | 8.09 | control | 7.12 | control | 10.55 |
| crc | 4.71 | control | 1.73 | control | 4.76 |
| crc | 6.54 | control | 4.42 | control | 3.60 |
| crc | 5.26 | control | 6.01 | control | 4.69 |
| crc | 3.60 | control | 4.26 | control | 5.08 |
| crc | 5.77 | control | 5.63 | control | 3.24 |
| crc | 5.55 | control | 3.13 | control | 4.70 |
| crc | 4.48 | control | 2.05 | control | 3.60 |
| crc | 6.07 | control | 7.77 | control | 4.69 |
| crc | 6.65 | control | 5.97 | control | 2.57 |
| crc | 6.29 | control | 10.67 | control | 6.94 |
| crc | 6.74 | control | 1.87 | control | 5.48 |
| crc | 7.33 | control | 6.25 | control | 4.62 |
| crc | 5.62 | control | 7.94 | control | 3.08 |
| crc | 6.99 | control | 4.15 | control | 5.66 |
| crc | 6.94 | control | 7.38 | control | 6.54 |
| crc | 7.93 | control | 4.95 | control | 3.03 |
| crc | 6.01 | control | 3.90 | control | 8.70 |
| crc | 5.77 | control | 5.48 | control | 5.97 |
| crc | 6.00 | control | 4.76 | control | 3.55 |
| crc | 5.58 | control | 3.37 | control | 3.33 |
| crc | 6.71 | control | 2.85 | control | 5.23 |
| crc | 10.15 | control | 3.52 | control | 5.91 |
| crc | 3.18 | control | 3.39 | control | 5.04 |
| crc | 4.63 | control | 3.16 | control | 3.93 |
| crc | 5.90 | control | 4.46 | control | 3.70 |
| crc | 8.84 | control | 3.29 | control | 3.21 |
| crc | 1.47 | control | 5.36 | control | 1.17 |
| crc | 7.61 | control | 4.24 | control | 4.36 |
| crc | 1.85 | control | 5.84 | control | 7.10 |
| crc | 7.16 | control | 2.55 | control | 2.70 |
| crc | 9.27 | control | 4.27 | control | 4.50 |
| crc | 4.99 | control | 5.16 | control | 3.65 |
| crc | 5.13 | control | 5.78 | control | 4.04 |
| crc | 7.69 | control | 4.46 | control | 5.31 |
| crc | 5.93 | control | 4.80 | control | 5.05 |
| crc | 6.92 | control | 2.98 | control | 5.14 |
| crc | 9.74 | control | 6.74 | control | 4.67 |
| crc | 5.83 | control | 6.77 | control | 7.44 |
| crc | 6.64 | control | 4.00 | control | 5.86 |
| crc | 1.90 | control | 6.46 | control | 4.51 |
| crc | 2.99 | control | 2.47 | control | 4.51 |
| crc | 7.38 | control | 6.05 | control | 2.08 |
| crc | 7.35 | control | 4.18 | control | 4.72 |
| crc | 5.59 | control | 5.30 | control | 3.16 |
| crc | 4.92 | control | 3.15 | control | 7.32 |
| crc | 15.05 | control | 3.79 | control | 5.11 |
| crc | 7.34 | control | 7.59 | control | 5.07 |
| crc | 5.50 | control | 4.06 | control | 4.43 |
| crc | 6.24 | control | 5.20 | control | 2.48 |
| crc | 3.53 | control | 3.22 | control | 6.26 |
| crc | 7.00 | control | 2.91 | control | 5.60 |
| crc | 10.81 | control | 2.99 | control | 3.88 |
| crc | 8.08 | control | 4.66 | control | 6.28 |
| crc | 9.23 | control | 5.56 | control | 4.87 |
| control | 5.85 | control | 4.83 | control | 5.22 |
| control | 0.83 | control | 5.09 | control | 4.67 |
| control | 4.11 | control | 2.07 | control | 4.00 |
| control | 1.60 | control | 3.92 | control | 5.19 |
| control | 5.94 | control | 4.01 | control | 3.83 |
| control | 6.14 | control | 5.07 | control | 7.19 |
| control | 3.14 | control | 4.09 | control | 2.73 |
| control | 4.07 | control | 4.99 | control | 4.91 |
| control | 6.88 | control | 5.19 | control | 5.46 |
| control | 4.70 | control | 1.09 | control | 4.69 |
| | | | | control | 2.39 |

TABLE 9
Performance summary and comparison for the single marker Haptoglobin
from Example 3 (row 1) and of different marker combinations from
Example 1 (row 2, 4, 6, 8) and Example 3 (row 3, 5, 7, 9) by logistic
regression analysis, characterized by Sensitivity (column 1) at
Specificity of 0.9 and by area under the curve (AUC) (column 3)
of receiver operating characteristic (ROC) analysis.

| | Sensitivity | Specificity | AUC |
|---|---|---|---|
| HG | 0.371 | 0.9 | 0.745 |
| ANB1 + ANB2 + S9B2 | 0.671 | 0.9 | 0.841 |
| ANB1 + ANB2 + S9B2 + HG | 0.686 | 0.9 | 0.860 |
| ANB1 + ANB2 + S9B2 + CEA | 0.757 | 0.9 | 0.887 |
| ANB1 + ANB2 + S9B2 + CEA + HG | 0.771 | 0.9 | 0.891 |
| ANB1 + ANB2 + S9B2 + AR | 0.714 | 0.9 | 0.875 |
| ANB1 + ANB2 + S9B2 + AR + HG | 0.757 | 0.9 | 0.880 |
| ANB1 + ANB2 + S9B2 + CEA + AR | 0.771 | 0.9 | 0.897 |
| ANB1 + ANB2 + S9B2 + CEA + | 0.814 | 0.9 | 0.901 |
| AR + HG | | | |

SEQUENCE LISTING

| SEQ ID NO | Description |
|---|---|
| 1 | ANKRD13BoB1 |
| | Assay genomic |
| | reference |
| 2 | ANKRD13BoB1 |
| | Assay converted C to |
| | T (bis1) |
| 3 | ANKRD13BoB1 |
| | Assay converted C to |
| | T (bis1) reverse |
| | complement |
| 4 | ANKRD13BoB1 |
| | Assay converted G to |
| | A (bis2 rc) |
| 5 | ANKRD13BoB1 |
| | Assay converted G to |
| | A (bis2 rc) reverse |
| | complement |
| 6 | ANKRD13B |
| | ExtendedAssayRegion |
| | A genomic reference |
| 7 | ANKRD13B |
| | ExtendedAssayRegion |
| | A converted C to T |
| | (bis1) |
| 8 | ANKRD13B |
| | ExtendedAssayRegion |
| | A converted C to T |
| | (bis1) reverse |
| | complement |
| 9 | ANKRD13B |
| | Extended AssayRegion |
| | A converted G to A |
| | (bis2 rc) |
| 10 | ANKRD13B |
| | ExtendedAssayRegion |
| | A converted G to A |
| | (bis2 rc) reverse |
| | complement |
| 11 | ANKRD13B |
| | ExtendedAssayRegion |
| | B genomic reference |
| 12 | ANKRD13B |
| | ExtendedAssayRegion |
| | B converted C to T |
| | (bis1) |
| 13 | ANKRD13B |
| | ExtendedAssayRegion |
| | B converted C to T |
| | (bis1) reverse |
| | complement |
| 14 | ANKRD13B |
| | ExtendedAssayRegion |
| | B converted G to A |
| | (bis2 rc) |
| 15 | ANKRD13B |
| | ExtendedAssayRegion |
| | B converted G to A |
| | (bis2 rc) reverse |
| | complement |
| 16 | ANKRD13BoB2 |
| | Assay genomic |
| | reference |
| 17 | ANKRD13BoB2 |
| | Assay converted C to |
| | T (bis1) |
| 18 | ANKRD13BoB2 |
| | Assay converted C to |
| | T (bis1) reverse |
| | complement |
| 19 | ANKRD13BoB2 |
| | Assay converted G to |
| | A (bis2 rc) |
| 20 | ANKRD13BoB2 |
| | Assay converted G to |
| | A (bis2 rc) reverse |
| | complement |
| 21 | SEPTIN9oB2 Assay |
| | genomic reference |
| 22 | SEPTIN9oB2 Assay |
| | converted C to T |
| | (bis1) |
| 23 | SEPTIN9oB2 Assay |
| | converted C to T |
| | (bis1) reverse |
| | complement |
| 24 | SEPTIN9oB2 Assay |
| | converted G to A (bis2 |
| | rc) |
| 25 | SEPTIN9oB2 Assay |
| | converted G to A (bis2 |
| | rc) reverse |
| | complement |
| 26 | SEPTIN9 |
| | Assay Region genomic |
| | reference |
| 27 | SEPTIN9 |
| | AssayRegion |
| | converted C to T |
| | (bis1) |
| 28 | SEPTIN9 |
| | AssayRegion |
| | converted C to T |
| | (bis1) reverse |
| | complement |
| 29 | SEPTIN9 |
| | AssayRegion |
| | converted G to A (bis2 |
| | rc) |
| 30 | SEPTIN9 |
| | AssayRegion |
| | converted G to A (bis2 |
| | rc) reverse |
| | complement |
| 31 | SEPTIN9 |
| | Assay + CpG-island |
| | genomic reference |
| 32 | SEPTIN9 |
| | Assay + CpG-island |
| | converted C to T |
| | (bis1) |
| 33 | SEPTIN9 |
| | Assay + CpG-island |
| | converted C to T |
| | (bis1) reverse |
| | complement |
| 34 | SEPTIN9 |
| | Assay + CpG-island |
| | converted G to A (bis2 |
| | rc) |
| 35 | SEPTIN9 |
| | Assay + CpG-island |
| | converted G to A (bis2 |
| | rc) reverse |
| | complement |
| 36 | SEPTIN9 |
| | ExtendedAssayRegion |
| | genomic reference |
| 37 | SEPTIN9 |
| | ExtendedAssayRegion |
| | converted C to T |
| | (bis1) |
| 38 | SEPTIN9 |
| | ExtendedAssayRegion |
| | converted C to T |
| | (bis1) reverse |
| | complement |
| 39 | SEPTIN9 |
| | ExtendedAssayRegion |
| | converted G to A (bis2 |
| | rc) |
| 40 | SEPTIN9 |
| | ExtendedAssayRegion |
| | converted G to A (bis2 |
| | rc) reverse |
| | complement |
| 41 | ANKRD13B |
| | ANKRD13BoB1-F4 |
| | CO6B1-F4 |
| 42 | ANKRD13B |
| | ANKRD13BoB1-R6 |
| | CO6B1-R6 |
| 43 | ANKRD13B |
| | ANKRD13BoB2-F3 |
| | CO6B2-F3 |
| 44 | ANKRD13B |
| | ANKRD13BoB2-R5 |
| | CO6B2-R5 |
| 45 | SEPTIN9 |
| | SEPTIN9oB2-F3 |
| | S9B2-F3 |
| 46 | SEPTIN9 |
| | SEPTIN9oB2-R11 |
| | S9B2-R11 |
| 47 | ANKRD13B |
| | ANKRD13BoB1-P4 |
| | CO6B1-P4 |
| 48 | ANKRD13B |
| | ANKRD13BoB2-P1 |
| | CO6B2-P1 |
| 49 | SEPTIN9 |
| | SEPTIN9oB2-P8 |
| | S9B2-P8 |
| 50 | Human |
| | carcinoembryonic |
| | antigen (CEA) |
| 51 | Human amphiregulin |
| 52 | Human CYFRA21-1 |
| 53 | Human haptoglobin |
| 54 | mADCYAP1 Assay + |
| | CpG Island genomic |
| | reference |
| 55 | mADCYAP1 Assay + |
| | CpG Island C to T |
| | (bis1) |
| 56 | mADCYAP1 Assay + |
| | CpG Island C to T |
| | (bis1) reverse |
| | complement |
| 57 | mADCYAP1 Assay + |
| | CpG Island G to A |
| | (bis2 rc) |
| 58 | mADCYAP1 Assay + |
| | CpG Island G to A |
| | (bis2 rc) reverse |
| | complement |
| 59 | mADCYAP1 |
| | Extended Assay |
| | genomic reference |
| 60 | mADCYAP1 |
| | Extended Assay C to |
| | T (bis1) |
| 61 | mADCYAP1 |
| | Extended Assay C to |
| | T (bis1) reverse |
| | complement |
| 62 | mADCYAP1 |
| | Extended Assay G to |
| | A (bis2 rc) |
| 63 | mADCYAP1 |
| | Extended Assay G to |
| | A (bis2 rc) reverse |
| | complement |
| 64 | mADCYAP1 Assay |
| | genomic reference |
| 65 | mADCYAP1 Assay C |
| | to T (bis1) |
| 66 | mADCYAP1 Assay C |
| | to T (bis1) reverse |
| | complement |
| 67 | mADCYAP1 Assay G |
| | to A (bis2 rc) |
| 68 | mADCYAP1 Assay G |
| | to A (bis2 rc) reverse |
| | complement |
| 69 | mKHDRBS2 |
| | Extended Assay |
| | genomic reference |
| 70 | mKHDRBS2 |
| | Extended Assay C to |
| | T (bis1) |
| 71 | mKHDRBS2 |
| | Extended Assay C to |
| | T (bis1) reverse |
| | complement |
| 72 | mKHDRBS2 |
| | Extended Assay G to |
| | A (bis2 rc) |
| 73 | mKHDRBS2 |
| | Extended Assay G to |
| | A (bis2 rc) reverse |
| | complement |
| 74 | mKHDRBS2 Assay |
| | genomic reference |
| 75 | mKHDRBS2 Assay C |
| | to T (bis1) |
| 76 | mKHDRBS2 Assay C |
| | to T (bis1) reverse |
| | complement |
| 77 | mKHDRBS2 |
| | mADCYAP1 Assay G |
| | to A (bis2 rc) |
| 78 | mKHDRBS2 Assay G |
| | to A (bis2 rc) reverse |
| | complement |
| 79 | mCLEC14A Assay + |
| | CpG Island genomic |
| | reference |
| 80 | mCLEC14A Assay + |
| | CpG Island C to T |
| | (bis1) |
| 81 | mCLEC14A Assay + |
| | CpG Island C to T |
| | (bis1) reverse |
| | complement |
| 82 | mCLEC14A Assay + |
| | CpG Island G to A |
| | (bis2 rc) |
| 83 | mCLEC14A Assay + |
| | CpG Island G to A |
| | (bis2 rc) reverse |
| | complement |
| 84 | mCLEC14A Extended |
| | Assay genomic |
| | reference |
| 85 | mCLEC14A Extended |
| | Assay C to T (bis1) |
| 86 | mCLEC14A Extended |
| | Assay C to T (bis1) |
| | reverse complement |
| 87 | mCLEC14A Extended |
| | Assay G to A (bis2 rc) |
| 88 | mCLEC14A Extended |
| | Assay G to A (bis2 rc) |
| | reverse complement |
| 89 | mCLEC14A Assay |
| | genomic reference |
| 90 | mCLEC14A Assay C |
| | to T (bis1) |
| 91 | mCLEC14A Assay C |
| | to T (bis1) reverse |
| | complement |
| 92 | mCLEC14A Assay G |
| | to A (bis2 rc) |
| 93 | mCLEC14A Assay G |
| | to A (bis2 rc) reverse |
| | complement |
| 94 | mCRMP1 Extended |
| | Assay genomic |
| | reference |
| 95 | mCRMP1 Extended |
| | Assay C to T (bis1) |
| 96 | mCRMP1 Extended |
| | Assay C to T (bis1) |
| | reverse complement |
| 97 | mCRMP1 Extended |
| | Assay G to A (bis2 rc) |
| 98 | mCRMP1 Extended |
| | Assay G to A (bis2 rc) |
| | reverse complement |
| 99 | mCRMP1 Assay |
| | genomic reference |
| 100 | mCRMP1 Assay Cto |
| | T (bis1) |
| 101 | mCRMP1 Assay C to |
| | T (bis1) reverse |
| | complement |
| 102 | mCRMP1 Assay G to |
| | A (bis2 rc) |
| 103 | mCRMP1 Assay G to |
| | A (bis2 rc) reverse |
| | complement |
| 104 | mEYA4 Assay + CpG |
| | Island genomic |
| | reference |
| 105 | mEYA4 Assay + CpG |
| | Island C to T (bis1) |
| 106 | mEYA4 Assay + CpG |
| | Island C to T (bis1) |
| | reverse complement |
| 107 | mEYA4 Assay + CpG |
| | Island G to A (bis2 rc) |
| 108 | mEYA4 Assay + CpG |
| | Island G to A (bis2 rc) |
| | reverse complement |
| 109 | mEYA4 Extended |
| | Assay genomic |
| | reference |
| 110 | mEYA4 Extended |
| | Assay C to T (bis1) |
| 111 | mEYA4 Extended |
| | Assay C to T (bis1) |
| | reverse complement |
| 112 | mEYA4 Extended |
| | Assay G to A (bis2 rc) |
| 113 | mEYA4 Extended |
| | Assay G to A (bis2 rc) |
| | reverse complement |
| 114 | mEYA4 Assay |
| | genomic reference |
| 115 | mEYA4 Assay C to T |
| | (bis1) |
| 116 | mEYA4 Assay C to T |
| | (bis1) reverse |
| | complement |
| 117 | mEYA4 Assay G to A |
| | (bis2 rc) |
| 118 | mEYA4 Assay G to A |
| | (bis2 rc) reverse |
| | complement |
| 119 | mMSC Assay + CpG |
| | Island genomic |
| | reference |
| 120 | mMSC Assay + CpG |
| | Island C to T (bis1) |
| 121 | mMSC Assay + CpG |
| | Island C to T (bis1) |
| | reverse complement |
| 122 | mMSC Assay + CpG |
| | Island G to A (bis2 rc) |
| 123 | mMSC Assay + CpG |
| | Island G to A (bis2 rc) |
| | reverse complement |
| 124 | mMSC Extended |
| | Assay genomic |
| | reference |
| 125 | mMSC Extended |
| | Assay C to T (bis1) |
| 126 | mMSC Extended |
| | Assay C to T (bis1) |
| | reverse complement |
| 127 | mMSC Extended |
| | Assay G to A (bis2 rc) |
| 128 | mMSC Extended |
| | Assay G to A (bis2 rc) |
| | reverse complement |
| 129 | mMSC Assay |
| | genomic reference |
| 130 | mMSC Assay C to T |
| | (bis1) |
| 131 | mMSC Assay C to T |
| | (bis1) reverse |
| | complement |
| 132 | mMSC Assay G to A |
| | (bis2 rc) |
| 133 | mMSC Assay G to A |
| | (bis2 rc) reverse |
| | complement |
| 134 | mNGFR Assay + CpG |
| | Island genomic |
| | reference |
| 135 | mNGFR Assay + CpG |
| | Island C to T (bis1) |
| 136 | mNGFR Assay + CpG |
| | Island C to T (bis1) |
| | reverse complement |
| 137 | mNGFR Assay + CpG |
| | Island G to A (bis2 rc) |
| 138 | mNGFR Assay + CpG |
| | Island G to A (bis2 rc) |
| | reverse complement |
| 139 | mNGFR Extended |
| | Assay genomic |
| | reference |
| 140 | mNGFR Extended |
| | Assay C to T (bis1) |
| 141 | mNGFR Extended |
| | Assay C to T (bis1) |
| | reverse complement |
| 142 | mNGFR Extended |
| | Assay G to A (bis2 rc) |
| 143 | mNGFR Extended |
| | Assay G to A (bis2 rc) |
| | reverse complement |
| 144 | mNGFR Assay |
| | genomic reference |
| 145 | mNGFR Assay C to T |
| | (bis1) |
| 146 | mNGFR Assay C to T |
| | (bis1) reverse |
| | complement |
| 147 | mNGFR Assay G to A |
| | (bis2 rc) |
| 148 | mNGFR Assay G to A |
| | (bis2 rc) reverse |
| | complement |
| 149 | mNKX2 Assay + CpG |
| | Island genomic |
| | reference |
| 150 | mNKX2 Assay + CpG |
| | Island C to T (bis1) |
| 151 | mNKX2 Assay + CpG |
| | Island C to T (bis1) |
| | reverse complement |
| 152 | mNKX2 Assay + CpG |
| | Island G to A (bis2 rc) |
| 153 | mNKX2 Assay + CpG |
| | Island G to A (bis2 rc) |
| | reverse complement |
| 154 | mNKX2 Extended |
| | Assay genomic |
| | reference |
| 155 | mNKX2 Extended |
| | Assay C to T (bis1) |
| 156 | mNKX2Extended |
| | Assay C to T (bis1) |
| | reverse complement |
| 157 | mNKX2 Extended |
| | Assay G to A (bis2 rc) |
| 158 | mNKX2 Extended |
| | Assay G to A (bis2 rc) |
| | reverse complement |
| 159 | mNKX2 Assay |
| | genomic reference |
| 160 | mNKX2 Assay C to T |
| | (bis1) |
| 161 | mNKX2 Assay C to T |
| | (bis1) reverse |
| | complement |
| 162 | mNKX2 Assay G to A |
| | (bis2 rc) |
| 163 | mNKX2 Assay G to A |
| | (bis2 rc) reverse |
| | complement |
| 164 | mRASSF2 Assay + |
| | CpG Island genomic |
| | reference |
| 165 | mRASSF2 Assay + |
| | CpG Island C to T |
| | (bis1) |
| 166 | mRASSF2 Assay + |
| | CpG Island C to T |
| | (bis1) reverse |
| | complement |
| 167 | mRASSF2 Assay + |
| | CpG Island G to A |
| | (bis2 rc) |
| 168 | mRASSF2 Assay + |
| | CpG Island G to A |
| | (bis2 rc) reverse |
| | complement |
| 169 | mRASSF2 Extended |
| | Assay genomic |
| | reference |
| 170 | mRASSF2 Extended |
| | Assay C to T (bis1) |
| 171 | mRASSF2 Extended |
| | Assay C to T (bis1) |
| | reverse complement |
| 172 | mRASSF2 Extended |
| | Assay G to A (bis2 rc) |
| 173 | mRASSF2 Extended |
| | Assay G to A (bis2 rc) |
| | reverse complement |
| 174 | mRASSF2 Assay |
| | genomic reference |
| 175 | mRASSF2 Assay C to |
| | T (bis1) |
| 176 | mRASSF2 Assay C to |
| | T (bis1) reverse |
| | complement |
| 177 | mRASSF2 Assay G to |
| | A (bis2 rc) |
| 178 | mRASSF2 Assay G to |
| | A (bis2 rc) reverse |
| | complement |
| 179 | mSND1 Assay + CpG |
| | Island genomic |
| | reference |
| 180 | mSND1 Assay + CpG |
| | Island C to T (bis1) |
| 181 | mSND1 Assay + CpG |
| | Island C to T (bis1) |
| | reverse complement |
| 182 | mSND1 Assay + CpG |
| | Island G to A (bis2 rc) |
| 183 | mSND1 Assay + CpG |
| | Island G to A (bis2 rc) |
| | reverse complement |
| 184 | mSND1 Extended |
| | Assay genomic |
| | reference |
| 185 | mSND1 Extended |
| | Assay C to T (bis1) |
| 186 | mSND1 Extended |
| | Assay C to T (bis1) |
| | reverse complement |
| 187 | mSND1 Extended |
| | Assay G to A (bis2 rc) |
| 188 | mSND1 Extended |
| | Assay G to A (bis2 rc) |
| | reverse complement |
| 189 | mSND1 Assay |
| | genomic reference |
| 190 | mSND1 Assay C to T |
| | (bis1) |
| 191 | mSND1 Assay C to T |
| | (bis1) reverse |
| | complement |
| 192 | mSND1 Assay G to A |
| | (bis2 rc) |
| 193 | mSND1 Assay G to A |
| | (bis2 rc) reverse |
| | complement |
| 194 | mTBX18 Assay + |
| | CpG Island genomic |
| | reference |
| 195 | mTBX18 Assay + |
| | CpG Island C to T |
| | (bis1) |
| 196 | mTBX18 Assay + |
| | CpG Island C to T |
| | (bis1) reverse |
| | complement |
| 197 | mTBX18 Assay + |
| | CpG Island G to A |
| | (bis2 rc) |
| 198 | mTBX18 Assay + |
| | CpG Island G to A |
| | (bis2 rc) reverse |
| | complement |
| 199 | mTBX18 Extended |
| | Assay genomic |
| | reference |
| 200 | mTBX18 Extended |
| | Assay C to T (bis1) |
| 201 | mTBX18 Extended |
| | Assay C to T (bis1) |
| | reverse complement |
| 202 | mTBX18 Extended |
| | Assay G to A (bis2 rc) |
| 203 | mTBX18 Extended |
| | Assay G to A (bis2 rc) |
| | reverse complement |
| 204 | mTBX18 Assay |
| | genomic reference |
| 205 | mTBX18 Assay C to |
| | T (bis1) |
| 206 | mTBX18 Assay C to |
| | T (bis1) reverse |
| | complement |
| 207 | mTBX18 Assay G to |
| | A (bis2 rc) |
| 208 | mTBX18 Assay G to |
| | A (bis2 rc) reverse |
| | complement |
| 209 | mTFAP2E Assay + |
| | CpG Island genomic |
| | reference |
| 210 | mTFAP2E Assay + |
| | CpG Island C to T |
| | (bis1) |
| 211 | mTFAP2E Assay + |
| | CpG Island C to T |
| | (bis1) reverse |
| | complement |
| 212 | mTFAP2E Assay + |
| | CpG Island G to A |
| | (bis2 rc) |
| 213 | mTFAP2E Assay + |
| | CpG Island G to A |
| | (bis2 rc) reverse |
| | complement |
| 214 | mTFAP2E Extended |
| | Assay genomic |
| | reference |
| 215 | mTFAP2E Extended |
| | Assay C to T (bis1) |
| 216 | mTFAP2E Extended |
| | Assay C to T (bis1) |
| | reverse complement |
| 217 | mTFAP2E Extended |
| | Assay G to A (bis2 rc) |
| 218 | mTFAP2E Extended |
| | Assay G to A (bis2 rc) |
| | reverse complement |
| 219 | mTFAP2E Assay |
| | genomic reference |
| 220 | mTFAP2E Assay C to |
| | T (bis1) |
| 221 | mTFAP2E Assay C to |
| | T (bis1) reverse |
| | complement |
| 222 | mTFAP2E Assay G to |
| | A (bis2 rc) |
| 223 | mTFAP2E Assay G to |
| | A (bis2 rc) reverse |
| | complement |
| 224 | mTMEFF2 Assay + |
| | CpG Island genomic |
| | reference |
| 225 | mTMEFF2 Assay + |
| | CpG Island C to T |
| | (bis1) |
| 226 | mTMEFF2 Assay + |
| | CpG Island C to T |
| | (bis1) reverse |
| | complement |
| 227 | mTMEFF2 Assay + |
| | CpG Island G to A |
| | (bis2 rc) |
| 228 | mTMEFF2 Assay + |
| | CpG Island G to A |
| | (bis2 rc) reverse |
| | complement |
| 229 | mTMEFF2 Extended |
| | Assay genomic |
| | reference |
| 230 | mTMEFF2 Extended |
| | Assay C to T (bis1) |
| 231 | mTMEFF2 Extended |
| | Assay C to T (bis1) |
| | reverse complement |
| 232 | mTMEFF2 Extended |
| | Assay G to A (bis2 rc) |
| 233 | mTMEFF2 Extended |
| | Assay G to A (bis2 rc) |
| | reverse complement |
| 234 | mTMEFF2 Assay |
| | genomic reference |
| 235 | mTMEFF2 Assay C to |
| | T (bis1) |
| 236 | mTMEFF2 Assay C to |
| | T (bis1) reverse |
| | complement |
| 237 | mTMEFF2 Assay G to |
| | A (bis2 rc) |
| 238 | mTMEFF2 Assay G to |
| | A (bis2 rc) reverse |
| | complement |
| 239 | mVAX1 Extended |
| | Assay genomic |
| | reference |
| 240 | mVAX1 Extended |
| | Assay C to T (bis1) |
| 241 | mVAX1 Extended |
| | Assay C to T (bis1) |
| | reverse complement |
| 242 | mVAX1 Extended |
| | Assay G to A (bis2 rc) |
| 243 | mVAX1 Extended |
| | Assay G to A (bis2 rc) |
| | reverse complement |
| 244 | mVAX1 Assay |
| | genomic reference |
| 245 | mVAX1 Assay C to T |
| | (bis1) |
| 246 | mVAX1 Assay C to T |
| | (bis1) reverse |
| | complement |
| 247 | mVAX1 Assay G to A |
| | (bis2 rc) |
| 248 | mVAX1 Assay G to A |
| | (bis2 rc) reverse |
| | complement |
| 249 | mADCYAP1 forward |
| | primer |
| 250 | mADCYAP1 reverse |
| | primer |
| 251 | mKHDRBS2 forward |
| | primer |
| 252 | mKHDRBS2 reverse |
| | primer |
| 253 | mCLEC14 forward |
| | primer |
| 254 | mCLEC14 reverse |
| | primer |
| 255 | mCRMP1 forward |
| | primer |
| 256 | mCRMP1 reverse |
| | primer |
| 257 | mEYA4 forward |
| | primer |
| 258 | mEYA4 reverse |
| | primer |
| 259 | mMSC forward primer |
| 260 | mMSC reverse primer |
| 261 | mNGFR forward |
| | primer |
| 262 | mNGFR reverse |
| | primer |
| 263 | mNKX2 forward |
| | primer |
| 264 | mNKX2 reverse |
| | primer |
| 265 | mRASSF2 forward |
| | primer |
| 266 | mRASSF2 reverse |
| | primer |
| 267 | mSND1 forward |
| | primer |
| 268 | mSND1 reverse |
| | primer |
| 269 | mTBX18 forward |
| | primer |
| 270 | mTBX18 reverse |
| | primer |
| 271 | mTFAP2E forward |
| | primer |
| 272 | mTFAP2E forward |
| | primer |
| 273 | mTMEFF2 forward |
| | primer |
| 274 | mTMEFF2 forward |
| | primer |
| 275 | mVAX1 forward |
| | primer |
| 276 | mVAX1 forward |
| | primer |

1. A method for detecting the presence or amount of methylated genomic DNA and protein in one or more biological samples from a subject, comprising:
(i) detecting synthetic DNA generated from one or more methylated genomic DNA sequences associated with a proliferative disease from the one or more biological samples, wherein the synthetic DNA is generated by converting cytosine unmethylated in the 5-position to uracil or another base that does not hybridize to guanine in the genomic DNA, and detecting unconverted cytosine in the synthetic DNA; and
(ii) detecting the presence or amount of one or more protein biomarkers in the one or more biological samples, wherein the one or more protein biomarkers comprise carcinoembryonic antigen (CEA) and/or amphiregulin.

2. The method of claim 1, wherein the proliferative disease is a cancer.

3. The method of claim 1, wherein the proliferative disease is a colorectal cancer or a colorectal cell proliferative disease.

4. The method of claim 3, wherein the one or more methylated genomic DNA sequences associated with a proliferative disease is methylated Septin-9 (mSEPT9).

5. The method of claim 4, wherein mSEPT9 comprises one or more CpG dinucleotides within the sequence set forth in SEQ ID NO: 31.

6. The method of claim 5, wherein the synthetic DNA is generated using at least one nucleic acid molecule comprising a contiguous sequence at least 9 nucleotides in length that is complementary to, or hybridizes to, SEQ ID NO: 31.

7. The method of claim 6, wherein the nucleic acid molecule is a methylation-specific oligonucleotide.

8. The method of any one of claims 3-7, wherein the one or more methylated genomic DNA sequences associated with a proliferative disease is methylated ANKRD13B (mANKRD13B).

9. The method of claim 8, wherein mANKRD13B comprises one or more CpG dinucleotides within the sequence set forth in SEQ ID NO: 6 or 11.

10. The method of claim 9, wherein the synthetic DNA is generated using at least one nucleic acid molecule comprising a contiguous sequence at least 9 nucleotides in length that is complementary to, or hybridizes to, SEQ ID NO: 6 or 11.

11. The method of claim 10, wherein the nucleic acid molecule is a methylation-specific oligonucleotide.

12. The method of any one of claims 1-11, wherein synthetic DNA is generated by treating the genomic DNA with bisulfite to produce sulfonated DNA.

13. The method of any one of claims 1-12, wherein the one or more protein biomarkers comprise CEA.

14. The method of any one of claims 1-12, wherein the one or more protein biomarkers comprise amphiregulin.

15. The method of any one of claims 1-12, wherein the one or more protein biomarkers comprise CEA and amphiregulin.

16. The method of any one of claims 1-15, wherein the one or more protein biomarkers further comprise CYFRA21-1.

17. The method of any one of claims 1-16, wherein the one or more protein biomarkers further comprise a galectin-3 ligand.

18. The method of claim 17, wherein the galectin-3 ligand is haptoglobin.

19. The method of any one of claims 1-18, wherein detecting the presence or amount of the one or more protein biomarkers comprises contacting the one or more biological samples with an antibody specific for the one or more protein biomarkers, and detecting the antibody.

20. The method of any one of claims 1-18, wherein the presence or amount of the one or more protein biomarkers is detecting by western blot, enzyme-linked immunosorbent assay (ELISA), immunobead-based format, proximity extension assay (PEA) or mass-spectrometry.

21. The method of any one of claims 1-20, wherein the biological sample comprises genomic DNA from a circulating cancer cell.

22. The method of any one of claims 1-21, wherein the one or more biological samples is a blood sample, a serum sample, a plasma sample, a urine sample, a saliva sample, a stool sample, or a combination thereof.

23. The method of any one of claims 1-22, wherein the one or more biological samples is a blood sample, a serum sample, a plasma sample, or a combination thereof.

24. The method of any one of claims 1-23, wherein the one or more biological samples are the same biological sample.

25. The method of any one of claims 1-23, wherein the one or more biological samples are different biological samples.

26. The method of any one of claims 1-25, further comprising diagnosing the subject as having a proliferative disease.

27. The method of any one of claims 1-26, wherein the presence of one or more methylated genomic DNA sequences and one or more protein biomarkers is indicative of the presence of a proliferative disease in a subject, or indicative of the presence of a risk of a subject having a proliferative disease.

28. The method of any one of claims 1-27, wherein the method detects proliferative disease in a subject with a sensitivity of at least 0.65 and/or a specificity of at least 0.8.

29. A method for detecting the presence or amount of methylated genomic DNA and protein in one or more biological samples, comprising:
(i) detecting DNA methylation within at least one genomic DNA polynucleotide in the one or more biological samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected, and
(ii) detecting the presence or amount of one or more protein biomarkers in the one or more biological samples, wherein the one or more protein biomarkers comprise CEA and/or amphiregulin.

30. A method for detecting the presence or amount of methylated genomic DNA and protein in one or more biological samples, comprising:
(i) detecting DNA methylation within SEQ ID NO: 31 (mSEPT9) and SEQ ID NOs: 6 and 11 (mANKDRD13b) in the one or more biological samples, wherein cytosine unmethylated in the 5-position is converted to uracil or another base that does not hybridize to guanine, and unconverted cytosine is detected; and
(ii) detecting the presence or amount of one or more protein biomarkers in the one or more biological samples, wherein the one or more protein biomarkers comprise CEA and/or amphiregulin.

31. The method of claim 29 or 30, wherein the biological sample is from a subject at risk or suspected of being at risk for having or developing colorectal cancer or a colorectal cell proliferative disease.

32. The method of any one of claims 29-31, wherein the one or more protein biomarkers comprise CEA.

33. The method of any one of claims 29-32, wherein the one or more protein biomarkers comprise amphiregulin.

34. The method of any one of claims 29-32, wherein the one or more protein biomarkers comprise CEA and amphiregulin.

35. The method of any one of claims 29-34, wherein the one or more protein biomarkers further comprise CYFRA21-1.

36. The method of any one of claims 29-35, wherein the one or more protein biomarkers further comprise a galectin-3 ligand.

37. The method of claim 36, wherein the galectin-3 ligand is haptoglobin.

38. A method for detecting the presence or amount of methylated genomic DNA and protein in one or more biological samples, comprising:
(i) detecting DNA methylation within at least one genomic DNA polynucleotide in the one or more biological samples, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin, and a galectin-3 ligand.

39. The method of claim 38, wherein the one or more protein biomarkers comprise (i) CEA and the galectin-3 ligand, (ii) amphiregulin and the galectin-3 ligand, or (iii) CEA, amphiregulin and the galectin-3 ligand.

40. The method of claim 38 or 39, wherein the galectin-3 ligand is haptoglobin.

41. The method of claim 39 or 40, wherein the method detects colorectal cancer or a colorectal cell proliferative disease in a subject with a sensitivity of at least 0.7 and/or a specificity of at least 0.8.

42. The method of any one of claims 29-41, wherein detecting the presence or amount of the one or more protein biomarkers comprises contacting the one or more biological samples with an antibody specific for the one or more protein biomarkers, and detecting the antibody.

43. The method of any one of claims 29-41, wherein the presence or amount of the one or more protein biomarkers is detecting by western blot, enzyme-linked immunosorbent assay (ELISA), immunobead-based format, proximity extension assay (PEA) or mass-spectrometry.

44. The method of any one of claims 29-43, wherein the one or more biological samples is a blood sample, a serum sample, a plasma sample, a urine sample, a saliva sample, a stool sample, or a combination thereof.

45. The method of any one of claims 29-44, wherein the one or more biological samples is a blood sample, a serum sample, a plasma sample, or a combination thereof.

46. The method of any one of claims 29-45, wherein the method detects colorectal cancer or a colorectal cell proliferative disease in a subject with a sensitivity of at least 0.65 and/or a specificity of at least 0.8.

47. The method of any one of claims 1-46, wherein the one or more protein biomarkers are detected by a manual, semi-automated, or fully automated system.

48. The method of any one of claim 147, wherein the methylated genomic DNA is detected by a manual, semi-automated, or fully automated system.

49. The method of any one of claims 1-48, wherein the one or more protein biomarkers are detected by a monoplex or multiplex system.

50. A method for detecting the presence or amount of methylated genomic DNA and protein in one or more biological samples from a subject, comprising:
(i) detecting one or more methylated genomic DNA sequences associated with a proliferative disease from the one or more biological samples; and
(ii) detecting the presence or amount of one or more protein biomarkers in the one or more biological samples, wherein the one or more protein biomarkers comprise CEA and/or amphiregulin.

51. A method for detecting the presence or amount of methylated genomic DNA and protein in one or more biological samples, comprising:
(i) detecting DNA methylation within at least one genomic DNA polynucleotide in the one or more biological samples, wherein the at least one genomic DNA polynucleotide comprises mSEPT9 and/or mANKDRD13b; and
(ii) detecting the presence or amount of one or more protein biomarkers, wherein the one or more protein biomarkers comprise CEA and/or amphiregulin.

52. A method for detecting the presence or amount of methylated genomic DNA and protein in one or more biological samples, comprising:
(i) detecting DNA methylation within SEQ ID NO: 31 (mSEPT9) and SEQ ID NOs: 6 and 11 (mANKDRD13b) in the one or more biological samples; and
(ii) detecting the presence or amount of one or more protein biomarkers in the one or more biological samples, wherein the one or more protein biomarkers comprise CEA and/or amphiregulin.

53. A method for detecting colorectal cancer or a colorectal cell proliferative disease in a subject, comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more biological samples, wherein the one or more protein biomarkers comprise CEA and/or amphiregulin,
wherein the presence of DNA methylation and one or more protein biomarkers in the one or more biological samples is indicative of the presence of colorectal cancer or a colorectal cell proliferative disease in the subject.

54. A method for detecting colorectal cancer or a colorectal cell proliferative disease in a subject, comprising:
(i) detecting DNA methylation within at least one genomic DNA polynucleotide in one or more plasma or serum samples from the subject, wherein the at least one genomic DNA polynucleotide comprises mSEPT9 and mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more plasma or serum samples, wherein the one or more protein biomarkers comprise CEA and/or amphiregulin,
wherein the presence of DNA methylation and one or more protein biomarkers in the one or more plasma or serum samples is indicative of the presence of colorectal cancer or a colorectal cell proliferative disease in the subject.

55. A method for detecting colorectal cancer or a colorectal cell proliferative disease in a subject, comprising:
(i) detecting DNA methylation within at least one genomic DNA polynucleotide in one or more plasma samples from the subject, wherein the at least one genomic DNA polynucleotide comprises mSEPT9 and mANKDRD13b, wherein cytosine unmethylated in the 5-position within the genomic DNA polynucleotide is converted to uracil or another base that does not hybridize to guanine in the genomic DNA polynucleotide, and unconverted cytosine is detected; and
(ii) detecting the presence or amount of one or more protein biomarkers in the one or more plasma samples, wherein the one or more protein biomarkers comprise CEA and/or amphiregulin, and a galectin-3 ligand,
wherein the presence of DNA methylation and one or more protein biomarkers in the one or more plasma samples is indicative of the presence of colorectal cancer or a colorectal cell proliferative disease in the subject.

56. The method of claim 55, wherein the galectin-3 ligand is haptoglobin.

57. The method of any one of claims 54-56, wherein colorectal cancer or the colorectal cell proliferative disease is detected in the subject with a sensitivity of at least 0.65 and/or a specificity of at least 0.8.

58. A method for prognosing colorectal cancer or a colorectal cell proliferative disease in a subject, comprising:
(i) detecting DNA methylation within at least one genomic DNA polynucleotide in one or more biological samples from the subject, wherein the at least one genomic DNA polynucleotide comprises mSEPT9 and/or mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more biological samples, wherein the one or more protein biomarkers comprise CEA and/or amphiregulin; and
(iii) prognosing the subject with colorectal cancer or a colorectal cell proliferative disease based on the presence of DNA methylation and one or more protein biomarkers in the one or more biological samples.

59. A method for prognosing colorectal cancer or a colorectal cell proliferative disease in a subject, comprising:
(i) detecting DNA methylation within at least one genomic DNA polynucleotide in one or more plasma or serum samples from the subject, wherein the at least one genomic DNA polynucleotide comprises mSEPT9 and mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more plasma or serum samples, wherein the one or more protein biomarkers comprise CEA and/or amphiregulin; and
(iii) prognosing the subject with colorectal cancer or a colorectal cell proliferative disease based on the presence of DNA methylation and one or more protein biomarkers in the one or more serum or plasma samples.

60. A method for prognosing colorectal cancer or a colorectal cell proliferative disease in a subject, comprising:
(i) detecting DNA methylation within at least one genomic DNA polynucleotide in one or more plasma samples from the subject, wherein the at least one genomic DNA polynucleotide comprises mSEPT9 and mANKDRD13b, wherein cytosine unmethylated in the 5-position within the genomic DNA polynucleotide is converted to uracil or another base that does not hybridize to guanine in the genomic DNA polynucleotide, and unconverted cytosine is detected;
(ii) detecting the presence or amount of one or more protein biomarkers in the one or more plasma samples, wherein the one or more protein biomarkers comprise CEA and/or amphiregulin, and a galectin-3 ligand; and
(iii) prognosing the subject with colorectal cancer or a colorectal cell proliferative disease based on the presence of DNA methylation and one or more protein biomarkers in the one or more plasma samples.

61. The method of claim 60, wherein the galectin-3 ligand is haptoglobin.

62. The method of any one of claims 53-61, further comprising performing a colonoscopy on the subject to confirm the presence of colorectal cancer or the colorectal cell proliferative disease.

63. The method of claim 62, further comprising treating the subject after confirmation of the presence of colorectal cancer or the colorectal cell proliferative disease in the subject.

64. The method of claim 63, wherein treating the subject comprises surgery, radiation therapy, chemotherapy, or immunotherapy.

65. A kit suitable for performing the method of any one of claims 1-54, comprising (i) an agent for converting cytosine unmethylated in the 5-position to uracil or another base that does not hybridize to guanine in the genomic DNA: (ii) at least one set of oligonucleotides complementary to the genomic DNA; and (iii) instructions for detecting methylation of genomic DNA in one or more biological samples in combination with instructions for detecting the one or more protein biomarkers, wherein the one or more protein biomarkers is CEA and/or amphiregulin in the one or more biological samples.

66. A kit suitable for performing the method of any one of claims 1-54, comprising (i) one or more reagents for detecting the one or more protein biomarkers, wherein the one or more protein biomarkers is CEA and/or amphiregulin; and (ii) instructions for detecting the presence or amount of the one or more protein biomarkers in one or more biological samples in combination with instructions for detecting methylated genomic DNA.

67. A kit suitable for performing the method of any one of claims 1-54, comprising (i) an agent for converting cytosine unmethylated in the 5-position to uracil or another base that does not hybridize to guanine in the genomic DNA: (ii) at least one set of oligonucleotides complementary to the genomic DNA; (iii) one or more reagents for detecting the one or more protein biomarkers, wherein the one or more protein biomarkers is CEA and/or amphiregulin; and (iv) instructions for detecting methylation of genomic DNA and the presence or amount of the one or more protein biomarkers in one or more biological samples.

68. The kit of any one of claims 65-67, wherein the instructions comprise steps for diagnosing, prognosing, or classifying colorectal cancer or a colorectal cell proliferative disease based on the detection of methylated genomic DNA and the one or more protein biomarkers.

69. The kit of any one of claims 65-68, wherein the one or more biological samples is a blood sample, a serum sample, a plasma sample, a urine sample, a saliva sample, or a stool sample.

70. The kit of any one of claims 65-69, wherein the one or more biological samples is a blood sample, a serum sample, or a plasma sample.

71. The kit of any one of claims 65-70, wherein the one or more biological samples is obtained from a subject at risk or suspected of being at risk for having or developing colorectal cancer or a colorectal cell proliferative disease.

72. The kit of any one of claims 65-71, wherein the one or more protein biomarkers comprises a galectin-3 ligand, optionally wherein the galectin-3 ligand is haptoglobin.


SEQUENCE LISTING (276 sequences)

SEQ ID NO: 1 - DNA, 64 bp, Homo sapiens
  source 1..64: mol_type = genomic DNA
ggaccctcgccagcgccacgcgcgccacgcccagggccaggagccagacaaaccccggcctgcg

SEQ ID NO: 2 - DNA, 64 bp, synthetic construct
  source 1..64: mol_type = other DNA
ggattttcgttagcgttacgcgcgttacgtttagggttaggagttagataaatttcggtttgcg

SEQ ID NO: 3 - DNA, 64 bp, synthetic construct
  source 1..64: mol_type = other DNA
cgcaaaccgaaatttatctaactcctaaccctaaacgtaacgcgcgtaacgctaacgaaaatcc

SEQ ID NO: 4 - DNA, 64 bp, synthetic construct
  source 1..64: mol_type = other DNA
aaaccctcgccaacgccacgcgcgccacgcccaaaaccaaaaaccaaacaaaccccgacctacg

SEQ ID NO: 5 - DNA, 64 bp, synthetic construct
  source 1..64: mol_type = other DNA
cgtaggtcggggtttgtttggtttttggttttgggcgtggcgcgcgtggcgttggcgagggttt

SEQ ID NO: 6 - DNA, 106 bp, Homo sapiens
  source 1..106: mol_type = genomic DNA
ggaccctcgccagcgccacgcgcgccacgcccagggccaggagccagacaaaccccggcctgcgcgcctgcagagcggcggctggagactggagccaccgcctcgc

SEQ ID NO: 7 - DNA, 106 bp, synthetic construct
  source 1..106: mol_type = other DNA
ggattttcgttagcgttacgcgcgttacgtttagggttaggagttagataaatttcggtttgcgcgtttgtagagcggcggttggagattggagttatcgtttcgt

SEQ ID NO: 8 - DNA, 106 bp, synthetic construct
  source 1..106: mol_type = other DNA
acgaaacgataactccaatctccaaccgccgctctacaaacgcgcaaaccgaaatttatctaactcctaaccctaaacgtaacgcgcgtaacgctaacgaaaatcc

SEQ ID NO: 9 - DNA, 106 bp, synthetic construct
  source 1..106: mol_type = other DNA
aaaccctcgccaacgccacgcgcgccacgcccaaaaccaaaaaccaaacaaaccccgacctacgcgcctacaaaacgacgactaaaaactaaaaccaccgcctcgc

SEQ ID NO: 10 - DNA, 106 bp, synthetic construct
  source 1..106: mol_type = other DNA
gcgaggcggtggttttagtttttagtcgtcgttttgtaggcgcgtaggtcggggtttgtttggtttttggttttgggcgtggcgcgcgtggcgttggcgagggttt

SEQ ID NO: 11 - DNA, 346 bp, Homo sapiens
  source 1..346: mol_type = genomic DNA
ggaccctcgccagcgccacgcgcgccacgcccagggccaggagccagacaaaccccggcctgcgcgcctgcagagcggcggctggagactggagccaccgcctcgcgggtgcagcagcacagcaggcacggttcggggagggattcggcatggccgcggggtaccttcccaggccagggccctggaggcaactggcacggcctggtccccctgctttgctgtattctgattccccaacccgctcccctgggctcagatctgtcctgtcctagggcggagccaggcggtcctgagggggagatgaatccttagaggagcgctgtccctatcccttgctccttctggc

SEQ ID NO: 12 - DNA, 346 bp, synthetic construct
  source 1..346: mol_type = other DNA
ggattttcgttagcgttacgcgcgttacgtttagggttaggagttagataaatttcggtttgcgcgtttgtagagcggcggttggagattggagttatcgtttcgcgggtgtagtagtatagtaggtacggttcggggagggattcggtatggtcgcggggtatttttttaggttagggttttggaggtaattggtacggtttggtttttttgttttgttgtattttgattttttaattcgtttttttgggtttagatttgttttgttttagggcggagttaggcggttttgagggggagatgaatttttagaggagcgttgtttttattttttgttttttttggt

SEQ ID NO: 13 - DNA, 346 bp, synthetic construct
  source 1..346: mol_type = other DNA
accaaaaaaaacaaaaaataaaaacaacgctcctctaaaaattcatctccccctcaaaaccgcctaactccgccctaaaacaaaacaaatctaaacccaaaaaaacgaattaaaaaatcaaaatacaacaaaacaaaaaaaccaaaccgtaccaattacctccaaaaccctaacctaaaaaaataccccgcgaccataccgaatccctccccgaaccgtacctactatactactacacccgcgaaacgataactccaatctccaaccgccgctctacaaacgcgcaaaccgaaatttatctaactcctaaccctaaacgtaacgcgcgtaacgctaacgaaaatcc

SEQ ID NO: 14 - DNA, 346 bp, synthetic construct
  source 1..346: mol_type = other DNA
aaaccctcgccaacgccacgcgcgccacgcccaaaaccaaaaaccaaacaaaccccgacctacgcgcctacaaaacgacgactaaaaactaaaaccaccgcctcgcgaatacaacaacacaacaaacacgattcgaaaaaaaattcgacataaccgcgaaataccttcccaaaccaaaaccctaaaaacaactaacacgacctaatccccctactttactatattctaattccccaacccgctcccctaaactcaaatctatcctatcctaaaacgaaaccaaacgatcctaaaaaaaaaataaatccttaaaaaaacgctatccctatcccttactccttctaac

SEQ ID NO: 15 - DNA, 346 bp, synthetic construct
  source 1..346: mol_type = other DNA
gttagaaggagtaagggatagggatagcgtttttttaaggatttattttttttttaggatcgtttggtttcgttttaggataggatagatttgagtttaggggagcgggttggggaattagaatatagtaaagtagggggattaggtcgtgttagttgtttttagggttttggtttgggaaggtatttcgcggttatgtcgaattttttttcgaatcgtgtttgttgtgttgttgtattcgcgaggcggtggttttagtttttagtcgtcgttttgtaggcgcgtaggtcggggtttgtttggtttttggttttgggcgtggcgcgcgtggcgttggcgagggttt

SEQ ID NO: 16 - DNA, 87 bp, Homo sapiens
  source 1..87: mol_type = genomic DNA
ggaccctcgccagcgccacgcgcgccacgcccagggccaggagccagacaaaccccggcctgcgcgcctgcagagcggcggctggag

SEQ ID NO: 17 - DNA, 87 bp, synthetic construct
  source 1..87: mol_type = other DNA
ggattttcgttagcgttacgcgcgttacgtttagggttaggagttagataaatttcggtttgcgcgtttgtagagcggcggttggag

SEQ ID NO: 18 - DNA, 87 bp, synthetic construct
  source 1..87: mol_type = other DNA
ctccaaccgccgctctacaaacgcgcaaaccgaaatttatctaactcctaaccctaaacgtaacgcgcgtaacgctaacgaaaatcc

SEQ ID NO: 19 - DNA, 87 bp, synthetic construct
  source 1..87: mol_type = other DNA
aaaccctcgccaacgccacgcgcgccacgcccaaaaccaaaaaccaaacaaaccccgacctacgcgcctacaaaacgacgactaaaa

SEQ ID NO: 20 - DNA, 87 bp, synthetic construct
  source 1..87: mol_type = other DNA
ttttagtcgtcgttttgtaggcgcgtaggtcggggtttgtttggtttttggttttgggcgtggcgcgcgtggcgttggcgagggttt

SEQ ID NO: 21 - DNA, 68 bp, Homo sapiens
  source 1..68: mol_type = genomic DNA
ctgcccaccagccatcatgtcggaccccgcggtcaacgcgcagctggatgggatcatttcggacttcg

SEQ ID NO: 22 - DNA, 68 bp, synthetic construct
  source 1..68: mol_type = other DNA
ttgtttattagttattatgtcggatttcgcggttaacgcgtagttggatgggattatttcggatttcg

SEQ ID NO: 23 - DNA, 68 bp, synthetic construct
  source 1..68: mol_type = other DNA
cgaaatccgaaataatcccatccaactacgcgttaaccgcgaaatccgacataataactaataaacaa

SEQ ID NO: 24 - DNA, 68 bp, synthetic construct
  source 1..68: mol_type = other DNA
ctacccaccaaccatcatatcgaaccccgcgatcaacgcgcaactaaataaaatcatttcgaacttcg

SEQ ID NO: 25 - DNA, 68 bp, synthetic construct
  source 1..68: mol_type = other DNA
cgaagttcgaaatgattttatttagttgcgcgttgatcgcggggttcgatatgatggttggtgggtag

SEQ ID NO: 26 - DNA, 75 bp, Homo sapiens
  source 1..75: mol_type = genomic DNA
cgacccgctgcccaccagccatcatgtcggaccccgcggtcaacgcgcagctggatgggatcatttcggacttcg

SEQ ID NO: 27 - DNA, 75 bp, synthetic construct
  source 1..75: mol_type = other DNA
cgattcgttgtttattagttattatgtcggatttcgcggttaacgcgtagttggatgggattatttcggatttcg

SEQ ID NO: 28 - DNA, 75 bp, synthetic construct
  source 1..75: mol_type = other DNA
cgaaatccgaaataatcccatccaactacgcgttaaccgcgaaatccgacataataactaataaacaacgaatcg

SEQ ID NO: 29 - DNA, 75 bp, synthetic construct
  source 1..75: mol_type = other DNA
cgacccgctacccaccaaccatcatatcgaaccccgcgatcaacgcgcaactaaataaaatcatttcgaacttcg

SEQ ID NO: 30 - DNA, 75 bp, synthetic construct
  source 1..75: mol_type = other DNA
cgaagttcgaaatgattttatttagttgcgcgttgatcgcggggttcgatatgatggttggtgggtagcgggtcg

SEQ ID NO: 31 - DNA, 1819 bp, Homo sapiens
  source 1..1819: mol_type = genomic DNA
gcgtcgcccgtccctggcttctctgacagccgtgttccatccccgccctgtgccccttctcccggacagtgccttctccagggctcacccaggagggtgcagcggtggcccccggggcggtggtcgtggtgggggtgttagctgcaggggtgccctcggtgggtgggagttggtggcctctcgctggtgccatgggactcgcatgttcgccctgcgcccctcggctcttgagcccacaggccgggatcctgcctgccagccgcgtgcgctgccgtttaacccttgcaggcgcagagcgcgcggcggcggtgacagagaactttgtttggctgcccaaatacagcctcctgcagaaggaccctgcgcccggggaaggggaggaatctcttcccctctgggcgcccgccctcctcgccatggcccggcctccacatccgcccacatctggccgcagcggggcgcccggggggaggggctgaggccgcgtctctcgccgtcccctgggcgcgggccaggcggggaggaggggggcgctccggtcgtgtgcccaggactgtcccccagcggccactcgggccccagccccccaggcctggccttgacaggcgggcggagcagccagtgcgagacagggaggccggtgcgggtgcgggaacctgatccgcccgggaggcgggggcggggcgggggcgcagcgcgcggggaggggccggcgcccgccttcctcccccattcattcagctgagccagggggcctaggggctcctccggcggctagctctgcactgcaggagcgcgggcgcggcgccccagccagcgcgcagggcccgggccccgccgggggcgcttcctcgccgctgccctccgcgcgacccgctgcccaccagccatcatgtcggaccccgcggtcaacgcgcagctggatgggatcatttcggacttcgaaggtgggtgctgggctggctgctgcggccgcggacgtgctggagaggaccctgcgggtgggcctggcgcgggacgggggtgcgctgaggggagacgggagtgcgctgaggggagacgggacccctaatccaggcgccctcccgctgagagcgccgcgcgcccccggccccgtgcccgcgccgcctacgtgggggaccctgttaggggcacccgcgtagaccctgcgcgccctcacaggaccctgtgctcgttctgcgcactgccgcctgggtttccttccttttattgttgtttgtgtttgccaagcgacagcgacctcctcgagggctcgcgaggctgcctcggaactctccaggacgcacagtttcactctgggaaatccatcggtcccctccctttggctctccccggcggctctcgggccccgcttggacccggcaacgggatagggaggtcgttcctcacctccgactgagtggacagccgcgtcctgctcgggtggacagccctcccctcccccacgccagtttcggggccgccaagttgtgcagcccgtgggccgggagcaccgaacggacacagcccaggtcgtggcagggtctagagtgggatgtcccatggcccccatccaggcctggggatatcctcatccgcctcccagaatcgggccgtgggggacagaaggggcctgcgtgcgggcagggagagtattttggctctctcctgtcttcggggtttacaaagtgtgttgggacttgcggggctgctctgtccaagcctgggtctggcgtccgcgtctctgagcctgtgagtgcgtgcgctttcctgcgtcctcttgactgccggtgctggggctctgcgtcctgcgtccgcg

SEQ ID NO: 32 - DNA, 1819 bp, synthetic construct
  source 1..1819: mol_type = other DNA
gcgtcgttcgtttttggtttttttgatagtcgtgttttattttcgttttgtgttttttttttcggatagtgttttttttagggtttatttaggagggtgtagcggtggttttcggggcggtggtcgtggtgggggtgttagttgtaggggtgttttcggtgggtgggagttggtggtttttcgttggtgttatgggattcgtatgttcgttttgcgtttttcggtttttgagtttataggtcgggattttgtttgttagtcgcgtgcgttgtcgtttaatttttgtaggcgtagagcgcgcggcggcggtgatagagaattttgtttggttgtttaaatatagttttttgtagaaggattttgcgttcggggaaggggaggaatttttttttttttgggcgttcgtttttttcgttatggttcggtttttatattcgtttatatttggtcgtagcggggcgttcggggggaggggttgaggtcgcgtttttcgtcgttttttgggcgcgggttaggcggggaggaggggggcgtttcggtcgtgtgtttaggattgttttttagcggttattcgggttttagttttttaggtttggttttgataggcgggcggagtagttagtgcgagatagggaggtcggtgcgggtgcgggaatttgattcgttcgggaggcgggggcggggcgggggcgtagcgcgcggggaggggtcggcgttcgttttttttttttatttatttagttgagttagggggtttaggggttttttcggcggttagttttgtattgtaggagcgcgggcgcggcgttttagttagcgcgtagggttcgggtttcgtcgggggcgttttttcgtcgttgtttttcgcgcgattcgttgtttattagttattatgtcggatttcgcggttaacgcgtagttggatgggattatttcggatttcgaaggtgggtgttgggttggttgttgcggtcgcggacgtgttggagaggattttgcgggtgggtttggcgcgggacgggggtgcgttgaggggagacgggagtgcgttgaggggagacgggatttttaatttaggcgttttttcgttgagagcgtcgcgcgttttcggtttcgtgttcgcgtcgtttacgtgggggattttgttaggggtattcgcgtagattttgcgcgtttttataggattttgtgttcgttttgcgtattgtcgtttgggtttttttttttttattgttgtttgtgtttgttaagcgatagcgattttttcgagggttcgcgaggttgtttcggaattttttaggacgtatagttttattttgggaaatttatcggttttttttttttggtttttttcggcggttttcgggtttcgtttggattcggtaacgggatagggaggtcgttttttattttcgattgagtggatagtcgcgttttgttcgggtggatagttttttttttttttacgttagtttcggggtcgttaagttgtgtagttcgtgggtcgggagtatcgaacggatatagtttaggtcgtggtagggtttagagtgggatgttttatggtttttatttaggtttggggatatttttattcgttttttagaatcgggtcgtgggggatagaaggggtttgcgtgcgggtagggagagtattttggtttttttttgttttcggggtttataaagtgtgttgggatttgcggggttgttttgtttaagtttgggtttggcgttcgcgtttttgagtttgtgagtgcgtgcgtttttttgcgtttttttgattgtcggtgttggggttttgcgttttgcgttcgcg

SEQ ID NO: 33 - DNA, 1819 bp, synthetic construct
  source 1..1819: mol_type = other DNA
cgcgaacgcaaaacgcaaaaccccaacaccgacaatcaaaaaaacgcaaaaaaacgcacgcactcacaaactcaaaaacgcgaacgccaaacccaaacttaaacaaaacaaccccgcaaatcccaacacactttataaaccccgaaaacaaaaaaaaaccaaaatactctccctacccgcacgcaaaccccttctatcccccacgacccgattctaaaaaacgaataaaaatatccccaaacctaaataaaaaccataaaacatcccactctaaaccctaccacgacctaaactatatccgttcgatactcccgacccacgaactacacaacttaacgaccccgaaactaacgtaaaaaaaaaaaaaactatccacccgaacaaaacgcgactatccactcaatcgaaaataaaaaacgacctccctatcccgttaccgaatccaaacgaaacccgaaaaccgccgaaaaaaaccaaaaaaaaaaaaccgataaatttcccaaaataaaactatacgtcctaaaaaattccgaaacaacctcgcgaaccctcgaaaaaatcgctatcgcttaacaaacacaaacaacaataaaaaaaaaaaaacccaaacgacaatacgcaaaacgaacacaaaatcctataaaaacgcgcaaaatctacgcgaatacccctaacaaaatcccccacgtaaacgacgcgaacacgaaaccgaaaacgcgcgacgctctcaacgaaaaaacgcctaaattaaaaatcccgtctcccctcaacgcactcccgtctcccctcaacgcacccccgtcccgcgccaaacccacccgcaaaatcctctccaacacgtccgcgaccgcaacaaccaacccaacacccaccttcgaaatccgaaataatcccatccaactacgcgttaaccgcgaaatccgacataataactaataaacaacgaatcgcgcgaaaaacaacgacgaaaaaacgcccccgacgaaacccgaaccctacgcgctaactaaaacgccgcgcccgcgctcctacaatacaaaactaaccgccgaaaaaacccctaaaccccctaactcaactaaataaataaaaaaaaaaaacgaacgccgacccctccccgcgcgctacgcccccgccccgcccccgcctcccgaacgaatcaaattcccgcacccgcaccgacctccctatctcgcactaactactccgcccgcctatcaaaaccaaacctaaaaaactaaaacccgaataaccgctaaaaaacaatcctaaacacacgaccgaaacgcccccctcctccccgcctaacccgcgcccaaaaaacgacgaaaaacgcgacctcaacccctccccccgaacgccccgctacgaccaaatataaacgaatataaaaaccgaaccataacgaaaaaaacgaacgcccaaaaaaaaaaaaattcctccccttccccgaacgcaaaatccttctacaaaaaactatatttaaacaaccaaacaaaattctctatcaccgccgccgcgcgctctacgcctacaaaaattaaacgacaacgcacgcgactaacaaacaaaatcccgacctataaactcaaaaaccgaaaaacgcaaaacgaacatacgaatcccataacaccaacgaaaaaccaccaactcccacccaccgaaaacacccctacaactaacacccccaccacgaccaccgccccgaaaaccaccgctacaccctcctaaataaaccctaaaaaaaacactatccgaaaaaaaaaacacaaaacgaaaataaaacacgactatcaaaaaaaccaaaaacgaacgacgc

SEQ ID NO: 34 - DNA, 1819 bp, synthetic construct
  source 1..1819: mol_type = other DNA
acgtcgcccgtccctaacttctctaacaaccgtattccatccccgccctataccccttctcccgaacaataccttctccaaaactcacccaaaaaaatacaacgataacccccgaaacgataatcgtaataaaaatattaactacaaaaataccctcgataaataaaaattaataacctctcgctaataccataaaactcgcatattcgccctacgcccctcgactcttaaacccacaaaccgaaatcctacctaccaaccgcgtacgctaccgtttaacccttacaaacgcaaaacgcgcgacgacgataacaaaaaactttatttaactacccaaatacaacctcctacaaaaaaaccctacgcccgaaaaaaaaaaaaaatctcttcccctctaaacgcccgccctcctcgccataacccgacctccacatccgcccacatctaaccgcaacgaaacgcccgaaaaaaaaaactaaaaccgcgtctctcgccgtcccctaaacgcgaaccaaacgaaaaaaaaaaaaacgctccgatcgtatacccaaaactatcccccaacgaccactcgaaccccaaccccccaaacctaaccttaacaaacgaacgaaacaaccaatacgaaacaaaaaaaccgatacgaatacgaaaacctaatccgcccgaaaaacgaaaacgaaacgaaaacgcaacgcgcgaaaaaaaaccgacgcccgccttcctcccccattcattcaactaaaccaaaaaacctaaaaactcctccgacgactaactctacactacaaaaacgcgaacgcgacgccccaaccaacgcgcaaaacccgaaccccgccgaaaacgcttcctcgccgctaccctccgcgcgacccgctacccaccaaccatcatatcgaaccccgcgatcaacgcgcaactaaataaaatcatttcgaacttcgaaaataaatactaaactaactactacgaccgcgaacgtactaaaaaaaaccctacgaataaacctaacgcgaaacgaaaatacgctaaaaaaaaacgaaaatacgctaaaaaaaaacgaaacccctaatccaaacgccctcccgctaaaaacgccgcgcgcccccgaccccgtacccgcgccgcctacgtaaaaaaccctattaaaaacacccgcgtaaaccctacgcgccctcacaaaaccctatactcgttctacgcactaccgcctaaatttccttccttttattattatttatatttaccaaacgacaacgacctcctcgaaaactcgcgaaactacctcgaaactctccaaaacgcacaatttcactctaaaaaatccatcgatcccctccctttaactctccccgacgactctcgaaccccgcttaaacccgacaacgaaataaaaaaatcgttcctcacctccgactaaataaacaaccgcgtcctactcgaataaacaaccctcccctcccccacgccaatttcgaaaccgccaaattatacaacccgtaaaccgaaaacaccgaacgaacacaacccaaatcgtaacaaaatctaaaataaaatatcccataacccccatccaaacctaaaaatatcctcatccgcctcccaaaatcgaaccgtaaaaaacaaaaaaaacctacgtacgaacaaaaaaaatattttaactctctcctatcttcgaaatttacaaaatatattaaaacttacgaaactactctatccaaacctaaatctaacgtccgcgtctctaaacctataaatacgtacgctttcctacgtcctcttaactaccgatactaaaactctacgtcctacgtccgcg

SEQ ID NO: 35 - DNA, 1819 bp, synthetic construct
  source 1..1819: mol_type = other DNA
cgcggacgtaggacgtagagttttagtatcggtagttaagaggacgtaggaaagcgtacgtatttataggtttagagacgcggacgttagatttaggtttggatagagtagtttcgtaagttttaatatattttgtaaatttcgaagataggagagagttaaaatattttttttgttcgtacgtaggttttttttgttttttacggttcgattttgggaggcggatgaggatatttttaggtttggatgggggttatgggatattttattttagattttgttacgatttgggttgtgttcgttcggtgttttcggtttacgggttgtataatttggcggtttcgaaattggcgtgggggaggggagggttgtttattcgagtaggacgcggttgtttatttagtcggaggtgaggaacgatttttttatttcgttgtcgggtttaagcggggttcgagagtcgtcggggagagttaaagggaggggatcgatggattttttagagtgaaattgtgcgttttggagagtttcgaggtagtttcgcgagttttcgaggaggtcgttgtcgtttggtaaatataaataataataaaaggaaggaaatttaggcggtagtgcgtagaacgagtatagggttttgtgagggcgcgtagggtttacgcgggtgtttttaatagggttttttacgtaggcggcgcgggtacggggtcgggggcgcgcggcgtttttagcgggagggcgtttggattaggggtttcgtttttttttagcgtattttcgtttttttttagcgtattttcgtttcgcgttaggtttattcgtagggttttttttagtacgttcgcggtcgtagtagttagtttagtatttattttcgaagttcgaaatgattttatttagttgcgcgttgatcgcggggttcgatatgatggttggtgggtagcgggtcgcgcggagggtagcggcgaggaagcgttttcggcggggttcgggttttgcgcgttggttggggcgtcgcgttcgcgtttttgtagtgtagagttagtcgtcggaggagtttttaggttttttggtttagttgaatgaatgggggaggaaggcgggcgtcggttttttttcgcgcgttgcgttttcgtttcgttttcgtttttcgggcggattaggttttcgtattcgtatcggtttttttgtttcgtattggttgtttcgttcgtttgttaaggttaggtttggggggttggggttcgagtggtcgttgggggatagttttgggtatacgatcggagcgtttttttttttttcgtttggttcgcgtttaggggacggcgagagacgcggttttagttttttttttcgggcgtttcgttgcggttagatgtgggcggatgtggaggtcgggttatggcgaggagggcgggcgtttagaggggaagagattttttttttttttcgggcgtagggtttttttgtaggaggttgtatttgggtagttaaataaagttttttgttatcgtcgtcgcgcgttttgcgtttgtaagggttaaacggtagcgtacgcggttggtaggtaggatttcggtttgtgggtttaagagtcgaggggcgtagggcgaatatgcgagttttatggtattagcgagaggttattaatttttatttatcgagggtatttttgtagttaatatttttattacgattatcgtttcgggggttatcgttgtatttttttgggtgagttttggagaaggtattgttcgggagaaggggtatagggcggggatggaatacggttgttagagaagttagggacgggcgacgt

SEQ ID NO: 36 - DNA, 1062 bp, Homo sapiens
  source 1..1062: mol_type = genomic DNA
aggggaggaatctcttcccctctgggcgcccgccctcctcgccatggcccggcctccacatccgcccacatctggccgcagcggggcgcccggggggaggggctgaggccgcgtctctcgccgtcccctgggcgcgggccaggcggggaggaggggggcgctccggtcgtgtgcccaggactgtcccccagcggccactcgggccccagccccccaggcctggccttgacaggcgggcggagcagccagtgcgagacagggaggccggtgcgggtgcgggaacctgatccgcccgggaggcgggggcggggcgggggcgcagcgcgcggggaggggccggcgcccgccttcctcccccattcattcagctgagccagggggcctaggggctcctccggcggctagctctgcactgcaggagcgcgggcgcggcgccccagccagcgcgcagggcccgggccccgccgggggcgcttcctcgccgctgccctccgcgcgacccgctgcccaccagccatcatgtcggaccccgcggtcaacgcgcagctggatgggatcatttcggacttcgaaggtgggtgctgggctggctgctgcggccgcggacgtgctggagaggaccctgcgggtgggcctggcgcgggacgggggtgcgctgaggggagacgggagtgcgctgaggggagacgggacccctaatccaggcgccctcccgctgagagcgccgcgcgcccccggccccgtgcccgcgccgcctacgtgggggaccctgttaggggcacccgcgtagaccctgcgcgccctcacaggaccctgtgctcgttctgcgcactgccgcctgggtttccttccttttattgttgtttgtgtttgccaagcgacagcgacctcctcgagggctcgcgaggctgcctcggaactctccaggacgcacagtttcactctgggaaatccatcggtcccctccctttggctctccccggcggctctcgggccccgcttggacccggcaacgggatagggaggtcgttcctcacctccgactgagtggacagccgcgtc

SEQ ID NO: 37 - DNA, 1062 bp, synthetic construct
  source 1..1062: mol_type = other DNA
aggggaggaatttttttttttttgggcgttcgtttttttcgttatggttcggtttttatattcgtttatatttggtcgtagcggggcgttcggggggaggggttgaggtcgcgtttttcgtcgttttttgggcgcgggttaggcggggaggaggggggcgtttcggtcgtgtgtttaggattgttttttagcggttattcgggttttagttttttaggtttggttttgataggcgggcggagtagttagtgcgagatagggaggtcggtgcgggtgcgggaatttgattcgttcgggaggcgggggcggggcgggggcgtagcgcgcggggaggggtcggcgttcgttttttttttttatttatttagttgagttagggggtttaggggttttttcggcggttagttttgtattgtaggagcgcgggcgcggcgttttagttagcgcgtagggttcgggtttcgtcgggggcgttttttcgtcgttgtttttcgcgcgattcgttgtttattagttattatgtcggatttcgcggttaacgcgtagttggatgggattatttcggatttcgaaggtgggtgttgggttggttgttgcggtcgcggacgtgttggagaggattttgcgggtgggtttggcgcgggacgggggtgcgttgaggggagacgggagtgcgttgaggggagacgggatttttaatttaggcgttttttcgttgagagcgtcgcgcgttttcggtttcgtgttcgcgtcgtttacgtgggggattttgttaggggtattcgcgtagattttgcgcgtttttataggattttgtgttcgttttgcgtattgtcgtttgggtttttttttttttattgttgtttgtgtttgttaagcgatagcgattttttcgagggttcgcgaggttgtttcggaattttttaggacgtatagttttattttgggaaatttatcggttttttttttttggtttttttcggcggttttcgggtttcgtttggattcggtaacgggatagggaggtcgttttttattttcgattgagtggatagtcgcgtt

SEQ ID NO: 38 - DNA, 1062 bp, synthetic construct
  source 1..1062: mol_type = other DNA
aacgcgactatccactcaatcgaaaataaaaaacgacctccctatcccgttaccgaatccaaacgaaacccgaaaaccgccgaaaaaaaccaaaaaaaaaaaaccgataaatttcccaaaataaaactatacgtcctaaaaaattccgaaacaacctcgcgaaccctcgaaaaaatcgctatcgcttaacaaacacaaacaacaataaaaaaaaaaaaacccaaacgacaatacgcaaaacgaacacaaaatcctataaaaacgcgcaaaatctacgcgaatacccctaacaaaatcccccacgtaaacgacgcgaacacgaaaccgaaaacgcgcgacgctctcaacgaaaaaacgcctaaattaaaaatcccgtctcccctcaacgcactcccgtctcccctcaacgcacccccgtcccgcgccaaacccacccgcaaaatcctctccaacacgtccgcgaccgcaacaaccaacccaacacccaccttcgaaatccgaaataatcccatccaactacgcgttaaccgcgaaatccgacataataactaataaacaacgaatcgcgcgaaaaacaacgacgaaaaaacgcccccgacgaaacccgaaccctacgcgctaactaaaacgccgcgcccgcgctcctacaatacaaaactaaccgccgaaaaaacccctaaaccccctaactcaactaaataaataaaaaaaaaaaacgaacgccgacccctccccgcgcgctacgcccccgccccgcccccgcctcccgaacgaatcaaattcccgcacccgcaccgacctccctatctcgcactaactactccgcccgcctatcaaaaccaaacctaaaaaactaaaacccgaataaccgctaaaaaacaatcctaaacacacgaccgaaacgcccccctcctccccgcctaacccgcgcccaaaaaacgacgaaaaacgcgacctcaacccctccccccgaacgccccgctacgaccaaatataaacgaatataaaaaccgaaccataacgaaaaaaacgaacgcccaaaaaaaaaaaaattcctcccct

SEQ ID NO: 39 - DNA, 1062 bp, synthetic construct
  source 1..1062: mol_type = other DNA
aaaaaaaaaatctcttcccctctaaacgcccgccctcctcgccataacccgacctccacatccgcccacatctaaccgcaacgaaacgcccgaaaaaaaaaactaaaaccgcgtctctcgccgtcccctaaacgcgaaccaaacgaaaaaaaaaaaaacgctccgatcgtatacccaaaactatcccccaacgaccactcgaaccccaaccccccaaacctaaccttaacaaacgaacgaaacaaccaatacgaaacaaaaaaaccgatacgaatacgaaaacctaatccgcccgaaaaacgaaaacgaaacgaaaacgcaacgcgcgaaaaaaaaccgacgcccgccttcctcccccattcattcaactaaaccaaaaaacctaaaaactcctccgacgactaactctacactacaaaaacgcgaacgcgacgccccaaccaacgcgcaaaacccgaaccccgccgaaaacgcttcctcgccgctaccctccgcgcgacccgctacccaccaaccatcatatcgaaccccgcgatcaacgcgcaactaaataaaatcatttcgaacttcgaaaataaatactaaactaactactacgaccgcgaacgtactaaaaaaaaccctacgaataaacctaacgcgaaacgaaaatacgctaaaaaaaaacgaaaatacgctaaaaaaaaacgaaacccctaatccaaacgccctcccgctaaaaacgccgcgcgcccccgaccccgtacccgcgccgcctacgtaaaaaaccctattaaaaacacccgcgtaaaccctacgcgccctcacaaaaccctatactcgttctacgcactaccgcctaaatttccttccttttattattatttatatttaccaaacgacaacgacctcctcgaaaactcgcgaaactacctcgaaactctccaaaacgcacaatttcactctaaaaaatccatcgatcccctccctttaactctccccgacgactctcgaaccccgcttaaacccgacaacgaaataaaaaaatcgttcctcacctccgactaaataaacaaccgcgtc

SEQ ID NO: 40 - DNA, 1062 bp, synthetic construct
  source 1..1062: mol_type = other DNA
gacgcggttgtttatttagtcggaggtgaggaacgatttttttatttcgttgtcgggtttaagcggggttcgagagtcgtcggggagagttaaagggaggggatcgatggattttttagagtgaaattgtgcgttttggagagtttcgaggtagtttcgcgagttttcgaggaggtcgttgtcgtttggtaaatataaataataataaaaggaaggaaatttaggcggtagtgcgtagaacgagtatagggttttgtgagggcgcgtagggtttacgcgggtgtttttaatagggttttttacgtaggcggcgcgggtacggggtcgggggcgcgcggcgtttttagcgggagggcgtttggattaggggtttcgtttttttttagcgtattttcgtttttttttagcgtattttcgtttcgcgttaggtttattcgtagggttttttttagtacgttcgcggtcgtagtagttagtttagtatttattttcgaagttcgaaatgattttatttagttgcgcgttgatcgcggggttcgatatgatggttggtgggtagcgggtcgcgcggagggtagcggcgaggaagcgttttcggcggggttcgggttttgcgcgttggttggggcgtcgcgttcgcgtttttgtagtgtagagttagtcgtcggaggagtttttaggttttttggtttagttgaatgaatgggggaggaaggcgggcgtcggttttttttcgcgcgttgcgttttcgtttcgttttcgtttttcgggcggattaggttttcgtattcgtatcggtttttttgtttcgtattggttgtttcgttcgtttgttaaggttaggtttggggggttggggttcgagtggtcgttgggggatagttttgggtatacgatcggagcgtttttttttttttcgtttggttcgcgtttaggggacggcgagagacgcggttttagttttttttttcgggcgtttcgttgcggttagatgtgggcggatgtggaggtcgggttatggcgaggagggcgggcgtttagaggggaagagatttttttttt

SEQ ID NO: 41 - DNA, 18 bp, synthetic construct
  source 1..18: mol_type = other DNA
ggattttcgttagcgtta

SEQ ID NO: 42 - DNA, 18 bp, synthetic construct
  source 1..18: mol_type = other DNA
cgcaaaccgaaatttatc

SEQ ID NO: 43 - DNA, 15 bp, synthetic construct
  source 1..15: mol_type = other DNA
aaaccctcgccaacg

SEQ ID NO: 44 - DNA, 22 bp, synthetic construct
  source 1..22: mol_type = other DNA
ttttagtcgtcgttttgtaggc

SEQ ID NO: 45 - DNA, 22 bp, synthetic construct
  source 1..22: mol_type = other DNA
cgaagttcgaaatgattttatt

SEQ ID NO: 46 - DNA, 21 bp, synthetic construct
  source 1..21: mol_type = other DNA
ctacccaccaaccatcatatc

SEQ ID NO: 47 - DNA, 23 bp, synthetic construct
  source 1..23: mol_type = other DNA
cgcgttacgtttagggttaggag

SEQ ID NO: 48 - DNA, 20 bp, synthetic construct
  source 1..20: mol_type = other DNA
cacgcgcgccacgcccaaaa

SEQ ID NO: 49 - DNA, 17 bp, synthetic construct
  source 1..17: mol_type = other DNA
cgttgatcgcggggttc

SEQ ID NO: 50 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 51 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 52 - protein, 400 aa, Homo sapiens
MTSYSYRQSSATSSFGGLGGGSVRFGPGVAFRAPSIHGGSGGRGVSVSSARFVSSSSSGAYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLGDVRADSERQNQEYQRLMDIKSRLEQEIATYRSLLEGQEDHYNNLSASKVL

SEQ ID NO: 53 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 54 - DNA, 3318 bp, Homo sapiens
  source 1..3318: mol_type = genomic DNA
ccgggtttctaaccggcaggcgcggcccaggttcccggaggcagccccagagtcgcggaccgatgtgccaggctgtggatgagccctgggtaggggagggttcgtaccagcggcgcctggggcagcgaggagcgcgcgttctgcctgcgaagctgccttctccgagccccgcccaggaacattagctctggggggccgctgatcattgatttggacggagagatgggttctgggttctgtattaggattccagcatctgggctcgaggcagggcaatatccagaaagaccccagggttcggggtacccgggccagggctgaggcgcatcgccgagcaaaggctgggtgcgaggcgtgcggaatgatgcgcttgccttgcccgggcctctccaaggatggagaaaaggcgagtgaagcagcgaagtacgactccaaccccgcccagagagtgctactagcgctggctgcacgccaagtctctccaggggtccaaagcgagagggatttgttttaacccatctctacccgtcctgtgtcaagaacggaggctgtagagggcgactgcgaagtcgccaggcactcgctggatctcggtccccctcctcgtgctctggggttgagatggggcaccgccatcgataacagatcagcgcgaactattcgtttagtggccttaaaacaccctggtttcaccctcagctattttcaagttcccgtgtgcctggcactttctccgtgcgagaagcaccggagggtgcggacgcgccacagtctgagccgccgccgaactggctaagtttaggggcatttattattcatgttcctgccagatcctcgcctgcccaaaatagaaaccgaggttctccgtgacctacatctgctcggagaagggctcccctgggctcggaggctggggtgggggtggctgaggagttggcccccgcacgccccacgcatcctctcctttgctttctgggcctccccattcgggtcttcgcgtgggtcagcgcccggtctcccagggcctttctcgtccccgcccgttgctgctttggggaggctcgggagccaggcggggaggggggcggtccttttccgtagacaggtgtgcgcgatcggcggagacgcctcggtttcccagcgcttgttgaggccgtggcccgcaggacgaccctttacccgcgaagggggggtgggcgggccgcccggcggggtaggagtggttgggtgtcgttgcctcctccttacctctgctcccacccccagtcctgggagaagagacaattctcagcggaggacttttatcacctgtgaaaatccgcgcgagccccttactttggatcctcgccgagctggggaggaacttgcactgaccacaccttctgtccccggccaccccgcaggccagaggaagaggcgtacggcgaggacggaaacccgctgccagacttcgatggctcggagccgccgggcgcagggagccccgcctccgcgccgcgcgccgccgccgcctggtaccgcccggccgggagaaggtgagattcgcgcggcctcgcgcacacccgcggctgggagctcgggactgcggtgacgggaggggcagtgtggtgacccacccaggatttttttttttttcccgtgaaagtcctcaagcctgtcctctccctggcccgatcctattgcagcgacagaaaatcagcagcgggcgggtctgtgtggacctgagggccgcgtggggaccgaggggggctgtggcccaaagagtggcagtgagtggcgtcaaggaacccacactccgcatctgccactcctagagccgggactagctcccgatcctagcagttgctctcgagatcatcccgggagttattggcgagttctgggcctctggaggtttccctgtcagcctccccggccgccgagggggcgcgcgcccaacaagggggtctctagcggccacctggggacagaaacagtgaccctgggcgcgcactttgcctccccgttagagatgtcgcccacgggatccttaacgaggcctaccgcaaagtgctggaccagctgtccgccgggaagcacctgcagtcgctcgtggcccggggcgtggggtaagagtttgtggaaggattaacctgcgcgcgccggggtgggtgcctgtgcggggcgcgcggggcgggcggcggtgggtgcccgtgggggccagggtgagtctgcgcccctgggtctggggtgggcatccgccacgggtcgcagttggagattttgaagtggcactttaaatttgcccagagagctctggaagaggcaaaaagggaacgcgagccagggagtttgatccgttttgaatgaaaagaaagagaaaccaaaccaaacctctcagtcatccaaaaccttcaggcttccagggaggttttgctataattttctctaagcatgactgtttctgggggaggggaaaggggtggttgtatttactgaaaattcaaatcgaaataataaatggccaaatgtggacacttatggacccaaacagttttgctcacgccagagaaactgagagcacagggcttgtgtgaagcctatctcggcagaaggcaacattctaataaagcccgtgggaaaacagattacattttcgccatgaataagtcatgcagtgaaaaatattgcctacagcctgtcgacttatattattatcacgtttttcaactcggcgtgaggagggagaggagtgttcatatttgactaggaattgcaggatcgatgcaaactccagggcagcagccagactggcatatgtagggctctccggttactttctctgtatgtcgcgggtgagaggaacagcgaggacaatttagcgcaaacacacgaagggtcggatctcaagggggcagcgctgggagaaaggttaggcttgaagcgcgcgtcgcctgcccggatcttatcccgggccccctccgcagggtttggtgccaggagatcctgcgtggggaggggggcatcgaggggctgccgtctcggccctccccacggctgcttccaggcagaggcgggcgacgcggtgggcagtgcgagccccgggccctccccgaaggctcccgcgtggggtggggcccgcctgctccccgcggcgattgaacctgtgtctcccgccccgccaccctcttcccgacccctttgcttgcagtgggagcctcggcggcggcgcgggggacgacgcggagccgctctccaagcgccactcggacgggatcttcacggacagctacagccgctaccggaaacaaatggctgtcaagaaatacttggcggccg

SEQ ID NO: 55 - DNA, 3318 bp, synthetic construct
  source 1..3318: mol_type = other DNA
tcgggtttttaatcggtaggcgcggtttaggttttcggaggtagttttagagtcgcggatcgatgtgttaggttgtggatgagttttgggtaggggagggttcgtattagcggcgtttggggtagcgaggagcgcgcgttttgtttgcgaagttgtttttttcgagtttcgtttaggaatattagttttggggggtcgttgattattgatttggacggagagatgggttttgggttttgtattaggattttagtatttgggttcgaggtagggtaatatttagaaagattttagggttcggggtattcgggttagggttgaggcgtatcgtcgagtaaaggttgggtgcgaggcgtgcggaatgatgcgtttgttttgttcgggtttttttaaggatggagaaaaggcgagtgaagtagcgaagtacgattttaatttcgtttagagagtgttattagcgttggttgtacgttaagtttttttaggggtttaaagcgagagggatttgttttaatttatttttattcgttttgtgttaagaacggaggttgtagagggcgattgcgaagtcgttaggtattcgttggatttcggttttttttttcgtgttttggggttgagatggggtatcgttatcgataatagattagcgcgaattattcgtttagtggttttaaaatattttggttttatttttagttatttttaagttttcgtgtgtttggtatttttttcgtgcgagaagtatcggagggtgcggacgcgttatagtttgagtcgtcgtcgaattggttaagtttaggggtatttattatttatgtttttgttagattttcgtttgtttaaaatagaaatcgaggtttttcgtgatttatatttgttcggagaagggtttttttgggttcggaggttggggtgggggtggttgaggagttggttttcgtacgttttacgtatttttttttttgttttttgggtttttttattcgggttttcgcgtgggttagcgttcggttttttagggtttttttcgttttcgttcgttgttgttttggggaggttcgggagttaggcggggaggggggcggttttttttcgtagataggtgtgcgcgatcggcggagacgtttcggttttttagcgtttgttgaggtcgtggttcgtaggacgattttttattcgcgaagggggggtgggcgggtcgttcggcggggtaggagtggttgggtgtcgttgttttttttttatttttgtttttatttttagttttgggagaagagataatttttagcggaggatttttattatttgtgaaaattcgcgcgagttttttattttggattttcgtcgagttggggaggaatttgtattgattatattttttgttttcggttatttcgtaggttagaggaagaggcgtacggcgaggacggaaattcgttgttagatttcgatggttcggagtcgtcgggcgtagggagtttcgttttcgcgtcgcgcgtcgtcgtcgtttggtatcgttcggtcgggagaaggtgagattcgcgcggtttcgcgtatattcgcggttgggagttcgggattgcggtgacgggaggggtagtgtggtgatttatttaggatttttttttttttttcgtgaaagtttttaagtttgttttttttttggttcgattttattgtagcgatagaaaattagtagcgggcgggtttgtgtggatttgagggtcgcgtggggatcgaggggggttgtggtttaaagagtggtagtgagtggcgttaaggaatttatatttcgtatttgttatttttagagtcgggattagttttcgattttagtagttgttttcgagattatttcgggagttattggcgagttttgggtttttggaggtttttttgttagttttttcggtcgtcgagggggcgcgcgtttaataagggggtttttagcggttatttggggatagaaatagtgattttgggcgcgtattttgttttttcgttagagatgtcgtttacgggatttttaacgaggtttatcgtaaagtgttggattagttgttcgtcgggaagtatttgtagtcgttcgtggttcggggcgtggggtaagagtttgtggaaggattaatttgcgcgcgtcggggtgggtgtttgtgcggggcgcgcggggcgggcggcggtgggtgttcgtgggggttagggtgagtttgcgtttttgggtttggggtgggtattcgttacgggtcgtagttggagattttgaagtggtattttaaatttgtttagagagttttggaagaggtaaaaagggaacgcgagttagggagtttgattcgttttgaatgaaaagaaagagaaattaaattaaattttttagttatttaaaatttttaggtttttagggaggttttgttataattttttttaagtatgattgtttttgggggaggggaaaggggtggttgtatttattgaaaatttaaatcgaaataataaatggttaaatgtggatatttatggatttaaatagttttgtttacgttagagaaattgagagtatagggtttgtgtgaagtttatttcggtagaaggtaatattttaataaagttcgtgggaaaatagattatattttcgttatgaataagttatgtagtgaaaaatattgtttatagtttgtcgatttatattattattacgttttttaattcggcgtgaggagggagaggagtgtttatatttgattaggaattgtaggatcgatgtaaattttagggtagtagttagattggtatatgtagggtttttcggttattttttttgtatgtcgcgggtgagaggaatagcgaggataatttagcgtaaatatacgaagggtcggattttaagggggtagcgttgggagaaaggttaggtttgaagcgcgcgtcgtttgttcggattttatttcgggtttttttcgtagggtttggtgttaggagattttgcgtggggaggggggtatcgaggggttgtcgtttcggttttttttacggttgtttttaggtagaggcgggcgacgcggtgggtagtgcgagtttcgggtttttttcgaaggttttcgcgtggggtggggttcgtttgtttttcgcggcgattgaatttgtgtttttcgtttcgttatttttttttcgatttttttgtttgtagtgggagtttcggcggcggcgcgggggacgacgcggagtcgttttttaagcgttattcggacgggatttttacggatagttatagtcgttatcggaaataaatggttgttaagaaatatttggcggtcg

SEQ ID NO: 56 - DNA, 3318 bp, synthetic construct
  source 1..3318: mol_type = other DNA
cgaccgccaaatatttcttaacaaccatttatttccgataacgactataactatccgtaaaaatcccgtccgaataacgcttaaaaaacgactccgcgtcgtcccccgcgccgccgccgaaactcccactacaaacaaaaaaatcgaaaaaaaaataacgaaacgaaaaacacaaattcaatcgccgcgaaaaacaaacgaaccccaccccacgcgaaaaccttcgaaaaaaacccgaaactcgcactacccaccgcgtcgcccgcctctacctaaaaacaaccgtaaaaaaaaccgaaacgacaacccctcgatacccccctccccacgcaaaatctcctaacaccaaaccctacgaaaaaaacccgaaataaaatccgaacaaacgacgcgcgcttcaaacctaacctttctcccaacgctacccccttaaaatccgacccttcgtatatttacgctaaattatcctcgctattcctctcacccgcgacatacaaaaaaaataaccgaaaaaccctacatataccaatctaactactaccctaaaatttacatcgatcctacaattcctaatcaaatataaacactcctctccctcctcacgccgaattaaaaaacgtaataataatataaatcgacaaactataaacaatatttttcactacataacttattcataacgaaaatataatctattttcccacgaactttattaaaatattaccttctaccgaaataaacttcacacaaaccctatactctcaatttctctaacgtaaacaaaactatttaaatccataaatatccacatttaaccatttattatttcgatttaaattttcaataaatacaaccacccctttcccctcccccaaaaacaatcatacttaaaaaaaattataacaaaacctccctaaaaacctaaaaattttaaataactaaaaaatttaatttaatttctctttcttttcattcaaaacgaatcaaactccctaactcgcgttccctttttacctcttccaaaactctctaaacaaatttaaaataccacttcaaaatctccaactacgacccgtaacgaatacccaccccaaacccaaaaacgcaaactcaccctaacccccacgaacacccaccgccgcccgccccgcgcgccccgcacaaacacccaccccgacgcgcgcaaattaatccttccacaaactcttaccccacgccccgaaccacgaacgactacaaatacttcccgacgaacaactaatccaacactttacgataaacctcgttaaaaatcccgtaaacgacatctctaacgaaaaaacaaaatacgcgcccaaaatcactatttctatccccaaataaccgctaaaaacccccttattaaacgcgcgccccctcgacgaccgaaaaaactaacaaaaaaacctccaaaaacccaaaactcgccaataactcccgaaataatctcgaaaacaactactaaaatcgaaaactaatcccgactctaaaaataacaaatacgaaatataaattccttaacgccactcactaccactctttaaaccacaacccccctcgatccccacgcgaccctcaaatccacacaaacccgcccgctactaattttctatcgctacaataaaatcgaaccaaaaaaaaaacaaacttaaaaactttcacgaaaaaaaaaaaaaaatcctaaataaatcaccacactacccctcccgtcaccgcaatcccgaactcccaaccgcgaatatacgcgaaaccgcgcgaatctcaccttctcccgaccgaacgataccaaacgacgacgacgcgcgacgcgaaaacgaaactccctacgcccgacgactccgaaccatcgaaatctaacaacgaatttccgtcctcgccgtacgcctcttcctctaacctacgaaataaccgaaaacaaaaaatataatcaatacaaattcctccccaactcgacgaaaatccaaaataaaaaactcgcgcgaattttcacaaataataaaaatcctccgctaaaaattatctcttctcccaaaactaaaaataaaaacaaaaataaaaaaaaaacaacgacacccaaccactcctaccccgccgaacgacccgcccacccccccttcgcgaataaaaaatcgtcctacgaaccacgacctcaacaaacgctaaaaaaccgaaacgtctccgccgatcgcgcacacctatctacgaaaaaaaaccgcccccctccccgcctaactcccgaacctccccaaaacaacaacgaacgaaaacgaaaaaaaccctaaaaaaccgaacgctaacccacgcgaaaacccgaataaaaaaacccaaaaaacaaaaaaaaaaatacgtaaaacgtacgaaaaccaactcctcaaccacccccaccccaacctccgaacccaaaaaaacccttctccgaacaaatataaatcacgaaaaacctcgatttctattttaaacaaacgaaaatctaacaaaaacataaataataaatacccctaaacttaaccaattcgacgacgactcaaactataacgcgtccgcaccctccgatacttctcgcacgaaaaaaataccaaacacacgaaaacttaaaaataactaaaaataaaaccaaaatattttaaaaccactaaacgaataattcgcgctaatctattatcgataacgataccccatctcaaccccaaaacacgaaaaaaaaaaccgaaatccaacgaatacctaacgacttcgcaatcgccctctacaacctccgttcttaacacaaaacgaataaaaataaattaaaacaaatccctctcgctttaaacccctaaaaaaacttaacgtacaaccaacgctaataacactctctaaacgaaattaaaatcgtacttcgctacttcactcgccttttctccatccttaaaaaaacccgaacaaaacaaacgcatcattccgcacgcctcgcacccaacctttactcgacgatacgcctcaaccctaacccgaataccccgaaccctaaaatctttctaaatattaccctacctcgaacccaaatactaaaatcctaatacaaaacccaaaacccatctctccgtccaaatcaataatcaacgaccccccaaaactaatattcctaaacgaaactcgaaaaaaacaacttcgcaaacaaaacgcgcgctcctcgctaccccaaacgccgctaatacgaaccctcccctacccaaaactcatccacaacctaacacatcgatccgcgactctaaaactacctccgaaaacctaaaccgcgcctaccgattaaaaacccga

SEQ ID NO: 57 - DNA, 3318 bp, synthetic construct
  source 1..3318: mol_type = other DNA
ccgaatttctaaccgacaaacgcgacccaaattcccgaaaacaaccccaaaatcgcgaaccgatataccaaactataaataaaccctaaataaaaaaaaattcgtaccaacgacgcctaaaacaacgaaaaacgcgcgttctacctacgaaactaccttctccgaaccccgcccaaaaacattaactctaaaaaaccgctaatcattaatttaaacgaaaaaataaattctaaattctatattaaaattccaacatctaaactcgaaacaaaacaatatccaaaaaaaccccaaaattcgaaatacccgaaccaaaactaaaacgcatcgccgaacaaaaactaaatacgaaacgtacgaaataatacgcttaccttacccgaacctctccaaaaataaaaaaaaaacgaataaaacaacgaaatacgactccaaccccgcccaaaaaatactactaacgctaactacacgccaaatctctccaaaaatccaaaacgaaaaaaatttattttaacccatctctacccgtcctatatcaaaaacgaaaactataaaaaacgactacgaaatcgccaaacactcgctaaatctcgatccccctcctcgtactctaaaattaaaataaaacaccgccatcgataacaaatcaacgcgaactattcgtttaataaccttaaaacaccctaatttcaccctcaactattttcaaattcccgtatacctaacactttctccgtacgaaaaacaccgaaaaatacgaacgcgccacaatctaaaccgccgccgaactaactaaatttaaaaacatttattattcatattcctaccaaatcctcgcctacccaaaataaaaaccgaaattctccgtaacctacatctactcgaaaaaaaactcccctaaactcgaaaactaaaataaaaataactaaaaaattaacccccgcacgccccacgcatcctctcctttactttctaaacctccccattcgaatcttcgcgtaaatcaacgcccgatctcccaaaacctttctcgtccccgcccgttactactttaaaaaaactcgaaaaccaaacgaaaaaaaaaacgatccttttccgtaaacaaatatacgcgatcgacgaaaacgcctcgatttcccaacgcttattaaaaccgtaacccgcaaaacgaccctttacccgcgaaaaaaaaataaacgaaccgcccgacgaaataaaaataattaaatatcgttacctcctccttacctctactcccacccccaatcctaaaaaaaaaaacaattctcaacgaaaaacttttatcacctataaaaatccgcgcgaaccccttactttaaatcctcgccgaactaaaaaaaaacttacactaaccacaccttctatccccgaccaccccgcaaaccaaaaaaaaaaacgtacgacgaaaacgaaaacccgctaccaaacttcgataactcgaaaccgccgaacgcaaaaaaccccgcctccgcgccgcgcgccgccgccgcctaataccgcccgaccgaaaaaaaataaaattcgcgcgacctcgcgcacacccgcgactaaaaactcgaaactacgataacgaaaaaaacaatataataacccacccaaaatttttttttttttcccgtaaaaatcctcaaacctatcctctccctaacccgatcctattacaacgacaaaaaatcaacaacgaacgaatctatataaacctaaaaaccgcgtaaaaaccgaaaaaaactataacccaaaaaataacaataaataacgtcaaaaaacccacactccgcatctaccactcctaaaaccgaaactaactcccgatcctaacaattactctcgaaatcatcccgaaaattattaacgaattctaaacctctaaaaatttccctatcaacctccccgaccgccgaaaaaacgcgcgcccaacaaaaaaatctctaacgaccacctaaaaacaaaaacaataaccctaaacgcgcactttacctccccgttaaaaatatcgcccacgaaatccttaacgaaacctaccgcaaaatactaaaccaactatccgccgaaaaacacctacaatcgctcgtaacccgaaacgtaaaataaaaatttataaaaaaattaacctacgcgcgccgaaataaatacctatacgaaacgcgcgaaacgaacgacgataaatacccgtaaaaaccaaaataaatctacgcccctaaatctaaaataaacatccgccacgaatcgcaattaaaaattttaaaataacactttaaatttacccaaaaaactctaaaaaaaacaaaaaaaaaacgcgaaccaaaaaatttaatccgttttaaataaaaaaaaaaaaaaaccaaaccaaacctctcaatcatccaaaaccttcaaacttccaaaaaaattttactataattttctctaaacataactatttctaaaaaaaaaaaaaaaaataattatatttactaaaaattcaaatcgaaataataaataaccaaatataaacacttataaacccaaacaattttactcacgccaaaaaaactaaaaacacaaaacttatataaaacctatctcgacaaaaaacaacattctaataaaacccgtaaaaaaacaaattacattttcgccataaataaatcatacaataaaaaatattacctacaacctatcgacttatattattatcacgtttttcaactcgacgtaaaaaaaaaaaaaaatattcatatttaactaaaaattacaaaatcgatacaaactccaaaacaacaaccaaactaacatatataaaactctccgattactttctctatatatcgcgaataaaaaaaacaacgaaaacaatttaacgcaaacacacgaaaaatcgaatctcaaaaaaacaacgctaaaaaaaaaattaaacttaaaacgcgcgtcgcctacccgaatcttatcccgaaccccctccgcaaaatttaataccaaaaaatcctacgtaaaaaaaaaaacatcgaaaaactaccgtctcgaccctccccacgactacttccaaacaaaaacgaacgacgcgataaacaatacgaaccccgaaccctccccgaaaactcccgcgtaaaataaaacccgcctactccccgcgacgattaaacctatatctcccgccccgccaccctcttcccgacccctttacttacaataaaaacctcgacgacgacgcgaaaaacgacgcgaaaccgctctccaaacgccactcgaacgaaatcttcacgaacaactacaaccgctaccgaaaacaaataactatcaaaaaatacttaacgaccg

SEQ ID NO: 58 - DNA, 3318 bp, synthetic construct
  source 1..3318: mol_type = other DNA
cggtcgttaagtattttttgatagttatttgttttcggtagcggttgtagttgttcgtgaagatttcgttcgagtggcgtttggagagcggtttcgcgtcgtttttcgcgtcgtcgtcgaggtttttattgtaagtaaaggggtcgggaagagggtggcggggcgggagatataggtttaatcgtcgcggggagtaggcgggttttattttacgcgggagttttcggggagggttcggggttcgtattgtttatcgcgtcgttcgtttttgtttggaagtagtcgtggggagggtcgagacggtagtttttcgatgtttttttttttacgtaggattttttggtattaaattttgcggagggggttcgggataagattcgggtaggcgacgcgcgttttaagtttaattttttttttagcgttgtttttttgagattcgatttttcgtgtgtttgcgttaaattgttttcgttgttttttttattcgcgatatatagagaaagtaatcggagagttttatatatgttagtttggttgttgttttggagtttgtatcgattttgtaatttttagttaaatatgaatatttttttttttttttacgtcgagttgaaaaacgtgataataatataagtcgataggttgtaggtaatattttttattgtatgatttatttatggcgaaaatgtaatttgtttttttacgggttttattagaatgttgttttttgtcgagataggttttatataagttttgtgtttttagtttttttggcgtgagtaaaattgtttgggtttataagtgtttatatttggttatttattatttcgatttgaatttttagtaaatataattatttttttttttttttttagaaatagttatgtttagagaaaattatagtaaaatttttttggaagtttgaaggttttggatgattgagaggtttggtttggtttttttttttttttatttaaaacggattaaattttttggttcgcgttttttttttgttttttttagagttttttgggtaaatttaaagtgttattttaaaatttttaattgcgattcgtggcggatgtttattttagatttaggggcgtagatttattttggtttttacgggtatttatcgtcgttcgtttcgcgcgtttcgtataggtatttatttcggcgcgcgtaggttaatttttttataaatttttattttacgtttcgggttacgagcgattgtaggtgtttttcggcggatagttggtttagtattttgcggtaggtttcgttaaggatttcgtgggcgatatttttaacggggaggtaaagtgcgcgtttagggttattgtttttgtttttaggtggtcgttagagatttttttgttgggcgcgcgttttttcggcggtcggggaggttgatagggaaatttttagaggtttagaattcgttaataattttcgggatgatttcgagagtaattgttaggatcgggagttagtttcggttttaggagtggtagatgcggagtgtgggttttttgacgttatttattgttattttttgggttatagtttttttcggtttttacgcggtttttaggtttatatagattcgttcgttgttgattttttgtcgttgtaataggatcgggttagggagaggataggtttgaggatttttacgggaaaaaaaaaaaaattttgggtgggttattatattgtttttttcgttatcgtagtttcgagtttttagtcgcgggtgtgcgcgaggtcgcgcgaattttattttttttcggtcgggcggtattaggcggcggcggcgcgcggcgcggaggcggggttttttgcgttcggcggtttcgagttatcgaagtttggtagcgggttttcgttttcgtcgtacgtttttttttttggtttgcggggtggtcggggatagaaggtgtggttagtgtaagtttttttttagttcggcgaggatttaaagtaaggggttcgcgcggatttttataggtgataaaagtttttcgttgagaattgtttttttttttaggattgggggtgggagtagaggtaaggaggaggtaacgatatttaattatttttatttcgtcgggcggttcgtttatttttttttcgcgggtaaagggtcgttttgcgggttacggttttaataagcgttgggaaatcgaggcgttttcgtcgatcgcgtatatttgtttacggaaaaggatcgttttttttttcgtttggttttcgagtttttttaaagtagtaacgggcggggacgagaaaggttttgggagatcgggcgttgatttacgcgaagattcgaatggggaggtttagaaagtaaaggagaggatgcgtggggcgtgcgggggttaattttttagttatttttattttagttttcgagtttaggggagttttttttcgagtagatgtaggttacggagaatttcggtttttattttgggtaggcgaggatttggtaggaatatgaataataaatgtttttaaatttagttagttcggcggcggtttagattgtggcgcgttcgtatttttcggtgtttttcgtacggagaaagtgttaggtatacgggaatttgaaaatagttgagggtgaaattagggtgttttaaggttattaaacgaatagttcgcgttgatttgttatcgatggcggtgttttattttaattttagagtacgaggagggggatcgagatttagcgagtgtttggcgatttcgtagtcgttttttatagttttcgtttttgatataggacgggtagagatgggttaaaataaatttttttcgttttggatttttggagagatttggcgtgtagttagcgttagtagtattttttgggcggggttggagtcgtatttcgttgttttattcgttttttttttatttttggagaggttcgggtaaggtaagcgtattatttcgtacgtttcgtatttagtttttgttcggcgatgcgttttagttttggttcgggtatttcgaattttggggtttttttggatattgttttgtttcgagtttagatgttggaattttaatatagaatttagaatttattttttcgtttaaattaatgattagcggttttttagagttaatgtttttgggcggggttcggagaaggtagtttcgtaggtagaacgcgcgtttttcgttgttttaggcgtcgttggtacgaatttttttttatttagggtttatttatagtttggtatatcggttcgcgattttggggttgttttcgggaatttgggtcgcgtttgtcggttagaaattcgg

SEQ ID NO: 59 - DNA, 1094 bp, Homo sapiens
  source 1..1094: mol_type = genomic DNA
gtaggggagggttcgtaccagcggcgcctggggcagcgaggagcgcgcgttctgcctgcgaagctgccttctccgagccccgcccaggaacattagctctggggggccgctgatcattgatttggacggagagatgggttctgggttctgtattaggattccagcatctgggctcgaggcagggcaatatccagaaagaccccagggttcggggtacccgggccagggctgaggcgcatcgccgagcaaaggctgggtgcgaggcgtgcggaatgatgcgcttgccttgcccgggcctctccaaggatggagaaaaggcgagtgaagcagcgaagtacgactccaaccccgcccagagagtgctactagcgctggctgcacgccaagtctctccaggggtccaaagcgagagggatttgttttaacccatctctacccgtcctgtgtcaagaacggaggctgtagagggcgactgcgaagtcgccaggcactcgctggatctcggtccccctcctcgtgctctggggttgagatggggcaccgccatcgataacagatcagcgcgaactattcgtttagtggccttaaaacaccctggtttcaccctcagctattttcaagttcccgtgtgcctggcactttctccgtgcgagaagcaccggagggtgcggacgcgccacagtctgagccgccgccgaactggctaagtttaggggcatttattattcatgttcctgccagatcctcgcctgcccaaaatagaaaccgaggttctccgtgacctacatctgctcggagaagggctcccctgggctcggaggctggggtgggggtggctgaggagttggcccccgcacgccccacgcatcctctcctttgctttctgggcctccccattcgggtcttcgcgtgggtcagcgcccggtctcccagggcctttctcgtccccgcccgttgctgctttggggaggctcgggagccaggcggggaggggggcggtccttttccgtagacaggtgtgcgcgatcggcggagacgcctcggtttcccagcgcttgttgaggccgtggcccgcaggacgaccctttacccgc

SEQ ID NO: 60 - DNA, 1094 bp, synthetic construct
  source 1..1094: mol_type = other DNA
gtaggggagggttcgtattagcggcgtttggggtagcgaggagcgcgcgttttgtttgcgaagttgtttttttcgagtttcgtttaggaatattagttttggggggtcgttgattattgatttggacggagagatgggttttgggttttgtattaggattttagtatttgggttcgaggtagggtaatatttagaaagattttagggttcggggtattcgggttagggttgaggcgtatcgtcgagtaaaggttgggtgcgaggcgtgcggaatgatgcgtttgttttgttcgggtttttttaaggatggagaaaaggcgagtgaagtagcgaagtacgattttaatttcgtttagagagtgttattagcgttggttgtacgttaagtttttttaggggtttaaagcgagagggatttgttttaatttatttttattcgttttgtgttaagaacggaggttgtagagggcgattgcgaagtcgttaggtattcgttggatttcggttttttttttcgtgttttggggttgagatggggtatcgttatcgataatagattagcgcgaattattcgtttagtggttttaaaatattttggttttatttttagttatttttaagttttcgtgtgtttggtatttttttcgtgcgagaagtatcggagggtgcggacgcgttatagtttgagtcgtcgtcgaattggttaagtttaggggtatttattatttatgtttttgttagattttcgtttgtttaaaatagaaatcgaggtttttcgtgatttatatttgttcggagaagggtttttttgggttcggaggttggggtgggggtggttgaggagttggttttcgtacgttttacgtatttttttttttgttttttgggtttttttattcgggttttcgcgtgggttagcgttcggttttttagggtttttttcgttttcgttcgttgttgttttggggaggttcgggagttaggcggggaggggggcggttttttttcgtagataggtgtgcgcgatcggcggagacgtttcggttttttagcgtttgttgaggtcgtggttcgtaggacgattttttattcgt

SEQ ID NO: 61 - DNA, 1094 bp, synthetic construct
  source 1..1094: mol_type = other DNA
acgaataaaaaatcgtcctacgaaccacgacctcaacaaacgctaaaaaaccgaaacgtctccgccgatcgcgcacacctatctacgaaaaaaaaccgcccccctccccgcctaactcccgaacctccccaaaacaacaacgaacgaaaacgaaaaaaaccctaaaaaaccgaacgctaacccacgcgaaaacccgaataaaaaaacccaaaaaacaaaaaaaaaaatacgtaaaacgtacgaaaaccaactcctcaaccacccccaccccaacctccgaacccaaaaaaacccttctccgaacaaatataaatcacgaaaaacctcgatttctattttaaacaaacgaaaatctaacaaaaacataaataataaatacccctaaacttaaccaattcgacgacgactcaaactataacgcgtccgcaccctccgatacttctcgcacgaaaaaaataccaaacacacgaaaacttaaaaataactaaaaataaaaccaaaatattttaaaaccactaaacgaataattcgcgctaatctattatcgataacgataccccatctcaaccccaaaacacgaaaaaaaaaaccgaaatccaacgaatacctaacgacttcgcaatcgccctctacaacctccgttcttaacacaaaacgaataaaaataaattaaaacaaatccctctcgctttaaacccctaaaaaaacttaacgtacaaccaacgctaataacactctctaaacgaaattaaaatcgtacttcgctacttcactcgccttttctccatccttaaaaaaacccgaacaaaacaaacgcatcattccgcacgcctcgcacccaacctttactcgacgatacgcctcaaccctaacccgaataccccgaaccctaaaatctttctaaatattaccctacctcgaacccaaatactaaaatcctaatacaaaacccaaaacccatctctccgtccaaatcaataatcaacgaccccccaaaactaatattcctaaacgaaactcgaaaaaaacaacttcgcaaacaaaacgcgcgctcctcgctaccccaaacgccgctaatacgaaccctcccctac

SEQ ID NO: 62 - DNA, 1094 bp, synthetic construct
  source 1..1094: mol_type = other DNA
ataaaaaaaaattcgtaccaacgacgcctaaaacaacgaaaaacgcgcgttctacctacgaaactaccttctccgaaccccgcccaaaaacattaactctaaaaaaccgctaatcattaatttaaacgaaaaaataaattctaaattctatattaaaattccaacatctaaactcgaaacaaaacaatatccaaaaaaaccccaaaattcgaaatacccgaaccaaaactaaaacgcatcgccgaacaaaaactaaatacgaaacgtacgaaataatacgcttaccttacccgaacctctccaaaaataaaaaaaaaacgaataaaacaacgaaatacgactccaaccccgcccaaaaaatactactaacgctaactacacgccaaatctctccaaaaatccaaaacgaaaaaaatttattttaacccatctctacccgtcctatatcaaaaacgaaaactataaaaaacgactacgaaatcgccaaacactcgctaaatctcgatccccctcctcgtactctaaaattaaaataaaacaccgccatcgataacaaatcaacgcgaactattcgtttaataaccttaaaacaccctaatttcaccctcaactattttcaaattcccgtatacctaacactttctccgtacgaaaaacaccgaaaaatacgaacgcgccacaatctaaaccgccgccgaactaactaaatttaaaaacatttattattcatattcctaccaaatcctcgcctacccaaaataaaaaccgaaattctccgtaacctacatctactcgaaaaaaaactcccctaaactcgaaaactaaaataaaaataactaaaaaattaacccccgcacgccccacgcatcctctcctttactttctaaacctccccattcgaatcttcgcgtaaatcaacgcccgatctcccaaaacctttctcgtccccgcccgttactactttaaaaaaactcgaaaaccaaacgaaaaaaaaaacgatccttttccgtaaacaaatatacgcgatcgacgaaaacgcctcgatttcccaacgcttattaaaaccgtaacccgcaaaacgaccctttacccgc

SEQ ID NO: 63 - DNA, 1094 bp, synthetic construct
  source 1..1094: mol_type = other DNA
gcgggtaaagggtcgttttgcgggttacggttttaataagcgttgggaaatcgaggcgttttcgtcgatcgcgtatatttgtttacggaaaaggatcgttttttttttcgtttggttttcgagtttttttaaagtagtaacgggcggggacgagaaaggttttgggagatcgggcgttgatttacgcgaagattcgaatggggaggtttagaaagtaaaggagaggatgcgtggggcgtgcgggggttaattttttagttatttttattttagttttcgagtttaggggagttttttttcgagtagatgtaggttacggagaatttcggtttttattttgggtaggcgaggatttggtaggaatatgaataataaatgtttttaaatttagttagttcggcggcggtttagattgtggcgcgttcgtatttttcggtgtttttcgtacggagaaagtgttaggtatacgggaatttgaaaatagttgagggtgaaattagggtgttttaaggttattaaacgaatagttcgcgttgatttgttatcgatggcggtgttttattttaattttagagtacgaggagggggatcgagatttagcgagtgtttggcgatttcgtagtcgttttttatagttttcgtttttgatataggacgggtagagatgggttaaaataaatttttttcgttttggatttttggagagatttggcgtgtagttagcgttagtagtattttttgggcggggttggagtcgtatttcgttgttttattcgttttttttttatttttggagaggttcgggtaaggtaagcgtattatttcgtacgtttcgtatttagtttttgttcggcgatgcgttttagttttggttcgggtatttcgaattttggggtttttttggatattgttttgtttcgagtttagatgttggaattttaatatagaatttagaatttattttttcgtttaaattaatgattagcggttttttagagttaatgtttttgggcggggttcggagaaggtagtttcgtaggtagaacgcgcgtttttcgttgttttaggcgtcgttggtacgaatttttttttat

SEQ ID NO: 64 - DNA, 94 bp, Homo sapiens
  source 1..94: mol_type = genomic DNA
ctcggtccccctcctcgtgctctggggttgagatggggcaccgccatcgataacagatcagcgcgaactattcgtttagtggccttaaaacacc

SEQ ID NO: 65 - DNA, 94 bp, synthetic construct
  source 1..94: mol_type = other DNA
ttcggttttttttttcgtgttttggggttgagatggggtatcgttatcgataatagattagcgcgaattattcgtttagtggttttaaaatatt

SEQ ID NO: 66 - DNA, 94 bp, synthetic construct
  source 1..94: mol_type = other DNA
aatattttaaaaccactaaacgaataattcgcgctaatctattatcgataacgataccccatctcaaccccaaaacacgaaaaaaaaaaccgaa

SEQ ID NO: 67 - DNA, 94 bp, synthetic construct
  source 1..94: mol_type = other DNA
ctcgatccccctcctcgtactctaaaattaaaataaaacaccgccatcgataacaaatcaacgcgaactattcgtttaataaccttaaaacacc

SEQ ID NO: 68 - DNA, 94 bp, synthetic construct
  source 1..94: mol_type = other DNA
ggtgttttaaggttattaaacgaatagttcgcgttgatttgttatcgatggcggtgttttattttaattttagagtacgaggagggggatcgag

SEQ ID NO: 69 - DNA, 1079 bp, Homo sapiens
  source 1..1079: mol_type = genomic DNA
ctctaggaatgaaatcccctttaacattacaggtacacaaaagaaaatgcacacttggtaaaacagttgatcaattatagagtgttcccagggaccaatatctcacagagtacccaatcatcacacttcaaataaatgtataagcactttagcctttacttaccacattaacatacatataagaacatgcatagtttaatgcataggttaattctaacactttccataggatagactttgattaaagggtatcaaaaatcacttcctaaatgcagcaaaaaataaaaataaaaataccgatctgtagctacgaagctcttagaacactacattgatcaccatttacttccaaactgctttcccttccctctgcgaagtgaaaatgtccctgtagaccgcgttacatacactttatggtttctctatcaatgatcctaacaggtcacatcgccactgccattagatctccgtagctttggtcttctccaaattaagacgtggagaaggagcactgctgtccgccccaagaacggggtctgtccgcccgagctctaaggcgagcatcttcagggggacagtttctgcgaaggcggggagaggagtccctccccaacttcactcccctaatcaagccgcgggtgagataggcagccggcggtttgtgcccatctgtgggggcaagtcctaccttctgccaaaaggcgcgacgcatgcacaaaagatggatccaggctatctttctctgccatcagctcaggcaaatatttctcctcttccatagcgcggacttcggattgtccccgggcgaagcgcgaggttccgctcgctcggacgcaggcagggtcttggggcagcgcctggctcccgcgctgctcctcctccgcgcggcgagggatctctgtgcgtcctcactggcccatgcacccagcacctgcgactcccgccgtcgggctgcgtggccccgcgcccacacctgcccgtcccttccgtcgtccctcgctcgcgcagagccccggctcacaccagcggccttaactggagaggcgggaacaggacgcggcccacctccgccaaccact

SEQ ID NO: 70 - DNA, 1079 bp, synthetic construct
  source 1..1079: mol_type = other DNA
ttttaggaatgaaattttttttaatattataggtatataaaagaaaatgtatatttggtaaaatagttgattaattatagagtgtttttagggattaatattttatagagtatttaattattatattttaaataaatgtataagtattttagtttttatttattatattaatatatatataagaatatgtatagtttaatgtataggttaattttaatattttttataggatagattttgattaaagggtattaaaaattattttttaaatgtagtaaaaaataaaaataaaaatatcgatttgtagttacgaagtttttagaatattatattgattattatttatttttaaattgtttttttttttttttgcgaagtgaaaatgtttttgtagatcgcgttatatatattttatggtttttttattaatgattttaataggttatatcgttattgttattagattttcgtagttttggttttttttaaattaagacgtggagaaggagtattgttgttcgttttaagaacggggtttgttcgttcgagttttaaggcgagtatttttagggggatagtttttgcgaaggcggggagaggagtttttttttaattttatttttttaattaagtcgcgggtgagataggtagtcggcggtttgtgtttatttgtgggggtaagttttattttttgttaaaaggcgcgacgtatgtataaaagatggatttaggttatttttttttgttattagtttaggtaaatatttttttttttttatagcgcggatttcggattgttttcgggcgaagcgcgaggtttcgttcgttcggacgtaggtagggttttggggtagcgtttggttttcgcgttgtttttttttcgcgcggcgagggatttttgtgcgtttttattggtttatgtatttagtatttgcgattttcgtcgtcgggttgcgtggtttcgcgtttatatttgttcgtttttttcgtcgtttttcgttcgcgtagagtttcggtttatattagcggttttaattggagaggcgggaataggacgcggtttattttcgttaattatt

SEQ ID NO: 71 - DNA, 1079 bp, synthetic construct
  source 1..1079: mol_type = other DNA
aataattaacgaaaataaaccgcgtcctattcccgcctctccaattaaaaccgctaatataaaccgaaactctacgcgaacgaaaaacgacgaaaaaaacgaacaaatataaacgcgaaaccacgcaacccgacgacgaaaatcgcaaatactaaatacataaaccaataaaaacgcacaaaaatccctcgccgcgcgaaaaaaaaacaacgcgaaaaccaaacgctaccccaaaaccctacctacgtccgaacgaacgaaacctcgcgcttcgcccgaaaacaatccgaaatccgcgctataaaaaaaaaaaaatatttacctaaactaataacaaaaaaaaataacctaaatccatcttttatacatacgtcgcgccttttaacaaaaaataaaacttacccccacaaataaacacaaaccgccgactacctatctcacccgcgacttaattaaaaaaataaaattaaaaaaaaactcctctccccgccttcgcaaaaactatccccctaaaaatactcgccttaaaactcgaacgaacaaaccccgttcttaaaacgaacaacaatactccttctccacgtcttaatttaaaaaaaaccaaaactacgaaaatctaataacaataacgatataacctattaaaatcattaataaaaaaaccataaaatatatataacgcgatctacaaaaacattttcacttcgcaaaaaaaaaaaaaaacaatttaaaaataaataataatcaatataatattctaaaaacttcgtaactacaaatcgatatttttatttttattttttactacatttaaaaaataatttttaataccctttaatcaaaatctatcctataaaaaatattaaaattaacctatacattaaactatacatattcttatatatatattaatataataaataaaaactaaaatacttatacatttatttaaaatataataattaaatactctataaaatattaatccctaaaaacactctataattaatcaactattttaccaaatatacattttcttttatatacctataatattaaaaaaaatttcattcctaaaa

SEQ ID NO: 72 - DNA, 1079 bp, synthetic construct
  source 1..1079: mol_type = other DNA
ctctaaaaataaaatcccctttaacattacaaatacacaaaaaaaaatacacacttaataaaacaattaatcaattataaaatattcccaaaaaccaatatctcacaaaatacccaatcatcacacttcaaataaatatataaacactttaacctttacttaccacattaacatacatataaaaacatacataatttaatacataaattaattctaacactttccataaaataaactttaattaaaaaatatcaaaaatcacttcctaaatacaacaaaaaataaaaataaaaataccgatctataactacgaaactcttaaaacactacattaatcaccatttacttccaaactactttcccttccctctacgaaataaaaatatccctataaaccgcgttacatacactttataatttctctatcaataatcctaacaaatcacatcgccactaccattaaatctccgtaactttaatcttctccaaattaaaacgtaaaaaaaaaacactactatccgccccaaaaacgaaatctatccgcccgaactctaaaacgaacatcttcaaaaaaacaatttctacgaaaacgaaaaaaaaaatccctccccaacttcactcccctaatcaaaccgcgaataaaataaacaaccgacgatttatacccatctataaaaacaaatcctaccttctaccaaaaaacgcgacgcatacacaaaaaataaatccaaactatctttctctaccatcaactcaaacaaatatttctcctcttccataacgcgaacttcgaattatccccgaacgaaacgcgaaattccgctcgctcgaacgcaaacaaaatcttaaaacaacgcctaactcccgcgctactcctcctccgcgcgacgaaaaatctctatacgtcctcactaacccatacacccaacacctacgactcccgccgtcgaactacgtaaccccgcgcccacacctacccgtcccttccgtcgtccctcgctcgcgcaaaaccccgactcacaccaacgaccttaactaaaaaaacgaaaacaaaacgcgacccacctccgccaaccact

SEQ ID NO: 73 - DNA, 1079 bp, synthetic construct
  source 1..1079: mol_type = other DNA
agtggttggcggaggtgggtcgcgttttgttttcgtttttttagttaaggtcgttggtgtgagtcggggttttgcgcgagcgagggacgacggaagggacgggtaggtgtgggcgcggggttacgtagttcgacggcgggagtcgtaggtgttgggtgtatgggttagtgaggacgtatagagatttttcgtcgcgcggaggaggagtagcgcgggagttaggcgttgttttaagattttgtttgcgttcgagcgagcggaatttcgcgtttcgttcggggataattcgaagttcgcgttatggaagaggagaaatatttgtttgagttgatggtagagaaagatagtttggatttattttttgtgtatgcgtcgcgttttttggtagaaggtaggatttgtttttatagatgggtataaatcgtcggttgtttattttattcgcggtttgattaggggagtgaagttggggagggattttttttttcgttttcgtagaaattgtttttttgaagatgttcgttttagagttcgggcggatagatttcgtttttggggcggatagtagtgttttttttttacgttttaatttggagaagattaaagttacggagatttaatggtagtggcgatgtgatttgttaggattattgatagagaaattataaagtgtatgtaacgcggtttatagggatatttttatttcgtagagggaagggaaagtagtttggaagtaaatggtgattaatgtagtgttttaagagtttcgtagttatagatcggtatttttatttttattttttgttgtatttaggaagtgatttttgatattttttaattaaagtttattttatggaaagtgttagaattaatttatgtattaaattatgtatgtttttatatgtatgttaatgtggtaagtaaaggttaaagtgtttatatatttatttgaagtgtgatgattgggtattttgtgagatattggtttttgggaatattttataattgattaattgttttattaagtgtgtatttttttttgtgtatttgtaatgttaaaggggattttatttttagag

SEQ ID NO: 74 - DNA, 79 bp, Homo sapiens
  source 1..79: mol_type = genomic DNA
gagaaggagcactgctgtccgccccaagaacggggtctgtccgcccgagctctaaggcgagcatcttcagggggacagt

SEQ ID NO: 75 - DNA, 79 bp, synthetic construct
  source 1..79: mol_type = other DNA
gagaaggagtattgttgttcgttttaagaacggggtttgttcgttcgagttttaaggcgagtatttttagggggatagt

SEQ ID NO: 76 - DNA, 79 bp, synthetic construct
  source 1..79: mol_type = other DNA
actatccccctaaaaatactcgccttaaaactcgaacgaacaaaccccgttcttaaaacgaacaacaatactccttctc

SEQ ID NO: 77 - DNA, 79 bp, synthetic construct
  source 1..79: mol_type = other DNA
aaaaaaaaacactactatccgccccaaaaacgaaatctatccgcccgaactctaaaacgaacatcttcaaaaaaacaat

SEQ ID NO: 78 - DNA, 79 bp, synthetic construct
  source 1..79: mol_type = other DNA
attgtttttttgaagatgttcgttttagagttcgggcggatagatttcgtttttggggcggatagtagtgttttttttt

SEQ ID NO: 79 - DNA, 1284 bp, Homo sapiens
  source 1..1284: mol_type = genomic DNA
tcgtcgaccctgattggccatgttctctgcggcacggggctggttgcagtggccggcgggcgcctggtgggcaccccggtccccccaagggtcggctgtccttccccactggtcacacaagagcggccgtccttccccagctcgaagcccgtagcacattcgcaggcaaagcctcccaagtcgtctaggcagttagggagctctgcgcatttgccagcacggaggtacctcccggggcagggacacaacacatcgcccgagagtttgtcccagcgagcgccgatttcgtccgcgatgcaagtaactgagatcgggagctgtccccggcagagcgcactcacctcggtcccaggtggactgaagtccagagcggcgctgtgcagctggaagggcgcgcgatagctcaagttagaggcggccccggggcgcggcgcaggacacaagacctcaaactggtacttgcacaggtagccgttggcgcgcaggtggcatcgcatctccttccagcctgcgggctcgaccccaccggtggcctggagtaccgcgcatctccgcgcggtgcaggagcgttggggctcctccacccactgcagcgtgtcgctttcgagaccgccggggtcggaggacagccaggagaaaccccgcaaaggctcgttctccagggtgcagtgggaacgcctgcgctccagtgcgacccagaacagcaggtctttggagccccctccgggccctgggcctgcccgcaggagcgcgagcacagcgcgcagctcggcgcccgcacgcacggtgctgagcgccccacctcgcaggatgcaggcctcctcggccgcctgccgcttcatggtagcgtggtgcaggctgtagcaggcccccgaggccgagcagccagcacggtcggcagtggggtgttcgccgccgcccggcccgggccagagcgcctgccagaggaggcacagggcgaacgccggcctcattctctgaggccccgcacgcagagctgctcccaacttggatctgtcccgctcgaggacgcagagctgtgtctggagcgcggtcaccgccctctacagctggcttccccaacccggttccccctcccttcccctccgccctcgctcccgacggaccccccaactgccaagaatccccccgcgggcccagcggccaacagtttcccttaggccgcgacaggaaatgtgtgcggagcgctgccaggcccggtgcagccctgagttattctggggtcaggaacacagctggcgccgggtacaggggacgcagtcg

SEQ ID NO: 80 - DNA, 1284 bp, synthetic construct
  source 1..1284: mol_type = other DNA
tcgtcgattttgattggttatgttttttgcggtacggggttggttgtagtggtcggcgggcgtttggtgggtatttcggtttttttaagggtcggttgtttttttttattggttatataagagcggtcgtttttttttagttcgaagttcgtagtatattcgtaggtaaagttttttaagtcgtttaggtagttagggagttttgcgtatttgttagtacggaggtatttttcggggtagggatataatatatcgttcgagagtttgttttagcgagcgtcgatttcgttcgcgatgtaagtaattgagatcgggagttgttttcggtagagcgtatttatttcggttttaggtggattgaagtttagagcggcgttgtgtagttggaagggcgcgcgatagtttaagttagaggcggtttcggggcgcggcgtaggatataagattttaaattggtatttgtataggtagtcgttggcgcgtaggtggtatcgtatttttttttagtttgcgggttcgattttatcggtggtttggagtatcgcgtattttcgcgcggtgtaggagcgttggggtttttttatttattgtagcgtgtcgttttcgagatcgtcggggtcggaggatagttaggagaaatttcgtaaaggttcgttttttagggtgtagtgggaacgtttgcgttttagtgcgatttagaatagtaggtttttggagtttttttcgggttttgggtttgttcgtaggagcgcgagtatagcgcgtagttcggcgttcgtacgtacggtgttgagcgttttatttcgtaggatgtaggttttttcggtcgtttgtcgttttatggtagcgtggtgtaggttgtagtaggttttcgaggtcgagtagttagtacggtcggtagtggggtgttcgtcgtcgttcggttcgggttagagcgtttgttagaggaggtatagggcgaacgtcggttttattttttgaggtttcgtacgtagagttgtttttaatttggatttgtttcgttcgaggacgtagagttgtgtttggagcgcggttatcgttttttatagttggtttttttaattcggtttttttttttttttttttcgttttcgttttcgacggattttttaattgttaagaattttttcgcgggtttagcggttaatagttttttttaggtcgcgataggaaatgtgtgcggagcgttgttaggttcggtgtagttttgagttattttggggttaggaatatagttggcgtcgggtataggggacgtagtcg

SEQ ID NO: 81 - DNA, 1284 bp, synthetic construct
  source 1..1284: mol_type = other DNA
cgactacgtcccctatacccgacgccaactatattcctaaccccaaaataactcaaaactacaccgaacctaacaacgctccgcacacatttcctatcgcgacctaaaaaaaactattaaccgctaaacccgcgaaaaaattcttaacaattaaaaaatccgtcgaaaacgaaaacgaaaaaaaaaaaaaaaaaaaccgaattaaaaaaaccaactataaaaaacgataaccgcgctccaaacacaactctacgtcctcgaacgaaacaaatccaaattaaaaacaactctacgtacgaaacctcaaaaaataaaaccgacgttcgccctatacctcctctaacaaacgctctaacccgaaccgaacgacgacgaacaccccactaccgaccgtactaactactcgacctcgaaaacctactacaacctacaccacgctaccataaaacgacaaacgaccgaaaaaacctacatcctacgaaataaaacgctcaacaccgtacgtacgaacgccgaactacgcgctatactcgcgctcctacgaacaaacccaaaacccgaaaaaaactccaaaaacctactattctaaatcgcactaaaacgcaaacgttcccactacaccctaaaaaacgaacctttacgaaatttctcctaactatcctccgaccccgacgatctcgaaaacgacacgctacaataaataaaaaaaccccaacgctcctacaccgcgcgaaaatacgcgatactccaaaccaccgataaaatcgaacccgcaaactaaaaaaaaatacgataccacctacgcgccaacgactacctatacaaataccaatttaaaatcttatatcctacgccgcgccccgaaaccgcctctaacttaaactatcgcgcgcccttccaactacacaacgccgctctaaacttcaatccacctaaaaccgaaataaatacgctctaccgaaaacaactcccgatctcaattacttacatcgcgaacgaaatcgacgctcgctaaaacaaactctcgaacgatatattatatccctaccccgaaaaatacctccgtactaacaaatacgcaaaactccctaactacctaaacgacttaaaaaactttacctacgaatatactacgaacttcgaactaaaaaaaaacgaccgctcttatataaccaataaaaaaaaacaaccgacccttaaaaaaaccgaaatacccaccaaacgcccgccgaccactacaaccaaccccgtaccgcaaaaaacataaccaatcaaaatcgacga

SEQ ID NO: 82 - DNA, 1284 bp, synthetic construct
  source 1..1284: mol_type = other DNA
tcgtcgaccctaattaaccatattctctacgacacgaaactaattacaataaccgacgaacgcctaataaacaccccgatccccccaaaaatcgactatccttccccactaatcacacaaaaacgaccgtccttccccaactcgaaacccgtaacacattcgcaaacaaaacctcccaaatcgtctaaacaattaaaaaactctacgcatttaccaacacgaaaatacctcccgaaacaaaaacacaacacatcgcccgaaaatttatcccaacgaacgccgatttcgtccgcgatacaaataactaaaatcgaaaactatccccgacaaaacgcactcacctcgatcccaaataaactaaaatccaaaacgacgctatacaactaaaaaaacgcgcgataactcaaattaaaaacgaccccgaaacgcgacgcaaaacacaaaacctcaaactaatacttacacaaataaccgttaacgcgcaaataacatcgcatctccttccaacctacgaactcgaccccaccgataacctaaaataccgcgcatctccgcgcgatacaaaaacgttaaaactcctccacccactacaacgtatcgctttcgaaaccgccgaaatcgaaaaacaaccaaaaaaaaccccgcaaaaactcgttctccaaaatacaataaaaacgcctacgctccaatacgacccaaaacaacaaatctttaaaaccccctccgaaccctaaacctacccgcaaaaacgcgaacacaacgcgcaactcgacgcccgcacgcacgatactaaacgccccacctcgcaaaatacaaacctcctcgaccgcctaccgcttcataataacgtaatacaaactataacaaacccccgaaaccgaacaaccaacacgatcgacaataaaatattcgccgccgcccgacccgaaccaaaacgcctaccaaaaaaaacacaaaacgaacgccgacctcattctctaaaaccccgcacgcaaaactactcccaacttaaatctatcccgctcgaaaacgcaaaactatatctaaaacgcgatcaccgccctctacaactaacttccccaacccgattccccctcccttcccctccgccctcgctcccgacgaaccccccaactaccaaaaatccccccgcgaacccaacgaccaacaatttcccttaaaccgcgacaaaaaatatatacgaaacgctaccaaacccgatacaaccctaaattattctaaaatcaaaaacacaactaacgccgaatacaaaaaacgcaatcg

SEQ ID NO: 83 - DNA, 1284 bp, synthetic construct
  source 1..1284: mol_type = other DNA
cgattgcgttttttgtattcggcgttagttgtgtttttgattttagaataatttagggttgtatcgggtttggtagcgtttcgtatatattttttgtcgcggtttaagggaaattgttggtcgttgggttcgcggggggatttttggtagttggggggttcgtcgggagcgagggcggaggggaagggagggggaatcgggttggggaagttagttgtagagggcggtgatcgcgttttagatatagttttgcgttttcgagcgggatagatttaagttgggagtagttttgcgtgcggggttttagagaatgaggtcggcgttcgttttgtgttttttttggtaggcgttttggttcgggtcgggcggcggcgaatattttattgtcgatcgtgttggttgttcggtttcgggggtttgttatagtttgtattacgttattatgaagcggtaggcggtcgaggaggtttgtattttgcgaggtggggcgtttagtatcgtgcgtgcgggcgtcgagttgcgcgttgtgttcgcgtttttgcgggtaggtttagggttcggagggggttttaaagatttgttgttttgggtcgtattggagcgtaggcgtttttattgtattttggagaacgagtttttgcggggttttttttggttgtttttcgatttcggcggtttcgaaagcgatacgttgtagtgggtggaggagttttaacgtttttgtatcgcgcggagatgcgcggtattttaggttatcggtggggtcgagttcgtaggttggaaggagatgcgatgttatttgcgcgttaacggttatttgtgtaagtattagtttgaggttttgtgttttgcgtcgcgtttcggggtcgtttttaatttgagttatcgcgcgtttttttagttgtatagcgtcgttttggattttagtttatttgggatcgaggtgagtgcgttttgtcggggatagttttcgattttagttatttgtatcgcggacgaaatcggcgttcgttgggataaattttcgggcgatgtgttgtgtttttgtttcgggaggtattttcgtgttggtaaatgcgtagagttttttaattgtttagacgatttgggaggttttgtttgcgaatgtgttacgggtttcgagttggggaaggacggtcgtttttgtgtgattagtggggaaggatagtcgatttttggggggatcggggtgtttattaggcgttcgtcggttattgtaattagtttcgtgtcgtagagaatatggttaattagggtcgacga

SEQ ID NO: 84 - DNA, 1102 bp, Homo sapiens
  source 1..1102: mol_type = genomic DNA
gtggactgaagtccagagcggcgctgtgcagctggaagggcgcgcgatagctcaagttagaggcggccccggggcgcggcgcaggacacaagacctcaaactggtacttgcacaggtagccgttggcgcgcaggtggcatcgcatctccttccagcctgcgggctcgaccccaccggtggcctggagtaccgcgcatctccgcgcggtgcaggagcgttggggctcctccacccactgcagcgtgtcgctttcgagaccgccggggtcggaggacagccaggagaaaccccgcaaaggctcgttctccagggtgcagtgggaacgcctgcgctccagtgcgacccagaacagcaggtctttggagccccctccgggccctgggcctgcccgcaggagcgcgagcacagcgcgcagctcggcgcccgcacgcacggtgctgagcgccccacctcgcaggatgcaggcctcctcggccgcctgccgcttcatggtagcgtggtgcaggctgtagcaggcccccgaggccgagcagccagcacggtcggcagtggggtgttcgccgccgcccggcccgggccagagcgcctgccagaggaggcacagggcgaacgccggcctcattctctgaggccccgcacgcagagctgctcccaacttggatctgtcccgctcgaggacgcagagctgtgtctggagcgcggtcaccgccctctacagctggcttccccaacccggttccccctcccttcccctccgccctcgctcccgacggaccccccaactgccaagaatccccccgcgggcccagcggccaacagtttcccttaggccgcgacaggaaatgtgtgcggagcgctgccaggcccggtgcagccctgagttattctggggtcaggaacacagctggcgccgggtacaggggacgcagtcgactagaaggtcaaacaccctgcttagagcaagaggagaaattcgccactgggatgaagtgtgtggtttttctccgttcagggagtttcatcaggaccgcagaacccataacacgtaagcgaacataggcaggaaggacttgagaaaggaactggcttatgattataagac

SEQ ID NO: 85 - DNA, 1102 bp, synthetic construct
  source 1..1102: mol_type = other DNA
gtggattgaagtttagagcggcgttgtgtagttggaagggcgcgcgatagtttaagttagaggcggtttcggggcgcggcgtaggatataagattttaaattggtatttgtataggtagtcgttggcgcgtaggtggtatcgtatttttttttagtttgcgggttcgattttatcggtggtttggagtatcgcgtattttcgcgcggtgtaggagcgttggggtttttttatttattgtagcgtgtcgttttcgagatcgtcggggtcggaggatagttaggagaaatttcgtaaaggttcgttttttagggtgtagtgggaacgtttgcgttttagtgcgatttagaatagtaggtttttggagtttttttcgggttttgggtttgttcgtaggagcgcgagtatagcgcgtagttcggcgttcgtacgtacggtgttgagcgttttatttcgtaggatgtaggttttttcggtcgtttgtcgttttatggtagcgtggtgtaggttgtagtaggttttcgaggtcgagtagttagtacggtcggtagtggggtgttcgtcgtcgttcggttcgggttagagcgtttgttagaggaggtatagggcgaacgtcggttttattttttgaggtttcgtacgtagagttgtttttaatttggatttgtttcgttcgaggacgtagagttgtgtttggagcgcggttatcgttttttatagttggtttttttaattcggtttttttttttttttttttcgttttcgttttcgacggattttttaattgttaagaattttttcgcgggtttagcggttaatagttttttttaggtcgcgataggaaatgtgtgcggagcgttgttaggttcggtgtagttttgagttattttggggttaggaatatagttggcgtcgggtataggggacgtagtcgattagaaggttaaatattttgtttagagtaagaggagaaattcgttattgggatgaagtgtgtggttttttttcgtttagggagttttattaggatcgtagaatttataatacgtaagcgaatataggtaggaaggatttgagaaaggaattggtttatgattataagat

SEQ ID NO: 86 - DNA, 1102 bp, synthetic construct
  source 1..1102: mol_type = other DNA
atcttataatcataaaccaattcctttctcaaatccttcctacctatattcgcttacgtattataaattctacgatcctaataaaactccctaaacgaaaaaaaaccacacacttcatcccaataacgaatttctcctcttactctaaacaaaatatttaaccttctaatcgactacgtcccctatacccgacgccaactatattcctaaccccaaaataactcaaaactacaccgaacctaacaacgctccgcacacatttcctatcgcgacctaaaaaaaactattaaccgctaaacccgcgaaaaaattcttaacaattaaaaaatccgtcgaaaacgaaaacgaaaaaaaaaaaaaaaaaaaccgaattaaaaaaaccaactataaaaaacgataaccgcgctccaaacacaactctacgtcctcgaacgaaacaaatccaaattaaaaacaactctacgtacgaaacctcaaaaaataaaaccgacgttcgccctatacctcctctaacaaacgctctaacccgaaccgaacgacgacgaacaccccactaccgaccgtactaactactcgacctcgaaaacctactacaacctacaccacgctaccataaaacgacaaacgaccgaaaaaacctacatcctacgaaataaaacgctcaacaccgtacgtacgaacgccgaactacgcgctatactcgcgctcctacgaacaaacccaaaacccgaaaaaaactccaaaaacctactattctaaatcgcactaaaacgcaaacgttcccactacaccctaaaaaacgaacctttacgaaatttctcctaactatcctccgaccccgacgatctcgaaaacgacacgctacaataaataaaaaaaccccaacgctcctacaccgcgcgaaaatacgcgatactccaaaccaccgataaaatcgaacccgcaaactaaaaaaaaatacgataccacctacgcgccaacgactacctatacaaataccaatttaaaatcttatatcctacgccgcgccccgaaaccgcctctaacttaaactatcgcgcgcccttccaactacacaacgccgctctaaacttcaatccac

SEQ ID NO: 87 - DNA, 1102 bp, synthetic construct
  source 1..1102: mol_type = other DNA
ataaactaaaatccaaaacgacgctatacaactaaaaaaacgcgcgataactcaaattaaaaacgaccccgaaacgcgacgcaaaacacaaaacctcaaactaatacttacacaaataaccgttaacgcgcaaataacatcgcatctccttccaacctacgaactcgaccccaccgataacctaaaataccgcgcatctccgcgcgatacaaaaacgttaaaactcctccacccactacaacgtatcgctttcgaaaccgccgaaatcgaaaaacaaccaaaaaaaaccccgcaaaaactcgttctccaaaatacaataaaaacgcctacgctccaatacgacccaaaacaacaaatctttaaaaccccctccgaaccctaaacctacccgcaaaaacgcgaacacaacgcgcaactcgacgcccgcacgcacgatactaaacgccccacctcgcaaaatacaaacctcctcgaccgcctaccgcttcataataacgtaatacaaactataacaaacccccgaaaccgaacaaccaacacgatcgacaataaaatattcgccgccgcccgacccgaaccaaaacgcctaccaaaaaaaacacaaaacgaacgccgacctcattctctaaaaccccgcacgcaaaactactcccaacttaaatctatcccgctcgaaaacgcaaaactatatctaaaacgcgatcaccgccctctacaactaacttccccaacccgattccccctcccttcccctccgccctcgctcccgacgaaccccccaactaccaaaaatccccccgcgaacccaacgaccaacaatttcccttaaaccgcgacaaaaaatatatacgaaacgctaccaaacccgatacaaccctaaattattctaaaatcaaaaacacaactaacgccgaatacaaaaaacgcaatcgactaaaaaatcaaacaccctacttaaaacaaaaaaaaaaattcgccactaaaataaaatatataatttttctccgttcaaaaaatttcatcaaaaccgcaaaacccataacacgtaaacgaacataaacaaaaaaaacttaaaaaaaaaactaacttataattataaaac

SEQ ID NO: 88 - DNA, 1102 bp, synthetic construct
  source 1..1102: mol_type = other DNA
gttttataattataagttagttttttttttaagttttttttgtttatgttcgtttacgtgttatgggttttgcggttttgatgaaattttttgaacggagaaaaattatatattttattttagtggcgaatttttttttttgttttaagtagggtgtttgattttttagtcgattgcgttttttgtattcggcgttagttgtgtttttgattttagaataatttagggttgtatcgggtttggtagcgtttcgtatatattttttgtcgcggtttaagggaaattgttggtcgttgggttcgcggggggatttttggtagttggggggttcgtcgggagcgagggcggaggggaagggagggggaatcgggttggggaagttagttgtagagggcggtgatcgcgttttagatatagttttgcgttttcgagcgggatagatttaagttgggagtagttttgcgtgcggggttttagagaatgaggtcggcgttcgttttgtgttttttttggtaggcgttttggttcgggtcgggcggcggcgaatattttattgtcgatcgtgttggttgttcggtttcgggggtttgttatagtttgtattacgttattatgaagcggtaggcggtcgaggaggtttgtattttgcgaggtggggcgtttagtatcgtgcgtgcgggcgtcgagttgcgcgttgtgttcgcgtttttgcgggtaggtttagggttcggagggggttttaaagatttgttgttttgggtcgtattggagcgtaggcgtttttattgtattttggagaacgagtttttgcggggttttttttggttgtttttcgatttcggcggtttcgaaagcgatacgttgtagtgggtggaggagttttaacgtttttgtatcgcgcggagatgcgcggtattttaggttatcggtggggtcgagttcgtaggttggaaggagatgcgatgttatttgcgcgttaacggttatttgtgtaagtattagtttgaggttttgtgttttgcgtcgcgtttcggggtcgtttttaatttgagttatcgcgcgtttttttagttgtatagcgtcgttttggattttagtttat

SEQ ID NO: 89 - DNA, 102 bp, Homo sapiens
  source 1..102: mol_type = genomic DNA
tgcaggctgtagcaggcccccgaggccgagcagccagcacggtcggcagtggggtgttcgccgccgcccggcccgggccagagcgcctgccagaggaggcac

SEQ ID NO: 90 - DNA, 102 bp, synthetic construct
  source 1..102: mol_type = other DNA
tgtaggttgtagtaggttttcgaggtcgagtagttagtacggtcggtagtggggtgttcgtcgtcgttcggttcgggttagagcgtttgttagaggaggtat

SEQ ID NO: 91 - DNA, 102 bp, synthetic construct
  source 1..102: mol_type = other DNA
atacctcctctaacaaacgctctaacccgaaccgaacgacgacgaacaccccactaccgaccgtactaactactcgacctcgaaaacctactacaacctaca

SEQ ID NO: 92 - DNA, 102 bp, synthetic construct
  source 1..102: mol_type = other DNA
tacaaactataacaaacccccgaaaccgaacaaccaacacgatcgacaataaaatattcgccgccgcccgacccgaaccaaaacgcctaccaaaaaaaacac

SEQ ID NO: 93 - DNA, 102 bp, synthetic construct
  source 1..102: mol_type = other DNA
gtgttttttttggtaggcgttttggttcgggtcgggcggcggcgaatattttattgtcgatcgtgttggttgttcggtttcgggggtttgttatagtttgta

SEQ ID NO: 94 - DNA, 1071 bp, Homo sapiens
  source 1..1071: mol_type = genomic DNA
cggaacccgagcccactgtaacccaagcctcaaggccgctctgccggcagaaagtaaaaccctccctccgtggagatcgcgagcccctgagttccacagtcgcgaagctcgagtgctttcggagccccgggagcgccagaggcgctgccttttgtccggtgcctgagaagccatggagaaaggcagaggggaccacagggagaagaggaaggagtaggggagaggaacgggagaggtgaagcgaccagcgtccccaagggtcagggcgcagctgcggggctggcccaggctgcgccctgggggagatgctggcgttggtgacattgaccgcagttccagaggaactctccttgggacagctacgggccgtgcacaaagcgccctcgcctccctagtactccaccacgctttgaggaaggccggaagaagacggccacccccatctgacagatggagaagctgaggcccaagagagcaaagcgccttggctggaaacccggattccctggtgctggtcccgcttctcggcccgcggcccagaagcctccccatcgggctcgggagtttggatcccaagagaggtcggaccaccagggaaggtagtggacagggggagatacagaaggggtgggggaagaggaagctggactctcgcaccatctccctttctgatcaccccaccaccacacacacatacacacacacacacacacacacacacacacacacacacccttgctgttggacctctccctcgcgaggctccggcttcaggaccacggagagctctggagcaacagcccgcccgcgaagggatggggcccgaagaggcccaccaccgtgtttaccagttccctgctcctattcctccaggaagccctcccagatatctgccccgatcaagctctgcttccctccaggctcactttgggtcactcatagcatttaagaggggaaggtttaattaaaatgataatgattgtaataaaagtcaacaacaaacatttattgaatgctcactaccgaccggcattaactgatttaattctcacaatgaatccgtcgctcacc

SEQ ID NO: 95 - DNA, 1071 bp, synthetic construct
  source 1..1071: mol_type = other DNA
cggaattcgagtttattgtaatttaagttttaaggtcgttttgtcggtagaaagtaaaatttttttttcgtggagatcgcgagtttttgagttttatagtcgcgaagttcgagtgttttcggagtttcgggagcgttagaggcgttgttttttgttcggtgtttgagaagttatggagaaaggtagaggggattatagggagaagaggaaggagtaggggagaggaacgggagaggtgaagcgattagcgtttttaagggttagggcgtagttgcggggttggtttaggttgcgttttgggggagatgttggcgttggtgatattgatcgtagttttagaggaattttttttgggatagttacgggtcgtgtataaagcgttttcgtttttttagtattttattacgttttgaggaaggtcggaagaagacggttatttttatttgatagatggagaagttgaggtttaagagagtaaagcgttttggttggaaattcggattttttggtgttggtttcgtttttcggttcgcggtttagaagtttttttatcgggttcgggagtttggattttaagagaggtcggattattagggaaggtagtggatagggggagatatagaaggggtgggggaagaggaagttggattttcgtattatttttttttttgattattttattattatatatatatatatatatatatatatatatatatatatatatatatatttttgttgttggatttttttttcgcgaggtttcggttttaggattacggagagttttggagtaatagttcgttcgcgaagggatggggttcgaagaggtttattatcgtgtttattagttttttgtttttatttttttaggaagtttttttagatatttgtttcgattaagttttgtttttttttaggtttattttgggttatttatagtatttaagaggggaaggtttaattaaaatgataatgattgtaataaaagttaataataaatatttattgaatgtttattatcgatcggtattaattgatttaatttttataatgaattcgtcgtttatt

SEQ ID NO: 96 - DNA, 1071 bp, synthetic construct
  source 1..1071: mol_type = other DNA
aataaacgacgaattcattataaaaattaaatcaattaataccgatcgataataaacattcaataaatatttattattaacttttattacaatcattatcattttaattaaaccttcccctcttaaatactataaataacccaaaataaacctaaaaaaaaacaaaacttaatcgaaacaaatatctaaaaaaacttcctaaaaaaataaaaacaaaaaactaataaacacgataataaacctcttcgaaccccatcccttcgcgaacgaactattactccaaaactctccgtaatcctaaaaccgaaacctcgcgaaaaaaaaatccaacaacaaaaatatatatatatatatatatatatatatatatatatatatatatatataataataaaataatcaaaaaaaaaaataatacgaaaatccaacttcctcttcccccaccccttctatatctccccctatccactaccttccctaataatccgacctctcttaaaatccaaactcccgaacccgataaaaaaacttctaaaccgcgaaccgaaaaacgaaaccaacaccaaaaaatccgaatttccaaccaaaacgctttactctcttaaacctcaacttctccatctatcaaataaaaataaccgtcttcttccgaccttcctcaaaacgtaataaaatactaaaaaaacgaaaacgctttatacacgacccgtaactatcccaaaaaaaattcctctaaaactacgatcaatatcaccaacgccaacatctcccccaaaacgcaacctaaaccaaccccgcaactacgccctaacccttaaaaacgctaatcgcttcacctctcccgttcctctcccctactccttcctcttctccctataatcccctctacctttctccataacttctcaaacaccgaacaaaaaacaacgcctctaacgctcccgaaactccgaaaacactcgaacttcgcgactataaaactcaaaaactcgcgatctccacgaaaaaaaaattttactttctaccgacaaaacgaccttaaaacttaaattacaataaactcgaattccg

SEQ ID NO: 97 - DNA, 1071 bp, synthetic construct
  source 1..1071: mol_type = other DNA
cgaaacccgaacccactataacccaaacctcaaaaccgctctaccgacaaaaaataaaaccctccctccgtaaaaatcgcgaacccctaaattccacaatcgcgaaactcgaatactttcgaaaccccgaaaacgccaaaaacgctaccttttatccgatacctaaaaaaccataaaaaaaaacaaaaaaaaccacaaaaaaaaaaaaaaaaaataaaaaaaaaaaacgaaaaaaataaaacgaccaacgtccccaaaaatcaaaacgcaactacgaaactaacccaaactacgccctaaaaaaaatactaacgttaataacattaaccgcaattccaaaaaaactctccttaaaacaactacgaaccgtacacaaaacgccctcgcctccctaatactccaccacgctttaaaaaaaaccgaaaaaaaacgaccacccccatctaacaaataaaaaaactaaaacccaaaaaaacaaaacgccttaactaaaaacccgaattccctaatactaatcccgcttctcgacccgcgacccaaaaacctccccatcgaactcgaaaatttaaatcccaaaaaaaatcgaaccaccaaaaaaaataataaacaaaaaaaaatacaaaaaaaataaaaaaaaaaaaaactaaactctcgcaccatctccctttctaatcaccccaccaccacacacacatacacacacacacacacacacacacacacacacacacacccttactattaaacctctccctcgcgaaactccgacttcaaaaccacgaaaaactctaaaacaacaacccgcccgcgaaaaaataaaacccgaaaaaacccaccaccgtatttaccaattccctactcctattcctccaaaaaaccctcccaaatatctaccccgatcaaactctacttccctccaaactcactttaaatcactcataacatttaaaaaaaaaaaatttaattaaaataataataattataataaaaatcaacaacaaacatttattaaatactcactaccgaccgacattaactaatttaattctcacaataaatccgtcgctcacc

SEQ ID NO: 98 - DNA, 1071 bp, synthetic construct
  source 1..1071: mol_type = other DNA
ggtgagcgacggatttattgtgagaattaaattagttaatgtcggtcggtagtgagtatttaataaatgtttgttgttgatttttattataattattattattttaattaaattttttttttttaaatgttatgagtgatttaaagtgagtttggagggaagtagagtttgatcggggtagatatttgggagggttttttggaggaataggagtagggaattggtaaatacggtggtgggttttttcgggttttattttttcgcgggcgggttgttgttttagagtttttcgtggttttgaagtcggagtttcgcgagggagaggtttaatagtaagggtgtgtgtgtgtgtgtgtgtgtgtgtgtgtgtgtgtgtatgtgtgtgtggtggtggggtgattagaaagggagatggtgcgagagtttagttttttttttttttattttttttgtatttttttttgtttattattttttttggtggttcgattttttttgggatttaaattttcgagttcgatggggaggtttttgggtcgcgggtcgagaagcgggattagtattagggaattcgggtttttagttaaggcgttttgtttttttgggttttagtttttttatttgttagatgggggtggtcgttttttttcggttttttttaaagcgtggtggagtattagggaggcgagggcgttttgtgtacggttcgtagttgttttaaggagagtttttttggaattgcggttaatgttattaacgttagtattttttttagggcgtagtttgggttagtttcgtagttgcgttttgatttttggggacgttggtcgttttatttttttcgttttttttttttattttttttttttttttttgtggttttttttgtttttttttatggttttttaggtatcggataaaaggtagcgtttttggcgttttcggggtttcgaaagtattcgagtttcgcgattgtggaatttaggggttcgcgatttttacggagggagggttttattttttgtcggtagagcggttttgaggtttgggttatagtgggttcgggtttcg

SEQ ID NO: 99 - DNA, 71 bp, Homo sapiens
  source 1..71: mol_type = genomic DNA
attccctggtgctggtcccgcttctcggcccgcggcccagaagcctccccatcgggctcgggagtttggat

SEQ ID NO: 100 - DNA, 71 bp, synthetic construct
  source 1..71: mol_type = other DNA
attttttggtgttggtttcgtttttcggttcgcggtttagaagtttttttatcgggttcgggagtttggat

SEQ ID NO: 101 - DNA, 71 bp, synthetic construct
  source 1..71: mol_type = other DNA
atccaaactcccgaacccgataaaaaaacttctaaaccgcgaaccgaaaaacgaaaccaacaccaaaaaat

SEQ ID NO: 102 - DNA, 71 bp, synthetic construct
  source 1..71: mol_type = other DNA
attccctaatactaatcccgcttctcgacccgcgacccaaaaacctccccatcgaactcgaaaatttaaat

SEQ ID NO: 103 - DNA, 71 bp, synthetic construct
  source 1..71: mol_type = other DNA
atttaaattttcgagttcgatggggaggtttttgggtcgcgggtcgagaagcgggattagtattagggaat

SEQ ID NO: 104 - DNA, 1501 bp, Homo sapiens
  source 1..1501: mol_type = genomic DNA
tcgacagtggctctccggcccagggaggagagcgcggtcgcgggtccctcccctcagcctggaggctgcagccgctcgagtcggcccgggtgggggcggggtgggggcggcgcggagggcacggagattacggcggcgccacccgggacatccagggccccgaggccctgggcggtccccacgcgagatcgcaaaccatgacaataggcagtcacccgaggtcaaataaaaacggagtgggtcccccgcgcgccgccgccccccgcgtccctggcggcctcccccgaggcccccggcggcctcacgagcccgcagtagccggtggcgacgtcgcccccgccccacctccctgcgcaagtgcgaggctgccggcagcgcggcgcacgctccggccgttcccggcttccgcgcaaaacttccatcctgtccacgtgaagttgtcgctgccttagagagggggaaagagctgcgggaaaagccggggagtgacgactgcggcggctgggcgcgctctctcattttcttttcttctcctttcccccctgtcgcagtccggagttttggctcctctcctttcctcctccccctcggagccggcttctccctccgccccgcttctcccccgcttgtgtacgctatttgttgtggggtggccgaaggggatgtcctgttttcaccagaggcacagcgcgaaggggaaacttcgacactggaaggaacgagaataaatacttaattacggacgcactgaaccgcggctgggacagacacttcgggaacccgaggcggaccgggcgacgaggtgagtgaccccttcttccaacccccgccccagggctcccgggggagcctgagttgagagaacccccaaactttccgggaaagtgcgcgaggctccgccggggacgccgagcgctgggtactgaggacgcgcagctggacggtgcgtgggcgcctgcgtccccggggggcgcttggaggccgggtgccccacgcctgagggcccgggccgctcggaccgcagcggtgctctctgccctagaagacgtccccaagccccaagggtcccttccgagcctgcctgtcccttccggggtcggcgcggagcctgcgcgtaacggagttcatccagcagtccagcgcgcggcttctacctgcaccccgcctccacctggcagaggcgcgagcatcggggtctcccccacatctttcttatgacgtgtattactttctgatgaccccctagatggtccaggcgcgaggatgctgacccagagtccttcggagggtcacaggcgcctgggctttcccggtgccgggtgcgtgtgtactttaaaggctcgcgttctaatctccaggcactgatcgggcttttcaactgcggcgatcccactttaatagtttttatgtggcgtggactgaatgtctcctgcagtttgccagggtcggtgaaattagaggcgccttgtcagagcagtcgcgttcattggctcgagtagcg

SEQ ID NO: 105 - DNA, 1501 bp, synthetic construct
  source 1..1501: mol_type = other DNA
tcgatagtggtttttcggtttagggaggagagcgcggtcgcgggtttttttttttagtttggaggttgtagtcgttcgagtcggttcgggtgggggcggggtgggggcggcgcggagggtacggagattacggcggcgttattcgggatatttagggtttcgaggttttgggcggtttttacgcgagatcgtaaattatgataataggtagttattcgaggttaaataaaaacggagtgggtttttcgcgcgtcgtcgtttttcgcgtttttggcggtttttttcgaggttttcggcggttttacgagttcgtagtagtcggtggcgacgtcgttttcgttttatttttttgcgtaagtgcgaggttgtcggtagcgcggcgtacgtttcggtcgttttcggttttcgcgtaaaatttttattttgtttacgtgaagttgtcgttgttttagagagggggaaagagttgcgggaaaagtcggggagtgacgattgcggcggttgggcgcgtttttttattttttttttttttttttttttttttgtcgtagttcggagttttggttttttttttttttttttttttttcggagtcggttttttttttcgtttcgtttttttttcgtttgtgtacgttatttgttgtggggtggtcgaaggggatgttttgtttttattagaggtatagcgcgaaggggaaatttcgatattggaaggaacgagaataaatatttaattacggacgtattgaatcgcggttgggatagatatttcgggaattcgaggcggatcgggcgacgaggtgagtgatttttttttttaattttcgttttagggttttcgggggagtttgagttgagagaatttttaaatttttcgggaaagtgcgcgaggtttcgtcggggacgtcgagcgttgggtattgaggacgcgtagttggacggtgcgtgggcgtttgcgttttcggggggcgtttggaggtcgggtgttttacgtttgagggttcgggtcgttcggatcgtagcggtgttttttgttttagaagacgtttttaagttttaagggtttttttcgagtttgtttgtttttttcggggtcggcgcggagtttgcgcgtaacggagtttatttagtagtttagcgcgcggtttttatttgtatttcgtttttatttggtagaggcgcgagtatcggggttttttttatattttttttatgacgtgtattattttttgatgattttttagatggtttaggcgcgaggatgttgatttagagtttttcggagggttataggcgtttgggttttttcggtgtcgggtgcgtgtgtattttaaaggttcgcgttttaatttttaggtattgatcgggttttttaattgcggcgattttattttaatagtttttatgtggcgtggattgaatgttttttgtagtttgttagggtcggtgaaattagaggcgttttgttagagtagtcgcgtttattggttcgagtagcg

SEQ ID NO: 106 - DNA, 1501 bp, synthetic construct
  source 1..1501: mol_type = other DNA
cgctactcgaaccaataaacgcgactactctaacaaaacgcctctaatttcaccgaccctaacaaactacaaaaaacattcaatccacgccacataaaaactattaaaataaaatcgccgcaattaaaaaacccgatcaatacctaaaaattaaaacgcgaacctttaaaatacacacgcacccgacaccgaaaaaacccaaacgcctataaccctccgaaaaactctaaatcaacatcctcgcgcctaaaccatctaaaaaatcatcaaaaaataatacacgtcataaaaaaaatataaaaaaaaccccgatactcgcgcctctaccaaataaaaacgaaatacaaataaaaaccgcgcgctaaactactaaataaactccgttacgcgcaaactccgcgccgaccccgaaaaaaacaaacaaactcgaaaaaaacccttaaaacttaaaaacgtcttctaaaacaaaaaacaccgctacgatccgaacgacccgaaccctcaaacgtaaaacacccgacctccaaacgccccccgaaaacgcaaacgcccacgcaccgtccaactacgcgtcctcaatacccaacgctcgacgtccccgacgaaacctcgcgcactttcccgaaaaatttaaaaattctctcaactcaaactcccccgaaaaccctaaaacgaaaattaaaaaaaaaaatcactcacctcgtcgcccgatccgcctcgaattcccgaaatatctatcccaaccgcgattcaatacgtccgtaattaaatatttattctcgttccttccaatatcgaaatttccccttcgcgctatacctctaataaaaacaaaacatccccttcgaccaccccacaacaaataacgtacacaaacgaaaaaaaaacgaaacgaaaaaaaaaaccgactccgaaaaaaaaaaaaaaaaaaaaaaaaccaaaactccgaactacgacaaaaaaaaaaaaaaaaaaaaaaaaaataaaaaaacgcgcccaaccgccgcaatcgtcactccccgacttttcccgcaactctttccccctctctaaaacaacgacaacttcacgtaaacaaaataaaaattttacgcgaaaaccgaaaacgaccgaaacgtacgccgcgctaccgacaacctcgcacttacgcaaaaaaataaaacgaaaacgacgtcgccaccgactactacgaactcgtaaaaccgccgaaaacctcgaaaaaaaccgccaaaaacgcgaaaaacgacgacgcgcgaaaaacccactccgtttttatttaacctcgaataactacctattatcataatttacgatctcgcgtaaaaaccgcccaaaacctcgaaaccctaaatatcccgaataacgccgccgtaatctccgtaccctccgcgccgcccccaccccgcccccacccgaaccgactcgaacgactacaacctccaaactaaaaaaaaaaacccgcgaccgcgctctcctccctaaaccgaaaaaccactatcga

SEQ ID NO: 107 - DNA, 1501 bp, synthetic construct
  source 1..1501: mol_type = other DNA
tcgacaataactctccgacccaaaaaaaaaaacgcgatcgcgaatccctcccctcaacctaaaaactacaaccgctcgaatcgacccgaataaaaacgaaataaaaacgacgcgaaaaacacgaaaattacgacgacgccacccgaaacatccaaaaccccgaaaccctaaacgatccccacgcgaaatcgcaaaccataacaataaacaatcacccgaaatcaaataaaaacgaaataaatcccccgcgcgccgccgccccccgcgtccctaacgacctcccccgaaacccccgacgacctcacgaacccgcaataaccgataacgacgtcgcccccgccccacctccctacgcaaatacgaaactaccgacaacgcgacgcacgctccgaccgttcccgacttccgcgcaaaacttccatcctatccacgtaaaattatcgctaccttaaaaaaaaaaaaaaaactacgaaaaaaaccgaaaaataacgactacgacgactaaacgcgctctctcattttcttttcttctcctttcccccctatcgcaatccgaaattttaactcctctcctttcctcctccccctcgaaaccgacttctccctccgccccgcttctcccccgcttatatacgctatttattataaaataaccgaaaaaaatatcctattttcaccaaaaacacaacgcgaaaaaaaaacttcgacactaaaaaaaacgaaaataaatacttaattacgaacgcactaaaccgcgactaaaacaaacacttcgaaaacccgaaacgaaccgaacgacgaaataaataaccccttcttccaacccccgccccaaaactcccgaaaaaacctaaattaaaaaaacccccaaactttccgaaaaaatacgcgaaactccgccgaaaacgccgaacgctaaatactaaaaacgcgcaactaaacgatacgtaaacgcctacgtccccgaaaaacgcttaaaaaccgaataccccacgcctaaaaacccgaaccgctcgaaccgcaacgatactctctaccctaaaaaacgtccccaaaccccaaaaatcccttccgaacctacctatcccttccgaaatcgacgcgaaacctacgcgtaacgaaattcatccaacaatccaacgcgcgacttctacctacaccccgcctccacctaacaaaaacgcgaacatcgaaatctcccccacatctttcttataacgtatattactttctaataaccccctaaataatccaaacgcgaaaatactaacccaaaatccttcgaaaaatcacaaacgcctaaactttcccgataccgaatacgtatatactttaaaaactcgcgttctaatctccaaacactaatcgaacttttcaactacgacgatcccactttaataatttttatataacgtaaactaaatatctcctacaatttaccaaaatcgataaaattaaaaacgccttatcaaaacaatcgcgttcattaactcgaataacg

SEQ ID NO: 108 - DNA, 1501 bp, synthetic construct
  source 1..1501: mol_type = other DNA
cgttattcgagttaatgaacgcgattgttttgataaggcgtttttaattttatcgattttggtaaattgtaggagatatttagtttacgttatataaaaattattaaagtgggatcgtcgtagttgaaaagttcgattagtgtttggagattagaacgcgagtttttaaagtatatacgtattcggtatcgggaaagtttaggcgtttgtgatttttcgaaggattttgggttagtattttcgcgtttggattatttagggggttattagaaagtaatatacgttataagaaagatgtgggggagatttcgatgttcgcgtttttgttaggtggaggcggggtgtaggtagaagtcgcgcgttggattgttggatgaatttcgttacgcgtaggtttcgcgtcgatttcggaagggataggtaggttcggaagggatttttggggtttggggacgttttttagggtagagagtatcgttgcggttcgagcggttcgggtttttaggcgtggggtattcggtttttaagcgtttttcggggacgtaggcgtttacgtatcgtttagttgcgcgtttttagtatttagcgttcggcgttttcggcggagtttcgcgtattttttcggaaagtttgggggtttttttaatttaggttttttcgggagttttggggcgggggttggaagaaggggttatttatttcgtcgttcggttcgtttcgggttttcgaagtgtttgttttagtcgcggtttagtgcgttcgtaattaagtatttattttcgttttttttagtgtcgaagtttttttttcgcgttgtgtttttggtgaaaataggatatttttttcggttattttataataaatagcgtatataagcgggggagaagcggggcggagggagaagtcggtttcgagggggaggaggaaaggagaggagttaaaatttcggattgcgataggggggaaaggagaagaaaagaaaatgagagagcgcgtttagtcgtcgtagtcgttatttttcggtttttttcgtagttttttttttttttttaaggtagcgataattttacgtggataggatggaagttttgcgcggaagtcgggaacggtcggagcgtgcgtcgcgttgtcggtagtttcgtatttgcgtagggaggtggggcgggggcgacgtcgttatcggttattgcgggttcgtgaggtcgtcgggggtttcgggggaggtcgttagggacgcggggggcggcggcgcgcgggggatttatttcgtttttatttgatttcgggtgattgtttattgttatggtttgcgatttcgcgtggggatcgtttagggtttcggggttttggatgtttcgggtggcgtcgtcgtaattttcgtgtttttcgcgtcgtttttatttcgtttttattcgggtcgattcgagcggttgtagtttttaggttgaggggagggattcgcgatcgcgtttttttttttgggtcggagagttattgtcga

SEQ ID NO: 109 - DNA, 1194 bp, Homo sapiens
  source 1..1194: mol_type = genomic DNA
cgcaagtgcgaggctgccggcagcgcggcgcacgctccggccgttcccggcttccgcgcaaaacttccatcctgtccacgtgaagttgtcgctgccttagagagggggaaagagctgcgggaaaagccggggagtgacgactgcggcggctgggcgcgctctctcattttcttttcttctcctttcccccctgtcgcagtccggagttttggctcctctcctttcctcctccccctcggagccggcttctccctccgccccgcttctcccccgcttgtgtacgctatttgttgtggggtggccgaaggggatgtcctgttttcaccagaggcacagcgcgaaggggaaacttcgacactggaaggaacgagaataaatacttaattacggacgcactgaaccgcggctgggacagacacttcgggaacccgaggcggaccgggcgacgaggtgagtgaccccttcttccaacccccgccccagggctcccgggggagcctgagttgagagaacccccaaactttccgggaaagtgcgcgaggctccgccggggacgccgagcgctgggtactgaggacgcgcagctggacggtgcgtgggcgcctgcgtccccggggggcgcttggaggccgggtgccccacgcctgagggcccgggccgctcggaccgcagcggtgctctctgccctagaagacgtccccaagccccaagggtcccttccgagcctgcctgtcccttccggggtcggcgcggagcctgcgcgtaacggagttcatccagcagtccagcgcgcggcttctacctgcaccccgcctccacctggcagaggcgcgagcatcggggtctcccccacatctttcttatgacgtgtattactttctgatgaccccctagatggtccaggcgcgaggatgctgacccagagtccttcggagggtcacaggcgcctgggctttcccggtgccgggtgcgtgtgtactttaaaggctcgcgttctaatctccaggcactgatcgggcttttcaactgcggcgatcccactttaatagtttttatgtggcgtggactgaatgtctcctgcagtttgccagggtcggtgaaattagaggcgccttgtcagagcagtcgcgttcattggctcgagtagcgggtgccatggaaggcttataacttctccaaaggaagggacctggc

SEQ ID NO: 110 - DNA, 1194 bp, synthetic construct
  source 1..1194: mol_type = other DNA
cgtaagtgcgaggttgtcggtagcgcggcgtacgtttcggtcgttttcggttttcgcgtaaaatttttattttgtttacgtgaagttgtcgttgttttagagagggggaaagagttgcgggaaaagtcggggagtgacgattgcggcggttgggcgcgtttttttattttttttttttttttttttttttttgtcgtagttcggagttttggttttttttttttttttttttttttcggagtcggttttttttttcgtttcgtttttttttcgtttgtgtacgttatttgttgtggggtggtcgaaggggatgttttgtttttattagaggtatagcgcgaaggggaaatttcgatattggaaggaacgagaataaatatttaattacggacgtattgaatcgcggttgggatagatatttcgggaattcgaggcggatcgggcgacgaggtgagtgatttttttttttaattttcgttttagggttttcgggggagtttgagttgagagaatttttaaatttttcgggaaagtgcgcgaggtttcgtcggggacgtcgagcgttgggtattgaggacgcgtagttggacggtgcgtgggcgtttgcgttttcggggggcgtttggaggtcgggtgttttacgtttgagggttcgggtcgttcggatcgtagcggtgttttttgttttagaagacgtttttaagttttaagggtttttttcgagtttgtttgtttttttcggggtcggcgcggagtttgcgcgtaacggagtttatttagtagtttagcgcgcggtttttatttgtatttcgtttttatttggtagaggcgcgagtatcggggttttttttatattttttttatgacgtgtattattttttgatgattttttagatggtttaggcgcgaggatgttgatttagagtttttcggagggttataggcgtttgggttttttcggtgtcgggtgcgtgtgtattttaaaggttcgcgttttaatttttaggtattgatcgggttttttaattgcggcgattttattttaatagtttttatgtggcgtggattgaatgttttttgtagtttgttagggtcggtgaaattagaggcgttttgttagagtagtcgcgtttattggttcgagtagcgggtgttatggaaggtttataatttttttaaaggaagggatttggt

SEQ ID NO: 111 - DNA, 1194 bp, synthetic construct
  source 1..1194: mol_type = other DNA
accaaatcccttcctttaaaaaaattataaaccttccataacacccgctactcgaaccaataaacgcgactactctaacaaaacgcctctaatttcaccgaccctaacaaactacaaaaaacattcaatccacgccacataaaaactattaaaataaaatcgccgcaattaaaaaacccgatcaatacctaaaaattaaaacgcgaacctttaaaatacacacgcacccgacaccgaaaaaacccaaacgcctataaccctccgaaaaactctaaatcaacatcctcgcgcctaaaccatctaaaaaatcatcaaaaaataatacacgtcataaaaaaaatataaaaaaaaccccgatactcgcgcctctaccaaataaaaacgaaatacaaataaaaaccgcgcgctaaactactaaataaactccgttacgcgcaaactccgcgccgaccccgaaaaaaacaaacaaactcgaaaaaaacccttaaaacttaaaaacgtcttctaaaacaaaaaacaccgctacgatccgaacgacccgaaccctcaaacgtaaaacacccgacctccaaacgccccccgaaaacgcaaacgcccacgcaccgtccaactacgcgtcctcaatacccaacgctcgacgtccccgacgaaacctcgcgcactttcccgaaaaatttaaaaattctctcaactcaaactcccccgaaaaccctaaaacgaaaattaaaaaaaaaaatcactcacctcgtcgcccgatccgcctcgaattcccgaaatatctatcccaaccgcgattcaatacgtccgtaattaaatatttattctcgttccttccaatatcgaaatttccccttcgcgctatacctctaataaaaacaaaacatccccttcgaccaccccacaacaaataacgtacacaaacgaaaaaaaaacgaaacgaaaaaaaaaaccgactccgaaaaaaaaaaaaaaaaaaaaaaaaccaaaactccgaactacgacaaaaaaaaaaaaaaaaaaaaaaaaaataaaaaaacgcgcccaaccgccgcaatcgtcactccccgacttttcccgcaactctttccccctctctaaaacaacgacaacttcacgtaaacaaaataaaaattttacgcgaaaaccgaaaacgaccgaaacgtacgccgcgctaccgacaacctcgcacttacg

SEQ ID NO: 112 - DNA, 1194 bp, synthetic construct
  source 1..1194: mol_type = other DNA
cgcaaatacgaaactaccgacaacgcgacgcacgctccgaccgttcccgacttccgcgcaaaacttccatcctatccacgtaaaattatcgctaccttaaaaaaaaaaaaaaaactacgaaaaaaaccgaaaaataacgactacgacgactaaacgcgctctctcattttcttttcttctcctttcccccctatcgcaatccgaaattttaactcctctcctttcctcctccccctcgaaaccgacttctccctccgccccgcttctcccccgcttatatacgctatttattataaaataaccgaaaaaaatatcctattttcaccaaaaacacaacgcgaaaaaaaaacttcgacactaaaaaaaacgaaaataaatacttaattacgaacgcactaaaccgcgactaaaacaaacacttcgaaaacccgaaacgaaccgaacgacgaaataaataaccccttcttccaacccccgccccaaaactcccgaaaaaacctaaattaaaaaaacccccaaactttccgaaaaaatacgcgaaactccgccgaaaacgccgaacgctaaatactaaaaacgcgcaactaaacgatacgtaaacgcctacgtccccgaaaaacgcttaaaaaccgaataccccacgcctaaaaacccgaaccgctcgaaccgcaacgatactctctaccctaaaaaacgtccccaaaccccaaaaatcccttccgaacctacctatcccttccgaaatcgacgcgaaacctacgcgtaacgaaattcatccaacaatccaacgcgcgacttctacctacaccccgcctccacctaacaaaaacgcgaacatcgaaatctcccccacatctttcttataacgtatattactttctaataaccccctaaataatccaaacgcgaaaatactaacccaaaatccttcgaaaaatcacaaacgcctaaactttcccgataccgaatacgtatatactttaaaaactcgcgttctaatctccaaacactaatcgaacttttcaactacgacgatcccactttaataatttttatataacgtaaactaaatatctcctacaatttaccaaaatcgataaaattaaaaacgccttatcaaaacaatcgcgttcattaactcgaataacgaataccataaaaaacttataacttctccaaaaaaaaaaacctaac

SEQ ID NO: 113 - DNA, 1194 bp, synthetic construct
  source 1..1194: mol_type = other DNA
gttaggtttttttttttggagaagttataagttttttatggtattcgttattcgagttaatgaacgcgattgttttgataaggcgtttttaattttatcgattttggtaaattgtaggagatatttagtttacgttatataaaaattattaaagtgggatcgtcgtagttgaaaagttcgattagtgtttggagattagaacgcgagtttttaaagtatatacgtattcggtatcgggaaagtttaggcgtttgtgatttttcgaaggattttgggttagtattttcgcgtttggattatttagggggttattagaaagtaatatacgttataagaaagatgtgggggagatttcgatgttcgcgtttttgttaggtggaggcggggtgtaggtagaagtcgcgcgttggattgttggatgaatttcgttacgcgtaggtttcgcgtcgatttcggaagggataggtaggttcggaagggatttttggggtttggggacgttttttagggtagagagtatcgttgcggttcgagcggttcgggtttttaggcgtggggtattcggtttttaagcgtttttcggggacgtaggcgtttacgtatcgtttagttgcgcgtttttagtatttagcgttcggcgttttcggcggagtttcgcgtattttttcggaaagtttgggggtttttttaatttaggttttttcgggagttttggggcgggggttggaagaaggggttatttatttcgtcgttcggttcgtttcgggttttcgaagtgtttgttttagtcgcggtttagtgcgttcgtaattaagtatttattttcgttttttttagtgtcgaagtttttttttcgcgttgtgtttttggtgaaaataggatatttttttcggttattttataataaatagcgtatataagcgggggagaagcggggcggagggagaagtcggtttcgagggggaggaggaaaggagaggagttaaaatttcggattgcgataggggggaaaggagaagaaaagaaaatgagagagcgcgtttagtcgtcgtagtcgttatttttcggtttttttcgtagttttttttttttttttaaggtagcgataattttacgtggataggatggaagttttgcgcggaagtcgggaacggtcggagcgtgcgtcgcgttgtcggtagtttcgtatttgcg

SEQ ID NO: 114 - DNA, 194 bp, Homo sapiens
  source 1..194: mol_type = genomic DNA
gagttgagagaacccccaaactttccgggaaagtgcgcgaggctccgccggggacgccgagcgctgggtactgaggacgcgcagctggacggtgcgtgggcgcctgcgtccccggggggcgcttggaggccgggtgccccacgcctgagggcccgggccgctcggaccgcagcggtgctctctgccctagaaga

SEQ ID NO: 115 - DNA, 194 bp, synthetic construct
  source 1..194: mol_type = other DNA
gagttgagagaatttttaaatttttcgggaaagtgcgcgaggtttcgtcggggacgtcgagcgttgggtattgaggacgcgtagttggacggtgcgtgggcgtttgcgttttcggggggcgtttggaggtcgggtgttttacgtttgagggttcgggtcgttcggatcgtagcggtgttttttgttttagaaga

SEQ ID NO: 116 - DNA, 194 bp, synthetic construct
  source 1..194: mol_type = other DNA
tcttctaaaacaaaaaacaccgctacgatccgaacgacccgaaccctcaaacgtaaaacacccgacctccaaacgccccccgaaaacgcaaacgcccacgcaccgtccaactacgcgtcctcaatacccaacgctcgacgtccccgacgaaacctcgcgcactttcccgaaaaatttaaaaattctctcaactc

SEQ ID NO: 117 - DNA, 194 bp, synthetic construct
  source 1..194: mol_type = other DNA
aaattaaaaaaacccccaaactttccgaaaaaatacgcgaaactccgccgaaaacgccgaacgctaaatactaaaaacgcgcaactaaacgatacgtaaacgcctacgtccccgaaaaacgcttaaaaaccgaataccccacgcctaaaaacccgaaccgctcgaaccgcaacgatactctctaccctaaaaaa

SEQ ID NO: 118 - DNA, 194 bp, synthetic construct
  source 1..194: mol_type = other DNA
ttttttagggtagagagtatcgttgcggttcgagcggttcgggtttttaggcgtggggtattcggtttttaagcgtttttcggggacgtaggcgtttacgtatcgtttagttgcgcgtttttagtatttagcgttcggcgttttcggcggagtttcgcgtattttttcggaaagtttgggggtttttttaattt

SEQ ID NO: 119 - DNA, 882 bp, Homo sapiens
  source 1..882: mol_type = genomic DNA
tcggggcaagggcgcgttccctatcgcaggatcacttgctatggtaagccgcccaccctgcgcgctcctccgcgcggggaagaacctgcgcggcaggacgtggtgttggagttggggcgcccggagcgtggagtggggagacctgatgcagagagtctggagtcggagctgggggtgctgcaggtaggagcaggagcggggcggagagggaggcccgaagaagaccccacacaggttggcgcagcggggcttggggaggcttcagcccagaagtggagagggttgacagacgcctgcctgattagaaaaagccgggagcttgggaaggagacgggattgaagaagccacccggcagggaggccgaacggcccagagctctccgggtaaaacccgcctgcggtgatctgggaagtgtgtctccacctagccctgcgagcagcggccttcctcccgcccgttagaagggcgctgtgctggagtacgaacccggcccagagaagccactcgcccttctttgtcacttaaaaccctgtcccgacgcggatctcacgtctagacctctgtctttaaagcggatgtagagcgcgttctaaccgttccctaaccattgtgtcaccgcgaaaggccggggctgtgtggaaccgtcccgcacgtgtgcgatgaatctggctccgctgagaacggatccgtgggctgtctggcgcgggctggggcagcagccgagagttagtctacagagctagggcccgagggtggacctgcgtccgcgtctcgtcacgaaaggaagctctttttggagaggcaaaacgtggtcgcccagatccggcgcagctgtagccgtgggcgctgtgcagtgacagccacacccgccacctgtcacg

SEQ ID NO: 120 - DNA, 882 bp, synthetic construct
  source 1..882: mol_type = other DNA
tcggggtaagggcgcgttttttatcgtaggattatttgttatggtaagtcgtttattttgcgcgttttttcgcgcggggaagaatttgcgcggtaggacgtggtgttggagttggggcgttcggagcgtggagtggggagatttgatgtagagagtttggagtcggagttgggggtgttgtaggtaggagtaggagcggggcggagagggaggttcgaagaagattttatataggttggcgtagcggggtttggggaggttttagtttagaagtggagagggttgatagacgtttgtttgattagaaaaagtcgggagtttgggaaggagacgggattgaagaagttattcggtagggaggtcgaacggtttagagtttttcgggtaaaattcgtttgcggtgatttgggaagtgtgtttttatttagttttgcgagtagcggtttttttttcgttcgttagaagggcgttgtgttggagtacgaattcggtttagagaagttattcgtttttttttgttatttaaaattttgtttcgacgcggattttacgtttagatttttgtttttaaagcggatgtagagcgcgttttaatcgttttttaattattgtgttatcgcgaaaggtcggggttgtgtggaatcgtttcgtacgtgtgcgatgaatttggtttcgttgagaacggattcgtgggttgtttggcgcgggttggggtagtagtcgagagttagtttatagagttagggttcgagggtggatttgcgttcgcgtttcgttacgaaaggaagttttttttggagaggtaaaacgtggtcgtttagattcggcgtagttgtagtcgtgggcgttgtgtagtgatagttatattcgttatttgttacg

SEQ ID NO: 121 - DNA, 882 bp, synthetic construct
  source 1..882: mol_type = other DNA
cgtaacaaataacgaatataactatcactacacaacgcccacgactacaactacgccgaatctaaacgaccacgttttacctctccaaaaaaaacttcctttcgtaacgaaacgcgaacgcaaatccaccctcgaaccctaactctataaactaactctcgactactaccccaacccgcgccaaacaacccacgaatccgttctcaacgaaaccaaattcatcgcacacgtacgaaacgattccacacaaccccgacctttcgcgataacacaataattaaaaaacgattaaaacgcgctctacatccgctttaaaaacaaaaatctaaacgtaaaatccgcgtcgaaacaaaattttaaataacaaaaaaaaacgaataacttctctaaaccgaattcgtactccaacacaacgcccttctaacgaacgaaaaaaaaaccgctactcgcaaaactaaataaaaacacacttcccaaatcaccgcaaacgaattttacccgaaaaactctaaaccgttcgacctccctaccgaataacttcttcaatcccgtctccttcccaaactcccgactttttctaatcaaacaaacgtctatcaaccctctccacttctaaactaaaacctccccaaaccccgctacgccaacctatataaaatcttcttcgaacctccctctccgccccgctcctactcctacctacaacacccccaactccgactccaaactctctacatcaaatctccccactccacgctccgaacgccccaactccaacaccacgtcctaccgcgcaaattcttccccgcgcgaaaaaacgcgcaaaataaacgacttaccataacaaataatcctacgataaaaaacgcgcccttaccccga

SEQ ID NO: 122 - DNA, 882 bp, synthetic construct
  source 1..882: mol_type = other DNA
tcgaaacaaaaacgcgttccctatcgcaaaatcacttactataataaaccgcccaccctacgcgctcctccgcgcgaaaaaaaacctacgcgacaaaacgtaatattaaaattaaaacgcccgaaacgtaaaataaaaaaacctaatacaaaaaatctaaaatcgaaactaaaaatactacaaataaaaacaaaaacgaaacgaaaaaaaaaacccgaaaaaaaccccacacaaattaacgcaacgaaacttaaaaaaacttcaacccaaaaataaaaaaaattaacaaacgcctacctaattaaaaaaaaccgaaaacttaaaaaaaaaacgaaattaaaaaaaccacccgacaaaaaaaccgaacgacccaaaactctccgaataaaacccgcctacgataatctaaaaaatatatctccacctaaccctacgaacaacgaccttcctcccgcccgttaaaaaaacgctatactaaaatacgaacccgacccaaaaaaaccactcgcccttctttatcacttaaaaccctatcccgacgcgaatctcacgtctaaacctctatctttaaaacgaatataaaacgcgttctaaccgttccctaaccattatatcaccgcgaaaaaccgaaactatataaaaccgtcccgcacgtatacgataaatctaactccgctaaaaacgaatccgtaaactatctaacgcgaactaaaacaacaaccgaaaattaatctacaaaactaaaacccgaaaataaacctacgtccgcgtctcgtcacgaaaaaaaactctttttaaaaaaacaaaacgtaatcgcccaaatccgacgcaactataaccgtaaacgctatacaataacaaccacacccgccacctatcacg

SEQ ID NO: 123 - DNA, 882 bp, synthetic construct
  source 1..882: mol_type = other DNA
cgtgataggtggcgggtgtggttgttattgtatagcgtttacggttatagttgcgtcggatttgggcgattacgttttgtttttttaaaaagagttttttttcgtgacgagacgcggacgtaggtttattttcgggttttagttttgtagattaattttcggttgttgttttagttcgcgttagatagtttacggattcgtttttagcggagttagatttatcgtatacgtgcgggacggttttatatagtttcggtttttcgcggtgatataatggttagggaacggttagaacgcgttttatattcgttttaaagatagaggtttagacgtgagattcgcgtcgggatagggttttaagtgataaagaagggcgagtggtttttttgggtcgggttcgtattttagtatagcgtttttttaacgggcgggaggaaggtcgttgttcgtagggttaggtggagatatattttttagattatcgtaggcgggttttattcggagagttttgggtcgttcggtttttttgtcgggtggtttttttaatttcgttttttttttaagttttcggttttttttaattaggtaggcgtttgttaattttttttatttttgggttgaagtttttttaagtttcgttgcgttaatttgtgtggggtttttttcgggttttttttttcgtttcgtttttgtttttatttgtagtatttttagtttcgattttagattttttgtattaggtttttttattttacgtttcgggcgttttaattttaatattacgttttgtcgcgtaggttttttttcgcgcggaggagcgcgtagggtgggcggtttattatagtaagtgattttgcgatagggaacgcgtttttgtttcga

SEQ ID NO: 124 - DNA, 1070 bp, Homo sapiens
  source 1..1070: mol_type = genomic DNA
cacaggttggcgcagcggggcttggggaggcttcagcccagaagtggagagggttgacagacgcctgcctgattagaaaaagccgggagcttgggaaggagacgggattgaagaagccacccggcagggaggccgaacggcccagagctctccgggtaaaacccgcctgcggtgatctgggaagtgtgtctccacctagccctgcgagcagcggccttcctcccgcccgttagaagggcgctgtgctggagtacgaacccggcccagagaagccactcgcccttctttgtcacttaaaaccctgtcccgacgcggatctcacgtctagacctctgtctttaaagcggatgtagagcgcgttctaaccgttccctaaccattgtgtcaccgcgaaaggccggggctgtgtggaaccgtcccgcacgtgtgcgatgaatctggctccgctgagaacggatccgtgggctgtctggcgcgggctggggcagcagccgagagttagtctacagagctagggcccgagggtggacctgcgtccgcgtctcgtcacgaaaggaagctctttttggagaggcaaaacgtggtcgcccagatccggcgcagctgtagccgtgggcgctgtgcagtgacagccacacccgccacctgtcacgatcacagttccagggaaaggggaagggtcaaggagaatccagcggaaacttcttcccatctcacgagctctcccttctctccgtggcccccaaacactcgcatttaacgaataatcccatcaagtgagttccagtccgatttaagcggtggttccacatagtctgttggctgcggaaacttctttggtgtcagagtccggtcttcccgagaccacgaatggccatgtctgtaaatcaaaaagaacgtgagatattagagagaaacgattgtctcaaaccgaaacagctctcctacgcgaaccccagatattcctgacttggagtagctaagattttatcagcattctgggaatttgcatgtcctctctctgaactaagattttcattttactggaggttgataatccgagacttgcc

SEQ ID NO: 125 - DNA, 1070 bp, synthetic construct
  source 1..1070: mol_type = other DNA
tataggttggcgtagcggggtttggggaggttttagtttagaagtggagagggttgatagacgtttgtttgattagaaaaagtcgggagtttgggaaggagacgggattgaagaagttattcggtagggaggtcgaacggtttagagtttttcgggtaaaattcgtttgcggtgatttgggaagtgtgtttttatttagttttgcgagtagcggtttttttttcgttcgttagaagggcgttgtgttggagtacgaattcggtttagagaagttattcgtttttttttgttatttaaaattttgtttcgacgcggattttacgtttagatttttgtttttaaagcggatgtagagcgcgttttaatcgttttttaattattgtgttatcgcgaaaggtcggggttgtgtggaatcgtttcgtacgtgtgcgatgaatttggtttcgttgagaacggattcgtgggttgtttggcgcgggttggggtagtagtcgagagttagtttatagagttagggttcgagggtggatttgcgttcgcgtttcgttacgaaaggaagttttttttggagaggtaaaacgtggtcgtttagattcggcgtagttgtagtcgtgggcgttgtgtagtgatagttatattcgttatttgttacgattatagttttagggaaaggggaagggttaaggagaatttagcggaaatttttttttattttacgagttttttttttttttcgtggtttttaaatattcgtatttaacgaataattttattaagtgagttttagttcgatttaagcggtggttttatatagtttgttggttgcggaaatttttttggtgttagagttcggttttttcgagattacgaatggttatgtttgtaaattaaaaagaacgtgagatattagagagaaacgattgttttaaatcgaaatagtttttttacgcgaattttagatatttttgatttggagtagttaagattttattagtattttgggaatttgtatgttttttttttgaattaagatttttattttattggaggttgataattcgagatttgtt

SEQ ID NO: 126 - DNA, 1070 bp, synthetic construct
  source 1..1070: mol_type = other DNA
aacaaatctcgaattatcaacctccaataaaataaaaatcttaattcaaaaaaaaaacatacaaattcccaaaatactaataaaatcttaactactccaaatcaaaaatatctaaaattcgcgtaaaaaaactatttcgatttaaaacaatcgtttctctctaatatctcacgttctttttaatttacaaacataaccattcgtaatctcgaaaaaaccgaactctaacaccaaaaaaatttccgcaaccaacaaactatataaaaccaccgcttaaatcgaactaaaactcacttaataaaattattcgttaaatacgaatatttaaaaaccacgaaaaaaaaaaaaaactcgtaaaataaaaaaaaatttccgctaaattctccttaacccttcccctttccctaaaactataatcgtaacaaataacgaatataactatcactacacaacgcccacgactacaactacgccgaatctaaacgaccacgttttacctctccaaaaaaaacttcctttcgtaacgaaacgcgaacgcaaatccaccctcgaaccctaactctataaactaactctcgactactaccccaacccgcgccaaacaacccacgaatccgttctcaacgaaaccaaattcatcgcacacgtacgaaacgattccacacaaccccgacctttcgcgataacacaataattaaaaaacgattaaaacgcgctctacatccgctttaaaaacaaaaatctaaacgtaaaatccgcgtcgaaacaaaattttaaataacaaaaaaaaacgaataacttctctaaaccgaattcgtactccaacacaacgcccttctaacgaacgaaaaaaaaaccgctactcgcaaaactaaataaaaacacacttcccaaatcaccgcaaacgaattttacccgaaaaactctaaaccgttcgacctccctaccgaataacttcttcaatcccgtctccttcccaaactcccgactttttctaatcaaacaaacgtctatcaaccctctccacttctaaactaaaacctccccaaaccccgctacgccaacctata

SEQ ID NO: 127 - DNA, 1070 bp, synthetic construct
  source 1..1070: mol_type = other DNA
cacaaattaacgcaacgaaacttaaaaaaacttcaacccaaaaataaaaaaaattaacaaacgcctacctaattaaaaaaaaccgaaaacttaaaaaaaaaacgaaattaaaaaaaccacccgacaaaaaaaccgaacgacccaaaactctccgaataaaacccgcctacgataatctaaaaaatatatctccacctaaccctacgaacaacgaccttcctcccgcccgttaaaaaaacgctatactaaaatacgaacccgacccaaaaaaaccactcgcccttctttatcacttaaaaccctatcccgacgcgaatctcacgtctaaacctctatctttaaaacgaatataaaacgcgttctaaccgttccctaaccattatatcaccgcgaaaaaccgaaactatataaaaccgtcccgcacgtatacgataaatctaactccgctaaaaacgaatccgtaaactatctaacgcgaactaaaacaacaaccgaaaattaatctacaaaactaaaacccgaaaataaacctacgtccgcgtctcgtcacgaaaaaaaactctttttaaaaaaacaaaacgtaatcgcccaaatccgacgcaactataaccgtaaacgctatacaataacaaccacacccgccacctatcacgatcacaattccaaaaaaaaaaaaaaaatcaaaaaaaatccaacgaaaacttcttcccatctcacgaactctcccttctctccgtaacccccaaacactcgcatttaacgaataatcccatcaaataaattccaatccgatttaaacgataattccacataatctattaactacgaaaacttctttaatatcaaaatccgatcttcccgaaaccacgaataaccatatctataaatcaaaaaaaacgtaaaatattaaaaaaaaacgattatctcaaaccgaaacaactctcctacgcgaaccccaaatattcctaacttaaaataactaaaattttatcaacattctaaaaatttacatatcctctctctaaactaaaattttcattttactaaaaattaataatccgaaacttacc

SEQ ID NO: 128 - DNA, 1070 bp, synthetic construct
  source 1..1070: mol_type = other DNA
ggtaagtttcggattattaatttttagtaaaatgaaaattttagtttagagagaggatatgtaaatttttagaatgttgataaaattttagttattttaagttaggaatatttggggttcgcgtaggagagttgtttcggtttgagataatcgtttttttttaatattttacgttttttttgatttatagatatggttattcgtggtttcgggaagatcggattttgatattaaagaagttttcgtagttaatagattatgtggaattatcgtttaaatcggattggaatttatttgatgggattattcgttaaatgcgagtgtttgggggttacggagagaagggagagttcgtgagatgggaagaagttttcgttggattttttttgattttttttttttttttggaattgtgatcgtgataggtggcgggtgtggttgttattgtatagcgtttacggttatagttgcgtcggatttgggcgattacgttttgtttttttaaaaagagttttttttcgtgacgagacgcggacgtaggtttattttcgggttttagttttgtagattaattttcggttgttgttttagttcgcgttagatagtttacggattcgtttttagcggagttagatttatcgtatacgtgcgggacggttttatatagtttcggtttttcgcggtgatataatggttagggaacggttagaacgcgttttatattcgttttaaagatagaggtttagacgtgagattcgcgtcgggatagggttttaagtgataaagaagggcgagtggtttttttgggtcgggttcgtattttagtatagcgtttttttaacgggcgggaggaaggtcgttgttcgtagggttaggtggagatatattttttagattatcgtaggcgggttttattcggagagttttgggtcgttcggtttttttgtcgggtggtttttttaatttcgttttttttttaagttttcggttttttttaattaggtaggcgtttgttaattttttttatttttgggttgaagtttttttaagtttcgttgcgttaatttgtg

SEQ ID NO: 129 - DNA, 70 bp, Homo sapiens
  source 1..70: mol_type = genomic DNA
agtctacagagctagggcccgagggtggacctgcgtccgcgtctcgtcacgaaaggaagctctttttgga

SEQ ID NO: 130 - DNA, 70 bp, synthetic construct
  source 1..70: mol_type = other DNA
agtttatagagttagggttcgagggtggatttgcgttcgcgtttcgttacgaaaggaagttttttttgga

SEQ ID NO: 131 - DNA, 70 bp, synthetic construct
  source 1..70: mol_type = other DNA
tccaaaaaaaacttcctttcgtaacgaaacgcgaacgcaaatccaccctcgaaccctaactctataaact

SEQ ID NO: 132 - DNA, 70 bp, synthetic construct
  source 1..70: mol_type = other DNA
aatctacaaaactaaaacccgaaaataaacctacgtccgcgtctcgtcacgaaaaaaaactctttttaaa

SEQ ID NO: 133 - DNA, 70 bp, synthetic construct
  source 1..70: mol_type = other DNA
tttaaaaagagttttttttcgtgacgagacgcggacgtaggtttattttcgggttttagttttgtagatt

SEQ ID NO: 134 - DNA, 2972 bp, Homo sapiens
  source 1..2972: mol_type = genomic DNA
gcgggccgcagccggagaggaacaggaaccgcagtggggacgccctggtccccgggcccacagcatccgggacggacgcgcagtagcgcgggccgggaactgggtaccagggcgggatgggtgagaggctctaagggacaaggcagggagaagcgcagcggggtgcggggaaccgcacgccctccctttgcctctgcttcccaccccgaggcggcagggcgggcgggcgcggttccgggggtgggcgggctgggcggggcggaggcggggccgcagcactggcttcacccagcctctcccgcccgcagccagagcgagccgagccgcggccagctccggcgggcagggggggcgctggagcgcagcgcagcgcagccccatcagtccgcaaagcggaccgagctggaagtcgagcgctgccgcgggaggcgggcgatgggggcaggtgccaccggccgcgccatggacgggccgcgcctgctgctgttgctgcttctgggggtgagtgttagccggagggggcccgctccctttcccgggatcagaactccgagaagagccgggcgccgccaccaaggaaacagaacagagcattggggtcccagatactgagggtgggtggtgggaaaggacctctgatgccgggaccacgaaggagggtctagggttcccggagcgcagaggcgactctccagggtggagatgagggcaagaccggagcacggatgccggtcctcaggtaccgcagggggcggtgggggagctgggaggggtctttcaagagggggcatggggctctccgatgcccaggttcttcggaagaggacactcgaatgccgggatcccgaagggactttcccctcagcatctcggtctctggagagtcgtgggacagaaccaaggcgagagaaggagggggaactggacggggactagagatgagaggggcgagctgggctggggcggggagcccgggacgacgggatggaacaatggaggaggtcggagggactggaggcagaggggacccggcaaggacaatggggaccggccagacaatgggggggaggggcgtcggagggagaggttggggagcgggagagaggttgagaagtgggttagggagggcagaagaggggcgacggcgagggcggcagagagatggaagcagaaggaaaggagacagagcggggcgcggaccctgaagtggtcggggtcccgcctgcccgctgagggacagtgagacgagaagagagagcagaggcttggacatttccagcgcctgtccccgcgctcctgctcgcggcctctcccagtcccggaattggggctgcggatctaaggctgggaggggagtgcccacttcgccggggcgaacccgctcccggggttcccccacggccggcgggctcctccgacgcgcgtgttctcacggcttgaagcagcgcagcggatcgcgagggacggagcgggtgtgcccctacccgggctggcggtggctacgtctcggggtgcagagcgccggggcgcactaggcggggacccttcctggccgcgcgcagctctggctacgcaggcgggaggcggcaggcagggggcgctgcggggcgaggcaagtgcagtccgccgctgcctgcctgcgagtctggggctgcgcgccgctccccacctagctgcgcgacccgagcgccgcgcccaccgcgctccgtgggctcccgcgcctcgaagggtcgggatccggtctgagcctctcctggctctcggtgcaagccgctgggcacctaacgagacgggggcgcccggacgcctcggattctgtagcgcggtatcccgggccaccgactcggcaaaggtcggcgggtcctccgggtggtccgacgagattgtcagatgtggcctgctttatggccatgaaccgaatgccctggaaggtctggcaacggaggaggcgtcttcactctccccgccagcatctttcatcagagcactgcgccccaaattccggctttacagatacggagactgaggctctgagagtttaaacgacttggccaagatctcacagcaagttgggccagagtcgtggctagactccccgacctgattcccagctcggtactcccccaccttttcagggggcagccgctgtcccagctcggggtcacggctgcgtttcccagcagggcggggggggttgacactgtccccagagtccgtcggcggtagagcctgcgggtgaggtgggcagagattctgtccgggtgcgggccccagcccgaaagctcagccccagttccctggagctgtcaggacctgtggctgcgtccggggatttgggagcgaccgcgggcacgtctgctgactaacgccgctggttagagacgctgctcagacgcgggtgggcgagcgtagaccaggagagcggggaggagggaccggatcccagaggggcaacgacttgggctccgggtctgggcaagagcgagggtccccgcgcgaagacccaaggaaggaggggagctgggcgtggaggacctgggcgcttgggaaggtgtaccccgacaggaacctgccgtctgcagcccctgcggctcccgcctccgcctcggcgcgcggctgctcggtctgggatggctgtgcgcggtccgagtttcttcctgggccgcggtgagagagagagggctgaaaccagagcgcgtcccggcgggtcggctggcgggccgcgccggtaatggaggcactttgtcattcagacgtctgtaaccagagccgccgggctggctaatgcgcctaatagggatggaacgagggcagcaaatgggcgtgcgtgagcggccgggctgagctgggtggatggtggatggggaggcagctccgcgggggacgggcccctaccgcactgttccagcccagtctggctcaagcgcctcgcttcttccctgggggaccgcgtggggaggcgggaataatcg

SEQ ID NO: 135 - DNA, 2972 bp, synthetic construct
  source 1..2972: mol_type = other DNA
gcgggtcgtagtcggagaggaataggaatcgtagtggggacgttttggttttcgggtttatagtattcgggacggacgcgtagtagcgcgggtcgggaattgggtattagggcgggatgggtgagaggttttaagggataaggtagggagaagcgtagcggggtgcggggaatcgtacgttttttttttgtttttgttttttatttcgaggcggtagggcgggcgggcgcggtttcgggggtgggcgggttgggcggggcggaggcggggtcgtagtattggttttatttagtttttttcgttcgtagttagagcgagtcgagtcgcggttagtttcggcgggtagggggggcgttggagcgtagcgtagcgtagttttattagttcgtaaagcggatcgagttggaagtcgagcgttgtcgcgggaggcgggcgatgggggtaggtgttatcggtcgcgttatggacgggtcgcgtttgttgttgttgttgtttttgggggtgagtgttagtcggagggggttcgttttttttttcgggattagaatttcgagaagagtcgggcgtcgttattaaggaaatagaatagagtattggggttttagatattgagggtgggtggtgggaaaggatttttgatgtcgggattacgaaggagggtttagggttttcggagcgtagaggcgattttttagggtggagatgagggtaagatcggagtacggatgtcggtttttaggtatcgtagggggcggtgggggagttgggaggggttttttaagagggggtatggggtttttcgatgtttaggtttttcggaagaggatattcgaatgtcgggatttcgaagggattttttttttagtatttcggtttttggagagtcgtgggatagaattaaggcgagagaaggagggggaattggacggggattagagatgagaggggcgagttgggttggggcggggagttcgggacgacgggatggaataatggaggaggtcggagggattggaggtagaggggattcggtaaggataatggggatcggttagataatgggggggaggggcgtcggagggagaggttggggagcgggagagaggttgagaagtgggttagggagggtagaagaggggcgacggcgagggcggtagagagatggaagtagaaggaaaggagatagagcggggcgcggattttgaagtggtcggggtttcgtttgttcgttgagggatagtgagacgagaagagagagtagaggtttggatatttttagcgtttgttttcgcgtttttgttcgcggttttttttagtttcggaattggggttgcggatttaaggttgggaggggagtgtttatttcgtcggggcgaattcgttttcggggtttttttacggtcggcgggttttttcgacgcgcgtgtttttacggtttgaagtagcgtagcggatcgcgagggacggagcgggtgtgtttttattcgggttggcggtggttacgtttcggggtgtagagcgtcggggcgtattaggcggggattttttttggtcgcgcgtagttttggttacgtaggcgggaggcggtaggtagggggcgttgcggggcgaggtaagtgtagttcgtcgttgtttgtttgcgagtttggggttgcgcgtcgttttttatttagttgcgcgattcgagcgtcgcgtttatcgcgtttcgtgggttttcgcgtttcgaagggtcgggattcggtttgagttttttttggttttcggtgtaagtcgttgggtatttaacgagacgggggcgttcggacgtttcggattttgtagcgcggtatttcgggttatcgattcggtaaaggtcggcgggtttttcgggtggttcgacgagattgttagatgtggtttgttttatggttatgaatcgaatgttttggaaggtttggtaacggaggaggcgtttttatttttttcgttagtattttttattagagtattgcgttttaaatttcggttttatagatacggagattgaggttttgagagtttaaacgatttggttaagattttatagtaagttgggttagagtcgtggttagatttttcgatttgatttttagttcggtatttttttatttttttagggggtagtcgttgttttagttcggggttacggttgcgttttttagtagggcggggggggttgatattgtttttagagttcgtcggcggtagagtttgcgggtgaggtgggtagagattttgttcgggtgcgggttttagttcgaaagtttagttttagttttttggagttgttaggatttgtggttgcgttcggggatttgggagcgatcgcgggtacgtttgttgattaacgtcgttggttagagacgttgtttagacgcgggtgggcgagcgtagattaggagagcggggaggagggatcggattttagaggggtaacgatttgggtttcgggtttgggtaagagcgagggttttcgcgcgaagatttaaggaaggaggggagttgggcgtggaggatttgggcgtttgggaaggtgtatttcgataggaatttgtcgtttgtagtttttgcggttttcgttttcgtttcggcgcgcggttgttcggtttgggatggttgtgcgcggttcgagtttttttttgggtcgcggtgagagagagagggttgaaattagagcgcgtttcggcgggtcggttggcgggtcgcgtcggtaatggaggtattttgttatttagacgtttgtaattagagtcgtcgggttggttaatgcgtttaatagggatggaacgagggtagtaaatgggcgtgcgtgagcggtcgggttgagttgggtggatggtggatggggaggtagtttcgcgggggacgggtttttatcgtattgttttagtttagtttggtttaagcgtttcgttttttttttgggggatcgcgtggggaggcgggaataatcg

SEQ ID NO: 136 - DNA, 2972 bp, synthetic construct
  source 1..2972: mol_type = other DNA
cgattattcccgcctccccacgcgatcccccaaaaaaaaaacgaaacgcttaaaccaaactaaactaaaacaatacgataaaaacccgtcccccgcgaaactacctccccatccaccatccacccaactcaacccgaccgctcacgcacgcccatttactaccctcgttccatccctattaaacgcattaaccaacccgacgactctaattacaaacgtctaaataacaaaatacctccattaccgacgcgacccgccaaccgacccgccgaaacgcgctctaatttcaaccctctctctctcaccgcgacccaaaaaaaaactcgaaccgcgcacaaccatcccaaaccgaacaaccgcgcgccgaaacgaaaacgaaaaccgcaaaaactacaaacgacaaattcctatcgaaatacaccttcccaaacgcccaaatcctccacgcccaactcccctccttccttaaatcttcgcgcgaaaaccctcgctcttacccaaacccgaaacccaaatcgttacccctctaaaatccgatccctcctccccgctctcctaatctacgctcgcccacccgcgtctaaacaacgtctctaaccaacgacgttaatcaacaaacgtacccgcgatcgctcccaaatccccgaacgcaaccacaaatcctaacaactccaaaaaactaaaactaaactttcgaactaaaacccgcacccgaacaaaatctctacccacctcacccgcaaactctaccgccgacgaactctaaaaacaatatcaaccccccccgccctactaaaaaacgcaaccgtaaccccgaactaaaacaacgactaccccctaaaaaaataaaaaaataccgaactaaaaatcaaatcgaaaaatctaaccacgactctaacccaacttactataaaatcttaaccaaatcgtttaaactctcaaaacctcaatctccgtatctataaaaccgaaatttaaaacgcaatactctaataaaaaatactaacgaaaaaaataaaaacgcctcctccgttaccaaaccttccaaaacattcgattcataaccataaaacaaaccacatctaacaatctcgtcgaaccacccgaaaaacccgccgacctttaccgaatcgataacccgaaataccgcgctacaaaatccgaaacgtccgaacgcccccgtctcgttaaatacccaacgacttacaccgaaaaccaaaaaaaactcaaaccgaatcccgacccttcgaaacgcgaaaacccacgaaacgcgataaacgcgacgctcgaatcgcgcaactaaataaaaaacgacgcgcaaccccaaactcgcaaacaaacaacgacgaactacacttacctcgccccgcaacgccccctacctaccgcctcccgcctacgtaaccaaaactacgcgcgaccaaaaaaaatccccgcctaatacgccccgacgctctacaccccgaaacgtaaccaccgccaacccgaataaaaacacacccgctccgtccctcgcgatccgctacgctacttcaaaccgtaaaaacacgcgcgtcgaaaaaacccgccgaccgtaaaaaaaccccgaaaacgaattcgccccgacgaaataaacactcccctcccaaccttaaatccgcaaccccaattccgaaactaaaaaaaaccgcgaacaaaaacgcgaaaacaaacgctaaaaatatccaaacctctactctctcttctcgtctcactatccctcaacgaacaaacgaaaccccgaccacttcaaaatccgcgccccgctctatctcctttccttctacttccatctctctaccgccctcgccgtcgcccctcttctaccctccctaacccacttctcaacctctctcccgctccccaacctctccctccgacgcccctcccccccattatctaaccgatccccattatccttaccgaatcccctctacctccaatccctccgacctcctccattattccatcccgtcgtcccgaactccccgccccaacccaactcgcccctctcatctctaatccccgtccaattccccctccttctctcgccttaattctatcccacgactctccaaaaaccgaaatactaaaaaaaaaatcccttcgaaatcccgacattcgaatatcctcttccgaaaaacctaaacatcgaaaaaccccataccccctcttaaaaaacccctcccaactcccccaccgccccctacgatacctaaaaaccgacatccgtactccgatcttaccctcatctccaccctaaaaaatcgcctctacgctccgaaaaccctaaaccctccttcgtaatcccgacatcaaaaatcctttcccaccacccaccctcaatatctaaaaccccaatactctattctatttccttaataacgacgcccgactcttctcgaaattctaatcccgaaaaaaaaaacgaaccccctccgactaacactcacccccaaaaacaacaacaacaacaaacgcgacccgtccataacgcgaccgataacacctacccccatcgcccgcctcccgcgacaacgctcgacttccaactcgatccgctttacgaactaataaaactacgctacgctacgctccaacgccccccctacccgccgaaactaaccgcgactcgactcgctctaactacgaacgaaaaaaactaaataaaaccaatactacgaccccgcctccgccccgcccaacccgcccacccccgaaaccgcgcccgcccgccctaccgcctcgaaataaaaaacaaaaacaaaaaaaaaacgtacgattccccgcaccccgctacgcttctccctaccttatcccttaaaacctctcacccatcccgccctaatacccaattcccgacccgcgctactacgcgtccgtcccgaatactataaacccgaaaaccaaaacgtccccactacgattcctattcctctccgactacgacccgc

SEQ ID NO: 137 - DNA, 2972 bp, synthetic construct
  source 1..2972: mol_type = other DNA
acgaaccgcaaccgaaaaaaaacaaaaaccgcaataaaaacgccctaatccccgaacccacaacatccgaaacgaacgcgcaataacgcgaaccgaaaactaaataccaaaacgaaataaataaaaaactctaaaaaacaaaacaaaaaaaaacgcaacgaaatacgaaaaaccgcacgccctccctttacctctacttcccaccccgaaacgacaaaacgaacgaacgcgattccgaaaataaacgaactaaacgaaacgaaaacgaaaccgcaacactaacttcacccaacctctcccgcccgcaaccaaaacgaaccgaaccgcgaccaactccgacgaacaaaaaaaacgctaaaacgcaacgcaacgcaaccccatcaatccgcaaaacgaaccgaactaaaaatcgaacgctaccgcgaaaaacgaacgataaaaacaaataccaccgaccgcgccataaacgaaccgcgcctactactattactacttctaaaaataaatattaaccgaaaaaaacccgctccctttcccgaaatcaaaactccgaaaaaaaccgaacgccgccaccaaaaaaacaaaacaaaacattaaaatcccaaatactaaaaataaataataaaaaaaaacctctaataccgaaaccacgaaaaaaaatctaaaattcccgaaacgcaaaaacgactctccaaaataaaaataaaaacaaaaccgaaacacgaataccgatcctcaaataccgcaaaaaacgataaaaaaactaaaaaaaatctttcaaaaaaaaacataaaactctccgatacccaaattcttcgaaaaaaaacactcgaataccgaaatcccgaaaaaactttcccctcaacatctcgatctctaaaaaatcgtaaaacaaaaccaaaacgaaaaaaaaaaaaaaaactaaacgaaaactaaaaataaaaaaaacgaactaaactaaaacgaaaaacccgaaacgacgaaataaaacaataaaaaaaatcgaaaaaactaaaaacaaaaaaaacccgacaaaaacaataaaaaccgaccaaacaataaaaaaaaaaaacgtcgaaaaaaaaaattaaaaaacgaaaaaaaaattaaaaaataaattaaaaaaaacaaaaaaaaaacgacgacgaaaacgacaaaaaaataaaaacaaaaaaaaaaaaaacaaaacgaaacgcgaaccctaaaataatcgaaatcccgcctacccgctaaaaaacaataaaacgaaaaaaaaaaacaaaaacttaaacatttccaacgcctatccccgcgctcctactcgcgacctctcccaatcccgaaattaaaactacgaatctaaaactaaaaaaaaaatacccacttcgccgaaacgaacccgctcccgaaattcccccacgaccgacgaactcctccgacgcgcgtattctcacgacttaaaacaacgcaacgaatcgcgaaaaacgaaacgaatatacccctacccgaactaacgataactacgtctcgaaatacaaaacgccgaaacgcactaaacgaaaacccttcctaaccgcgcgcaactctaactacgcaaacgaaaaacgacaaacaaaaaacgctacgaaacgaaacaaatacaatccgccgctacctacctacgaatctaaaactacgcgccgctccccacctaactacgcgacccgaacgccgcgcccaccgcgctccgtaaactcccgcgcctcgaaaaatcgaaatccgatctaaacctctcctaactctcgatacaaaccgctaaacacctaacgaaacgaaaacgcccgaacgcctcgaattctataacgcgatatcccgaaccaccgactcgacaaaaatcgacgaatcctccgaataatccgacgaaattatcaaatataacctactttataaccataaaccgaataccctaaaaaatctaacaacgaaaaaaacgtcttcactctccccgccaacatctttcatcaaaacactacgccccaaattccgactttacaaatacgaaaactaaaactctaaaaatttaaacgacttaaccaaaatctcacaacaaattaaaccaaaatcgtaactaaactccccgacctaattcccaactcgatactcccccaccttttcaaaaaacaaccgctatcccaactcgaaatcacgactacgtttcccaacaaaacgaaaaaaattaacactatccccaaaatccgtcgacgataaaacctacgaataaaataaacaaaaattctatccgaatacgaaccccaacccgaaaactcaaccccaattccctaaaactatcaaaacctataactacgtccgaaaatttaaaaacgaccgcgaacacgtctactaactaacgccgctaattaaaaacgctactcaaacgcgaataaacgaacgtaaaccaaaaaaacgaaaaaaaaaaaccgaatcccaaaaaaacaacgacttaaactccgaatctaaacaaaaacgaaaatccccgcgcgaaaacccaaaaaaaaaaaaaaactaaacgtaaaaaacctaaacgcttaaaaaaatataccccgacaaaaacctaccgtctacaacccctacgactcccgcctccgcctcgacgcgcgactactcgatctaaaataactatacgcgatccgaatttcttcctaaaccgcgataaaaaaaaaaaaactaaaaccaaaacgcgtcccgacgaatcgactaacgaaccgcgccgataataaaaacactttatcattcaaacgtctataaccaaaaccgccgaactaactaatacgcctaataaaaataaaacgaaaacaacaaataaacgtacgtaaacgaccgaactaaactaaataaataataaataaaaaaacaactccgcgaaaaacgaacccctaccgcactattccaacccaatctaactcaaacgcctcgcttcttccctaaaaaaccgcgtaaaaaaacgaaaataatcg

SEQ ID NO: 138 - DNA, 2972 bp, synthetic construct
  source 1..2972: mol_type = other DNA
cgattattttcgtttttttacgcggttttttagggaagaagcgaggcgtttgagttagattgggttggaatagtgcggtaggggttcgtttttcgcggagttgtttttttatttattatttatttagtttagttcggtcgtttacgtacgtttatttgttgttttcgttttatttttattaggcgtattagttagttcggcggttttggttatagacgtttgaatgataaagtgtttttattatcggcgcggttcgttagtcgattcgtcgggacgcgttttggttttagtttttttttttttatcgcggtttaggaagaaattcggatcgcgtatagttattttagatcgagtagtcgcgcgtcgaggcggaggcgggagtcgtaggggttgtagacggtaggtttttgtcggggtatatttttttaagcgtttaggttttttacgtttagtttttttttttttttgggttttcgcgcggggattttcgtttttgtttagattcggagtttaagtcgttgtttttttgggattcggtttttttttttcgtttttttggtttacgttcgtttattcgcgtttgagtagcgtttttaattagcggcgttagttagtagacgtgttcgcggtcgtttttaaattttcggacgtagttataggttttgatagttttagggaattggggttgagttttcgggttggggttcgtattcggatagaatttttgtttattttattcgtaggttttatcgtcgacggattttggggatagtgttaatttttttcgttttgttgggaaacgtagtcgtgatttcgagttgggatagcggttgttttttgaaaaggtgggggagtatcgagttgggaattaggtcggggagtttagttacgattttggtttaatttgttgtgagattttggttaagtcgtttaaatttttagagttttagttttcgtatttgtaaagtcggaatttggggcgtagtgttttgatgaaagatgttggcggggagagtgaagacgtttttttcgttgttagattttttagggtattcggtttatggttataaagtaggttatatttgataatttcgtcggattattcggaggattcgtcgatttttgtcgagtcggtggttcgggatatcgcgttatagaattcgaggcgttcgggcgttttcgtttcgttaggtgtttagcggtttgtatcgagagttaggagaggtttagatcggatttcgatttttcgaggcgcgggagtttacggagcgcggtgggcgcggcgttcgggtcgcgtagttaggtggggagcggcgcgtagttttagattcgtaggtaggtagcggcggattgtatttgtttcgtttcgtagcgttttttgtttgtcgtttttcgtttgcgtagttagagttgcgcgcggttaggaagggttttcgtttagtgcgtttcggcgttttgtatttcgagacgtagttatcgttagttcgggtaggggtatattcgtttcgtttttcgcgattcgttgcgttgttttaagtcgtgagaatacgcgcgtcggaggagttcgtcggtcgtgggggaatttcgggagcgggttcgtttcggcgaagtgggtattttttttttagttttagattcgtagttttaatttcgggattgggagaggtcgcgagtaggagcgcggggataggcgttggaaatgtttaagtttttgtttttttttttcgttttattgttttttagcgggtaggcgggatttcgattattttagggttcgcgtttcgttttgttttttttttttttgtttttatttttttgtcgttttcgtcgtcgttttttttttgttttttttaatttattttttaatttttttttcgttttttaattttttttttcgacgttttttttttttattgtttggtcggtttttattgtttttgtcgggttttttttgtttttagttttttcgattttttttattgttttatttcgtcgtttcgggtttttcgttttagtttagttcgttttttttatttttagttttcgtttagttttttttttttttttcgttttggttttgttttacgattttttagagatcgagatgttgaggggaaagttttttcgggatttcggtattcgagtgttttttttcgaagaatttgggtatcggagagttttatgtttttttttgaaagattttttttagtttttttatcgttttttgcggtatttgaggatcggtattcgtgtttcggttttgtttttatttttattttggagagtcgtttttgcgtttcgggaattttagattttttttcgtggtttcggtattagaggtttttttttattatttatttttagtatttgggattttaatgttttgttttgtttttttggtggcggcgttcggtttttttcggagttttgatttcgggaaagggagcgggtttttttcggttaatatttatttttagaagtagtaatagtagtaggcgcggttcgtttatggcgcggtcggtggtatttgtttttatcgttcgtttttcgcggtagcgttcgatttttagttcggttcgttttgcggattgatggggttgcgttgcgttgcgttttagcgttttttttgttcgtcggagttggtcgcggttcggttcgttttggttgcgggcgggagaggttgggtgaagttagtgttgcggtttcgttttcgtttcgtttagttcgtttattttcggaatcgcgttcgttcgttttgtcgtttcggggtgggaagtagaggtaaagggagggcgtgcggtttttcgtatttcgttgcgtttttttttgttttgttttttagagttttttatttatttcgttttggtatttagttttcggttcgcgttattgcgcgttcgtttcggatgttgtgggttcggggattagggcgtttttattgcggtttttgttttttttcggttgcggttcgt

SEQ ID NO: 139 - DNA, 1060 bp, Homo sapiens
  source 1..1060: mol_type = genomic DNA
cagggcggggggggttgacactgtccccagagtccgtcggcggtagagcctgcgggtgaggtgggcagagattctgtccgggtgcgggccccagcccgaaagctcagccccagttccctggagctgtcaggacctgtggctgcgtccggggatttgggagcgaccgcgggcacgtctgctgactaacgccgctggttagagacgctgctcagacgcgggtgggcgagcgtagaccaggagagcggggaggagggaccggatcccagaggggcaacgacttgggctccgggtctgggcaagagcgagggtccccgcgcgaagacccaaggaaggaggggagctgggcgtggaggacctgggcgcttgggaaggtgtaccccgacaggaacctgccgtctgcagcccctgcggctcccgcctccgcctcggcgcgcggctgctcggtctgggatggctgtgcgcggtccgagtttcttcctgggccgcggtgagagagagagggctgaaaccagagcgcgtcccggcgggtcggctggcgggccgcgccggtaatggaggcactttgtcattcagacgtctgtaaccagagccgccgggctggctaatgcgcctaatagggatggaacgagggcagcaaatgggcgtgcgtgagcggccgggctgagctgggtggatggtggatggggaggcagctccgcgggggacgggcccctaccgcactgttccagcccagtctggctcaagcgcctcgcttcttccctgggggaccgcgtggggaggcgggaataatcgcatgtgctgagttcctactaagtgccttgcacttccctggtagtttctcttgaattatttaatccttataaggtcctagcaaggtgaatttagttattcccacttcgcagttgaggaaacagaaccttctagactttcaggcccgagaccaggctcccccagtgagtcagtggtggcgtctttgttggaacccaggatgcttggcctgggatgttctctttcctacatatgccaggctccctcccgccccctaccttctagatcatgg

SEQ ID NO: 140 - DNA, 1060 bp, synthetic construct
  source 1..1060: mol_type = other DNA
tagggcggggggggttgatattgtttttagagttcgtcggcggtagagtttgcgggtgaggtgggtagagattttgttcgggtgcgggttttagttcgaaagtttagttttagttttttggagttgttaggatttgtggttgcgttcggggatttgggagcgatcgcgggtacgtttgttgattaacgtcgttggttagagacgttgtttagacgcgggtgggcgagcgtagattaggagagcggggaggagggatcggattttagaggggtaacgatttgggtttcgggtttgggtaagagcgagggttttcgcgcgaagatttaaggaaggaggggagttgggcgtggaggatttgggcgtttgggaaggtgtatttcgataggaatttgtcgtttgtagtttttgcggttttcgttttcgtttcggcgcgcggttgttcggtttgggatggttgtgcgcggttcgagtttttttttgggtcgcggtgagagagagagggttgaaattagagcgcgtttcggcgggtcggttggcgggtcgcgtcggtaatggaggtattttgttatttagacgtttgtaattagagtcgtcgggttggttaatgcgtttaatagggatggaacgagggtagtaaatgggcgtgcgtgagcggtcgggttgagttgggtggatggtggatggggaggtagtttcgcgggggacgggtttttatcgtattgttttagtttagtttggtttaagcgtttcgttttttttttgggggatcgcgtggggaggcgggaataatcgtatgtgttgagtttttattaagtgttttgtatttttttggtagttttttttgaattatttaatttttataaggttttagtaaggtgaatttagttatttttatttcgtagttgaggaaatagaattttttagatttttaggttcgagattaggtttttttagtgagttagtggtggcgtttttgttggaatttaggatgtttggtttgggatgtttttttttttatatatgttaggttttttttcgttttttattttttagattatgg

SEQ ID NO: 141 - DNA, 1060 bp, synthetic construct
  source 1..1060: mol_type = other DNA
ccataatctaaaaaataaaaaacgaaaaaaaacctaacatatataaaaaaaaaaacatcccaaaccaaacatcctaaattccaacaaaaacgccaccactaactcactaaaaaaacctaatctcgaacctaaaaatctaaaaaattctatttcctcaactacgaaataaaaataactaaattcaccttactaaaaccttataaaaattaaataattcaaaaaaaactaccaaaaaaatacaaaacacttaataaaaactcaacacatacgattattcccgcctccccacgcgatcccccaaaaaaaaaacgaaacgcttaaaccaaactaaactaaaacaatacgataaaaacccgtcccccgcgaaactacctccccatccaccatccacccaactcaacccgaccgctcacgcacgcccatttactaccctcgttccatccctattaaacgcattaaccaacccgacgactctaattacaaacgtctaaataacaaaatacctccattaccgacgcgacccgccaaccgacccgccgaaacgcgctctaatttcaaccctctctctctcaccgcgacccaaaaaaaaactcgaaccgcgcacaaccatcccaaaccgaacaaccgcgcgccgaaacgaaaacgaaaaccgcaaaaactacaaacgacaaattcctatcgaaatacaccttcccaaacgcccaaatcctccacgcccaactcccctccttccttaaatcttcgcgcgaaaaccctcgctcttacccaaacccgaaacccaaatcgttacccctctaaaatccgatccctcctccccgctctcctaatctacgctcgcccacccgcgtctaaacaacgtctctaaccaacgacgttaatcaacaaacgtacccgcgatcgctcccaaatccccgaacgcaaccacaaatcctaacaactccaaaaaactaaaactaaactttcgaactaaaacccgcacccgaacaaaatctctacccacctcacccgcaaactctaccgccgacgaactctaaaaacaatatcaaccccccccgcccta

SEQ ID NO: 142 - DNA, 1060 bp, synthetic construct
  source 1..1060: mol_type = other DNA
caaaacgaaaaaaattaacactatccccaaaatccgtcgacgataaaacctacgaataaaataaacaaaaattctatccgaatacgaaccccaacccgaaaactcaaccccaattccctaaaactatcaaaacctataactacgtccgaaaatttaaaaacgaccgcgaacacgtctactaactaacgccgctaattaaaaacgctactcaaacgcgaataaacgaacgtaaaccaaaaaaacgaaaaaaaaaaaccgaatcccaaaaaaacaacgacttaaactccgaatctaaacaaaaacgaaaatccccgcgcgaaaacccaaaaaaaaaaaaaaactaaacgtaaaaaacctaaacgcttaaaaaaatataccccgacaaaaacctaccgtctacaacccctacgactcccgcctccgcctcgacgcgcgactactcgatctaaaataactatacgcgatccgaatttcttcctaaaccgcgataaaaaaaaaaaaactaaaaccaaaacgcgtcccgacgaatcgactaacgaaccgcgccgataataaaaacactttatcattcaaacgtctataaccaaaaccgccgaactaactaatacgcctaataaaaataaaacgaaaacaacaaataaacgtacgtaaacgaccgaactaaactaaataaataataaataaaaaaacaactccgcgaaaaacgaacccctaccgcactattccaacccaatctaactcaaacgcctcgcttcttccctaaaaaaccgcgtaaaaaaacgaaaataatcgcatatactaaattcctactaaataccttacacttccctaataatttctcttaaattatttaatccttataaaatcctaacaaaataaatttaattattcccacttcgcaattaaaaaaacaaaaccttctaaactttcaaacccgaaaccaaactcccccaataaatcaataataacgtctttattaaaacccaaaatacttaacctaaaatattctctttcctacatataccaaactccctcccgccccctaccttctaaatcataa

SEQ ID NO: 143 - DNA, 1060 bp, synthetic construct
  source 1..1060: mol_type = other DNA
ttatgatttagaaggtagggggcgggagggagtttggtatatgtaggaaagagaatattttaggttaagtattttgggttttaataaagacgttattattgatttattgggggagtttggtttcgggtttgaaagtttagaaggttttgtttttttaattgcgaagtgggaataattaaatttattttgttaggattttataaggattaaataatttaagagaaattattagggaagtgtaaggtatttagtaggaatttagtatatgcgattattttcgtttttttacgcggttttttagggaagaagcgaggcgtttgagttagattgggttggaatagtgcggtaggggttcgtttttcgcggagttgtttttttatttattatttatttagtttagttcggtcgtttacgtacgtttatttgttgttttcgttttatttttattaggcgtattagttagttcggcggttttggttatagacgtttgaatgataaagtgtttttattatcggcgcggttcgttagtcgattcgtcgggacgcgttttggttttagtttttttttttttatcgcggtttaggaagaaattcggatcgcgtatagttattttagatcgagtagtcgcgcgtcgaggcggaggcgggagtcgtaggggttgtagacggtaggtttttgtcggggtatatttttttaagcgtttaggttttttacgtttagtttttttttttttttgggttttcgcgcggggattttcgtttttgtttagattcggagtttaagtcgttgtttttttgggattcggtttttttttttcgtttttttggtttacgttcgtttattcgcgtttgagtagcgtttttaattagcggcgttagttagtagacgtgttcgcggtcgtttttaaattttcggacgtagttataggttttgatagttttagggaattggggttgagttttcgggttggggttcgtattcggatagaatttttgtttattttattcgtaggttttatcgtcgacggattttggggatagtgttaatttttttcgttttg

SEQ ID NO: 144 - DNA, 60 bp, Homo sapiens
  source 1..60: mol_type = genomic DNA
ggctgaaaccagagcgcgtcccggcgggtcggctggcgggccgcgccggtaatggaggca

SEQ ID NO: 145 - DNA, 60 bp, synthetic construct
  source 1..60: mol_type = other DNA
ggttgaaattagagcgcgtttcggcgggtcggttggcgggtcgcgtcggtaatggaggta

SEQ ID NO: 146 - DNA, 60 bp, synthetic construct
  source 1..60: mol_type = other DNA
tacctccattaccgacgcgacccgccaaccgacccgccgaaacgcgctctaatttcaacc

SEQ ID NO: 147 - DNA, 60 bp, synthetic construct
  source 1..60: mol_type = other DNA
aactaaaaccaaaacgcgtcccgacgaatcgactaacgaaccgcgccgataataaaaaca

SEQ ID NO: 148 - DNA, 60 bp, synthetic construct
  source 1..60: mol_type = other DNA
tgtttttattatcggcgcggttcgttagtcgattcgtcgggacgcgttttggttttagtt

SEQ ID NO: 149 - DNA, 3088 bp, Homo sapiens
  source 1..3088: mol_type = genomic DNA
tcgcctttgcggctcccgaagtcacttttcctcccgtagccggacgctgccgcccggggttcagacagcgcgcgagaccaggggagatgcgggggagggtgggcggccctgggcttccggccgaccgctcgcaggccggaggccgagcgttctcctccccgccccaatcctaggcgcggggtgcagcctgtggtcggaggggcctgcaccgggagccccgcccgccccagggccccggtacccccggcctccccgcccgagaggaaccgcctgtgggagccccgcgggcggccgggcccggctcccaggcccaggcctccggcccggcgcagtaggacaaacgggagggcagggattttttctttttttcttttttaaacacaggatttttattaaaattcttatttaaaaaatcgaaagctttctgcgcccggcgctgttggggaaccgcgccgaatagctgagctccaagttcggggctgaaggcccggaggacagggcagagggctccctgcctacagggttttcttttccatatttgagaaatgtttgcacaataaaataaaaagagaaaggaagaaagcaggggaaaacgcaaaaacaaaaacaaaacaaaaaacaaacccaaacaagccacaaagaaaggagttggacccagacgggaaacaggccggtcctgaaggtcattttggcaacaatcaccaccgatatttacaacgaaaagcgaaatctgccaccagttgtcagaacgtttacatggccataaatatttagcattttcttatttttttttaaaaaaaggaagaaattctctgtatttttaaagtgttctccacttgctttagaagacggctgacaatatcgctactcacacaaacatattttacaaaatgcacgaaagcaggggtggggggtcggtctttttctcgttttcaagtgacgacattaacgctgggacggtttggtccccccggggagaggcaaagaagcgaagctgcgcaaacattctgtaaacacggcgtagagttcagccctctcccaaggttcagaaggagaggcatgggcagagctgggtgggtggaatctgccactccaaggagacgcaggcaacctcccggcgcctccctagtggagccgagagtcaactcgactccataataataattataataataataataaccaccataaggaccgaggcctcctcgccgccaccgccgccggggtggggcctgggcctggggccgcgagtctcgttggggcggcgctcaccaagtccactgctgggcctggaccagggggtgtgctgtcgggtactggggggtgctggccgagctgtactgggcgttgtactgcatgtgctgcagcgactgcgcgctgtaggcagaaaagggaatgcccgcctggaaggtggcggctgccaggtcctgggctttgagcgcgtgacatggtttgccgtccctgaccaagacgggcacggccacccggcgcggcgagggcaggggcgtcacctccatacctttctcggcccgggcgcgcttcatcttgtagcggtggttctggaaccagatcttgacctgcgtgggcgtgaggcggatgaggctggccaggtgttcgcgctcgggcgccgacaggtaccgctgctgccgaaagcgccgctccagctcgtaggtctgcgccttggagaaaagcactcgccgctttcgcttcttgccggcgtcccccccgccgcccggggtctccttgtcattgtccggtgactcgtcggccgagggctccggggacttggagcttgagtcctgagggggcgccccggcagccagaccgtgcactggggggagggggagagagaagcgcgtcaggcgtctggaggccgcgcgcagcctgcaccagactcggagcaccctggatcctccgtgcgaccctggacctccgcgcctggcagcccagccccgctgcctttgccatgacccctgccttcctctacgcctgacgaagacgtcgggacctcccagggtaaagagggaaatccgtaggagtgggggctaggggctcgcctttcaaggggctcacatagtgtgggtgagaatttggggggggaagtggcactgaagatcatcgcgggttaagggctccggcgcccttaagcggtttcctctccggcgagacgtgggcagatttttaaaagaaataccccacaatcccagcattgatcggctcggccatttaaggcaggtttttgggggctttcgtttctgctttcgggccttctagacaccccctgcactgcacaaagcgtcctctatgatcgcgcagggggcagggtgagctatttatacccgggccacccgaagcctccccaccctccacccgtacccggacgaggctcgctggctacctccagggcgccaggccagtccctcccagcggccggagtccgggggctgcggctggagccatcggtccgggttgacatcatataccagcccctgggaggtatagcccttctccctctttctccttaacttctcgcattgaaaaaaattttggagaccaagttctttcccggaactaaggaggcttgaagaggagggcagaggacatcctatacaggtgttaaaaatctttctacggatccgagtgagggggtccgggcttacatggccccttcccctttcactcccagcgtccaacccgggctgcggctgcaggaatggaggggaccacggcctcggcagccaagttttgctacttacgggagtactgaaggccctcggtgctggccagccagcgcgtgtacgggttgtcgctgctgtcgtagaaggggttcttcaggggcaggctctgcaccgcgtccagggcgccctgccccagcggcccggccctcttggctggctcgggcccctcgttctcttcctccggaccttcggccacagagccctcctcatcgttggtgtccggcaggtctaagatgtccttgaccgaaaaccccgtctttgtgttggtcagcgacatggttcgagaccccaaaatttatgtcgcaaagttgtagcttcacttggtcaattcgtggcgctcccctgccccg

SEQ ID NO: 150 - DNA, 3088 bp, synthetic construct
  source 1..3088: mol_type = other DNA
tcgtttttgcggttttcgaagttattttttttttcgtagtcggacgttgtcgttcggggtttagatagcgcgcgagattaggggagatgcgggggagggtgggcggttttgggttttcggtcgatcgttcgtaggtcggaggtcgagcgttttttttttcgttttaattttaggcgcggggtgtagtttgtggtcggaggggtttgtatcgggagtttcgttcgttttagggtttcggtattttcggttttttcgttcgagaggaatcgtttgtgggagtttcgcgggcggtcgggttcggtttttaggtttaggttttcggttcggcgtagtaggataaacgggagggtagggatttttttttttttttttttttaaatataggatttttattaaaatttttatttaaaaaatcgaaagttttttgcgttcggcgttgttggggaatcgcgtcgaatagttgagttttaagttcggggttgaaggttcggaggatagggtagagggttttttgtttatagggtttttttttttatatttgagaaatgtttgtataataaaataaaaagagaaaggaagaaagtaggggaaaacgtaaaaataaaaataaaataaaaaataaatttaaataagttataaagaaaggagttggatttagacgggaaataggtcggttttgaaggttattttggtaataattattatcgatatttataacgaaaagcgaaatttgttattagttgttagaacgtttatatggttataaatatttagtatttttttatttttttttaaaaaaaggaagaaattttttgtatttttaaagtgttttttatttgttttagaagacggttgataatatcgttatttatataaatatattttataaaatgtacgaaagtaggggtggggggtcggtttttttttcgtttttaagtgacgatattaacgttgggacggtttggttttttcggggagaggtaaagaagcgaagttgcgtaaatattttgtaaatacggcgtagagtttagtttttttttaaggtttagaaggagaggtatgggtagagttgggtgggtggaatttgttattttaaggagacgtaggtaatttttcggcgtttttttagtggagtcgagagttaattcgattttataataataattataataataataataattattataaggatcgaggttttttcgtcgttatcgtcgtcggggtggggtttgggtttggggtcgcgagtttcgttggggcggcgtttattaagtttattgttgggtttggattagggggtgtgttgtcgggtattggggggtgttggtcgagttgtattgggcgttgtattgtatgtgttgtagcgattgcgcgttgtaggtagaaaagggaatgttcgtttggaaggtggcggttgttaggttttgggttttgagcgcgtgatatggtttgtcgtttttgattaagacgggtacggttattcggcgcggcgagggtaggggcgttatttttatatttttttcggttcgggcgcgttttattttgtagcggtggttttggaattagattttgatttgcgtgggcgtgaggcggatgaggttggttaggtgttcgcgttcgggcgtcgataggtatcgttgttgtcgaaagcgtcgttttagttcgtaggtttgcgttttggagaaaagtattcgtcgttttcgttttttgtcggcgtttttttcgtcgttcggggtttttttgttattgttcggtgattcgtcggtcgagggtttcggggatttggagtttgagttttgagggggcgtttcggtagttagatcgtgtattggggggagggggagagagaagcgcgttaggcgtttggaggtcgcgcgtagtttgtattagattcggagtattttggatttttcgtgcgattttggattttcgcgtttggtagtttagtttcgttgtttttgttatgatttttgtttttttttacgtttgacgaagacgtcgggattttttagggtaaagagggaaattcgtaggagtgggggttaggggttcgttttttaaggggtttatatagtgtgggtgagaatttggggggggaagtggtattgaagattatcgcgggttaagggtttcggcgtttttaagcggtttttttttcggcgagacgtgggtagatttttaaaagaaatattttataattttagtattgatcggttcggttatttaaggtaggtttttgggggttttcgtttttgttttcgggttttttagatattttttgtattgtataaagcgttttttatgatcgcgtagggggtagggtgagttatttatattcgggttattcgaagtttttttattttttattcgtattcggacgaggttcgttggttatttttagggcgttaggttagttttttttagcggtcggagttcgggggttgcggttggagttatcggttcgggttgatattatatattagtttttgggaggtatagtttttttttttttttttttttaatttttcgtattgaaaaaaattttggagattaagttttttttcggaattaaggaggtttgaagaggagggtagaggatattttatataggtgttaaaaatttttttacggattcgagtgagggggttcgggtttatatggttttttttttttttatttttagcgtttaattcgggttgcggttgtaggaatggaggggattacggtttcggtagttaagttttgttatttacgggagtattgaaggttttcggtgttggttagttagcgcgtgtacgggttgtcgttgttgtcgtagaaggggttttttaggggtaggttttgtatcgcgtttagggcgttttgttttagcggttcggtttttttggttggttcgggtttttcgtttttttttttcggattttcggttatagagttttttttatcgttggtgttcggtaggtttaagatgtttttgatcgaaaatttcgtttttgtgttggttagcgatatggttcgagattttaaaatttatgtcgtaaagttgtagttttatttggttaattcgtggcgtttttttgtttcg

SEQ ID NO: 151 - DNA, 3088 bp, synthetic construct
  source 1..3088: mol_type = other DNA
cgaaacaaaaaaacgccacgaattaaccaaataaaactacaactttacgacataaattttaaaatctcgaaccatatcgctaaccaacacaaaaacgaaattttcgatcaaaaacatcttaaacctaccgaacaccaacgataaaaaaaactctataaccgaaaatccgaaaaaaaaaaacgaaaaacccgaaccaaccaaaaaaaccgaaccgctaaaacaaaacgccctaaacgcgatacaaaacctacccctaaaaaaccccttctacgacaacaacgacaacccgtacacgcgctaactaaccaacaccgaaaaccttcaatactcccgtaaataacaaaacttaactaccgaaaccgtaatcccctccattcctacaaccgcaacccgaattaaacgctaaaaataaaaaaaaaaaaaaccatataaacccgaaccccctcactcgaatccgtaaaaaaatttttaacacctatataaaatatcctctaccctcctcttcaaacctccttaattccgaaaaaaaacttaatctccaaaatttttttcaatacgaaaaattaaaaaaaaaaaaaaaaaaaaactatacctcccaaaaactaatatataatatcaacccgaaccgataactccaaccgcaacccccgaactccgaccgctaaaaaaaactaacctaacgccctaaaaataaccaacgaacctcgtccgaatacgaataaaaaataaaaaaacttcgaataacccgaatataaataactcaccctaccccctacgcgatcataaaaaacgctttatacaatacaaaaaatatctaaaaaacccgaaaacaaaaacgaaaacccccaaaaacctaccttaaataaccgaaccgatcaatactaaaattataaaatatttcttttaaaaatctacccacgtctcgccgaaaaaaaaaccgcttaaaaacgccgaaacccttaacccgcgataatcttcaataccacttccccccccaaattctcacccacactatataaaccccttaaaaaacgaacccctaacccccactcctacgaatttccctctttaccctaaaaaatcccgacgtcttcgtcaaacgtaaaaaaaaacaaaaatcataacaaaaacaacgaaactaaactaccaaacgcgaaaatccaaaatcgcacgaaaaatccaaaatactccgaatctaatacaaactacgcgcgacctccaaacgcctaacgcgcttctctctccccctccccccaatacacgatctaactaccgaaacgccccctcaaaactcaaactccaaatccccgaaaccctcgaccgacgaatcaccgaacaataacaaaaaaaccccgaacgacgaaaaaaacgccgacaaaaaacgaaaacgacgaatacttttctccaaaacgcaaacctacgaactaaaacgacgctttcgacaacaacgatacctatcgacgcccgaacgcgaacacctaaccaacctcatccgcctcacgcccacgcaaatcaaaatctaattccaaaaccaccgctacaaaataaaacgcgcccgaaccgaaaaaaatataaaaataacgcccctaccctcgccgcgccgaataaccgtacccgtcttaatcaaaaacgacaaaccatatcacgcgctcaaaacccaaaacctaacaaccgccaccttccaaacgaacattcccttttctacctacaacgcgcaatcgctacaacacatacaatacaacgcccaatacaactcgaccaacaccccccaatacccgacaacacaccccctaatccaaacccaacaataaacttaataaacgccgccccaacgaaactcgcgaccccaaacccaaaccccaccccgacgacgataacgacgaaaaaacctcgatccttataataattattattattattataattattattataaaatcgaattaactctcgactccactaaaaaaacgccgaaaaattacctacgtctccttaaaataacaaattccacccacccaactctacccatacctctccttctaaaccttaaaaaaaaactaaactctacgccgtatttacaaaatatttacgcaacttcgcttctttacctctccccgaaaaaaccaaaccgtcccaacgttaatatcgtcacttaaaaacgaaaaaaaaaccgaccccccacccctactttcgtacattttataaaatatatttatataaataacgatattatcaaccgtcttctaaaacaaataaaaaacactttaaaaatacaaaaaatttcttcctttttttaaaaaaaaataaaaaaatactaaatatttataaccatataaacgttctaacaactaataacaaatttcgcttttcgttataaatatcgataataattattaccaaaataaccttcaaaaccgacctatttcccgtctaaatccaactcctttctttataacttatttaaatttattttttattttatttttatttttacgttttcccctactttcttcctttctctttttattttattatacaaacatttctcaaatataaaaaaaaaaaccctataaacaaaaaaccctctaccctatcctccgaaccttcaaccccgaacttaaaactcaactattcgacgcgattccccaacaacgccgaacgcaaaaaactttcgattttttaaataaaaattttaataaaaatcctatatttaaaaaaaaaaaaaaaaaaaaatccctaccctcccgtttatcctactacgccgaaccgaaaacctaaacctaaaaaccgaacccgaccgcccgcgaaactcccacaaacgattcctctcgaacgaaaaaaccgaaaataccgaaaccctaaaacgaacgaaactcccgatacaaacccctccgaccacaaactacaccccgcgcctaaaattaaaacgaaaaaaaaaacgctcgacctccgacctacgaacgatcgaccgaaaacccaaaaccgcccaccctcccccgcatctcccctaatctcgcgcgctatctaaaccccgaacgacaacgtccgactacgaaaaaaaaaataacttcgaaaaccgcaaaaacga

SEQ ID NO: 152 - DNA, 3088 bp, synthetic construct
  source 1..3088: mol_type = other DNA
tcgcctttacgactcccgaaatcacttttcctcccgtaaccgaacgctaccgcccgaaattcaaacaacgcgcgaaaccaaaaaaaatacgaaaaaaaataaacgaccctaaacttccgaccgaccgctcgcaaaccgaaaaccgaacgttctcctccccgccccaatcctaaacgcgaaatacaacctataatcgaaaaaacctacaccgaaaaccccgcccgccccaaaaccccgatacccccgacctccccgcccgaaaaaaaccgcctataaaaaccccgcgaacgaccgaacccgactcccaaacccaaacctccgacccgacgcaataaaacaaacgaaaaaacaaaaattttttctttttttcttttttaaacacaaaatttttattaaaattcttatttaaaaaatcgaaaactttctacgcccgacgctattaaaaaaccgcgccgaataactaaactccaaattcgaaactaaaaacccgaaaaacaaaacaaaaaactccctacctacaaaattttcttttccatatttaaaaaatatttacacaataaaataaaaaaaaaaaaaaaaaaaacaaaaaaaaacgcaaaaacaaaaacaaaacaaaaaacaaacccaaacaaaccacaaaaaaaaaaattaaacccaaacgaaaaacaaaccgatcctaaaaatcattttaacaacaatcaccaccgatatttacaacgaaaaacgaaatctaccaccaattatcaaaacgtttacataaccataaatatttaacattttcttatttttttttaaaaaaaaaaaaaaattctctatatttttaaaatattctccacttactttaaaaaacgactaacaatatcgctactcacacaaacatattttacaaaatacacgaaaacaaaaataaaaaatcgatctttttctcgttttcaaataacgacattaacgctaaaacgatttaatccccccgaaaaaaaacaaaaaaacgaaactacgcaaacattctataaacacgacgtaaaattcaaccctctcccaaaattcaaaaaaaaaaacataaacaaaactaaataaataaaatctaccactccaaaaaaacgcaaacaacctcccgacgcctccctaataaaaccgaaaatcaactcgactccataataataattataataataataataaccaccataaaaaccgaaacctcctcgccgccaccgccgccgaaataaaacctaaacctaaaaccgcgaatctcgttaaaacgacgctcaccaaatccactactaaacctaaaccaaaaaatatactatcgaatactaaaaaatactaaccgaactatactaaacgttatactacatatactacaacgactacgcgctataaacaaaaaaaaaaatacccgcctaaaaaataacgactaccaaatcctaaactttaaacgcgtaacataatttaccgtccctaaccaaaacgaacacgaccacccgacgcgacgaaaacaaaaacgtcacctccatacctttctcgacccgaacgcgcttcatcttataacgataattctaaaaccaaatcttaacctacgtaaacgtaaaacgaataaaactaaccaaatattcgcgctcgaacgccgacaaataccgctactaccgaaaacgccgctccaactcgtaaatctacgccttaaaaaaaaacactcgccgctttcgcttcttaccgacgtcccccccgccgcccgaaatctccttatcattatccgataactcgtcgaccgaaaactccgaaaacttaaaacttaaatcctaaaaaaacgccccgacaaccaaaccgtacactaaaaaaaaaaaaaaaaaaaaacgcgtcaaacgtctaaaaaccgcgcgcaacctacaccaaactcgaaacaccctaaatcctccgtacgaccctaaacctccgcgcctaacaacccaaccccgctacctttaccataacccctaccttcctctacgcctaacgaaaacgtcgaaacctcccaaaataaaaaaaaaaatccgtaaaaataaaaactaaaaactcgcctttcaaaaaactcacataatataaataaaaatttaaaaaaaaaaataacactaaaaatcatcgcgaattaaaaactccgacgcccttaaacgatttcctctccgacgaaacgtaaacaaatttttaaaaaaaataccccacaatcccaacattaatcgactcgaccatttaaaacaaatttttaaaaactttcgtttctactttcgaaccttctaaacaccccctacactacacaaaacgtcctctataatcgcgcaaaaaacaaaataaactatttatacccgaaccacccgaaacctccccaccctccacccgtacccgaacgaaactcgctaactacctccaaaacgccaaaccaatccctcccaacgaccgaaatccgaaaactacgactaaaaccatcgatccgaattaacatcatataccaacccctaaaaaatataacccttctccctctttctccttaacttctcgcattaaaaaaaattttaaaaaccaaattctttcccgaaactaaaaaaacttaaaaaaaaaaacaaaaaacatcctatacaaatattaaaaatctttctacgaatccgaataaaaaaatccgaacttacataaccccttcccctttcactcccaacgtccaacccgaactacgactacaaaaataaaaaaaaccacgacctcgacaaccaaattttactacttacgaaaatactaaaaaccctcgatactaaccaaccaacgcgtatacgaattatcgctactatcgtaaaaaaaattcttcaaaaacaaactctacaccgcgtccaaaacgccctaccccaacgacccgaccctcttaactaactcgaacccctcgttctcttcctccgaaccttcgaccacaaaaccctcctcatcgttaatatccgacaaatctaaaatatccttaaccgaaaaccccgtctttatattaatcaacgacataattcgaaaccccaaaatttatatcgcaaaattataacttcacttaatcaattcgtaacgctcccctaccccg

SEQ ID NO: 153 - DNA, 3088 bp, synthetic construct
  source 1..3088: mol_type = other DNA
cggggtaggggagcgttacgaattgattaagtgaagttataattttgcgatataaattttggggtttcgaattatgtcgttgattaatataaagacggggttttcggttaaggatattttagatttgtcggatattaacgatgaggagggttttgtggtcgaaggttcggaggaagagaacgaggggttcgagttagttaagagggtcgggtcgttggggtagggcgttttggacgcggtgtagagtttgtttttgaagaattttttttacgatagtagcgataattcgtatacgcgttggttggttagtatcgagggtttttagtattttcgtaagtagtaaaatttggttgtcgaggtcgtggttttttttatttttgtagtcgtagttcgggttggacgttgggagtgaaaggggaaggggttatgtaagttcggatttttttattcggattcgtagaaagatttttaatatttgtataggatgttttttgtttttttttttaagtttttttagtttcgggaaagaatttggtttttaaaattttttttaatgcgagaagttaaggagaaagagggagaagggttatattttttaggggttggtatatgatgttaattcggatcgatggttttagtcgtagttttcggatttcggtcgttgggagggattggtttggcgttttggaggtagttagcgagtttcgttcgggtacgggtggagggtggggaggtttcgggtggttcgggtataaatagtttattttgttttttgcgcgattatagaggacgttttgtgtagtgtagggggtgtttagaaggttcgaaagtagaaacgaaagtttttaaaaatttgttttaaatggtcgagtcgattaatgttgggattgtggggtattttttttaaaaatttgtttacgtttcgtcggagaggaaatcgtttaagggcgtcggagtttttaattcgcgatgatttttagtgttatttttttttttaaatttttatttatattatgtgagttttttgaaaggcgagtttttagtttttatttttacggatttttttttttattttgggaggtttcgacgttttcgttaggcgtagaggaaggtaggggttatggtaaaggtagcggggttgggttgttaggcgcggaggtttagggtcgtacggaggatttagggtgtttcgagtttggtgtaggttgcgcgcggtttttagacgtttgacgcgtttttttttttttttttttttagtgtacggtttggttgtcggggcgtttttttaggatttaagttttaagttttcggagttttcggtcgacgagttatcggataatgataaggagatttcgggcggcgggggggacgtcggtaagaagcgaaagcggcgagtgtttttttttaaggcgtagatttacgagttggagcggcgttttcggtagtagcggtatttgtcggcgttcgagcgcgaatatttggttagttttattcgttttacgtttacgtaggttaagatttggttttagaattatcgttataagatgaagcgcgttcgggtcgagaaaggtatggaggtgacgtttttgttttcgtcgcgtcgggtggtcgtgttcgttttggttagggacggtaaattatgttacgcgtttaaagtttaggatttggtagtcgttattttttaggcgggtatttttttttttgtttatagcgcgtagtcgttgtagtatatgtagtataacgtttagtatagttcggttagtattttttagtattcgatagtatattttttggtttaggtttagtagtggatttggtgagcgtcgttttaacgagattcgcggttttaggtttaggttttatttcggcggcggtggcggcgaggaggtttcggtttttatggtggttattattattattataattattattatggagtcgagttgattttcggttttattagggaggcgtcgggaggttgtttgcgtttttttggagtggtagattttatttatttagttttgtttatgtttttttttttgaattttgggagagggttgaattttacgtcgtgtttatagaatgtttgcgtagtttcgtttttttgttttttttcggggggattaaatcgttttagcgttaatgtcgttatttgaaaacgagaaaaagatcgattttttatttttgttttcgtgtattttgtaaaatatgtttgtgtgagtagcgatattgttagtcgttttttaaagtaagtggagaatattttaaaaatatagagaatttttttttttttttaaaaaaaaataagaaaatgttaaatatttatggttatgtaaacgttttgataattggtggtagatttcgtttttcgttgtaaatatcggtggtgattgttgttaaaatgatttttaggatcggtttgtttttcgtttgggtttaatttttttttttgtggtttgtttgggtttgttttttgttttgtttttgtttttgcgtttttttttgttttttttttttttttttttattttattgtgtaaatattttttaaatatggaaaagaaaattttgtaggtagggagttttttgttttgtttttcgggtttttagtttcgaatttggagtttagttattcggcgcggttttttaatagcgtcgggcgtagaaagttttcgattttttaaataagaattttaataaaaattttgtgtttaaaaaagaaaaaaagaaaaaatttttgttttttcgtttgttttattgcgtcgggtcggaggtttgggtttgggagtcgggttcggtcgttcgcggggtttttataggcggtttttttcgggcggggaggtcgggggtatcggggttttggggcgggcggggttttcggtgtaggttttttcgattataggttgtatttcgcgtttaggattggggcggggaggagaacgttcggttttcggtttgcgagcggtcggtcggaagtttagggtcgtttattttttttcgtattttttttggtttcgcgcgttgtttgaatttcgggcggtagcgttcggttacgggaggaaaagtgatttcgggagtcgtaaaggcga

SEQ ID NO: 154 - DNA, 1067 bp, Homo sapiens
  source 1..1067: mol_type = genomic DNA
ggctggccaggtgttcgcgctcgggcgccgacaggtaccgctgctgccgaaagcgccgctccagctcgtaggtctgcgccttggagaaaagcactcgccgctttcgcttcttgccggcgtcccccccgccgcccggggtctccttgtcattgtccggtgactcgtcggccgagggctccggggacttggagcttgagtcctgagggggcgccccggcagccagaccgtgcactggggggagggggagagagaagcgcgtcaggcgtctggaggccgcgcgcagcctgcaccagactcggagcaccctggatcctccgtgcgaccctggacctccgcgcctggcagcccagccccgctgcctttgccatgacccctgccttcctctacgcctgacgaagacgtcgggacctcccagggtaaagagggaaatccgtaggagtgggggctaggggctcgcctttcaaggggctcacatagtgtgggtgagaatttggggggggaagtggcactgaagatcatcgcgggttaagggctccggcgcccttaagcggtttcctctccggcgagacgtgggcagatttttaaaagaaataccccacaatcccagcattgatcggctcggccatttaaggcaggtttttgggggctttcgtttctgctttcgggccttctagacaccccctgcactgcacaaagcgtcctctatgatcgcgcagggggcagggtgagctatttatacccgggccacccgaagcctccccaccctccacccgtacccggacgaggctcgctggctacctccagggcgccaggccagtccctcccagcggccggagtccgggggctgcggctggagccatcggtccgggttgacatcatataccagcccctgggaggtatagcccttctccctctttctccttaacttctcgcattgaaaaaaattttggagaccaagttctttcccggaactaaggaggcttgaagaggagggcagaggacatcctatacaggtgttaaaaatctttctacggatccgagtgagggggtccgggcttacatggccc

SEQ ID NO: 155 - DNA, 1067 bp, synthetic construct
  source 1..1067: mol_type = other DNA
ggttggttaggtgttcgcgttcgggcgtcgataggtatcgttgttgtcgaaagcgtcgttttagttcgtaggtttgcgttttggagaaaagtattcgtcgttttcgttttttgtcggcgtttttttcgtcgttcggggtttttttgttattgttcggtgattcgtcggtcgagggtttcggggatttggagtttgagttttgagggggcgtttcggtagttagatcgtgtattggggggagggggagagagaagcgcgttaggcgtttggaggtcgcgcgtagtttgtattagattcggagtattttggatttttcgtgcgattttggattttcgcgtttggtagtttagtttcgttgtttttgttatgatttttgtttttttttacgtttgacgaagacgtcgggattttttagggtaaagagggaaattcgtaggagtgggggttaggggttcgttttttaaggggtttatatagtgtgggtgagaatttggggggggaagtggtattgaagattatcgcgggttaagggtttcggcgtttttaagcggtttttttttcggcgagacgtgggtagatttttaaaagaaatattttataattttagtattgatcggttcggttatttaaggtaggtttttgggggttttcgtttttgttttcgggttttttagatattttttgtattgtataaagcgttttttatgatcgcgtagggggtagggtgagttatttatattcgggttattcgaagtttttttattttttattcgtattcggacgaggttcgttggttatttttagggcgttaggttagttttttttagcggtcggagttcgggggttgcggttggagttatcggttcgggttgatattatatattagtttttgggaggtatagtttttttttttttttttttttaatttttcgtattgaaaaaaattttggagattaagttttttttcggaattaaggaggtttgaagaggagggtagaggatattttatataggtgttaaaaatttttttacggattcgagtgagggggttcgggtttatatggttt

SEQ ID NO: 156 - DNA, 1067 bp, synthetic construct
  source 1..1067: mol_type = other DNA
aaaccatataaacccgaaccccctcactcgaatccgtaaaaaaatttttaacacctatataaaatatcctctaccctcctcttcaaacctccttaattccgaaaaaaaacttaatctccaaaatttttttcaatacgaaaaattaaaaaaaaaaaaaaaaaaaaactatacctcccaaaaactaatatataatatcaacccgaaccgataactccaaccgcaacccccgaactccgaccgctaaaaaaaactaacctaacgccctaaaaataaccaacgaacctcgtccgaatacgaataaaaaataaaaaaacttcgaataacccgaatataaataactcaccctaccccctacgcgatcataaaaaacgctttatacaatacaaaaaatatctaaaaaacccgaaaacaaaaacgaaaacccccaaaaacctaccttaaataaccgaaccgatcaatactaaaattataaaatatttcttttaaaaatctacccacgtctcgccgaaaaaaaaaccgcttaaaaacgccgaaacccttaacccgcgataatcttcaataccacttccccccccaaattctcacccacactatataaaccccttaaaaaacgaacccctaacccccactcctacgaatttccctctttaccctaaaaaatcccgacgtcttcgtcaaacgtaaaaaaaaacaaaaatcataacaaaaacaacgaaactaaactaccaaacgcgaaaatccaaaatcgcacgaaaaatccaaaatactccgaatctaatacaaactacgcgcgacctccaaacgcctaacgcgcttctctctccccctccccccaatacacgatctaactaccgaaacgccccctcaaaactcaaactccaaatccccgaaaccctcgaccgacgaatcaccgaacaataacaaaaaaaccccgaacgacgaaaaaaacgccgacaaaaaacgaaaacgacgaatacttttctccaaaacgcaaacctacgaactaaaacgacgctttcgacaacaacgatacctatcgacgcccgaacgcgaacacctaaccaacc

SEQ ID NO: 157 - DNA, 1067 bp, synthetic construct
  source 1..1067: mol_type = other DNA
aactaaccaaatattcgcgctcgaacgccgacaaataccgctactaccgaaaacgccgctccaactcgtaaatctacgccttaaaaaaaaacactcgccgctttcgcttcttaccgacgtcccccccgccgcccgaaatctccttatcattatccgataactcgtcgaccgaaaactccgaaaacttaaaacttaaatcctaaaaaaacgccccgacaaccaaaccgtacactaaaaaaaaaaaaaaaaaaaaacgcgtcaaacgtctaaaaaccgcgcgcaacctacaccaaactcgaaacaccctaaatcctccgtacgaccctaaacctccgcgcctaacaacccaaccccgctacctttaccataacccctaccttcctctacgcctaacgaaaacgtcgaaacctcccaaaataaaaaaaaaaatccgtaaaaataaaaactaaaaactcgcctttcaaaaaactcacataatataaataaaaatttaaaaaaaaaaataacactaaaaatcatcgcgaattaaaaactccgacgcccttaaacgatttcctctccgacgaaacgtaaacaaatttttaaaaaaaataccccacaatcccaacattaatcgactcgaccatttaaaacaaatttttaaaaactttcgtttctactttcgaaccttctaaacaccccctacactacacaaaacgtcctctataatcgcgcaaaaaacaaaataaactatttatacccgaaccacccgaaacctccccaccctccacccgtacccgaacgaaactcgctaactacctccaaaacgccaaaccaatccctcccaacgaccgaaatccgaaaactacgactaaaaccatcgatccgaattaacatcatataccaacccctaaaaaatataacccttctccctctttctccttaacttctcgcattaaaaaaaattttaaaaaccaaattctttcccgaaactaaaaaaacttaaaaaaaaaaacaaaaaacatcctatacaaatattaaaaatctttctacgaatccgaataaaaaaatccgaacttacataaccc

SEQ ID NO: 158 - DNA, 1067 bp, synthetic construct
  source 1..1067: mol_type = other DNA
gggttatgtaagttcggatttttttattcggattcgtagaaagatttttaatatttgtataggatgttttttgtttttttttttaagtttttttagtttcgggaaagaatttggtttttaaaattttttttaatgcgagaagttaaggagaaagagggagaagggttatattttttaggggttggtatatgatgttaattcggatcgatggttttagtcgtagttttcggatttcggtcgttgggagggattggtttggcgttttggaggtagttagcgagtttcgttcgggtacgggtggagggtggggaggtttcgggtggttcgggtataaatagtttattttgttttttgcgcgattatagaggacgttttgtgtagtgtagggggtgtttagaaggttcgaaagtagaaacgaaagtttttaaaaatttgttttaaatggtcgagtcgattaatgttgggattgtggggtattttttttaaaaatttgtttacgtttcgtcggagaggaaatcgtttaagggcgtcggagtttttaattcgcgatgatttttagtgttatttttttttttaaatttttatttatattatgtgagttttttgaaaggcgagtttttagtttttatttttacggatttttttttttattttgggaggtttcgacgttttcgttaggcgtagaggaaggtaggggttatggtaaaggtagcggggttgggttgttaggcgcggaggtttagggtcgtacggaggatttagggtgtttcgagtttggtgtaggttgcgcgcggtttttagacgtttgacgcgtttttttttttttttttttttagtgtacggtttggttgtcggggcgtttttttaggatttaagttttaagttttcggagttttcggtcgacgagttatcggataatgataaggagatttcgggcggcgggggggacgtcggtaagaagcgaaagcggcgagtgtttttttttaaggcgtagatttacgagttggagcggcgttttcggtagtagcggtatttgtcggcgttcgagcgcgaatatttggttagtt

SEQ ID NO: 159 - DNA, 67 bp, Homo sapiens
  source 1..67: mol_type = genomic DNA
aagtggcactgaagatcatcgcgggttaagggctccggcgcccttaagcggtttcctctccggcgag

SEQ ID NO: 160 - DNA, 67 bp, synthetic construct
  source 1..67: mol_type = other DNA
aagtggtattgaagattatcgcgggttaagggtttcggcgtttttaagcggtttttttttcggcgag

SEQ ID NO: 161 - DNA, 67 bp, synthetic construct
  source 1..67: mol_type = other DNA
ctcgccgaaaaaaaaaccgcttaaaaacgccgaaacccttaacccgcgataatcttcaataccactt

SEQ ID NO: 162 - DNA, 67 bp, synthetic construct
  source 1..67: mol_type = other DNA
aaataacactaaaaatcatcgcgaattaaaaactccgacgcccttaaacgatttcctctccgacgaa

SEQ ID NO: 163 - DNA, 67 bp, synthetic construct
  source 1..67: mol_type = other DNA
ttcgtcggagaggaaatcgtttaagggcgtcggagtttttaattcgcgatgatttttagtgttattt

SEQ ID NO: 164 - DNA, 1120 bp, Homo sapiens
  source 1..1120: mol_type = genomic DNA
ccgaaaaacacgttctaggcgccgggattccagatacctgggaaatagagtgcacgcagctgttgagaggcctcgcgcttggcttctcctatcactgaggcgcagaggtgctgtggacagcccagacccacacggcgcccgaggtgaaacagaaccctcagtctccctatgaggccactggcactctcggctgtccccagagctctccgacttagagctgaatgcaaagtaagcgctcgaaatgcagaagtagccggggccgcccacggcacctgcctcgctcggggcgagagaagacgccaggctgaggtcccagcgacctcaggcaccagctccgaaggagggcggggagaccgcaaaggggaagtgcccggagggccaacggcccccgcgcaccctgcgcccctctgaagcgcgccgcctccccgcgccggggactgggacctgcctctggggaatccgcctagaagacggcggcggactggggtcgggcactctccagggctgtcaggccctccccagccctgcacctgccgcgccgccccacctcgccaggaagtctcagagaccccggggatggggtgggagcgccttcccatcgcgggctcaaaaagaaggaaggacgcccccaggggtcgtagaaggaggactagctccaagccacaactttcttcggacccaaggcaggccggctggggctccgcgcctacacggcccctggcgggggtccgcgcgccccgggagccccgcggctcggggaggaaagaggagacaagagacaggcgaggattacggggctgacccagccggggtagggaccatcgtggaaaaactttggcgaggtggggggacgcggaaagagagcggcccgcgccctgcaccttgcgccgggcatcccgcgccagtgcctcgctcccagtgccccgcgccccgcgccccgcgccttgccttcaccccgggccagctgcatcgcgcccgcgccgcaggaaccgtggagttggaaagtgggggcgccgcggctggggggctgcttcagctgcgcctcggccagcgatcggcgggccgggctcaaatccagccaggctgggcaggcggtggccgcgcgactggggaccgggcg

SEQ ID NO: 165 - DNA, 1120 bp, synthetic construct
  source 1..1120: mol_type = other DNA
tcgaaaaatacgttttaggcgtcgggattttagatatttgggaaatagagtgtacgtagttgttgagaggtttcgcgtttggttttttttattattgaggcgtagaggtgttgtggatagtttagatttatacggcgttcgaggtgaaatagaatttttagtttttttatgaggttattggtattttcggttgtttttagagtttttcgatttagagttgaatgtaaagtaagcgttcgaaatgtagaagtagtcggggtcgtttacggtatttgtttcgttcggggcgagagaagacgttaggttgaggttttagcgattttaggtattagtttcgaaggagggcggggagatcgtaaaggggaagtgttcggagggttaacggttttcgcgtattttgcgtttttttgaagcgcgtcgttttttcgcgtcggggattgggatttgtttttggggaattcgtttagaagacggcggcggattggggtcgggtattttttagggttgttaggttttttttagttttgtatttgtcgcgtcgttttatttcgttaggaagttttagagatttcggggatggggtgggagcgtttttttatcgcgggtttaaaaagaaggaaggacgtttttaggggtcgtagaaggaggattagttttaagttataatttttttcggatttaaggtaggtcggttggggtttcgcgtttatacggtttttggcgggggttcgcgcgtttcgggagtttcgcggttcggggaggaaagaggagataagagataggcgaggattacggggttgatttagtcggggtagggattatcgtggaaaaattttggcgaggtggggggacgcggaaagagagcggttcgcgttttgtattttgcgtcgggtatttcgcgttagtgtttcgtttttagtgtttcgcgtttcgcgtttcgcgttttgtttttatttcgggttagttgtatcgcgttcgcgtcgtaggaatcgtggagttggaaagtgggggcgtcgcggttggggggttgttttagttgcgtttcggttagcgatcggcgggtcgggtttaaatttagttaggttgggtaggcggtggtcgcgcgattggggatcgggcg

SEQ ID NO: 166 - DNA, 1120 bp, synthetic construct
  source 1..1120: mol_type = other DNA
cgcccgatccccaatcgcgcgaccaccgcctacccaacctaactaaatttaaacccgacccgccgatcgctaaccgaaacgcaactaaaacaaccccccaaccgcgacgcccccactttccaactccacgattcctacgacgcgaacgcgatacaactaacccgaaataaaaacaaaacgcgaaacgcgaaacgcgaaacactaaaaacgaaacactaacgcgaaatacccgacgcaaaatacaaaacgcgaaccgctctctttccgcgtccccccacctcgccaaaatttttccacgataatccctaccccgactaaatcaaccccgtaatcctcgcctatctcttatctcctctttcctccccgaaccgcgaaactcccgaaacgcgcgaacccccgccaaaaaccgtataaacgcgaaaccccaaccgacctaccttaaatccgaaaaaaattataacttaaaactaatcctccttctacgacccctaaaaacgtccttccttctttttaaacccgcgataaaaaaacgctcccaccccatccccgaaatctctaaaacttcctaacgaaataaaacgacgcgacaaatacaaaactaaaaaaaacctaacaaccctaaaaaatacccgaccccaatccgccgccgtcttctaaacgaattccccaaaaacaaatcccaatccccgacgcgaaaaaacgacgcgcttcaaaaaaacgcaaaatacgcgaaaaccgttaaccctccgaacacttcccctttacgatctccccgccctccttcgaaactaatacctaaaatcgctaaaacctcaacctaacgtcttctctcgccccgaacgaaacaaataccgtaaacgaccccgactacttctacatttcgaacgcttactttacattcaactctaaatcgaaaaactctaaaaacaaccgaaaataccaataacctcataaaaaaactaaaaattctatttcacctcgaacgccgtataaatctaaactatccacaacacctctacgcctcaataataaaaaaaaccaaacgcgaaacctctcaacaactacgtacactctatttcccaaatatctaaaatcccgacgcctaaaacgtatttttcga

SEQ ID NO: 167 - DNA, 1120 bp, synthetic construct
  source 1..1120: mol_type = other DNA
ccgaaaaacacgttctaaacgccgaaattccaaatacctaaaaaataaaatacacgcaactattaaaaaacctcgcgcttaacttctcctatcactaaaacgcaaaaatactataaacaacccaaacccacacgacgcccgaaataaaacaaaaccctcaatctccctataaaaccactaacactctcgactatccccaaaactctccgacttaaaactaaatacaaaataaacgctcgaaatacaaaaataaccgaaaccgcccacgacacctacctcgctcgaaacgaaaaaaaacgccaaactaaaatcccaacgacctcaaacaccaactccgaaaaaaaacgaaaaaaccgcaaaaaaaaaatacccgaaaaaccaacgacccccgcgcaccctacgcccctctaaaacgcgccgcctccccgcgccgaaaactaaaacctacctctaaaaaatccgcctaaaaaacgacgacgaactaaaatcgaacactctccaaaactatcaaaccctccccaaccctacacctaccgcgccgccccacctcgccaaaaaatctcaaaaaccccgaaaataaaataaaaacgccttcccatcgcgaactcaaaaaaaaaaaaaaacgcccccaaaaatcgtaaaaaaaaaactaactccaaaccacaactttcttcgaacccaaaacaaaccgactaaaactccgcgcctacacgacccctaacgaaaatccgcgcgccccgaaaaccccgcgactcgaaaaaaaaaaaaaaaacaaaaaacaaacgaaaattacgaaactaacccaaccgaaataaaaaccatcgtaaaaaaactttaacgaaataaaaaaacgcgaaaaaaaaacgacccgcgccctacaccttacgccgaacatcccgcgccaatacctcgctcccaataccccgcgccccgcgccccgcgccttaccttcaccccgaaccaactacatcgcgcccgcgccgcaaaaaccgtaaaattaaaaaataaaaacgccgcgactaaaaaactacttcaactacgcctcgaccaacgatcgacgaaccgaactcaaatccaaccaaactaaacaaacgataaccgcgcgactaaaaaccgaacg

SEQ ID NO: 168 - DNA, 1120 bp, synthetic construct
  source 1..1120: mol_type = other DNA
cgttcggtttttagtcgcgcggttatcgtttgtttagtttggttggatttgagttcggttcgtcgatcgttggtcgaggcgtagttgaagtagttttttagtcgcggcgtttttattttttaattttacggtttttgcggcgcgggcgcgatgtagttggttcggggtgaaggtaaggcgcggggcgcggggcgcggggtattgggagcgaggtattggcgcgggatgttcggcgtaaggtgtagggcgcgggtcgtttttttttcgcgtttttttatttcgttaaagtttttttacgatggtttttatttcggttgggttagtttcgtaattttcgtttgttttttgttttttttttttttttcgagtcgcggggttttcggggcgcgcggattttcgttaggggtcgtgtaggcgcggagttttagtcggtttgttttgggttcgaagaaagttgtggtttggagttagttttttttttacgatttttgggggcgtttttttttttttttgagttcgcgatgggaaggcgtttttattttattttcggggtttttgagattttttggcgaggtggggcggcgcggtaggtgtagggttggggagggtttgatagttttggagagtgttcgattttagttcgtcgtcgttttttaggcggattttttagaggtaggttttagttttcggcgcggggaggcggcgcgttttagaggggcgtagggtgcgcgggggtcgttggtttttcgggtattttttttttgcggttttttcgttttttttcggagttggtgtttgaggtcgttgggattttagtttggcgttttttttcgtttcgagcgaggtaggtgtcgtgggcggtttcggttatttttgtatttcgagcgtttattttgtatttagttttaagtcggagagttttggggatagtcgagagtgttagtggttttatagggagattgagggttttgttttatttcgggcgtcgtgtgggtttgggttgtttatagtatttttgcgttttagtgataggagaagttaagcgcgaggttttttaatagttgcgtgtattttattttttaggtatttggaatttcggcgtttagaacgtgtttttcgg

SEQ ID NO: 169 - DNA, 1077 bp, Homo sapiens
  source 1..1077: mol_type = genomic DNA
cctggtaagtaaatgtggatttattagtaaaataagcctagctgcagattcctcatccagatgttcctgaaagaaggcccctggctctgtcttcccggctgtttaaaagaatcattgtttctaattctttcctcccattgtactaagatcaactgtggcaggaactgggtccccctccaggacaaattgttgtttgtcagctgattggttcatacattcattcatcatttacaaggtacttactatatccccaggctgccgtagacacagcctttgctctcccgaaaaacacgttctaggcgccgggattccagatacctgggaaatagagtgcacgcagctgttgagaggcctcgcgcttggcttctcctatcactgaggcgcagaggtgctgtggacagcccagacccacacggcgcccgaggtgaaacagaaccctcagtctccctatgaggccactggcactctcggctgtccccagagctctccgacttagagctgaatgcaaagtaagcgctcgaaatgcagaagtagccggggccgcccacggcacctgcctcgctcggggcgagagaagacgccaggctgaggtcccagcgacctcaggcaccagctccgaaggagggcggggagaccgcaaaggggaagtgcccggagggccaacggcccccgcgcaccctgcgcccctctgaagcgcgccgcctccccgcgccggggactgggacctgcctctggggaatccgcctagaagacggcggcggactggggtcgggcactctccagggctgtcaggccctccccagccctgcacctgccgcgccgccccacctcgccaggaagtctcagagaccccggggatggggtgggagcgccttcccatcgcgggctcaaaaagaaggaaggacgcccccaggggtcgtagaaggaggactagctccaagccacaactttcttcggacccaaggcaggccggctggggctccgcgcctacacggcccctggcgggggtccgcgcgccccgggagccccgcggctcggggaggaaagaggagacaagagacaggcgaggattacggg

SEQ ID NO: 170 - DNA, 1077 bp, synthetic construct
  source 1..1077: mol_type = other DNA
tttggtaagtaaatgtggatttattagtaaaataagtttagttgtagattttttatttagatgtttttgaaagaaggtttttggttttgttttttcggttgtttaaaagaattattgtttttaattttttttttttattgtattaagattaattgtggtaggaattgggtttttttttaggataaattgttgtttgttagttgattggtttatatatttatttattatttataaggtatttattatatttttaggttgtcgtagatatagtttttgttttttcgaaaaatacgttttaggcgtcgggattttagatatttgggaaatagagtgtacgtagttgttgagaggtttcgcgtttggttttttttattattgaggcgtagaggtgttgtggatagtttagatttatacggcgttcgaggtgaaatagaatttttagtttttttatgaggttattggtattttcggttgtttttagagtttttcgatttagagttgaatgtaaagtaagcgttcgaaatgtagaagtagtcggggtcgtttacggtatttgtttcgttcggggcgagagaagacgttaggttgaggttttagcgattttaggtattagtttcgaaggagggcggggagatcgtaaaggggaagtgttcggagggttaacggttttcgcgtattttgcgtttttttgaagcgcgtcgttttttcgcgtcggggattgggatttgtttttggggaattcgtttagaagacggcggcggattggggtcgggtattttttagggttgttaggttttttttagttttgtatttgtcgcgtcgttttatttcgttaggaagttttagagatttcggggatggggtgggagcgtttttttatcgcgggtttaaaaagaaggaaggacgtttttaggggtcgtagaaggaggattagttttaagttataatttttttcggatttaaggtaggtcggttggggtttcgcgtttatacggtttttggcgggggttcgcgcgtttcgggagtttcgcggttcggggaggaaagaggagataagagataggcgaggattacggg

SEQ ID NO: 171 - DNA, 1077 bp, synthetic construct
  source 1..1077: mol_type = other DNA
cccgtaatcctcgcctatctcttatctcctctttcctccccgaaccgcgaaactcccgaaacgcgcgaacccccgccaaaaaccgtataaacgcgaaaccccaaccgacctaccttaaatccgaaaaaaattataacttaaaactaatcctccttctacgacccctaaaaacgtccttccttctttttaaacccgcgataaaaaaacgctcccaccccatccccgaaatctctaaaacttcctaacgaaataaaacgacgcgacaaatacaaaactaaaaaaaacctaacaaccctaaaaaatacccgaccccaatccgccgccgtcttctaaacgaattccccaaaaacaaatcccaatccccgacgcgaaaaaacgacgcgcttcaaaaaaacgcaaaatacgcgaaaaccgttaaccctccgaacacttcccctttacgatctccccgccctccttcgaaactaatacctaaaatcgctaaaacctcaacctaacgtcttctctcgccccgaacgaaacaaataccgtaaacgaccccgactacttctacatttcgaacgcttactttacattcaactctaaatcgaaaaactctaaaaacaaccgaaaataccaataacctcataaaaaaactaaaaattctatttcacctcgaacgccgtataaatctaaactatccacaacacctctacgcctcaataataaaaaaaaccaaacgcgaaacctctcaacaactacgtacactctatttcccaaatatctaaaatcccgacgcctaaaacgtatttttcgaaaaaacaaaaactatatctacgacaacctaaaaatataataaataccttataaataataaataaatatataaaccaatcaactaacaaacaacaatttatcctaaaaaaaaacccaattcctaccacaattaatcttaatacaataaaaaaaaaaaattaaaaacaataattcttttaaacaaccgaaaaaacaaaaccaaaaaccttctttcaaaaacatctaaataaaaaatctacaactaaacttattttactaataaatccacatttacttaccaaa

SEQ ID NO: 172 - DNA, 1077 bp, synthetic construct
  source 1..1077: mol_type = other DNA
cctaataaataaatataaatttattaataaaataaacctaactacaaattcctcatccaaatattcctaaaaaaaaacccctaactctatcttcccgactatttaaaaaaatcattatttctaattctttcctcccattatactaaaatcaactataacaaaaactaaatccccctccaaaacaaattattatttatcaactaattaattcatacattcattcatcatttacaaaatacttactatatccccaaactaccgtaaacacaacctttactctcccgaaaaacacgttctaaacgccgaaattccaaatacctaaaaaataaaatacacgcaactattaaaaaacctcgcgcttaacttctcctatcactaaaacgcaaaaatactataaacaacccaaacccacacgacgcccgaaataaaacaaaaccctcaatctccctataaaaccactaacactctcgactatccccaaaactctccgacttaaaactaaatacaaaataaacgctcgaaatacaaaaataaccgaaaccgcccacgacacctacctcgctcgaaacgaaaaaaaacgccaaactaaaatcccaacgacctcaaacaccaactccgaaaaaaaacgaaaaaaccgcaaaaaaaaaatacccgaaaaaccaacgacccccgcgcaccctacgcccctctaaaacgcgccgcctccccgcgccgaaaactaaaacctacctctaaaaaatccgcctaaaaaacgacgacgaactaaaatcgaacactctccaaaactatcaaaccctccccaaccctacacctaccgcgccgccccacctcgccaaaaaatctcaaaaaccccgaaaataaaataaaaacgccttcccatcgcgaactcaaaaaaaaaaaaaaacgcccccaaaaatcgtaaaaaaaaaactaactccaaaccacaactttcttcgaacccaaaacaaaccgactaaaactccgcgcctacacgacccctaacgaaaatccgcgcgccccgaaaaccccgcgactcgaaaaaaaaaaaaaaaacaaaaaacaaacgaaaattacgaa

SEQ ID NO: 173 - DNA, 1077 bp, synthetic construct
  source 1..1077: mol_type = other DNA
ttcgtaattttcgtttgttttttgttttttttttttttttcgagtcgcggggttttcggggcgcgcggattttcgttaggggtcgtgtaggcgcggagttttagtcggtttgttttgggttcgaagaaagttgtggtttggagttagttttttttttacgatttttgggggcgtttttttttttttttgagttcgcgatgggaaggcgtttttattttattttcggggtttttgagattttttggcgaggtggggcggcgcggtaggtgtagggttggggagggtttgatagttttggagagtgttcgattttagttcgtcgtcgttttttaggcggattttttagaggtaggttttagttttcggcgcggggaggcggcgcgttttagaggggcgtagggtgcgcgggggtcgttggtttttcgggtattttttttttgcggttttttcgttttttttcggagttggtgtttgaggtcgttgggattttagtttggcgttttttttcgtttcgagcgaggtaggtgtcgtgggcggtttcggttatttttgtatttcgagcgtttattttgtatttagttttaagtcggagagttttggggatagtcgagagtgttagtggttttatagggagattgagggttttgttttatttcgggcgtcgtgtgggtttgggttgtttatagtatttttgcgttttagtgataggagaagttaagcgcgaggttttttaatagttgcgtgtattttattttttaggtatttggaatttcggcgtttagaacgtgtttttcgggagagtaaaggttgtgtttacggtagtttggggatatagtaagtattttgtaaatgatgaatgaatgtatgaattaattagttgataaataataatttgttttggagggggatttagtttttgttatagttgattttagtataatgggaggaaagaattagaaataatgatttttttaaatagtcgggaagatagagttaggggtttttttttaggaatatttggatgaggaatttgtagttaggtttattttattaataaatttatatttatttattagg

SEQ ID NO: 174 - DNA, 77 bp, Homo sapiens
  source 1..77: mol_type = genomic DNA
gaatgcaaagtaagcgctcgaaatgcagaagtagccggggccgcccacggcacctgcctcgctcggggcgagagaag

SEQ ID NO: 175 - DNA, 77 bp, synthetic construct
  source 1..77: mol_type = other DNA
gaatgtaaagtaagcgttcgaaatgtagaagtagtcggggtcgtttacggtatttgtttcgttcggggcgagagaag

SEQ ID NO: 176 - DNA, 77 bp, synthetic construct
  source 1..77: mol_type = other DNA
cttctctcgccccgaacgaaacaaataccgtaaacgaccccgactacttctacatttcgaacgcttactttacattc

SEQ ID NO: 177 - DNA, 77 bp, synthetic construct
  source 1..77: mol_type = other DNA
aaatacaaaataaacgctcgaaatacaaaaataaccgaaaccgcccacgacacctacctcgctcgaaacgaaaaaaa

SEQ ID NO: 178 - DNA, 77 bp, synthetic construct
  source 1..77: mol_type = other DNA
tttttttcgtttcgagcgaggtaggtgtcgtgggcggtttcggttatttttgtatttcgagcgtttattttgtattt

SEQ ID NO: 179 - DNA, 361 bp, Homo sapiens
  source 1..361: mol_type = genomic DNA
ccgcttcggcccccagcgcagctgcagaggggcccccctcgacgcatacactcaagagcccgaccgcgcggctgaaatcgcggagctcggagccgcggctggctgagcgatcgcggttcctgggctgcgtgcgcgccccttggagctgaaaggagcgccaggatcgggggcgctgcaccgggctgggcccctcaacgctcgcagaccgggccgggctgcagctggagatggcagcaatcccgggaggtctccgggcctcttcagggtgcgtccaggaggcgggttccgtgcgacgcggcgcagcccacccccacgagaccgcttaacttcgcgggggcagcctcgggcgctcggagacgcg

SEQ ID NO: 180 - DNA, 361 bp, synthetic construct
  source 1..361: mol_type = other DNA
tcgtttcggtttttagcgtagttgtagaggggtttttttcgacgtatatatttaagagttcgatcgcgcggttgaaatcgcggagttcggagtcgcggttggttgagcgatcgcggtttttgggttgcgtgcgcgttttttggagttgaaaggagcgttaggatcgggggcgttgtatcgggttgggttttttaacgttcgtagatcgggtcgggttgtagttggagatggtagtaatttcgggaggttttcgggtttttttagggtgcgtttaggaggcgggtttcgtgcgacgcggcgtagtttatttttacgagatcgtttaatttcgcgggggtagtttcgggcgttcggagacgcg

SEQ ID NO: 181 - DNA, 361 bp, synthetic construct
  source 1..361: mol_type = other DNA
cgcgtctccgaacgcccgaaactacccccgcgaaattaaacgatctcgtaaaaataaactacgccgcgtcgcacgaaacccgcctcctaaacgcaccctaaaaaaacccgaaaacctcccgaaattactaccatctccaactacaacccgacccgatctacgaacgttaaaaaacccaacccgatacaacgcccccgatcctaacgctcctttcaactccaaaaaacgcgcacgcaacccaaaaaccgcgatcgctcaaccaaccgcgactccgaactccgcgatttcaaccgcgcgatcgaactcttaaatatatacgtcgaaaaaaacccctctacaactacgctaaaaaccgaaacga

SEQ ID NO: 182 - DNA, 361 bp, synthetic construct
  source 1..361: mol_type = other DNA
ccgcttcgacccccaacgcaactacaaaaaaacccccctcgacgcatacactcaaaaacccgaccgcgcgactaaaatcgcgaaactcgaaaccgcgactaactaaacgatcgcgattcctaaactacgtacgcgccccttaaaactaaaaaaaacgccaaaatcgaaaacgctacaccgaactaaacccctcaacgctcgcaaaccgaaccgaactacaactaaaaataacaacaatcccgaaaaatctccgaacctcttcaaaatacgtccaaaaaacgaattccgtacgacgcgacgcaacccacccccacgaaaccgcttaacttcgcgaaaacaacctcgaacgctcgaaaacgcg

SEQ ID NO: 183 - DNA, 361 bp, synthetic construct
  source 1..361: mol_type = other DNA
cgcgttttcgagcgttcgaggttgttttcgcgaagttaagcggtttcgtgggggtgggttgcgtcgcgtcgtacggaattcgttttttggacgtattttgaagaggttcggagatttttcgggattgttgttatttttagttgtagttcggttcggtttgcgagcgttgaggggtttagttcggtgtagcgttttcgattttggcgttttttttagttttaaggggcgcgtacgtagtttaggaatcgcgatcgtttagttagtcgcggtttcgagtttcgcgattttagtcgcgcggtcgggtttttgagtgtatgcgtcgaggggggtttttttgtagttgcgttgggggtcgaagcgg

SEQ ID NO: 184 - DNA, 1097 bp, Homo sapiens
  source 1..1097: mol_type = genomic DNA
ggcagtgcggatgctgcctatgggggaggcagccaaggacggagggcgagaggcggttcttccaaggtcaccctcttccgggttgcaagcaaaggtcaggggatcccggaatggttagtgcaggagcttctctgtgccttccacgtcctagatcctcagagcctcagaaacggagatcatcgtccccacccccattttacagatgaagaaactgagccgaggaaaggaagcgacttggccaaggtcggagagctcattctttgcagggcggggtttggaacccggggtctggctctcggcaacgcgccctcggcccgcagcctcctgccccctgtgccccgcttcggcccccagcgcagctgcagaggggcccccctcgacgcatacactcaagagcccgaccgcgcggctgaaatcgcggagctcggagccgcggctggctgagcgatcgcggttcctgggctgcgtgcgcgccccttggagctgaaaggagcgccaggatcgggggcgctgcaccgggctgggcccctcaacgctcgcagaccgggccgggctgcagctggagatggcagcaatcccgggaggtctccgggcctcttcagggtgcgtccaggaggcgggttccgtgcgacgcggcgcagcccacccccacgagaccgcttaacttcgcgggggcagcctcgggcgctcggagacgcggaggcccagactgcagcctccggatgctggaagcccagactccctggggtcaccggctctcccgccaccccagctgcaaagagtcccattgcttcaccgtccggagcttagtctccttgttcctctaccagtccctccctccgcaggtctctggggacttctgaccgcctgttcttactctccccctgcccccatacttcccgcccttgtctcaggaacggtgatacagtcatcggattgctctccatctcctgttagtctacactgcacacaactcaataatccgctcccttcatccgggtgacagagacacagataatctgagctagtggtgctcaaagtaccggtcccagaacagcagcatcagcatctcttgggaacttgttaaaaatgagaat

SEQ ID NO: 185 - DNA, 1097 bp, synthetic construct
  source 1..1097: mol_type = other DNA
ggtagtgcggatgttgtttatgggggaggtagttaaggacggagggcgagaggcggtttttttaaggttattttttttcgggttgtaagtaaaggttaggggatttcggaatggttagtgtaggagtttttttgtgttttttacgttttagatttttagagttttagaaacggagattatcgtttttatttttattttatagatgaagaaattgagtcgaggaaaggaagcgatttggttaaggtcggagagtttattttttgtagggcggggtttggaattcggggtttggttttcggtaacgcgttttcggttcgtagttttttgttttttgtgtttcgtttcggtttttagcgtagttgtagaggggtttttttcgacgtatatatttaagagttcgatcgcgcggttgaaatcgcggagttcggagtcgcggttggttgagcgatcgcggtttttgggttgcgtgcgcgttttttggagttgaaaggagcgttaggatcgggggcgttgtatcgggttgggttttttaacgttcgtagatcgggtcgggttgtagttggagatggtagtaatttcgggaggttttcgggtttttttagggtgcgtttaggaggcgggtttcgtgcgacgcggcgtagtttatttttacgagatcgtttaatttcgcgggggtagtttcgggcgttcggagacgcggaggtttagattgtagttttcggatgttggaagtttagattttttggggttatcggttttttcgttattttagttgtaaagagttttattgttttatcgttcggagtttagtttttttgtttttttattagttttttttttcgtaggtttttggggatttttgatcgtttgtttttattttttttttgtttttatatttttcgtttttgttttaggaacggtgatatagttatcggattgttttttattttttgttagtttatattgtatataatttaataattcgttttttttattcgggtgatagagatatagataatttgagttagtggtgtttaaagtatcggttttagaatagtagtattagtattttttgggaatttgttaaaaatgagaat

SEQ ID NO: 186 - DNA, 1097 bp, synthetic construct
  source 1..1097: mol_type = other DNA
attctcatttttaacaaattcccaaaaaatactaatactactattctaaaaccgatactttaaacaccactaactcaaattatctatatctctatcacccgaataaaaaaaacgaattattaaattatatacaatataaactaacaaaaaataaaaaacaatccgataactatatcaccgttcctaaaacaaaaacgaaaaatataaaaacaaaaaaaaaataaaaacaaacgatcaaaaatccccaaaaacctacgaaaaaaaaaactaataaaaaaacaaaaaaactaaactccgaacgataaaacaataaaactctttacaactaaaataacgaaaaaaccgataaccccaaaaaatctaaacttccaacatccgaaaactacaatctaaacctccgcgtctccgaacgcccgaaactacccccgcgaaattaaacgatctcgtaaaaataaactacgccgcgtcgcacgaaacccgcctcctaaacgcaccctaaaaaaacccgaaaacctcccgaaattactaccatctccaactacaacccgacccgatctacgaacgttaaaaaacccaacccgatacaacgcccccgatcctaacgctcctttcaactccaaaaaacgcgcacgcaacccaaaaaccgcgatcgctcaaccaaccgcgactccgaactccgcgatttcaaccgcgcgatcgaactcttaaatatatacgtcgaaaaaaacccctctacaactacgctaaaaaccgaaacgaaacacaaaaaacaaaaaactacgaaccgaaaacgcgttaccgaaaaccaaaccccgaattccaaaccccgccctacaaaaaataaactctccgaccttaaccaaatcgcttcctttcctcgactcaatttcttcatctataaaataaaaataaaaacgataatctccgtttctaaaactctaaaaatctaaaacgtaaaaaacacaaaaaaactcctacactaaccattccgaaatcccctaacctttacttacaacccgaaaaaaaataaccttaaaaaaaccgcctctcgccctccgtccttaactacctcccccataaacaacatccgcactacc

SEQ ID NO: 187 - DNA, 1097 bp, synthetic construct
  source 1..1097: mol_type = other DNA
aacaatacgaatactacctataaaaaaaacaaccaaaaacgaaaaacgaaaaacgattcttccaaaatcaccctcttccgaattacaaacaaaaatcaaaaaatcccgaaataattaatacaaaaacttctctataccttccacgtcctaaatcctcaaaacctcaaaaacgaaaatcatcgtccccacccccattttacaaataaaaaaactaaaccgaaaaaaaaaaacgacttaaccaaaatcgaaaaactcattctttacaaaacgaaatttaaaacccgaaatctaactctcgacaacgcgccctcgacccgcaacctcctaccccctataccccgcttcgacccccaacgcaactacaaaaaaacccccctcgacgcatacactcaaaaacccgaccgcgcgactaaaatcgcgaaactcgaaaccgcgactaactaaacgatcgcgattcctaaactacgtacgcgccccttaaaactaaaaaaaacgccaaaatcgaaaacgctacaccgaactaaacccctcaacgctcgcaaaccgaaccgaactacaactaaaaataacaacaatcccgaaaaatctccgaacctcttcaaaatacgtccaaaaaacgaattccgtacgacgcgacgcaacccacccccacgaaaccgcttaacttcgcgaaaacaacctcgaacgctcgaaaacgcgaaaacccaaactacaacctccgaatactaaaaacccaaactccctaaaatcaccgactctcccgccaccccaactacaaaaaatcccattacttcaccgtccgaaacttaatctccttattcctctaccaatccctccctccgcaaatctctaaaaacttctaaccgcctattcttactctccccctacccccatacttcccgcccttatctcaaaaacgataatacaatcatcgaattactctccatctcctattaatctacactacacacaactcaataatccgctcccttcatccgaataacaaaaacacaaataatctaaactaataatactcaaaataccgatcccaaaacaacaacatcaacatctcttaaaaacttattaaaaataaaaat

SEQ ID NO: 188 - DNA, 1097 bp, synthetic construct
  source 1..1097: mol_type = other DNA
atttttatttttaataagtttttaagagatgttgatgttgttgttttgggatcggtattttgagtattattagtttagattatttgtgtttttgttattcggatgaagggagcggattattgagttgtgtgtagtgtagattaataggagatggagagtaattcgatgattgtattatcgtttttgagataagggcgggaagtatgggggtagggggagagtaagaataggcggttagaagtttttagagatttgcggagggagggattggtagaggaataaggagattaagtttcggacggtgaagtaatgggattttttgtagttggggtggcgggagagtcggtgattttagggagtttgggtttttagtattcggaggttgtagtttgggttttcgcgttttcgagcgttcgaggttgttttcgcgaagttaagcggtttcgtgggggtgggttgcgtcgcgtcgtacggaattcgttttttggacgtattttgaagaggttcggagatttttcgggattgttgttatttttagttgtagttcggttcggtttgcgagcgttgaggggtttagttcggtgtagcgttttcgattttggcgttttttttagttttaaggggcgcgtacgtagtttaggaatcgcgatcgtttagttagtcgcggtttcgagtttcgcgattttagtcgcgcggtcgggtttttgagtgtatgcgtcgaggggggtttttttgtagttgcgttgggggtcgaagcggggtatagggggtaggaggttgcgggtcgagggcgcgttgtcgagagttagatttcgggttttaaatttcgttttgtaaagaatgagtttttcgattttggttaagtcgtttttttttttcggtttagtttttttatttgtaaaatgggggtggggacgatgattttcgtttttgaggttttgaggatttaggacgtggaaggtatagagaagtttttgtattaattatttcgggattttttgatttttgtttgtaattcggaagagggtgattttggaagaatcgtttttcgtttttcgtttttggttgttttttttataggtagtattcgtattgtt

SEQ ID NO: 189 - DNA, 97 bp, Homo sapiens
  source 1..97: mol_type = genomic DNA
atcgggggcgctgcaccgggctgggcccctcaacgctcgcagaccgggccgggctgcagctggagatggcagcaatcccgggaggtctccgggcctc

SEQ ID NO: 190 - DNA, 97 bp, synthetic construct
  source 1..97: mol_type = other DNA
atcgggggcgttgtatcgggttgggttttttaacgttcgtagatcgggtcgggttgtagttggagatggtagtaatttcgggaggttttcgggtttt

SEQ ID NO: 191 - DNA, 96 bp, synthetic construct
  source 1..96: mol_type = other DNA
aaacccgaaaacctcccgaaattactaccatctccaactacaacccgacccgatctacgaacgttaaaaaacccaacccgatacaacgcccccgat

SEQ ID NO: 192 - DNA, 97 bp, synthetic construct
  source 1..97: mol_type = other DNA
atcgaaaacgctacaccgaactaaacccctcaacgctcgcaaaccgaaccgaactacaactaaaaataacaacaatcccgaaaaatctccgaacctc

SEQ ID NO: 193 - DNA, 97 bp, synthetic construct
  source 1..97: mol_type = other DNA
gaggttcggagatttttcgggattgttgttatttttagttgtagttcggttcggtttgcgagcgttgaggggtttagttcggtgtagcgttttcgat

SEQ ID NO: 194 - DNA, 1431 bp, Homo sapiens
  source 1..1431: mol_type = genomic DNA
ccgggagaggcgcgcgggcagcgtccaccccacccgctgggcctccgcagggccaaggccccagcagaaaaggcttcacgccgccgccggggtctgggacgcttgcccgacggagtcagaggagctcccgggtccagagtccccagtgcaaactccgacgcaacctgcgcctcgaagcgcaagcagcaaaagcgcccggactctggtcccaagagcctgggcctccttaagccataagcgtctgcggcgcctcgctttgggccttcttttgggccgggccggaggcatcttctagaaggctcttcagaccccgctttcgccaaactcccggcgccctgcgcttccagcccagcagagaaaagtgtgaaaagcaagcccgcggtcgccgtcggccttggcagagaaatcaagaggagaagggaagggaaccgctcaactacccttcgggaaaccaagtttccaaatatgccgccctcttcctggtttgcacaaacggtttagggcattcgttccggtttcagggtggggtatgccgtcgctcccctcctccccgccctgtgcttttaaaagttaggaaacaaaaaagagcacccattggctggaaccccaagggaggcagatgcaggaagcacagagctgcaccgctaggcgcagcaaacagccgcggccgaaggcgcgggtcgccgagtgggcggcggccgcccgcgaggcgagcctggaagcagtatctgaaccgtctggagggtgaccaggttgcgcggcgagctttgcgactgacacggccaatgcgctagtggctgagtggccttggccatgtagggacgcggcgcgcacgcacacccacagactcgcagaagttcaggttcgcgccggagagaagttataggcaggaaggggcccactcacccgcggctccgcgctccaggtctgcgccactccgagccggcccagacgtcgccccagccggcggcgggagcgcagcgccttcgtctccctcagaagaacccttttcgcccgcgccgccgccgcggctgcagcctccgtcgtccacggcccccgccgcctcttcggcgcccagttttcgccgcttcttctgaagctgttgctgcttctcggcgccgatcagcgcctccaccgagaaagcgtgcgccttgaggcttagcatgctgcacggcgagccccttcgcttctcggccatcccccccgccccgcgcccgcccgcccctctctcatatacactcacgcgggcacacgcgcgctctcgctcttcccccaccaaaaactaaaaggctctcggggcctcccgagatctgcccccttccccaccgcgggcaaaaaacagatttggcgtttccgctttctcgcttgtgttgggatccaggaaccggcgacgcgccggccaagtctcctttcctgggtctctctcgcgcg

SEQ ID NO: 195 - DNA, 1431 bp, synthetic construct
  source 1..1431: mol_type = other DNA
tcgggagaggcgcgcgggtagcgtttattttattcgttgggttttcgtagggttaaggttttagtagaaaaggttttacgtcgtcgtcggggtttgggacgtttgttcgacggagttagaggagttttcgggtttagagtttttagtgtaaatttcgacgtaatttgcgtttcgaagcgtaagtagtaaaagcgttcggattttggttttaagagtttgggtttttttaagttataagcgtttgcggcgtttcgttttgggtttttttttgggtcgggtcggaggtattttttagaaggttttttagatttcgttttcgttaaattttcggcgttttgcgtttttagtttagtagagaaaagtgtgaaaagtaagttcgcggtcgtcgtcggttttggtagagaaattaagaggagaagggaagggaatcgtttaattatttttcgggaaattaagtttttaaatatgtcgttttttttttggtttgtataaacggtttagggtattcgtttcggttttagggtggggtatgtcgtcgtttttttttttttcgttttgtgtttttaaaagttaggaaataaaaaagagtatttattggttggaattttaagggaggtagatgtaggaagtatagagttgtatcgttaggcgtagtaaatagtcgcggtcgaaggcgcgggtcgtcgagtgggcggcggtcgttcgcgaggcgagtttggaagtagtatttgaatcgtttggagggtgattaggttgcgcggcgagttttgcgattgatacggttaatgcgttagtggttgagtggttttggttatgtagggacgcggcgcgtacgtatatttatagattcgtagaagtttaggttcgcgtcggagagaagttataggtaggaaggggtttatttattcgcggtttcgcgttttaggtttgcgttatttcgagtcggtttagacgtcgttttagtcggcggcgggagcgtagcgttttcgttttttttagaagaatttttttcgttcgcgtcgtcgtcgcggttgtagttttcgtcgtttacggttttcgtcgttttttcggcgtttagttttcgtcgttttttttgaagttgttgttgtttttcggcgtcgattagcgtttttatcgagaaagcgtgcgttttgaggtttagtatgttgtacggcgagttttttcgtttttcggttatttttttcgtttcgcgttcgttcgttttttttttatatatatttacgcgggtatacgcgcgttttcgttttttttttattaaaaattaaaaggttttcggggtttttcgagatttgtttttttttttatcgcgggtaaaaaatagatttggcgttttcgttttttcgtttgtgttgggatttaggaatcggcgacgcgtcggttaagtttttttttttgggtttttttcgcgcg

SEQ ID NO: 196 - DNA, 1431 bp, synthetic construct
  source 1..1431: mol_type = other DNA
cgcgcgaaaaaaacccaaaaaaaaaaacttaaccgacgcgtcgccgattcctaaatcccaacacaaacgaaaaaacgaaaacgccaaatctattttttacccgcgataaaaaaaaaaacaaatctcgaaaaaccccgaaaaccttttaatttttaataaaaaaaaaacgaaaacgcgcgtatacccgcgtaaatatatataaaaaaaaaacgaacgaacgcgaaacgaaaaaaataaccgaaaaacgaaaaaactcgccgtacaacatactaaacctcaaaacgcacgctttctcgataaaaacgctaatcgacgccgaaaaacaacaacaacttcaaaaaaaacgacgaaaactaaacgccgaaaaaacgacgaaaaccgtaaacgacgaaaactacaaccgcgacgacgacgcgaacgaaaaaaattcttctaaaaaaaacgaaaacgctacgctcccgccgccgactaaaacgacgtctaaaccgactcgaaataacgcaaacctaaaacgcgaaaccgcgaataaataaaccccttcctacctataacttctctccgacgcgaacctaaacttctacgaatctataaatatacgtacgcgccgcgtccctacataaccaaaaccactcaaccactaacgcattaaccgtatcaatcgcaaaactcgccgcgcaacctaatcaccctccaaacgattcaaatactacttccaaactcgcctcgcgaacgaccgccgcccactcgacgacccgcgccttcgaccgcgactatttactacgcctaacgatacaactctatacttcctacatctacctcccttaaaattccaaccaataaatactcttttttatttcctaacttttaaaaacacaaaacgaaaaaaaaaaaaacgacgacataccccaccctaaaaccgaaacgaataccctaaaccgtttatacaaaccaaaaaaaaaacgacatatttaaaaacttaatttcccgaaaaataattaaacgattcccttcccttctcctcttaatttctctaccaaaaccgacgacgaccgcgaacttacttttcacacttttctctactaaactaaaaacgcaaaacgccgaaaatttaacgaaaacgaaatctaaaaaaccttctaaaaaatacctccgacccgacccaaaaaaaaacccaaaacgaaacgccgcaaacgcttataacttaaaaaaacccaaactcttaaaaccaaaatccgaacgcttttactacttacgcttcgaaacgcaaattacgtcgaaatttacactaaaaactctaaacccgaaaactcctctaactccgtcgaacaaacgtcccaaaccccgacgacgacgtaaaaccttttctactaaaaccttaaccctacgaaaacccaacgaataaaataaacgctacccgcgcgcctctcccga

SEQ ID NO: 197 - DNA, 1431 bp, synthetic construct
  source 1..1431: mol_type = other DNA
ccgaaaaaaacgcgcgaacaacgtccaccccacccgctaaacctccgcaaaaccaaaaccccaacaaaaaaaacttcacgccgccgccgaaatctaaaacgcttacccgacgaaatcaaaaaaactcccgaatccaaaatccccaatacaaactccgacgcaacctacgcctcgaaacgcaaacaacaaaaacgcccgaactctaatcccaaaaacctaaacctccttaaaccataaacgtctacgacgcctcgctttaaaccttcttttaaaccgaaccgaaaacatcttctaaaaaactcttcaaaccccgctttcgccaaactcccgacgccctacgcttccaacccaacaaaaaaaaatataaaaaacaaacccgcgatcgccgtcgaccttaacaaaaaaatcaaaaaaaaaaaaaaaaaaaaccgctcaactacccttcgaaaaaccaaatttccaaatataccgccctcttcctaatttacacaaacgatttaaaacattcgttccgatttcaaaataaaatataccgtcgctcccctcctccccgccctatacttttaaaaattaaaaaacaaaaaaaaacacccattaactaaaaccccaaaaaaaacaaatacaaaaaacacaaaactacaccgctaaacgcaacaaacaaccgcgaccgaaaacgcgaatcgccgaataaacgacgaccgcccgcgaaacgaacctaaaaacaatatctaaaccgtctaaaaaataaccaaattacgcgacgaactttacgactaacacgaccaatacgctaataactaaataaccttaaccatataaaaacgcgacgcgcacgcacacccacaaactcgcaaaaattcaaattcgcgccgaaaaaaaattataaacaaaaaaaaacccactcacccgcgactccgcgctccaaatctacgccactccgaaccgacccaaacgtcgccccaaccgacgacgaaaacgcaacgccttcgtctccctcaaaaaaacccttttcgcccgcgccgccgccgcgactacaacctccgtcgtccacgacccccgccgcctcttcgacgcccaattttcgccgcttcttctaaaactattactacttctcgacgccgatcaacgcctccaccgaaaaaacgtacgccttaaaacttaacatactacacgacgaaccccttcgcttctcgaccatcccccccgccccgcgcccgcccgcccctctctcatatacactcacgcgaacacacgcgcgctctcgctcttcccccaccaaaaactaaaaaactctcgaaacctcccgaaatctacccccttccccaccgcgaacaaaaaacaaatttaacgtttccgctttctcgcttatattaaaatccaaaaaccgacgacgcgccgaccaaatctcctttcctaaatctctctcgcgcg

SEQ ID NO: 198 - DNA, 1431 bp, synthetic construct
  source 1..1431: mol_type = other DNA
cgcgcgagagagatttaggaaaggagatttggtcggcgcgtcgtcggtttttggattttaatataagcgagaaagcggaaacgttaaatttgttttttgttcgcggtggggaagggggtagatttcgggaggtttcgagagttttttagtttttggtgggggaagagcgagagcgcgcgtgtgttcgcgtgagtgtatatgagagaggggcgggcgggcgcggggcgggggggatggtcgagaagcgaaggggttcgtcgtgtagtatgttaagttttaaggcgtacgttttttcggtggaggcgttgatcggcgtcgagaagtagtaatagttttagaagaagcggcgaaaattgggcgtcgaagaggcggcgggggtcgtggacgacggaggttgtagtcgcggcggcggcgcgggcgaaaagggtttttttgagggagacgaaggcgttgcgttttcgtcgtcggttggggcgacgtttgggtcggttcggagtggcgtagatttggagcgcggagtcgcgggtgagtgggtttttttttgtttataattttttttcggcgcgaatttgaatttttgcgagtttgtgggtgtgcgtgcgcgtcgcgtttttatatggttaaggttatttagttattagcgtattggtcgtgttagtcgtaaagttcgtcgcgtaatttggttattttttagacggtttagatattgtttttaggttcgtttcgcgggcggtcgtcgtttattcggcgattcgcgttttcggtcgcggttgtttgttgcgtttagcggtgtagttttgtgttttttgtatttgttttttttggggttttagttaatgggtgtttttttttgttttttaatttttaaaagtatagggcggggaggaggggagcgacggtatattttattttgaaatcggaacgaatgttttaaatcgtttgtgtaaattaggaagagggcggtatatttggaaatttggtttttcgaagggtagttgagcggttttttttttttttttttttgatttttttgttaaggtcgacggcgatcgcgggtttgttttttatatttttttttgttgggttggaagcgtagggcgtcgggagtttggcgaaagcggggtttgaagagttttttagaagatgttttcggttcggtttaaaagaaggtttaaagcgaggcgtcgtagacgtttatggtttaaggaggtttaggtttttgggattagagttcgggcgtttttgttgtttgcgtttcgaggcgtaggttgcgtcggagtttgtattggggattttggattcgggagtttttttgatttcgtcgggtaagcgttttagatttcggcggcggcgtgaagttttttttgttggggttttggttttgcggaggtttagcgggtggggtggacgttgttcgcgcgtttttttcgg

SEQ ID NO: 199 - DNA, 1087 bp, Homo sapiens
  source 1..1087: mol_type = genomic DNA
cagaccccgctttcgccaaactcccggcgccctgcgcttccagcccagcagagaaaagtgtgaaaagcaagcccgcggtcgccgtcggccttggcagagaaatcaagaggagaagggaagggaaccgctcaactacccttcgggaaaccaagtttccaaatatgccgccctcttcctggtttgcacaaacggtttagggcattcgttccggtttcagggtggggtatgccgtcgctcccctcctccccgccctgtgcttttaaaagttaggaaacaaaaaagagcacccattggctggaaccccaagggaggcagatgcaggaagcacagagctgcaccgctaggcgcagcaaacagccgcggccgaaggcgcgggtcgccgagtgggcggcggccgcccgcgaggcgagcctggaagcagtatctgaaccgtctggagggtgaccaggttgcgcggcgagctttgcgactgacacggccaatgcgctagtggctgagtggccttggccatgtagggacgcggcgcgcacgcacacccacagactcgcagaagttcaggttcgcgccggagagaagttataggcaggaaggggcccactcacccgcggctccgcgctccaggtctgcgccactccgagccggcccagacgtcgccccagccggcggcgggagcgcagcgccttcgtctccctcagaagaacccttttcgcccgcgccgccgccgcggctgcagcctccgtcgtccacggcccccgccgcctcttcggcgcccagttttcgccgcttcttctgaagctgttgctgcttctcggcgccgatcagcgcctccaccgagaaagcgtgcgccttgaggcttagcatgctgcacggcgagccccttcgcttctcggccatcccccccgccccgcgcccgcccgcccctctctcatatacactcacgcgggcacacgcgcgctctcgctcttcccccaccaaaaactaaaaggctctcggggcctcccgagatctgcccccttccccaccgcgggcaaaaaacagatttggcgtttccgctttctcgcttgtgttgggatccaggaaccggcga

SEQ ID NO: 200 - DNA, 1087 bp, synthetic construct
  source 1..1087: mol_type = other DNA
tagatttcgttttcgttaaattttcggcgttttgcgtttttagtttagtagagaaaagtgtgaaaagtaagttcgcggtcgtcgtcggttttggtagagaaattaagaggagaagggaagggaatcgtttaattatttttcgggaaattaagtttttaaatatgtcgttttttttttggtttgtataaacggtttagggtattcgtttcggttttagggtggggtatgtcgtcgtttttttttttttcgttttgtgtttttaaaagttaggaaataaaaaagagtatttattggttggaattttaagggaggtagatgtaggaagtatagagttgtatcgttaggcgtagtaaatagtcgcggtcgaaggcgcgggtcgtcgagtgggcggcggtcgttcgcgaggcgagtttggaagtagtatttgaatcgtttggagggtgattaggttgcgcggcgagttttgcgattgatacggttaatgcgttagtggttgagtggttttggttatgtagggacgcggcgcgtacgtatatttatagattcgtagaagtttaggttcgcgtcggagagaagttataggtaggaaggggtttatttattcgcggtttcgcgttttaggtttgcgttatttcgagtcggtttagacgtcgttttagtcggcggcgggagcgtagcgttttcgttttttttagaagaatttttttcgttcgcgtcgtcgtcgcggttgtagttttcgtcgtttacggttttcgtcgttttttcggcgtttagttttcgtcgttttttttgaagttgttgttgtttttcggcgtcgattagcgtttttatcgagaaagcgtgcgttttgaggtttagtatgttgtacggcgagttttttcgtttttcggttatttttttcgtttcgcgttcgttcgttttttttttatatatatttacgcgggtatacgcgcgttttcgttttttttttattaaaaattaaaaggttttcggggtttttcgagatttgtttttttttttatcgcgggtaaaaaatagatttggcgttttcgttttttcgtttgtgttgggatttaggaatcggcga

SEQ ID NO: 201 - DNA, 1087 bp, synthetic construct
  source 1..1087: mol_type = other DNA
tcgccgattcctaaatcccaacacaaacgaaaaaacgaaaacgccaaatctattttttacccgcgataaaaaaaaaaacaaatctcgaaaaaccccgaaaaccttttaatttttaataaaaaaaaaacgaaaacgcgcgtatacccgcgtaaatatatataaaaaaaaaacgaacgaacgcgaaacgaaaaaaataaccgaaaaacgaaaaaactcgccgtacaacatactaaacctcaaaacgcacgctttctcgataaaaacgctaatcgacgccgaaaaacaacaacaacttcaaaaaaaacgacgaaaactaaacgccgaaaaaacgacgaaaaccgtaaacgacgaaaactacaaccgcgacgacgacgcgaacgaaaaaaattcttctaaaaaaaacgaaaacgctacgctcccgccgccgactaaaacgacgtctaaaccgactcgaaataacgcaaacctaaaacgcgaaaccgcgaataaataaaccccttcctacctataacttctctccgacgcgaacctaaacttctacgaatctataaatatacgtacgcgccgcgtccctacataaccaaaaccactcaaccactaacgcattaaccgtatcaatcgcaaaactcgccgcgcaacctaatcaccctccaaacgattcaaatactacttccaaactcgcctcgcgaacgaccgccgcccactcgacgacccgcgccttcgaccgcgactatttactacgcctaacgatacaactctatacttcctacatctacctcccttaaaattccaaccaataaatactcttttttatttcctaacttttaaaaacacaaaacgaaaaaaaaaaaaacgacgacataccccaccctaaaaccgaaacgaataccctaaaccgtttatacaaaccaaaaaaaaaacgacatatttaaaaacttaatttcccgaaaaataattaaacgattcccttcccttctcctcttaatttctctaccaaaaccgacgacgaccgcgaacttacttttcacacttttctctactaaactaaaaacgcaaaacgccgaaaatttaacgaaaacgaaatcta

SEQ ID NO: 202 - DNA, 1087 bp, synthetic construct
  source 1..1087: mol_type = other DNA
caaaccccgctttcgccaaactcccgacgccctacgcttccaacccaacaaaaaaaaatataaaaaacaaacccgcgatcgccgtcgaccttaacaaaaaaatcaaaaaaaaaaaaaaaaaaaaccgctcaactacccttcgaaaaaccaaatttccaaatataccgccctcttcctaatttacacaaacgatttaaaacattcgttccgatttcaaaataaaatataccgtcgctcccctcctccccgccctatacttttaaaaattaaaaaacaaaaaaaaacacccattaactaaaaccccaaaaaaaacaaatacaaaaaacacaaaactacaccgctaaacgcaacaaacaaccgcgaccgaaaacgcgaatcgccgaataaacgacgaccgcccgcgaaacgaacctaaaaacaatatctaaaccgtctaaaaaataaccaaattacgcgacgaactttacgactaacacgaccaatacgctaataactaaataaccttaaccatataaaaacgcgacgcgcacgcacacccacaaactcgcaaaaattcaaattcgcgccgaaaaaaaattataaacaaaaaaaaacccactcacccgcgactccgcgctccaaatctacgccactccgaaccgacccaaacgtcgccccaaccgacgacgaaaacgcaacgccttcgtctccctcaaaaaaacccttttcgcccgcgccgccgccgcgactacaacctccgtcgtccacgacccccgccgcctcttcgacgcccaattttcgccgcttcttctaaaactattactacttctcgacgccgatcaacgcctccaccgaaaaaacgtacgccttaaaacttaacatactacacgacgaaccccttcgcttctcgaccatcccccccgccccgcgcccgcccgcccctctctcatatacactcacgcgaacacacgcgcgctctcgctcttcccccaccaaaaactaaaaaactctcgaaacctcccgaaatctacccccttccccaccgcgaacaaaaaacaaatttaacgtttccgctttctcgcttatattaaaatccaaaaaccgacga

SEQ ID NO: 203 - DNA, 1087 bp, synthetic construct
  source 1..1087: mol_type = other DNA
tcgtcggtttttggattttaatataagcgagaaagcggaaacgttaaatttgttttttgttcgcggtggggaagggggtagatttcgggaggtttcgagagttttttagtttttggtgggggaagagcgagagcgcgcgtgtgttcgcgtgagtgtatatgagagaggggcgggcgggcgcggggcgggggggatggtcgagaagcgaaggggttcgtcgtgtagtatgttaagttttaaggcgtacgttttttcggtggaggcgttgatcggcgtcgagaagtagtaatagttttagaagaagcggcgaaaattgggcgtcgaagaggcggcgggggtcgtggacgacggaggttgtagtcgcggcggcggcgcgggcgaaaagggtttttttgagggagacgaaggcgttgcgttttcgtcgtcggttggggcgacgtttgggtcggttcggagtggcgtagatttggagcgcggagtcgcgggtgagtgggtttttttttgtttataattttttttcggcgcgaatttgaatttttgcgagtttgtgggtgtgcgtgcgcgtcgcgtttttatatggttaaggttatttagttattagcgtattggtcgtgttagtcgtaaagttcgtcgcgtaatttggttattttttagacggtttagatattgtttttaggttcgtttcgcgggcggtcgtcgtttattcggcgattcgcgttttcggtcgcggttgtttgttgcgtttagcggtgtagttttgtgttttttgtatttgttttttttggggttttagttaatgggtgtttttttttgttttttaatttttaaaagtatagggcggggaggaggggagcgacggtatattttattttgaaatcggaacgaatgttttaaatcgtttgtgtaaattaggaagagggcggtatatttggaaatttggtttttcgaagggtagttgagcggttttttttttttttttttttgatttttttgttaaggtcgacggcgatcgcgggtttgttttttatatttttttttgttgggttggaagcgtagggcgtcgggagtttggcgaaagcggggtttg

SEQ ID NO: 204 - DNA, 87 bp, Homo sapiens
  source 1..87: mol_type = genomic DNA
gccttggccatgtagggacgcggcgcgcacgcacacccacagactcgcagaagttcaggttcgcgccggagagaagttataggcagg

SEQ ID NO: 205 - DNA, 87 bp, synthetic construct
  source 1..87: mol_type = other DNA
gttttggttatgtagggacgcggcgcgtacgtatatttatagattcgtagaagtttaggttcgcgtcggagagaagttataggtagg

SEQ ID NO: 206 - DNA, 87 bp, synthetic construct
  source 1..87: mol_type = other DNA
cctacctataacttctctccgacgcgaacctaaacttctacgaatctataaatatacgtacgcgccgcgtccctacataaccaaaac

SEQ ID NO: 207 - DNA, 87 bp, synthetic construct
  source 1..87: mol_type = other DNA
accttaaccatataaaaacgcgacgcgcacgcacacccacaaactcgcaaaaattcaaattcgcgccgaaaaaaaattataaacaaa

SEQ ID NO: 208 - DNA, 87 bp, synthetic construct
  source 1..87: mol_type = other DNA
tttgtttataattttttttcggcgcgaatttgaatttttgcgagtttgtgggtgtgcgtgcgcgtcgcgtttttatatggttaaggt

SEQ ID NO: 209 - DNA, 1013 bp, Homo sapiens
  source 1..1013: mol_type = genomic DNA
gcggcgcgctggctccctggagcggggcgggacgcggccgcgcggactcacgtgcacaaccgcgcgggacggggccacgcggactcacgtgcacaaccgcgggaccccagcgccagcgggaccccagcgccagcgggaccccagcgccagcgggaccccagcgccagcgggaccccagcgccagcgggaccccagcgccagcgggaccccagcgccagcgggtctgtggcccagtggagcgagtggagcgctggcgacctgagcggagactgcgccctggacgccccagcctagacgtcaagttacagcccgcgcagcagcagcaaaggggaaggggcaggagccgggcacagttggatccggaggtcgtgacccaggggaaagcgtgggcggtcgacccagggcagctgcggcggcgaggcaggtgggctccttgctccctggagccgcccctccccacacctgccctcggcgcccccagcagttttcaccttggccctccgcggtcactgcgggattcggcgttgccgccagcccagtggggagtgaattagcgccctccttcgtcctcggcccttccgacggcacgaggaactcctgtcctgccccacagaccttcggcctccgccgagtgcggtactggagcctgccccgccagggccctggaatcagagaaagtcgctctttggccacctgaagcgtcggatccctacagtgcctcccagcctgggcgggagcggcggctgcgtcgctgaaggttggggtccttggtgcgaaagggaggcagctgcagcctcagccccaccccagaagcggccttcgcatcgctgcggtgggcgttctcgggcttcgacttcgccagcgccgcggggcagaggcacctggagctcgcagggcccagacctgggttggaaaagcttcgctgactgcaggcaagcgtccgggaggggcggccaggcgaagccccggcgctttaccacacacttccgggtcccatgccagttgcatccgcg

SEQ ID NO: 210 - DNA, 1013 bp, synthetic construct
  source 1..1013: mol_type = other DNA
gcggcgcgttggttttttggagcggggcgggacgcggtcgcgcggatttacgtgtataatcgcgcgggacggggttacgcggatttacgtgtataatcgcgggattttagcgttagcgggattttagcgttagcgggattttagcgttagcgggattttagcgttagcgggattttagcgttagcgggattttagcgttagcgggattttagcgttagcgggtttgtggtttagtggagcgagtggagcgttggcgatttgagcggagattgcgttttggacgttttagtttagacgttaagttatagttcgcgtagtagtagtaaaggggaaggggtaggagtcgggtatagttggattcggaggtcgtgatttaggggaaagcgtgggcggtcgatttagggtagttgcggcggcgaggtaggtgggttttttgttttttggagtcgtttttttttatatttgttttcggcgtttttagtagtttttattttggtttttcgcggttattgcgggattcggcgttgtcgttagtttagtggggagtgaattagcgttttttttcgttttcggttttttcgacggtacgaggaatttttgttttgttttatagattttcggttttcgtcgagtgcggtattggagtttgtttcgttagggttttggaattagagaaagtcgttttttggttatttgaagcgtcggatttttatagtgttttttagtttgggcgggagcggcggttgcgtcgttgaaggttggggtttttggtgcgaaagggaggtagttgtagttttagttttattttagaagcggttttcgtatcgttgcggtgggcgttttcgggtttcgatttcgttagcgtcgcggggtagaggtatttggagttcgtagggtttagatttgggttggaaaagtttcgttgattgtaggtaagcgttcgggaggggcggttaggcgaagtttcggcgttttattatatattttcgggttttatgttagttgtattcgcg

SEQ ID NO: 211 - DNA, 1013 bp, synthetic construct
  source 1..1013: mol_type = other DNA
cgcgaatacaactaacataaaacccgaaaatatataataaaacgccgaaacttcgcctaaccgcccctcccgaacgcttacctacaatcaacgaaacttttccaacccaaatctaaaccctacgaactccaaatacctctaccccgcgacgctaacgaaatcgaaacccgaaaacgcccaccgcaacgatacgaaaaccgcttctaaaataaaactaaaactacaactacctccctttcgcaccaaaaaccccaaccttcaacgacgcaaccgccgctcccgcccaaactaaaaaacactataaaaatccgacgcttcaaataaccaaaaaacgactttctctaattccaaaaccctaacgaaacaaactccaataccgcactcgacgaaaaccgaaaatctataaaacaaaacaaaaattcctcgtaccgtcgaaaaaaccgaaaacgaaaaaaaacgctaattcactccccactaaactaacgacaacgccgaatcccgcaataaccgcgaaaaaccaaaataaaaactactaaaaacgccgaaaacaaatataaaaaaaaacgactccaaaaaacaaaaaacccacctacctcgccgccgcaactaccctaaatcgaccgcccacgctttcccctaaatcacgacctccgaatccaactatacccgactcctaccccttcccctttactactactacgcgaactataacttaacgtctaaactaaaacgtccaaaacgcaatctccgctcaaatcgccaacgctccactcgctccactaaaccacaaacccgctaacgctaaaatcccgctaacgctaaaatcccgctaacgctaaaatcccgctaacgctaaaatcccgctaacgctaaaatcccgctaacgctaaaatcccgctaacgctaaaatcccgcgattatacacgtaaatccgcgtaaccccgtcccgcgcgattatacacgtaaatccgcgcgaccgcgtcccgccccgctccaaaaaaccaacgcgccgc

SEQ ID NO: 212 - DNA, 1013 bp, synthetic construct
  source 1..1013: mol_type = other DNA
acgacgcgctaactccctaaaacgaaacgaaacgcgaccgcgcgaactcacgtacacaaccgcgcgaaacgaaaccacgcgaactcacgtacacaaccgcgaaaccccaacgccaacgaaaccccaacgccaacgaaaccccaacgccaacgaaaccccaacgccaacgaaaccccaacgccaacgaaaccccaacgccaacgaaaccccaacgccaacgaatctataacccaataaaacgaataaaacgctaacgacctaaacgaaaactacgccctaaacgccccaacctaaacgtcaaattacaacccgcgcaacaacaacaaaaaaaaaaaaacaaaaaccgaacacaattaaatccgaaaatcgtaacccaaaaaaaaacgtaaacgatcgacccaaaacaactacgacgacgaaacaaataaactccttactccctaaaaccgcccctccccacacctaccctcgacgcccccaacaattttcaccttaaccctccgcgatcactacgaaattcgacgttaccgccaacccaataaaaaataaattaacgccctccttcgtcctcgacccttccgacgacacgaaaaactcctatcctaccccacaaaccttcgacctccgccgaatacgatactaaaacctaccccgccaaaaccctaaaatcaaaaaaaatcgctctttaaccacctaaaacgtcgaatccctacaatacctcccaacctaaacgaaaacgacgactacgtcgctaaaaattaaaatccttaatacgaaaaaaaaacaactacaacctcaaccccaccccaaaaacgaccttcgcatcgctacgataaacgttctcgaacttcgacttcgccaacgccgcgaaacaaaaacacctaaaactcgcaaaacccaaacctaaattaaaaaaacttcgctaactacaaacaaacgtccgaaaaaaacgaccaaacgaaaccccgacgctttaccacacacttccgaatcccataccaattacatccgcg

SEQ ID NO: 213 - DNA, 1013 bp, synthetic construct
  source 1..1013: mol_type = other DNA
cgcggatgtaattggtatgggattcggaagtgtgtggtaaagcgtcggggtttcgtttggtcgtttttttcggacgtttgtttgtagttagcgaagtttttttaatttaggtttgggttttgcgagttttaggtgtttttgtttcgcggcgttggcgaagtcgaagttcgagaacgtttatcgtagcgatgcgaaggtcgtttttggggtggggttgaggttgtagttgtttttttttcgtattaaggattttaatttttagcgacgtagtcgtcgttttcgtttaggttgggaggtattgtagggattcgacgttttaggtggttaaagagcgattttttttgattttagggttttggcggggtaggttttagtatcgtattcggcggaggtcgaaggtttgtggggtaggataggagtttttcgtgtcgtcggaagggtcgaggacgaaggagggcgttaatttattttttattgggttggcggtaacgtcgaatttcgtagtgatcgcggagggttaaggtgaaaattgttgggggcgtcgagggtaggtgtggggaggggcggttttagggagtaaggagtttatttgtttcgtcgtcgtagttgttttgggtcgatcgtttacgtttttttttgggttacgattttcggatttaattgtgttcggtttttgtttttttttttttgttgttgttgcgcgggttgtaatttgacgtttaggttggggcgtttagggcgtagttttcgtttaggtcgttagcgttttattcgttttattgggttatagattcgttggcgttggggtttcgttggcgttggggtttcgttggcgttggggtttcgttggcgttggggtttcgttggcgttggggtttcgttggcgttggggtttcgttggcgttggggtttcgcggttgtgtacgtgagttcgcgtggtttcgtttcgcgcggttgtgtacgtgagttcgcgcggtcgcgtttcgtttcgttttagggagttagcgcgtcgt

SEQ ID NO: 214 - DNA, 1069 bp, Homo sapiens
  source 1..1069: mol_type = genomic DNA
ggcaggtgggctccttgctccctggagccgcccctccccacacctgccctcggcgcccccagcagttttcaccttggccctccgcggtcactgcgggattcggcgttgccgccagcccagtggggagtgaattagcgccctccttcgtcctcggcccttccgacggcacgaggaactcctgtcctgccccacagaccttcggcctccgccgagtgcggtactggagcctgccccgccagggccctggaatcagagaaagtcgctctttggccacctgaagcgtcggatccctacagtgcctcccagcctgggcgggagcggcggctgcgtcgctgaaggttggggtccttggtgcgaaagggaggcagctgcagcctcagccccaccccagaagcggccttcgcatcgctgcggtgggcgttctcgggcttcgacttcgccagcgccgcggggcagaggcacctggagctcgcagggcccagacctgggttggaaaagcttcgctgactgcaggcaagcgtccgggaggggcggccaggcgaagccccggcgctttaccacacacttccgggtcccatgccagttgcatccgcggtattgggcaggaaatggcagggctgaggccgaccctaggagtataagggagccctccatttcctgcccacatttgtcacctccagttttgcaacctatcccagacacacagaaagcaagcaggactggtggggagacggagcttaacaggaatattttccagcagtgagcaggggctgtatgggacgcgggaggagctcagaggaggcgcggagagtgcccgaggttgggtgagtgcctagaggggagatagttgaaccgggttcaagaggtgcttagtgggtgtttgttgaatgaatgagtgatgggctttgaagtctgagtgcattgaaagagggggtgtgtaaaaagggctcctttcatcacacaggacacagcatatgcaaatcctctccctgtggaaaagccagacaggttaaaaaggttacaaacaaattagccgggcatggtggtgcgcgtctgtagtcccagctac

SEQ ID NO: 215 - DNA, 1069 bp, synthetic construct
  source 1..1069: mol_type = other DNA
ggtaggtgggttttttgttttttggagtcgtttttttttatatttgttttcggcgtttttagtagtttttattttggtttttcgcggttattgcgggattcggcgttgtcgttagtttagtggggagtgaattagcgttttttttcgttttcggttttttcgacggtacgaggaatttttgttttgttttatagattttcggttttcgtcgagtgcggtattggagtttgtttcgttagggttttggaattagagaaagtcgttttttggttatttgaagcgtcggatttttatagtgttttttagtttgggcgggagcggcggttgcgtcgttgaaggttggggtttttggtgcgaaagggaggtagttgtagttttagttttattttagaagcggttttcgtatcgttgcggtgggcgttttcgggtttcgatttcgttagcgtcgcggggtagaggtatttggagttcgtagggtttagatttgggttggaaaagtttcgttgattgtaggtaagcgttcgggaggggcggttaggcgaagtttcggcgttttattatatattttcgggttttatgttagttgtattcgcggtattgggtaggaaatggtagggttgaggtcgattttaggagtataagggagttttttattttttgtttatatttgttatttttagttttgtaatttattttagatatatagaaagtaagtaggattggtggggagacggagtttaataggaatattttttagtagtgagtaggggttgtatgggacgcgggaggagtttagaggaggcgcggagagtgttcgaggttgggtgagtgtttagaggggagatagttgaatcgggtttaagaggtgtttagtgggtgtttgttgaatgaatgagtgatgggttttgaagtttgagtgtattgaaagagggggtgtgtaaaaagggttttttttattatataggatatagtatatgtaaattttttttttgtggaaaagttagataggttaaaaaggttataaataaattagtcgggtatggtggtgcgcgtttgtagttttagttat

SEQ ID NO: 216 - DNA, 1069 bp, synthetic construct
  source 1..1069: mol_type = other DNA
ataactaaaactacaaacgcgcaccaccatacccgactaatttatttataacctttttaacctatctaacttttccacaaaaaaaaaatttacatatactatatcctatataataaaaaaaaccctttttacacaccccctctttcaatacactcaaacttcaaaacccatcactcattcattcaacaaacacccactaaacacctcttaaacccgattcaactatctcccctctaaacactcacccaacctcgaacactctccgcgcctcctctaaactcctcccgcgtcccatacaacccctactcactactaaaaaatattcctattaaactccgtctccccaccaatcctacttactttctatatatctaaaataaattacaaaactaaaaataacaaatataaacaaaaaataaaaaactcccttatactcctaaaatcgacctcaaccctaccatttcctacccaataccgcgaatacaactaacataaaacccgaaaatatataataaaacgccgaaacttcgcctaaccgcccctcccgaacgcttacctacaatcaacgaaacttttccaacccaaatctaaaccctacgaactccaaatacctctaccccgcgacgctaacgaaatcgaaacccgaaaacgcccaccgcaacgatacgaaaaccgcttctaaaataaaactaaaactacaactacctccctttcgcaccaaaaaccccaaccttcaacgacgcaaccgccgctcccgcccaaactaaaaaacactataaaaatccgacgcttcaaataaccaaaaaacgactttctctaattccaaaaccctaacgaaacaaactccaataccgcactcgacgaaaaccgaaaatctataaaacaaaacaaaaattcctcgtaccgtcgaaaaaaccgaaaacgaaaaaaaacgctaattcactccccactaaactaacgacaacgccgaatcccgcaataaccgcgaaaaaccaaaataaaaactactaaaaacgccgaaaacaaatataaaaaaaaacgactccaaaaaacaaaaaacccacctacc

SEQ ID NO: 217 - DNA, 1069 bp, synthetic construct
  source 1..1069: mol_type = other DNA
aacaaataaactccttactccctaaaaccgcccctccccacacctaccctcgacgcccccaacaattttcaccttaaccctccgcgatcactacgaaattcgacgttaccgccaacccaataaaaaataaattaacgccctccttcgtcctcgacccttccgacgacacgaaaaactcctatcctaccccacaaaccttcgacctccgccgaatacgatactaaaacctaccccgccaaaaccctaaaatcaaaaaaaatcgctctttaaccacctaaaacgtcgaatccctacaatacctcccaacctaaacgaaaacgacgactacgtcgctaaaaattaaaatccttaatacgaaaaaaaaacaactacaacctcaaccccaccccaaaaacgaccttcgcatcgctacgataaacgttctcgaacttcgacttcgccaacgccgcgaaacaaaaacacctaaaactcgcaaaacccaaacctaaattaaaaaaacttcgctaactacaaacaaacgtccgaaaaaaacgaccaaacgaaaccccgacgctttaccacacacttccgaatcccataccaattacatccgcgatattaaacaaaaaataacaaaactaaaaccgaccctaaaaatataaaaaaaccctccatttcctacccacatttatcacctccaattttacaacctatcccaaacacacaaaaaacaaacaaaactaataaaaaaacgaaacttaacaaaaatattttccaacaataaacaaaaactatataaaacgcgaaaaaaactcaaaaaaaacgcgaaaaatacccgaaattaaataaatacctaaaaaaaaaataattaaaccgaattcaaaaaatacttaataaatatttattaaataaataaataataaactttaaaatctaaatacattaaaaaaaaaaatatataaaaaaaactcctttcatcacacaaaacacaacatatacaaatcctctccctataaaaaaaccaaacaaattaaaaaaattacaaacaaattaaccgaacataataatacgcgtctataatcccaactac

SEQ ID NO: 218 - DNA, 1069 bp, synthetic construct
  source 1..1069: mol_type = other DNA
gtagttgggattatagacgcgtattattatgttcggttaatttgtttgtaatttttttaatttgtttggtttttttatagggagaggatttgtatatgttgtgttttgtgtgatgaaaggagttttttttatatatttttttttttaatgtatttagattttaaagtttattatttatttatttaataaatatttattaagtattttttgaattcggtttaattattttttttttaggtatttatttaatttcgggtatttttcgcgttttttttgagtttttttcgcgttttatatagtttttgtttattgttggaaaatatttttgttaagtttcgtttttttattagttttgtttgttttttgtgtgtttgggataggttgtaaaattggaggtgataaatgtgggtaggaaatggagggtttttttatatttttagggtcggttttagttttgttattttttgtttaatatcgcggatgtaattggtatgggattcggaagtgtgtggtaaagcgtcggggtttcgtttggtcgtttttttcggacgtttgtttgtagttagcgaagtttttttaatttaggtttgggttttgcgagttttaggtgtttttgtttcgcggcgttggcgaagtcgaagttcgagaacgtttatcgtagcgatgcgaaggtcgtttttggggtggggttgaggttgtagttgtttttttttcgtattaaggattttaatttttagcgacgtagtcgtcgttttcgtttaggttgggaggtattgtagggattcgacgttttaggtggttaaagagcgattttttttgattttagggttttggcggggtaggttttagtatcgtattcggcggaggtcgaaggtttgtggggtaggataggagtttttcgtgtcgtcggaagggtcgaggacgaaggagggcgttaatttattttttattgggttggcggtaacgtcgaatttcgtagtgatcgcggagggttaaggtgaaaattgttgggggcgtcgagggtaggtgtggggaggggcggttttagggagtaaggagtttatttgtt

SEQ ID NO: 219 - DNA, 69 bp, Homo sapiens
  source 1..69: mol_type = genomic DNA
tcgctgactgcaggcaagcgtccgggaggggcggccaggcgaagccccggcgctttaccacacacttcc

SEQ ID NO: 220 - DNA, 69 bp, synthetic construct
  source 1..69: mol_type = other DNA
tcgttgattgtaggtaagcgttcgggaggggcggttaggcgaagtttcggcgttttattatatattttt

SEQ ID NO: 221 - DNA, 69 bp, synthetic construct
  source 1..69: mol_type = other DNA
aaaaatatataataaaacgccgaaacttcgcctaaccgcccctcccgaacgcttacctacaatcaacga

SEQ ID NO: 222 - DNA, 69 bp, synthetic construct
  source 1..69: mol_type = other DNA
tcgctaactacaaacaaacgtccgaaaaaaacgaccaaacgaaaccccgacgctttaccacacacttcc

SEQ ID NO: 223 - DNA, 69 bp, synthetic construct
  source 1..69: mol_type = other DNA
ggaagtgtgtggtaaagcgtcggggtttcgtttggtcgtttttttcggacgtttgtttgtagttagcga

SEQ ID NO: 224 - DNA, 1818 bp, Homo sapiens
  source 1..1818: mol_type = genomic DNA
gcgcgctagggtaggctggtctgtgctggatacgcgtgttcttctgcggagttaaagggtcggggacgggggttctggacttaccagagcaattccagccggtgggcgtttggcagtcacttaaggaggtagggaaagcagcgagcttcaccgggcgggctacgatgagtagcatgacgggcagcagcagcagccagcaaaagccctcgcaaagtgtccagctgctgcactgccgcggggactcccacagcaccatgactagttcgtgcaactctgcagcagcaaacggcttccgaggaacacaggatcgcgggggccgggcagcgggctactgagcatcccgcggacggcggcagcagaggcggcggcggtggcagtggcacccggcggggaagcagcagccaaacccgcgcatgatctcgagagtttcagcaacatccagggactgggctcagccccggagcgagagggtcgtccgctgagaagctgcgccggagacgcgggaagctgctgccataaggagggagctctgggaagccggaggacaggaggagacgggagtccaggggcagacgagtggagcccgaggaggcagggtggagggagagtcaaggcgccccgcagcccggcagccgcctctcgagctctgccgcccgcatccctctggcgtttgggaagcagcaggtcctcagcccgcccggggtcacgtgggaagaggcagtcgggctctgattggtggagcaggatgcaggtcccgggagggaggggtcgacgaggaggtgcaaggatgcaaggaggaggcggccgcggaagccacagatgggctcgctcgccaggcgctggcccgagtggggctaggcggggatggctcaaatgagaagctcgggcttcagggtgggctacccgcacactcatataccattcgcctcactctccgctccaggacgccccctaccgaaggcggggtccggactagcgcccctcttccgcgcgtgaccccgggccgcgagtgcgggccgcggctgggtggcgtctctccgagctggagatggtgggggcggaggtgtcagaggagcagcagcagcagggcagagaggggcgagtcggcgcgggagagggcgtcctgctggcgaccggcgctccagcgtgcgggagcgcgccgcctaggctgtagggggatgcaggctgggaatgtcgcggcggagaggccagggacgtttctctagggatttacaggaaagagggtgagaggcgatggtgttagaaccgctcttgccgacctggaagcaacagcagcatctcccacaagagcgtgcaaccccaaggctgctcgccgaggcagctcagccatcccggcaggcgctctccttccttctctcttctcccctctctcctcccaggccccccgcagctccgacccagcccaagcgttcgcaggtttgaatccctctcctcatcacccgctcctctccagcccgtagcctattagtgtgtccacctgggaggtgcggtcagatgtgtttggaaggtcagattggtcgggacaagtggtctgagagaaagagaaaggctcctctgcatacgccgcgggtgggttgccgggagcatcggccgggcagcggcgtccgggaaggggagagcgggctccatttgttggcccaggcagtgaccctgcgttccttactcgggtctttgccggatggccggtgacctggggcgacgagagaaggtctaactcggcaggagtctctggctctgcgcgtttctttcattctctccagcgggaagggcaaacggcatagcgggacccgccttccg

SEQ ID NO: 225 - DNA, 1818 bp, synthetic construct
  source 1..1818: mol_type = other DNA
gcgcgttagggtaggttggtttgtgttggatacgcgtgtttttttgcggagttaaagggtcggggacgggggttttggatttattagagtaattttagtcggtgggcgtttggtagttatttaaggaggtagggaaagtagcgagttttatcgggcgggttacgatgagtagtatgacgggtagtagtagtagttagtaaaagttttcgtaaagtgtttagttgttgtattgtcgcggggatttttatagtattatgattagttcgtgtaattttgtagtagtaaacggttttcgaggaatataggatcgcgggggtcgggtagcgggttattgagtatttcgcggacggcggtagtagaggcggcggcggtggtagtggtattcggcggggaagtagtagttaaattcgcgtatgatttcgagagttttagtaatatttagggattgggtttagtttcggagcgagagggtcgttcgttgagaagttgcgtcggagacgcgggaagttgttgttataaggagggagttttgggaagtcggaggataggaggagacgggagtttaggggtagacgagtggagttcgaggaggtagggtggagggagagttaaggcgtttcgtagttcggtagtcgtttttcgagttttgtcgttcgtatttttttggcgtttgggaagtagtaggtttttagttcgttcggggttacgtgggaagaggtagtcgggttttgattggtggagtaggatgtaggtttcgggagggaggggtcgacgaggaggtgtaaggatgtaaggaggaggcggtcgcggaagttatagatgggttcgttcgttaggcgttggttcgagtggggttaggcggggatggtttaaatgagaagttcgggttttagggtgggttattcgtatatttatatattattcgttttatttttcgttttaggacgttttttatcgaaggcggggttcggattagcgtttttttttcgcgcgtgatttcgggtcgcgagtgcgggtcgcggttgggtggcgttttttcgagttggagatggtgggggcggaggtgttagaggagtagtagtagtagggtagagaggggcgagtcggcgcgggagagggcgttttgttggcgatcggcgttttagcgtgcgggagcgcgtcgtttaggttgtagggggatgtaggttgggaatgtcgcggcggagaggttagggacgtttttttagggatttataggaaagagggtgagaggcgatggtgttagaatcgtttttgtcgatttggaagtaatagtagtattttttataagagcgtgtaattttaaggttgttcgtcgaggtagtttagttatttcggtaggcgttttttttttttttttttttttttttttttttttttaggtttttcgtagtttcgatttagtttaagcgttcgtaggtttgaatttttttttttattattcgtttttttttagttcgtagtttattagtgtgtttatttgggaggtgcggttagatgtgtttggaaggttagattggtcgggataagtggtttgagagaaagagaaaggtttttttgtatacgtcgcgggtgggttgtcgggagtatcggtcgggtagcggcgttcgggaaggggagagcgggttttatttgttggtttaggtagtgattttgcgttttttattcgggtttttgtcggatggtcggtgatttggggcgacgagagaaggtttaattcggtaggagtttttggttttgcgcgttttttttattttttttagcgggaagggtaaacggtatagcgggattcgtttttcg

SEQ ID NO: 226 - DNA, 1818 bp, synthetic construct
  source 1..1818: mol_type = other DNA
cgaaaaacgaatcccgctataccgtttacccttcccgctaaaaaaaataaaaaaaacgcgcaaaaccaaaaactcctaccgaattaaaccttctctcgtcgccccaaatcaccgaccatccgacaaaaacccgaataaaaaacgcaaaatcactacctaaaccaacaaataaaacccgctctccccttcccgaacgccgctacccgaccgatactcccgacaacccacccgcgacgtatacaaaaaaacctttctctttctctcaaaccacttatcccgaccaatctaaccttccaaacacatctaaccgcacctcccaaataaacacactaataaactacgaactaaaaaaaaacgaataataaaaaaaaaaattcaaacctacgaacgcttaaactaaatcgaaactacgaaaaacctaaaaaaaaaaaaaaaaaaaaaaaaaaaaaaaaaaaacgcctaccgaaataactaaactacctcgacgaacaaccttaaaattacacgctcttataaaaaatactactattacttccaaatcgacaaaaacgattctaacaccatcgcctctcaccctctttcctataaatccctaaaaaaacgtccctaacctctccgccgcgacattcccaacctacatccccctacaacctaaacgacgcgctcccgcacgctaaaacgccgatcgccaacaaaacgccctctcccgcgccgactcgcccctctctaccctactactactactcctctaacacctccgcccccaccatctccaactcgaaaaaacgccacccaaccgcgacccgcactcgcgacccgaaatcacgcgcgaaaaaaaaacgctaatccgaaccccgccttcgataaaaaacgtcctaaaacgaaaaataaaacgaataatatataaatatacgaataacccaccctaaaacccgaacttctcatttaaaccatccccgcctaaccccactcgaaccaacgcctaacgaacgaacccatctataacttccgcgaccgcctcctccttacatccttacacctcctcgtcgacccctccctcccgaaacctacatcctactccaccaatcaaaacccgactacctcttcccacgtaaccccgaacgaactaaaaacctactacttcccaaacgccaaaaaaatacgaacgacaaaactcgaaaaacgactaccgaactacgaaacgccttaactctccctccaccctacctcctcgaactccactcgtctacccctaaactcccgtctcctcctatcctccgacttcccaaaactccctccttataacaacaacttcccgcgtctccgacgcaacttctcaacgaacgaccctctcgctccgaaactaaacccaatccctaaatattactaaaactctcgaaatcatacgcgaatttaactactacttccccgccgaataccactaccaccgccgccgcctctactaccgccgtccgcgaaatactcaataacccgctacccgacccccgcgatcctatattcctcgaaaaccgtttactactacaaaattacacgaactaatcataatactataaaaatccccgcgacaatacaacaactaaacactttacgaaaacttttactaactactactactacccgtcatactactcatcgtaacccgcccgataaaactcgctactttccctacctccttaaataactaccaaacgcccaccgactaaaattactctaataaatccaaaacccccgtccccgaccctttaactccgcaaaaaaacacgcgtatccaacacaaaccaacctaccctaacgcgc

SEQ ID NO: 227 - DNA, 1818 bp, synthetic construct
  source 1..1818: mol_type = other DNA
acgcgctaaaataaactaatctatactaaatacgcgtattcttctacgaaattaaaaaatcgaaaacgaaaattctaaacttaccaaaacaattccaaccgataaacgtttaacaatcacttaaaaaaataaaaaaaacaacgaacttcaccgaacgaactacgataaataacataacgaacaacaacaacaaccaacaaaaaccctcgcaaaatatccaactactacactaccgcgaaaactcccacaacaccataactaattcgtacaactctacaacaacaaacgacttccgaaaaacacaaaatcgcgaaaaccgaacaacgaactactaaacatcccgcgaacgacgacaacaaaaacgacgacgataacaataacacccgacgaaaaaacaacaaccaaacccgcgcataatctcgaaaatttcaacaacatccaaaaactaaactcaaccccgaaacgaaaaaatcgtccgctaaaaaactacgccgaaaacgcgaaaaactactaccataaaaaaaaaactctaaaaaaccgaaaaacaaaaaaaaacgaaaatccaaaaacaaacgaataaaacccgaaaaaacaaaataaaaaaaaaatcaaaacgccccgcaacccgacaaccgcctctcgaactctaccgcccgcatccctctaacgtttaaaaaacaacaaatcctcaacccgcccgaaatcacgtaaaaaaaaacaatcgaactctaattaataaaacaaaatacaaatcccgaaaaaaaaaaatcgacgaaaaaatacaaaaatacaaaaaaaaaacgaccgcgaaaaccacaaataaactcgctcgccaaacgctaacccgaataaaactaaacgaaaataactcaaataaaaaactcgaacttcaaaataaactacccgcacactcatataccattcgcctcactctccgctccaaaacgccccctaccgaaaacgaaatccgaactaacgcccctcttccgcgcgtaaccccgaaccgcgaatacgaaccgcgactaaataacgtctctccgaactaaaaataataaaaacgaaaatatcaaaaaaacaacaacaacaaaacaaaaaaaaacgaatcgacgcgaaaaaaaacgtcctactaacgaccgacgctccaacgtacgaaaacgcgccgcctaaactataaaaaaatacaaactaaaaatatcgcgacgaaaaaaccaaaaacgtttctctaaaaatttacaaaaaaaaaaataaaaaacgataatattaaaaccgctcttaccgacctaaaaacaacaacaacatctcccacaaaaacgtacaaccccaaaactactcgccgaaacaactcaaccatcccgacaaacgctctccttccttctctcttctcccctctctcctcccaaaccccccgcaactccgacccaacccaaacgttcgcaaatttaaatccctctcctcatcacccgctcctctccaacccgtaacctattaatatatccacctaaaaaatacgatcaaatatatttaaaaaatcaaattaatcgaaacaaataatctaaaaaaaaaaaaaaaactcctctacatacgccgcgaataaattaccgaaaacatcgaccgaacaacgacgtccgaaaaaaaaaaaacgaactccatttattaacccaaacaataaccctacgttccttactcgaatctttaccgaataaccgataacctaaaacgacgaaaaaaaatctaactcgacaaaaatctctaactctacgcgtttctttcattctctccaacgaaaaaaacaaacgacataacgaaacccgccttccg

SEQ ID NO: 228 - DNA, 1818 bp, synthetic construct
  source 1..1818: mol_type = other DNA
cggaaggcgggtttcgttatgtcgtttgtttttttcgttggagagaatgaaagaaacgcgtagagttagagatttttgtcgagttagattttttttcgtcgttttaggttatcggttattcggtaaagattcgagtaaggaacgtagggttattgtttgggttaataaatggagttcgttttttttttttcggacgtcgttgttcggtcgatgttttcggtaatttattcgcggcgtatgtagaggagttttttttttttttttagattatttgtttcgattaatttgattttttaaatatatttgatcgtattttttaggtggatatattaataggttacgggttggagaggagcgggtgatgaggagagggatttaaatttgcgaacgtttgggttgggtcggagttgcggggggtttgggaggagagaggggagaagagagaaggaaggagagcgtttgtcgggatggttgagttgtttcggcgagtagttttggggttgtacgtttttgtgggagatgttgttgttgtttttaggtcggtaagagcggttttaatattatcgttttttatttttttttttgtaaatttttagagaaacgtttttggttttttcgtcgcgatatttttagtttgtatttttttatagtttaggcggcgcgttttcgtacgttggagcgtcggtcgttagtaggacgttttttttcgcgtcgattcgtttttttttgttttgttgttgttgtttttttgatattttcgtttttattatttttagttcggagagacgttatttagtcgcggttcgtattcgcggttcggggttacgcgcggaagaggggcgttagttcggatttcgttttcggtagggggcgttttggagcggagagtgaggcgaatggtatatgagtgtgcgggtagtttattttgaagttcgagttttttatttgagttattttcgtttagttttattcgggttagcgtttggcgagcgagtttatttgtggttttcgcggtcgttttttttttgtatttttgtattttttcgtcgatttttttttttcgggatttgtattttgttttattaattagagttcgattgtttttttttacgtgatttcgggcgggttgaggatttgttgttttttaaacgttagagggatgcgggcggtagagttcgagaggcggttgtcgggttgcggggcgttttgattttttttttattttgttttttcgggttttattcgtttgtttttggattttcgtttttttttgtttttcggttttttagagttttttttttatggtagtagtttttcgcgttttcggcgtagttttttagcggacgattttttcgtttcggggttgagtttagtttttggatgttgttgaaattttcgagattatgcgcgggtttggttgttgttttttcgtcgggtgttattgttatcgtcgtcgtttttgttgtcgtcgttcgcgggatgtttagtagttcgttgttcggttttcgcgattttgtgtttttcggaagtcgtttgttgttgtagagttgtacgaattagttatggtgttgtgggagttttcgcggtagtgtagtagttggatattttgcgagggtttttgttggttgttgttgttgttcgttatgttatttatcgtagttcgttcggtgaagttcgttgttttttttatttttttaagtgattgttaaacgtttatcggttggaattgttttggtaagtttagaattttcgttttcgattttttaatttcgtagaagaatacgcgtatttagtatagattagtttattttagcgcgt

SEQ ID NO: 229 - DNA, 1074 bp, Homo sapiens
  source 1..1074: mol_type = genomic DNA
cagaggagcagcagcagcagggcagagaggggcgagtcggcgcgggagagggcgtcctgctggcgaccggcgctccagcgtgcgggagcgcgccgcctaggctgtagggggatgcaggctgggaatgtcgcggcggagaggccagggacgtttctctagggatttacaggaaagagggtgagaggcgatggtgttagaaccgctcttgccgacctggaagcaacagcagcatctcccacaagagcgtgcaaccccaaggctgctcgccgaggcagctcagccatcccggcaggcgctctccttccttctctcttctcccctctctcctcccaggccccccgcagctccgacccagcccaagcgttcgcaggtttgaatccctctcctcatcacccgctcctctccagcccgtagcctattagtgtgtccacctgggaggtgcggtcagatgtgtttggaaggtcagattggtcgggacaagtggtctgagagaaagagaaaggctcctctgcatacgccgcgggtgggttgccgggagcatcggccgggcagcggcgtccgggaaggggagagcgggctccatttgttggcccaggcagtgaccctgcgttccttactcgggtctttgccggatggccggtgacctggggcgacgagagaaggtctaactcggcaggagtctctggctctgcgcgtttctttcattctctccagcgggaagggcaaacggcatagcgggacccgccttccgtctgctgcattcttcaggcagttagacacactctttagcctaatggaattttagtcgccagtaacgggaccaagagctttcggggacaagggtggagaggaacatctttcttccatgaccggggtcactattgcagtctcagtgttttggatgccccatagggaagagctttctttttggtgtgtgattattcagtgattcctgtttttgtttttgtttatcttctctccgctctccttctctattcctctctgttatccctcctccttcttctccccccgctttcaaaagcctccggatcctccctttttctatttaaattc

SEQ ID NO: 230 - DNA, 1074 bp, synthetic construct
  source 1..1074: mol_type = other DNA
tagaggagtagtagtagtagggtagagaggggcgagtcggcgcgggagagggcgttttgttggcgatcggcgttttagcgtgcgggagcgcgtcgtttaggttgtagggggatgtaggttgggaatgtcgcggcggagaggttagggacgtttttttagggatttataggaaagagggtgagaggcgatggtgttagaatcgtttttgtcgatttggaagtaatagtagtattttttataagagcgtgtaattttaaggttgttcgtcgaggtagtttagttatttcggtaggcgttttttttttttttttttttttttttttttttttttaggtttttcgtagtttcgatttagtttaagcgttcgtaggtttgaatttttttttttattattcgtttttttttagttcgtagtttattagtgtgtttatttgggaggtgcggttagatgtgtttggaaggttagattggtcgggataagtggtttgagagaaagagaaaggtttttttgtatacgtcgcgggtgggttgtcgggagtatcggtcgggtagcggcgttcgggaaggggagagcgggttttatttgttggtttaggtagtgattttgcgttttttattcgggtttttgtcggatggtcggtgatttggggcgacgagagaaggtttaattcggtaggagtttttggttttgcgcgttttttttattttttttagcgggaagggtaaacggtatagcgggattcgtttttcgtttgttgtattttttaggtagttagatatattttttagtttaatggaattttagtcgttagtaacgggattaagagttttcggggataagggtggagaggaatattttttttttatgatcggggttattattgtagttttagtgttttggatgttttatagggaagagttttttttttggtgtgtgattatttagtgatttttgtttttgtttttgtttatttttttttcgttttttttttttatttttttttgttattttttttttttttttttttttcgtttttaaaagttttcggattttttttttttttatttaaattt

SEQ ID NO: 231 - DNA, 1074 bp, synthetic construct
  source 1..1074: mol_type = other DNA
aaatttaaataaaaaaaaaaaaaatccgaaaacttttaaaaacgaaaaaaaaaaaaaaaaaaaaaataacaaaaaaaaataaaaaaaaaaaacgaaaaaaaaataaacaaaaacaaaaacaaaaatcactaaataatcacacaccaaaaaaaaaactcttccctataaaacatccaaaacactaaaactacaataataaccccgatcataaaaaaaaaatattcctctccacccttatccccgaaaactcttaatcccgttactaacgactaaaattccattaaactaaaaaatatatctaactacctaaaaaatacaacaaacgaaaaacgaatcccgctataccgtttacccttcccgctaaaaaaaataaaaaaaacgcgcaaaaccaaaaactcctaccgaattaaaccttctctcgtcgccccaaatcaccgaccatccgacaaaaacccgaataaaaaacgcaaaatcactacctaaaccaacaaataaaacccgctctccccttcccgaacgccgctacccgaccgatactcccgacaacccacccgcgacgtatacaaaaaaacctttctctttctctcaaaccacttatcccgaccaatctaaccttccaaacacatctaaccgcacctcccaaataaacacactaataaactacgaactaaaaaaaaacgaataataaaaaaaaaaattcaaacctacgaacgcttaaactaaatcgaaactacgaaaaacctaaaaaaaaaaaaaaaaaaaaaaaaaaaaaaaaaaaacgcctaccgaaataactaaactacctcgacgaacaaccttaaaattacacgctcttataaaaaatactactattacttccaaatcgacaaaaacgattctaacaccatcgcctctcaccctctttcctataaatccctaaaaaaacgtccctaacctctccgccgcgacattcccaacctacatccccctacaacctaaacgacgcgctcccgcacgctaaaacgccgatcgccaacaaaacgccctctcccgcgccgactcgcccctctctaccctactactactactcctcta

SEQ ID NO: 232 - DNA, 1074 bp, synthetic construct
  source 1..1074: mol_type = other DNA
caaaaaaacaacaacaacaaaacaaaaaaaaacgaatcgacgcgaaaaaaaacgtcctactaacgaccgacgctccaacgtacgaaaacgcgccgcctaaactataaaaaaatacaaactaaaaatatcgcgacgaaaaaaccaaaaacgtttctctaaaaatttacaaaaaaaaaaataaaaaacgataatattaaaaccgctcttaccgacctaaaaacaacaacaacatctcccacaaaaacgtacaaccccaaaactactcgccgaaacaactcaaccatcccgacaaacgctctccttccttctctcttctcccctctctcctcccaaaccccccgcaactccgacccaacccaaacgttcgcaaatttaaatccctctcctcatcacccgctcctctccaacccgtaacctattaatatatccacctaaaaaatacgatcaaatatatttaaaaaatcaaattaatcgaaacaaataatctaaaaaaaaaaaaaaaactcctctacatacgccgcgaataaattaccgaaaacatcgaccgaacaacgacgtccgaaaaaaaaaaaacgaactccatttattaacccaaacaataaccctacgttccttactcgaatctttaccgaataaccgataacctaaaacgacgaaaaaaaatctaactcgacaaaaatctctaactctacgcgtttctttcattctctccaacgaaaaaaacaaacgacataacgaaacccgccttccgtctactacattcttcaaacaattaaacacactctttaacctaataaaattttaatcgccaataacgaaaccaaaaactttcgaaaacaaaaataaaaaaaaacatctttcttccataaccgaaatcactattacaatctcaatattttaaataccccataaaaaaaaactttctttttaatatataattattcaataattcctatttttatttttatttatcttctctccgctctccttctctattcctctctattatccctcctccttcttctccccccgctttcaaaaacctccgaatcctccctttttctatttaaattc

SEQ ID NO: 233 - DNA, 1074 bp, synthetic construct
  source 1..1074: mol_type = other DNA
gaatttaaatagaaaaagggaggattcggaggtttttgaaagcggggggagaagaaggaggagggataatagagaggaatagagaaggagagcggagagaagataaataaaaataaaaataggaattattgaataattatatattaaaaagaaagtttttttttatggggtatttaaaatattgagattgtaatagtgatttcggttatggaagaaagatgtttttttttatttttgttttcgaaagtttttggtttcgttattggcgattaaaattttattaggttaaagagtgtgtttaattgtttgaagaatgtagtagacggaaggcgggtttcgttatgtcgtttgtttttttcgttggagagaatgaaagaaacgcgtagagttagagatttttgtcgagttagattttttttcgtcgttttaggttatcggttattcggtaaagattcgagtaaggaacgtagggttattgtttgggttaataaatggagttcgttttttttttttcggacgtcgttgttcggtcgatgttttcggtaatttattcgcggcgtatgtagaggagttttttttttttttttagattatttgtttcgattaatttgattttttaaatatatttgatcgtattttttaggtggatatattaataggttacgggttggagaggagcgggtgatgaggagagggatttaaatttgcgaacgtttgggttgggtcggagttgcggggggtttgggaggagagaggggagaagagagaaggaaggagagcgtttgtcgggatggttgagttgtttcggcgagtagttttggggttgtacgtttttgtgggagatgttgttgttgtttttaggtcggtaagagcggttttaatattatcgttttttatttttttttttgtaaatttttagagaaacgtttttggttttttcgtcgcgatatttttagtttgtatttttttatagtttaggcggcgcgttttcgtacgttggagcgtcggtcgttagtaggacgttttttttcgcgtcgattcgtttttttttgttttgttgttgttgtttttttg

SEQ ID NO: 234 - DNA, 74 bp, Homo sapiens
  source 1..74: mol_type = genomic DNA
aggctcctctgcatacgccgcgggtgggttgccgggagcatcggccgggcagcggcgtccgggaaggggagagc

SEQ ID NO: 235 - DNA, 74 bp, synthetic construct
  source 1..74: mol_type = other DNA
aggtttttttgtatacgtcgcgggtgggttgtcgggagtatcggtcgggtagcggcgttcgggaaggggagagt

SEQ ID NO: 236 - DNA, 74 bp, synthetic construct
  source 1..74: mol_type = other DNA
actctccccttcccgaacgccgctacccgaccgatactcccgacaacccacccgcgacgtatacaaaaaaacct

SEQ ID NO: 237 - DNA, 74 bp, synthetic construct
  source 1..74: mol_type = other DNA
aaactcctctacatacgccgcgaataaattaccgaaaacatcgaccgaacaacgacgtccgaaaaaaaaaaaac

SEQ ID NO: 238 - DNA, 74 bp, synthetic construct
  source 1..74: mol_type = other DNA
gttttttttttttcggacgtcgttgttcggtcgatgttttcggtaatttattcgcggcgtatgtagaggagttt

SEQ ID NO: 239 - DNA, 1131 bp, Homo sapiens
  source 1..1131: mol_type = genomic DNA
aggtgtgcaggcgctacaagaaggagccggggccgctgcccccgcagcgcggacttcgccccgtgctgcacacgctttagaagagcgtgtccggaagtgtcctccccggccctcctcgcttcccaagatttgtttgcaacgtctacacccgtcaccaccgcataagcaccagggtgaggaggtctctttgcaggaattaatatcgatttcagtctcctgctccttcaaagtttctctttgacgatggcgagacttatattctaagtcagaagaaaataaaaccttgcctattctgggcccatgattgcacaaaatccaaaatgctgccggcatgaagatgcagctggaagactctagaaacctagcattaggaggcagagactttatagtgcccagagatgagttcctaaataaactaagaattacgcagtgggaaattaaggaggagggtctgaggggccgacctcagagggacagatagagaaatcgagagcgaaacactcaaggaagtggaacaaatcgtcgtagccaggcgtctctgcctctccggaaacccgaaatccgcccaaacctcatcccaggcgctcacggtaccagtcgcagggccccgcactataggggctgccaccctctgcccccggagtccccacgccgagactcatcatttgctggctctttcttccttttcttcttttgtttttttatccttcaaatataagacaaaaatacacctcatttatcatcatagatatatatatttcactctcaccacataaatacaaaacatccaaatatccaaaacattcagaacaaagttagtagcctggattcagtgacacctacccgcgtttgaatcgttcattattttcttcactatttgcctagaaaaaaaaaaaagatgaaaaagagggggagagtttccgacgtaaataatttacaataaaattactattttaaaaaagtgtttttcgcttgcttcagatggatgaatacattctagacactgcggcacctcgcagtctgggtctgtgattttggttaaaatgtgggtggccgtggctgtagggacgatcgccgctgcgcagggtaaggaaagagggtgtcgcggttcgtgttctgatttgggaccac

SEQ ID NO: 240 - DNA, 1131 bp, synthetic construct
  source 1..1131: mol_type = other DNA
aggtgtgtaggcgttataagaaggagtcggggtcgttgttttcgtagcgcggatttcgtttcgtgttgtatacgttttagaagagcgtgttcggaagtgtttttttcggtttttttcgttttttaagatttgtttgtaacgtttatattcgttattatcgtataagtattagggtgaggaggtttttttgtaggaattaatatcgattttagttttttgtttttttaaagtttttttttgacgatggcgagatttatattttaagttagaagaaaataaaattttgtttattttgggtttatgattgtataaaatttaaaatgttgtcggtatgaagatgtagttggaagattttagaaatttagtattaggaggtagagattttatagtgtttagagatgagtttttaaataaattaagaattacgtagtgggaaattaaggaggagggtttgaggggtcgattttagagggatagatagagaaatcgagagcgaaatatttaaggaagtggaataaatcgtcgtagttaggcgtttttgttttttcggaaattcgaaattcgtttaaattttattttaggcgtttacggtattagtcgtagggtttcgtattataggggttgttattttttgttttcggagtttttacgtcgagatttattatttgttggtttttttttttttttttttttttgtttttttattttttaaatataagataaaaatatattttatttattattatagatatatatattttatttttattatataaatataaaatatttaaatatttaaaatatttagaataaagttagtagtttggatttagtgatatttattcgcgtttgaatcgtttattattttttttattatttgtttagaaaaaaaaaaaagatgaaaaagagggggagagttttcgacgtaaataatttataataaaattattattttaaaaaagtgtttttcgtttgttttagatggatgaatatattttagatattgcggtatttcgtagtttgggtttgtgattttggttaaaatgtgggtggtcgtggttgtagggacgatcgtcgttgcgtagggtaaggaaagagggtgtcgcggttcgtgttttgatttgggattat

SEQ ID NO: 241 - DNA, 1131 bp, synthetic construct
  source 1..1131: mol_type = other DNA
ataatcccaaatcaaaacacgaaccgcgacaccctctttccttaccctacgcaacgacgatcgtccctacaaccacgaccacccacattttaaccaaaatcacaaacccaaactacgaaataccgcaatatctaaaatatattcatccatctaaaacaaacgaaaaacacttttttaaaataataattttattataaattatttacgtcgaaaactctccccctctttttcatcttttttttttttctaaacaaataataaaaaaaataataaacgattcaaacgcgaataaatatcactaaatccaaactactaactttattctaaatattttaaatatttaaatattttatatttatataataaaaataaaatatatatatctataataataaataaaatatatttttatcttatatttaaaaaataaaaaaacaaaaaaaaaaaaaaaaaaaaaaaccaacaaataataaatctcgacgtaaaaactccgaaaacaaaaaataacaacccctataatacgaaaccctacgactaataccgtaaacgcctaaaataaaatttaaacgaatttcgaatttccgaaaaaacaaaaacgcctaactacgacgatttattccacttccttaaatatttcgctctcgatttctctatctatccctctaaaatcgacccctcaaaccctcctccttaatttcccactacgtaattcttaatttatttaaaaactcatctctaaacactataaaatctctacctcctaatactaaatttctaaaatcttccaactacatcttcataccgacaacattttaaattttatacaatcataaacccaaaataaacaaaattttattttcttctaacttaaaatataaatctcgccatcgtcaaaaaaaaactttaaaaaaacaaaaaactaaaatcgatattaattcctacaaaaaaacctcctcaccctaatacttatacgataataacgaatataaacgttacaaacaaatcttaaaaaacgaaaaaaaccgaaaaaaacacttccgaacacgctcttctaaaacgtatacaacacgaaacgaaatccgcgctacgaaaacaacgaccccgactccttcttataacgcctacacacct

SEQ ID NO: 242 - DNA, 1131 bp, synthetic construct
  source 1..1131: mol_type = other DNA
aaatatacaaacgctacaaaaaaaaaccgaaaccgctacccccgcaacgcgaacttcgccccgtactacacacgctttaaaaaaacgtatccgaaaatatcctccccgaccctcctcgcttcccaaaatttatttacaacgtctacacccgtcaccaccgcataaacaccaaaataaaaaaatctctttacaaaaattaatatcgatttcaatctcctactccttcaaaatttctctttaacgataacgaaacttatattctaaatcaaaaaaaaataaaaccttacctattctaaacccataattacacaaaatccaaaatactaccgacataaaaatacaactaaaaaactctaaaaacctaacattaaaaaacaaaaactttataatacccaaaaataaattcctaaataaactaaaaattacgcaataaaaaattaaaaaaaaaaatctaaaaaaccgacctcaaaaaaacaaataaaaaaatcgaaaacgaaacactcaaaaaaataaaacaaatcgtcgtaaccaaacgtctctacctctccgaaaacccgaaatccgcccaaacctcatcccaaacgctcacgataccaatcgcaaaaccccgcactataaaaactaccaccctctacccccgaaatccccacgccgaaactcatcatttactaactctttcttccttttcttcttttatttttttatccttcaaatataaaacaaaaatacacctcatttatcatcataaatatatatatttcactctcaccacataaatacaaaacatccaaatatccaaaacattcaaaacaaaattaataacctaaattcaataacacctacccgcgtttaaatcgttcattattttcttcactatttacctaaaaaaaaaaaaaaaataaaaaaaaaaaaaaaaatttccgacgtaaataatttacaataaaattactattttaaaaaaatatttttcgcttacttcaaataaataaatacattctaaacactacgacacctcgcaatctaaatctataattttaattaaaatataaataaccgtaactataaaaacgatcgccgctacgcaaaataaaaaaaaaaaatatcgcgattcgtattctaatttaaaaccac

SEQ ID NO: 243 - DNA, 1131 bp, synthetic construct
  source 1..1131: mol_type = other DNA
gtggttttaaattagaatacgaatcgcgatattttttttttttattttgcgtagcggcgatcgtttttatagttacggttatttatattttaattaaaattatagatttagattgcgaggtgtcgtagtgtttagaatgtatttatttatttgaagtaagcgaaaaatatttttttaaaatagtaattttattgtaaattatttacgtcggaaatttttttttttttttttattttttttttttttttaggtaaatagtgaagaaaataatgaacgatttaaacgcgggtaggtgttattgaatttaggttattaattttgttttgaatgttttggatatttggatgttttgtatttatgtggtgagagtgaaatatatatatttatgatgataaatgaggtgtatttttgttttatatttgaaggataaaaaaataaaagaagaaaaggaagaaagagttagtaaatgatgagtttcggcgtggggatttcgggggtagagggtggtagtttttatagtgcggggttttgcgattggtatcgtgagcgtttgggatgaggtttgggcggatttcgggttttcggagaggtagagacgtttggttacgacgatttgttttatttttttgagtgtttcgttttcgatttttttatttgtttttttgaggtcggttttttagatttttttttttaattttttattgcgtaatttttagtttatttaggaatttatttttgggtattataaagtttttgttttttaatgttaggtttttagagttttttagttgtatttttatgtcggtagtattttggattttgtgtaattatgggtttagaataggtaaggttttatttttttttgatttagaatataagtttcgttatcgttaaagagaaattttgaaggagtaggagattgaaatcgatattaatttttgtaaagagatttttttattttggtgtttatgcggtggtgacgggtgtagacgttgtaaataaattttgggaagcgaggagggtcggggaggatattttcggatacgtttttttaaagcgtgtgtagtacggggcgaagttcgcgttgcgggggtagcggtttcggtttttttttgtagcgtttgtatattt

SEQ ID NO: 244 - DNA, 131 bp, Homo sapiens
  source 1..131: mol_type = genomic DNA
ctcaaggaagtggaacaaatcgtcgtagccaggcgtctctgcctctccggaaacccgaaatccgcccaaacctcatcccaggcgctcacggtaccagtcgcagggccccgcactataggggctgccaccct

SEQ ID NO: 245 - DNA, 131 bp, synthetic construct
  source 1..131: mol_type = other DNA
tttaaggaagtggaataaatcgtcgtagttaggcgtttttgttttttcggaaattcgaaattcgtttaaattttattttaggcgtttacggtattagtcgtagggtttcgtattataggggttgttatttt

SEQ ID NO: 246 - DNA, 131 bp, synthetic construct
  source 1..131: mol_type = other DNA
aaaataacaacccctataatacgaaaccctacgactaataccgtaaacgcctaaaataaaatttaaacgaatttcgaatttccgaaaaaacaaaaacgcctaactacgacgatttattccacttccttaaa

SEQ ID NO: 247 - DNA, 131 bp, synthetic construct
  source 1..131: mol_type = other DNA
ctcaaaaaaataaaacaaatcgtcgtaaccaaacgtctctacctctccgaaaacccgaaatccgcccaaacctcatcccaaacgctcacgataccaatcgcaaaaccccgcactataaaaactaccaccct

SEQ ID NO: 248 - DNA, 131 bp, synthetic construct
  source 1..131: mol_type = other DNA
agggtggtagtttttatagtgcggggttttgcgattggtatcgtgagcgtttgggatgaggtttgggcggatttcgggttttcggagaggtagagacgtttggttacgacgatttgttttatttttttgag

SEQ ID NO: 249 - DNA, 21 bp, synthetic construct
  source 1..21: mol_type = other DNA
ggtgttttaaggttattaaac

SEQ ID NO: 250 - DNA, 19 bp, synthetic construct
  source 1..19: mol_type = other DNA
ctcgatccccctcctcgta

SEQ ID NO: 251 - DNA, 20 bp, synthetic construct
  source 1..20: mol_type = other DNA
gagaaggagtattgttgttc

SEQ ID NO: 252 - DNA, 22 bp, synthetic construct
  source 1..22: mol_type = other DNA
actatccccctaaaaatactcg

SEQ ID NO: 253 - DNA, 21 bp, synthetic construct
  source 1..21: mol_type = other DNA
tgtaggttgtagtaggttttc

SEQ ID NO: 254 - DNA, 19 bp, synthetic construct
  source 1..19: mol_type = other DNA
atacctcctctaacaaacg

SEQ ID NO: 255 - DNA, 19 bp, synthetic construct
  source 1..19: mol_type = other DNA
atttaaattttcgagttcg

SEQ ID NO: 256 - DNA, 19 bp, synthetic construct
  source 1..19: mol_type = other DNA
attccctaatactaatccc

SEQ ID NO: 257 - DNA, 26 bp, synthetic construct
  source 1..26: mol_type = other DNA
gagttgagagaatttttaaatttttc

SEQ ID NO: 258 - DNA, 22 bp, synthetic construct
  source 1..22: mol_type = other DNA
tcttctaaaacaaaaaacaccg

SEQ ID NO: 259 - DNA, 20 bp, synthetic construct
  source 1..20: mol_type = other DNA
agtttatagagttagggttc

SEQ ID NO: 260 - DNA, 21 bp, synthetic construct
  source 1..21: mol_type = other DNA
tccaaaaaaaacttcctttcg

SEQ ID NO: 261 - DNA, 17 bp, synthetic construct
  source 1..17: mol_type = other DNA
ggttgaaattagagcgc

SEQ ID NO: 262 - DNA, 17 bp, synthetic construct
  source 1..17: mol_type = other DNA
tacctccattaccgacg

SEQ ID NO: 263 - DNA, 20 bp, synthetic construct
  source 1..20: mol_type = other DNA
aagtggtattgaagattatc

SEQ ID NO: 264 - DNA, 19 bp, synthetic construct
  source 1..19: mol_type = other DNA
ctcgccgaaaaaaaaaccg

SEQ ID NO: 265 - DNA, 19 bp, synthetic construct
  source 1..19: mol_type = other DNA
gaatgtaaagtaagcgttc

SEQ ID NO: 266 - DNA, 18 bp, synthetic construct
  source 1..18: mol_type = other DNA
cttctctcgccccgaacg

SEQ ID NO: 267 - DNA, 18 bp, synthetic construct
  source 1..18: mol_type = other DNA
atcgaaaacgctacaccg

SEQ ID NO: 268 - DNA, 19 bp, synthetic construct
  source 1..19: mol_type = other DNA
gaggttcggagatttttcg

SEQ ID NO: 269 - DNA, 19 bp, synthetic construct
  source 1..19: mol_type = other DNA
gttttggttatgtagggac

SEQ ID NO: 270 - DNA, 21 bp, synthetic construct
  source 1..21: mol_type = other DNA
cctacctataacttctctccg

SEQ ID NO: 271 - DNA, 19 bp, synthetic construct
  source 1..19: mol_type = other DNA
tcgctaactacaaacaaac

SEQ ID NO: 272 - DNA, 19 bp, synthetic construct
  source 1..19: mol_type = other DNA
ggaagtgtgtggtaaagcg

SEQ ID NO: 273 - DNA, 19 bp, synthetic construct
  source 1..19: mol_type = other DNA
aggtttttttgtatacgtc

SEQ ID NO: 274 - DNA, 19 bp, synthetic construct
  source 1..19: mol_type = other DNA
gctctccccttcccgaacg

SEQ ID NO: 275 - DNA, 21 bp, synthetic construct
  source 1..21: mol_type = other DNA
tttaaggaagtggaataaatc

SEQ ID NO: 276 - DNA, 23 bp, synthetic construct
  source 1..23: mol_type = other DNA
aaaataacaacccctataatacg